(19) United States
(12) Patent Application Publication
(10) Pub. No.: US 2025/0354108 A1
(43) Pub. Date: Nov. 20, 2025

(54) METHODS FOR PRODUCING MONOTERPENE INDOLE ALKALOIDS

(71) Applicant: Danmarks Tekniske Universitet, Kongens Lyngby (DK)

(72) Inventors: Michael Krogh Jensen, Kongens Lyngby (DK); Lea Gram Hansen, Kongens Lyngby (DK); Jie Zhang, Kongens Lyngby (DK); Jay D. Keasling, Kongens Lyngby (DK); Samuel Alan Bradley, Kongens Lyngby (DK); Vasil D'Ambrosio, Kongens Lyngby (DK); Nils Emil Junge Marcussen, Kongens Lyndby (DK)

(21) Appl. No.: 18/860,052

(22) PCT Filed: May 19, 2023
(86) PCT No.: PCT/EP2023/063481

§ 371 Date: Oct. 25, 2024

Related U.S. Application Data
(60) Provisional application No. 63/343,622, filed on May 19, 2022.

(30) Foreign Application Priority Data
Jul. 7, 2022 (EP) 22183656.2

(51) Int. Cl. C12N 1/18; A61K 31/437; A61K 31/4375; C12N 9/02; C12N 9/10; C12N 9/16; C12N 9/24; C12P 17/18; C12R 1/85

(52) U.S. Cl. CPC C12N 1/18; A61K 31/437; A61K 31/4375; C12N 9/004; C12N 9/0042; C12N 9/0071; C12N 9/1029; C12N 9/16; C12N 9/2405; C12P 17/182; C12P 17/188; C12R 2001/85; C12Y 114/19; C12Y 203/0116; C12Y 301/07011; C12Y 302/01105

(57) ABSTRACT

The present invention relates to microorganisms for producing monoterpene indole al-kaloids (MIAs) and derivatives thereof de novo, including halogenated MIAs and halo-genated derivatives thereof. Also provided herein are methods for producing MIAs and derivatives thereof de novo, in particular halogenated MIAs and derivatives thereof, in a 5 microorganism, as well as useful nucleic acids, vectors and host cells for performing the present methods.

### TECHNICAL FIELD

The present invention relates to microorganisms for producing monoterpene indole alkaloids (MIAs) and derivatives thereof de novo, including halogenated MIAs and halogenated derivatives thereof. Also provided herein are methods for producing MIAs and derivatives thereof de novo, in particular halogenated MIAs and derivatives thereof, in a microorganism, as well as useful nucleic acids, vectors and host cells for performing the present methods.

### BACKGROUND

The monoterpene indole alkaloids (MIAs) are a significant group of plant secondary metabolites possessing various medicinal properties. Producing MIAs at scale for medicinal use is challenging. Source extraction from plants can suffer from supply chain shortages and generally poor yields, while total chemical synthesis is plagued by difficulties separating stereoisomers.

Microbial cell factories, engineered to produce phytochemicals in large scale fermentations, are an emerging solution to production of both natural and modified MIAs, due to the ability of enzymes to catalyse reactions not observed in their natural repertoire. Expression of plant genes in baker's yeast Saccharomyces cerevisiae has facilitated heterologous production of various plant-derived pharmaceuticals, including MIAs (Thodey et al. 2014, Billingsley et al. 2017, Li et al. 2018, Liu et al. 2022, Misa et al. 2022). Strictosidine is the branch point metabolite from which all >3,000 naturally occurring MIAs are derived. In 2015, Brown et al. (2015) refactored the 12-step strictosidine pathway in S. cerevisiae. Additionally, separate modules for valorization of commercially available MIA precursors to down-stream active pharmaceutical ingredients have been demonstrated (Qu et al. 2015, Kulagina et al. 2021).

Several studies have investigated enzymatic production of unnatural MIAs in vitro and in planta. Early investigations into the promiscuity of individual enzymes were followed by heterologous enzyme expression for directly introducing unnatural elements into MIA precursors (Runguphan et al. 2010). More specifically, two tryptophan halogenases were expressed in the MIA-producing plant Catharanthus roseus, resulting in de novo production of chlorinated and brominated MIAs. However, poor promiscuity of C. roseus tryptophan decarboxylase (CroTDC) caused chlorotryptophan accumulation. A subsequent study, in which the substrate specificity of a Lechevalieria aerocolonigenes halogenase was switched to tryptamine, reported improved yields of chloro-substituted MIAs. Yet, as these new-to-nature chemical spaces include both regio-selective considerations as well as choice of halogen, it remains a challenge to mitigate barriers within new-to-nature chemistries in slow-growing plants with limited genetic tractability.

### SUMMARY

The invention is as defined in the claims.

The invention concerns a method for producing monoterpene indole alkaloids (MIAs) and derivatives thereof de novo in a microorganism, including new-to-nature MIAs such as halogenated MIAs and halogenated derivatives thereof, by expression of a heterologous biosynthesis pathway sourced from various organisms. The inventors have achieved de novo production of strictosidine aglycone and derivatives thereof, in vivo production of halogenated MIAs in a microorganism as well as improved product titers for example by co-localisation of pathway enzymes. Microbial based production of strictosidine aglycone and derivatives thereof, including stemmadenine acetate and halogenated derivatives such as halogenated stemmadenine acetate, can be performed at reduced financial and environmental cost compared to methods known in the art.

In one aspect is provided a microorganism capable of producing and/or producing strictosidine aglycone and/or halogenated strictosidine aglycone and/or derivatives thereof, in the presence of geranyl diphosphate (GPP) and tryptamine, and/or geranyl diphosphate and halogenated tryptamine, respectively, said microorganism expressing a geraniol synthase (GES, EC 3.1.7.11) and a strictosidine-O-β-D-glucosidase (SGD, EC 3.2.1.105), preferably wherein GES is as set forth in SEQ ID NO: 65 and/or as set forth in SEQ ID NO: 66, and/or said SGD is RseSGD as set forth in SEQ ID NO: 82 or functional variants thereof having at least 70% homology, similarity or identity to SEQ ID NO: 65, SEQ ID NO: 66, SEQ ID NO: 82, respectively. The GES is a GES capable of converting GPP to geraniol. The SGD is a SGD capable of converting strictosidine or halogenated strictosidine to strictosidine aglycone or halogenated strictosidine aglycone, respectively.

Also provided herein is a nucleic acid construct comprising a nucleic acid sequence identical to or having at least 70% homology, similarity or identity to SEQ ID NO: 3, 4 and/or 20.

Provided is also a vector comprising the above nucleic acid construct, as well as microorganisms comprising said vector and/or said nucleic acid.

Also provided is a method of producing strictosidine aglycone and/or halogenated strictosidine aglycone in a microorganism, said method comprising the steps of:
i. providing a microorganism, said microorganism expressing:
   a geraniol synthase (GES, EC 3.1.7.11) and
   a strictosidine-O-β-D-glucosidase (SGD, EC 3.2.1.105);
ii. incubating said microorganism in a medium comprising a substrate and optionally a halogen atom source, which can be converted to strictosidine aglycone and/or halogenated strictosidine aglycone by said microorganism in the presence of geranyl diphosphate and tryptamine and/or halogenated tryptamine;
iii. optionally recovering the strictosidine aglycone;
iv. optionally further converting the strictosidine aglycone to one or more monoterpene indole alkaloids (MIAs),
wherein said GES is capable of converting geranyl diphosphate to geraniol, optionally said GES is a heterologous GES, preferably said GES is as set forth in SEQ ID NO: 65 and/or in SEQ ID NO: 66; and/or
wherein said SGD is capable of converting strictosidine and/or halogenated strictosidine to strictosidine aglycone and/or halogenated strictosidine aglycone, respectively, optionally said SGD is a heterologous SGD, preferably said SGD is RseSGD (SEQ ID NO: 82), or functional variants thereof having at least 70% homology, similarity or identity to SEQ ID NO: 65, SEQ ID NO: 66 and/or SEQ ID NO: 82, respectively.

Also provided is a composition comprising one of more derivatives of strictosidine aglycone and/or halogenated strictosidine aglycone obtained by the methods described herein.

Also provided are strictosidine aglycone, stemmadenine acetate, alstonine, tetrahydroalstonine, ajmalicine, serpentine, catharanthine, tabersonine, vindoline, halogenated strictosidine aglycone, halogenated stemmadenine acetate, halogenated alstonine, halogenated tetrahydroalstonine, halogenated ajmalicine, halogenated serpentine, halogenated catharanthine, halogenated tabersonine, halogenated vindoline and/or derivatives thereof obtained by the methods described herein.

Also provided are strictosidine, halogenated strictosidine and/or derivatives thereof obtained by the methods described herein, preferably wherein said halogenated strictosidine and/or derivatives thereof is 4-fluorostrictosidine, 5-fluorostrictosidine, 6-fluorostrictosidine, 7-fluorostrictosidine, 4-chlorostrictosidine, 5-chlorostrictosidine, 6-chlorostrictosidine, 7-chlorostrictosidine, 4-bromostrictosidine, 5-bromostrictosidine, 6-bromostrictosidine, 7-bromostrictosidine and/or derivatives thereof, preferably said halogenated strictosidine and/or derivatives thereof is 4-fluorostrictosidine, 5-fluorostrictosidine, 6-fluorostrictosidine, 7-fluorostrictosidine, 7-chlorostrictosidine, 7-bromostrictosidine and/or derivatives thereof.

Also provided are ajmalicine, serpentine, tetrahydroalstonine, alstonine and/or derivatives thereof and/or halogenated ajmalicine, serpentine, tetrahydroalstonine, alstonine and/or derivatives thereof, obtained by the methods described herein, preferably wherein said halogenated tetrahydroalstonine, ajmalicine, serpentine, alstonine and/or derivatives thereof is 7-chloroajmalicine, 7-chloroserpentine, 7-chloroalstonine and/or derivatives thereof, respectively.

Also provided are tetrahydroalstonine and/or derivatives thereof and/or halogenated tetrahydroalstonine and/or derivatives thereof obtained by the methods described herein, preferably wherein said halogenated tetrahydroalstonine and/or derivatives thereof is 7-chlorotetrahydroalstonine, 4-fluorotetrahydroalstonine, 5-fluorotetrahydroalstonine, 6-fluorotetrahydroalstonine, 7-fluorotetrahydroalstonine and/or derivatives thereof.

Also provided are geissoschizine and/or halogenated geissoschizine and/or derivatives thereof obtained by the methods described herein, preferably wherein said halogenated geissoschizine and/or derivatives thereof is fluorinated geissoschizine and/or derivatives thereof such as 4-fluorogeissoschizine, 5-fluorogeissoschizine, 6-fluorogeissoschizine and/or 7-fluorogeissoschizine and/or derivatives thereof, preferably said halogenated geissoschizine and/or derivatives thereof is 4-fluorogeissoschizine, 6-fluorogeissoschizine and/or 7-fluorogeissoschizine and/or derivatives thereof.

Also provided are stemmadenine acetate and/or halogenated stemmadenine acetate and/or derivatives thereof obtained by the methods described herein, preferably wherein said halogenated stemmadenine acetate and/or derivatives thereof is fluorinated stemmadenine acetate and/or derivatives thereof such as 4-fluorostemmadenine acetate, 5-fluorostemmadenine acetate, 6-fluorostemmadenine acetate, 7-fluorostemmadenine acetate and/or derivatives thereof, preferably said halogenated stemmadenine acetate and/or derivatives thereof is 4-fluorostemmadenine acetate, 6-fluorostemmadenine acetate, 7-fluorostemmadenine acetate and/or derivatives thereof.

Also provided are tabersonine and/or halogenated tabersonine and/or derivatives thereof obtained by the methods described herein, preferably wherein said halogenated tabersonine and/or derivatives thereof is fluorinated tabersonine and/or derivatives thereof such as 4-fluorotabersonine, 5-fluorotabersonine, 6-fluorotabersonine, 7-fluorotabersonine and/or derivatives thereof, preferably said halogenated tabersonine and/or derivatives thereof is 6-fluorotabersonine, 7-fluorotabersonine and/or derivatives thereof.

Also provided is a microorganism comprising a nucleic acid construct as described herein and/or a vector as described herein, preferably wherein the microorganism is a bacterium such as Escherichia coli or a yeast such as Saccharomyces cerevisiae.

Also provided is a method for manufacturing a monoterpene indole alkaloid (MIA) and/or a halogenated MIA and/or derivatives thereof of interest, said method comprising the steps of:
i. providing a MIA and/or a halogenated MIA and/or derivatives thereof; and
ii. optionally converting said MIA and/or halogenated MIA and/or derivatives thereof to the MIA and/or halogenated MIA and/or derivatives thereof of interest.

Also provided is a kit of parts comprising a microorganism as described herein, and/or at least one nucleic acid construct as described herein and/or at least one vector at described herein, and optionally instructions for use.

Provided herein is also the use of one or more nucleic acid constructs, microorganisms and/or vectors described herein, for the production of strictosidine aglycone, geissoschizine, stemmadenine acetate, tetrahydroalstonine, alstonine, ajmalicine, serpentine, catharanthine, tabersonine and/or vindoline and/or derivatives thereof in a microorganism, optionally wherein said strictosidine aglycone, geissoschizine, stemmadenine acetate, tetrahydroalstonine, alstonine, ajmalicine, serpentine, catharanthine, tabersonine and/or vindoline and/or derivatives thereof are halogenated.

Also provided herein are methods for treating a disorder such as a cancer, arrhythmia, malaria, fibrosis, pain, anxiety, Parkinson's disease, schizophrenia, bipoloar disorder, psychotic diseases or disorders, hypertension, depression, Alzheimer's disease, addiction, neuronal diseases and/or withdrawal symptoms, comprising administration of a therapeutic sufficient amount of a MIA, a halogenated MIA or a pharmaceutical compound obtained by the methods described herein.

### DESCRIPTION OF DRAWINGS

FIG. 1: De novo strictosidine pathway from geranyl diphosphate (GPP) and tryptophan. Abbreviations: Isopentyl pyrophosphate (IPP); dimethylallyl pyrophosphate (DMAPP); geranyl diphosphate synthase (GPPS); geranyl diphosphate (GPP); farnesyl pyrophosphate synthetase (ERG20); farnesyl diphosphate (FPP); geraniol synthase (GES); geraniol 8-hydroxylase (G8H); NADPH-cytochrome P450 reductase (CPR); cytochrome b5 (CYB5); 8-hydroxygeraniol oxidoreductase (8HGO); iridoid synthase (ISY); iridoid cyclase (CYC); iridoid oxidase (IO); CYP enzymes assisting alcohol dehydrogenase (CYPADH); 7-deoxyloganetic acid glucosyl transferase (7DLGT); 7-deoxyloganic acid hydroxylase (7DLH); loganic acid O-methyltransferase (LAMT); secologanin synthase (SLS); tryptophan decarboxylase (TDC); strictosidine synthase (STR).

FIG. 2: Pathway from strictosidine to alstonine and serpentine. Abbreviations are as in FIG. 1 and as follows: strictosidine-O-β-D-glucosidase (SGD); tetrahydroalstonine synthase (THAS); heteroyohimbine synthase (HYS); serpentine synthase (SS).

FIG. 3: Pathway from strictosidine to stemmadenine acetate, tabersonine and catharanthine. Abbreviations are as in the previous figures and as follows: geissoschizine synthase (GS); geissoschizine oxidase (GO); protein Redox 1 (Redox1); protein Redox 2 (Redox2); stemmadenine-O-acetyltransferase (SAT); O-acetylstemmadenine oxidase/precondylocarpine acetate synthase (PAS/ASO); dihydroprecondylocarpine acetate synthase (DPAS); tabersonine synthase (TS); catharanthine synthase (CS).

FIG. 4: Pathway from tabersonine to vindoline. Abbreviations are as in the previous figures and as follows: tabersonine 16-hydroxylase (T16H); tabersonine 16-O-methyltransferase (16OMT); tabersonine 3-oxygenase (T3O); 16-methoxy-2,3-dihydro-3-hydroxytabersonine synthase (T3R); 3-hydroxy-16-methoxy-2,3-dihydrotabersonine N-methyltransferase (NMT); deacetoxyvindoline 4-hydroxylase (D4H), deacetylvindoline O-acetyltransferase (DAT).

FIG. 5: LC-MS extracted ion chromatogram (EIC) of ion m/z 297.21 matching the exact mass of stemmadenine acetate. Stemmadenine acetate eluding with retention time 6.61 min was detected in spent medium from yeast strain MIA-EM-2 and not strain MIA-CM-5.

FIG. 6: MS/MS spectrum of stemmadenine acetate produced de novo by yeast strain MIA-EM-2. The four most abundant ions—m/Z 168.08, 228.10, 122.09, and 337.19—match MS/MS fragments of semi-synthetic stemmadenine acetate reported by Farrow et al. 2019.

FIG. 7: Pathway for production of halogenated strictosidine analogues from halogenated indole analogues in yeast. The tested indole analogues had one or two hydrogen-to-halogen(s) substitution at carbon position 4, 5, 6, and/or 7 as indicated with arrows on the indole ring. The halogen(s) were either fluorine, chlorine, or bromine. New abbreviations: tryptophan synthase (TRP5).

FIG. 8: Production of halogenated tryptophan by feeding fluoro-, chloro- or bromoindole analogues to yeast strains MIA-CM-10 (black) and MIA-CM-3 (grey). Halogenated tryptophan titer was quantified as total peak area. Strain MIA-CM-3 and MIA-CM-10 were able to produce both fluoro-, chloro-, and bromotryptophan with the halogen atom on all four positions tested. This demonstrates that a wide range of tryptophan analogues can be produced in yeast by indole feeding.

FIG. 9: Production of halogenated tryptamine by feeding fluoro-, chloro- or bromoindole analogues to yeast strains MIA-CM-10 (black) and MIA-CM-3 (grey). Halogenated tryptamine titre was quantified as total peak area. MIA-CM-10 could produce 4-, 5-, 6-, and 7-chlorotryptamine and 6- and 7-bromotryptamine, while MIA-CM-3 only produced 4- and 7-chlorotryptamine and 4-bromotryptamine.

FIG. 10: Production of halogenated strictosidine by feeding fluoro-, chloro- or bromoindole analogues to yeast strains MIA-CM-10 (black) and MIA-CM-3 (grey). Halogenated strictosidine titre was quantified as total peak area. MIA-CM-3 and MIA-CM-10 can produce 4-, 5-, 6-, and 7-fluorostrictosidine. MIA-CM-3 also produced 4- and 7-chlorostrictosidine. MIA-CM-10 produced 7-chlorostrictosidine and 7-bromostrictosidine.

FIG. 11: Representative LC-MS EIC of ion m/z 415.20 matching exact mass of fluorostemmadenine acetate. Fluorostemmadenine acetate was detected in in spent medium of MIA-EM-2 with feeding of secologanin and 6-fluoroindole or 7-fluoroindole.

As expected, retention time, 6.76 min, was slightly longer than natural stemmadenine acetate, 6.61 min. Exact mass and retention time shift support that the detected compound was fluorostemmadenine acetate.

FIG. 12: Representative LC-MS EIC of MS/MS fragment ions m/Z 415>186.0, 415.2>355.2, 415.2>246.2 corresponding to the expected mass shift from hydrogen-to-fluorine substitution of stemmadenine acetate. The chromatogram corresponding to each fragment ion is indicated by arrows. All three fragments co-elude at 6.36 min which shows they are all derived in the same compound. This supported identification of yeast produced 6- and 7-fluorostemmadenine acetate.

FIG. 13: Representative LC-MS EIC of ion m/z 355.18 matching exact mass of fluorotabersonine. The compound was detected in MIA-EM-2 with feeding of secologanin and 6-fluoroindole or 7-fluoroindole. As expected, retention time, 6.97 min, was slightly longer than natural tabersonine. Exact mass and retention time shift support that the detected compound was fluorotabersonine.

FIG. 14: Representative LC-MS EIC of MS/MS fragment ions m/Z 355.2>323.1, 355.2>246.0, 355.2>154.1 corresponding to the expected mass shift from hydrogen-to-fluorine substitution of tabersonine. The chromatogram corresponding to each fragment ion is indicated by arrows. All three fragments co-elude at 6.88 min which shows they were derived from the same compound. This supported identification of 6- and 7-fluorotabersonine produced by yeast strain MIA-EM-2.

FIG. 15: Representative LC-MS EIC of 4-fluorotryptophan fragment ion m/Z 223.3>205.9 comparing retention time of authentic 4-fluorotryptophan standard (black) with 4-fluorotryptophan produced by strain MIA-CM-5 from feeding 4-fluoroindole (grey). Retention time of the yeast produced 4-fluorotryptophan matches the standard confirming conservation of the fluorine-carbon bond in yeast.

FIG. 16: Representative LC-MS EIC of 6-fluorotryptophan fragment ion m/Z 223.3>205.9 comparing retention time of authentic 6-fluorotryptophan standard (black) with 6-fluorotryptophan produced by strain MIA-CM-5 from feeding 6-fluoroindole (grey). Retention time of the yeast produced 6-fluorotryptophan matches the standard confirming conservation of the fluorine-carbon bond in yeast.

FIG. 17: Representative LC-MS EIC of spent medium from yeast strain Sc113 (top) and ScH135 (bottom) fed with 300 mM NaCl. Alstonine standard and extracellular medium from strain Sc112 when fed NaCl were included as controls. The shift in retention time supports that the detected compound with the expected mass was chlorinated alstonine.

FIG. 18: Mirrored MS/MS spectra show major fragments of chloroalstonine with shifts consistent with a hydrogen-chlorine substitution.

FIG. 19: Representative LC-MS EIC of spent medium from yeast strain Sc114 when 300 mM NaCl was fed. Serpentine standard and extracellular medium from strain Sc85 when fed NaCl were included as controls. The shift in retention time support that the detected compound with the expected mass was chlorinated serpentine.

FIG. 20: Mirrored MS/MS spectra show major fragments of chloroserpentine MS/MS spectrum with shifts consistent with a hydrogen-fluorine substitution.

FIG. 21: Representative MS/MS spectra of fluorinated, difluorinated, chlorinated and brominated derivatives of L-tryptophan, tryptamine and strictosidine produced by MIA-CM-10 and MIA-CM-3 through feeding of fluoroindole, difluoroindole, chloroindole or bromoindole, with the halogen atom(s) on C4, C5, C6 and/or C7. Halolgenation is identified by a characteristic mass shift of indole containing fragments according to the hydrogen-to-halogen substitution(s).

FIG. 22: Representative MS/MS spectra of fluorinated, difluorinated, chlorinated and brominated derivatives of serpentine produced by SAB125 through feeding of fluoroindole, difluoroin-dole, chloroindole or bromoindole, with the halogen atom(s) on C4, C5, C6 and/or C7. Halolgenation is identified by the characteristic mass shift of indole containing fragments according to the hydrogen-to-halogen substitution(s).

FIG. 23: Representative mirrored MS/MS spectra of fluorinated, difluorinated, chlorinated and brominated derivatives of alstonine produced by Sc161 through feeding of fluoroindole, difluoroindole, chloroindole or bromoindole, with the halogen atom(s) on C4, C5, C6 and/or C7. Halolgenation is identified by the characteristic mass shift of indole containing fragments according to the hydrogen-to-halogen substitution(s).

### DETAILED DESCRIPTION

### Definitions

Homology, similarity or identity with respect to a polynucleotide (or polypeptide), are defined herein as the percentage of nucleotides (or amino acids) in the candidate sequence that are homologous, similar or identical, respectively, to the residues of a corresponding native nucleotide (or amino acid) sequence, after aligning the sequences and introducing gaps, if necessary, to achieve the maximum percent identity/similarity, and considering any conservative substitutions according to the NCIUB rules ([hftp://www.chem.qmul.ac.uk/iubmb/misc/naseq.html; NC-IUB, Eur J Biochem (1985)]) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given similarity between two sequences implies that the identity between these sequences is at least equal to the similarity; for example, if two sequences are 70% similar to one another, they cannot be less than 70% identical to one another—but could be sharing 80% identity. It follows that the term similarity encompasses both homology and identity, and that the term homology encompasses the term identity. Accordingly, two sequences sharing at least 70% homology will always share at least 70% identity.

Thus, throughout the present disclosure, it will be understood that any variant, such as a functional variant, or homologue said to have at least 70% identity, homology, or similarity to a specified sequence (polynucleotide or polypeptide) refers to a sequence having at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

Functional variant as term refers herein to functional variants of an enzyme, which retain at least some of the activity of the parent enzyme. Thus, a functional variant of a fluorinase, a phosphorylase, a nucleosidase can catalyse the same conversion as a fluorinase, a phosphorylase, or a nucleosidase, respectively, from which they are derived, although the efficiency of the conversion reaction may be different, e.g. the efficiency is decreased or increased compared to the parent enzyme or the substrate specificity is modified. A functional variant can also be a variant of an enzyme, in which variant e.g. the cellular localisation of the enzyme has been modified by including a localisation signal also known as a signal peptide, as is known in the art, or a variant having altered kinetic properties.

Native as term when referring to a polypeptide, such as a protein or an enzyme, or to a polynucleotide, such as a gene, coding sequence of a gene or genetic element, shall herein be construed to refer to a polypeptide or a polynucleotide which is naturally present in a wild type cell.

Heterologous as term when referring to a polypeptide, such as a protein or an enzyme, or to a polynucleotide, such as a gene, coding sequence of a gene or genetic element, shall herein be construed to refer to a polypeptide or a polynucleotide which is not naturally present in a wild type microorganism.

Mutation as term when used herein in the context of nucleic acid sequences refers to a change in nucleic acid sequence compared to the parent nucleic acid sequence. The term mutation covers single nucleotide mutations, but also insertions and deletions of multiple nucleotides, i.e. any change that leads to a different nucleic acid sequence than the parent nucleic acid sequence. The term mutation thus encompasses deletions, such as deletions of a whole gene or of a coding sequence of a gene, or a fragment/fraction of a gene or of a coding sequence of a gene. Preferably, a mutation resulting in altered activity of a protein is a mutation in the gene encoding said protein.

Derived from as term when referring to a polypeptide or a polynucleotide derived from an organism means that said polypeptide or polynucleotide is native to said organism, i.e. that it is naturally found in said organism.

Titer as term herein refers to the concentration of a compound or product that accumulates inside a cell and/or in the extracellular media during cultivation of the cell.

Downstream product and MIA or halogenated MIA of interest as terms herein refer to any molecule, compound, product and/or derivative that has undergone any conversion, either obtained by means of chemicals (chemical synthesis) and/or by enzymatic catalysis (enzymatic conversion) and/or a combination thereof, whereby another molecule, compound, product and/or derivative is being produced and/or synthesized. Said produced and/or synthesized other molecule, compound, product and/or derivative may be volatile, non-volatile, stable and/or unstable. Said produced and/or synthesized other molecule, compound, product and/or derivative may be volatile, non-volatile, stable and/or unstable depending on the condition. In other words, the volatility, non-volatility, stability and/or instability may be condition-dependent. The enzymatic catalysis may be one or more enzyme catalysed reactions. Downstream products may also be referred to as analogues.

Derivative as term herein refers to any molecule, compound, and/or product that has undergone any conversion, either obtained by means of chemicals (chemical synthesis) and/or by enzymatic catalysis (enzymatic conversion) and/or a combination thereof, whereby another molecule, compound and/or product is being produced and/or synthesized. Said produced and/or synthesized other molecule, compound and/or product may be volatile, non-volatile, stable and/or unstable. Said produced and/or synthesized other molecule, compound and/or product may be volatile, non-volatile, stable and/or unstable depending on the condition. In other words, the volatility, non-volatility, stability and/or instability may be condition-dependent. The enzymatic catalysis may be one or more enzyme catalysed reactions. Derivatives may also be referred to as analogues.

MIA as abbreviation herein refers to a monoterpene indole alkaloid. MIAs may also sometimes be referred to as monoterpenoid indole alkaloids.

Stemmadenine acetate may herein and elsewhere also be referred to as O-acetylstemmadenine. Stemmadenine acetate and O-acetylstemmadenine are identical.

Halogenated as term herein refers to a compound or a molecule with one or more halogen atoms introduced in the place of hydrogen, i.e. a compound substituted with one or more halogen atoms. A compound is halogenated if it is substituted with at least one halogen atom. A compound is monohalogenated if one halogen atom is present in said compound. A compound is dihalogenated if two halogen atoms are present in said compound. A compound is trihalogenated if three halogen atoms are present. A compound is tetrahalogenated if four halogen atoms are present. In the context of the present disclosure, the halogen atom(s) may be present in position 4, 5, 6 and/or 7.

When referencing halogenated positions of compounds or molecules containing an indole moiety, the numbering of the indole moiety is used herein, even if said indole-containing compound or molecule uses a different numbering scheme by IUPAC convention. Therefore, it follows that position 6 of the indole moiety of stemmadenine acetate is fluorinated for 6-fluorostemmadenine acetate, that position 4 of the indole moiety of tryptophan is brominated for 4-bromotryptophan, that position 7 of the indole moiety of strictosidine aglycone is chlorinated for 7-chlorostrictosidine aglycone and that position 5 of the indole moiety of tabersonine is fluorinated for 5-fluorotabersonine.

Corresponding halogenated compound as term herein refers to a halogenated compound derived from another halogenated compound by the action of an enzyme, e.g. a tryptophan decarboxylase (TDC) and/or tryptophan synthase (TRP). Such a halogenated compound contains the same halogen atom(s) in the same position(s) of the indole ring as the halogenated compound from which it is derived. For example, the compound corresponding to halogenated tryptamine of 5-chlorotryptophan is 5-chlorotryptamine and the corresponding halogenated strictosidine aglycone of 5,7-difluorotryptamine is 5,7-difluorostrictosidine aglycone.

The skilled person will understand that a microorganism capable of producing a specific compound will produce said specific compound under conditions allowing for its production. Thus, any microorganism capable of producing a compound as described herein actually produces the compound when the necessary substrates and conditions are present, e.g. upon provision of the substrates in the medium or as native metabolites of the microorganism. For example, a halogenated tryptophan or a halogen atom source can be provided to the growth medium.

### Microorganism

The present disclosure provides microorganisms which can produce MIAs and derivatives thereof, where said MIAs and derivatives thereof can be halogenated as detailed herein elsewhere. The present microorganisms express a geraniol synthase (GES) and a strictosidine-O-β-D-glucosidase (SGD), which allows them to produce strictosidine aglycone and/or halogenated strictosidine aglycone and/or derivatives thereof in the presence of geranyl diphosphate and tryptamine. Halogenated derivatives can be obtained by feeding halogenated substrates, such as indoles, to the microorganism, or by employing a tryptophan halogenase, e.g. expressed in the microorganism, or a combination of both.

### Useful Microorganisms

The microorganisms disclosed herein capable of producing compounds of interest such as strictosidine aglycone, stemmadenine acetate, halogenated strictosidine aglycone and/or halogenated stemmadenine acetate and/or derivatives thereof might be referred to as production organisms, production host cells, microbial cell factories, hosts, host cells, production hosts, cell factories etc.

Various microorganisms may be useful as production organisms of stemmadenine acetate, halogenated stemmadenine acetate and/or derivatives thereof according to the present disclosure. Thus, in some embodiments, the microorganism is selected from the group consisting of yeasts, bacteria, archaea, fungi, protozoa, algae, and viruses, preferably the microorganism is a yeast or a bacterium.

In preferred embodiments, the microorganism is a yeast. In some embodiments, the microorganism is a yeast, preferably the genus of said yeast is selected from the group consisting of Saccharomyces, Pichia, Komagataella, Yarrowia, Kluyveromyces, Candida, Rhodotorula, Rhodosporidium, Cryptococcus, Trichosporon and Lipomyces. In other embodiments, the microorganism is a yeast, preferably the yeast is selected from the group consisting of Saccharomyces cerevisiae, Saccharomyces boulardii, Komagataella phaffi (Pichia pastoris), Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica. In preferred embodiments, the microorganism is a yeast, for example said yeast is Saccharomyces cerevisiae. In other preferred embodiments, the microorganism is a yeast, for example said yeast is an engineered Saccharomyces cerevisiae, such as a modified Saccharomyces cerevisiae.

In other embodiments, the microorganism is a bacterium. In some embodiments, the microorganism is a bacterium, preferably the genus of said bacterium is selected from the groups consisting of Escherichia, Corynebacterium, Pseudomonas, Bacillus, Streptomyces, Lactococcus, Lactobacillus, Halomonas, Bifidobacterium and Enterococcus.

In some embodiments, the microorganism is a bacterium, preferably the bacterium is selected from the group consisting of Escherichia coli, Corynebacterium glutamicum, Pseudomonas putida, Bacillus subtilis, Streptomyces albus, Lactococcus bacillus, Halomonas elongate, Bifidobacterium infantis and Enterococcus faecal. In other embodiments, the microorganism is an Escherichia coli.

Throughout the present disclosure, it will be understood that the microorganisms can produce the compounds of interest listed herein when incubated in a medium such as a cultivation medium under conditions that enable the microorganism to grow and produce a desired compound. From the description of the production microorganisms and/or host cells provided herein, the skilled person will have no difficulties in identifying suitable cultivation media and conditions to achieve production of the desired compound. For example, if production of 4-fluorostrictosidine aglycone is desired, 4-fluoroindole may be supplied to the cultivation medium to a microorganism capable of producing 4-fluorostrictosidine from 4-fluoroindole. Such microorganism is described herein below.

### Geranyl Diphosphate

Geranyl diphosphate may be provided to the microorganism, for example as part of the cultivation medium the microorganism is incubated in. In preferred embodiments, the microorganism is capable of synthesising geranyl diphosphate natively, for example geranyl diphosphate is a native metabolite of said microorganism. Geranyl diphosphate may also sometimes be referred to as geranyl pyrophosphate (GPP). Geranyl diphosphate synthase (GPPS) has an EC number EC 2.5.1.1 and converts isopentenyl diphosphate (IPP) and dimethylallyl diphosphate (DMAPP) to GPP. GPPS is known to catalyse the following reaction:

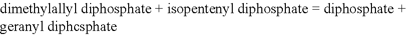

In other words, GPPS catalyses trans-addition of isopentenyl diphosphate (IPP) onto dimethylallyl diphosphate (DMAPP) to form GPP. The microorganism when expressing GPPS is thus capable of converting IPP and DMAPP to GPP, thus producing GPP.

In preferred embodiments, the microorganism is further engineered to improve the synthesis of geranyl diphosphate. Said microorganism may also be further engineered in order to decrease the consumption of geranyl diphosphate by competing cellular pathways such as pathways wherein geranyl diphosphate is converted to another product than geraniol. Strategies and/or other modifications of said microorganism to increase geranyl diphosphate availability are further disclosed herein elsewhere such as in the section “Other modifications”. Geranyl diphosphate (GPP) can be synthesised from isopentenyl diphosphate (IPP) and dimethylallyl diphosphate (DMAPP). In some embodiments, the microorganism expresses an enzyme such as an engineered and/or heterologous enzyme that can convert IPP and DMAPP to GPP.

In some embodiments, the microorganism expresses GES and SGD, and expresses geranyl diphosphate synthase (GPPS), which may not be natively present in the microorganism. In other words, the microorganism may natively express a GPPS, and can optionally be modified so that said native GPPS is overexpressed, or it may express a modified version of the native GPPS, or it may be modified to express a heterologous GPPS, optionally the heterologous GPPS is modified and/or overexpressed.

The SGDs are described in detail further below, in the section “Strictosidine aglycone”.

In some embodiments, the microorganism expresses GES and SGD, and GPPS.

In some embodiments, the GPPS is a GPPS native to Abies grandis such as AgrGPPS2 as set forth in SEQ ID NO: 63 or a functional variant thereof having at least 70% homology, similarity or identity to SEQ ID NO: 63. In other embodiments, GPPS is a GPPS native to Gallus gallus or a functional variant thereof such as GgaFPS*(N144W) as set forth in SEQ ID NO: 64 or a functional variant thereof having at least 70% homology, similarity or identity to SEQ ID NO: 64. In other embodiments, GPPS is a farnesyl pyrophosphate synthase variant F96W-N127W native to Saccharomyces cerevisiae or a functional variant thereof such as ERG20** as set forth in SEQ ID NO: 139 or a functional variant thereof having at least 70% homology, similarity or identity to SEQ ID NO: 139. Functional variants of GPPS, AgrGPPS2, GgaFPS*(N144W) or ERG20** may be identified by expressing said enzyme in a host cell, purifying the enzyme and performing an enzyme assay to measure the conversion from IPP and DMAP to GPP using LC-MS/MS described by Chhonker et al., 2018.

The GPPS may be expressed in the microorganism by introducing a nucleic acid sequence as detailed further below, which encodes a GPPS. In particular, the nucleic acid sequence may be identical to or have at least 70% homology, similarity or identity to SEQ ID NO: 1, SEQ ID NO: 2 and/or SEQ ID NO: 128.

The microorganism described in this section can be used in a method for production of geranyl diphosphate.

### Geraniol

Geraniol may be provided to the microorganism, for example as part of the medium the microorganism is incubated in. However, in addition or as alternative to the above, the microorganism may be further engineered so that it can produce geraniol. Geraniol can be synthesised from geranyl diphosphate (GPP). In some embodiments, the microorganism expresses an enzyme that can convert GPP to geraniol.

In some embodiments, the microorganism expresses SGD and optionally GPPS, and expresses geraniol synthase (GES), which is not natively present in the microorganism.

GES has an EC number EC 3.1.7.11. GES converts GPP to geraniol. GES is known to catalyse the following reaction:

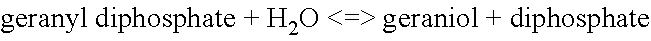

In other words, GES catalyses the conversion of GPP to geraniol. The microorganism when expressing GES is thus capable of converting GPP to geraniol, thus producing geraniol.

In some embodiments, the microorganism expresses GPPS, SGD and GES.

Thus, in some embodiments, the GES is trunCroGES as set forth in SEQ ID NO: 65 or a functional variant thereof having at least 70% homology, similarity or identity to SEQ ID NO: 65. In other embodiments, the GES is ERG20**-GS-trunCroGES as set forth in SEQ ID NO: 66 or a functional variant thereof having at least 70% homology, similarity or identity to SEQ ID NO: 66. ERG20**-GS-trunCroGES (SEQ ID NO: 66) is a fusion protein wherein the GPPS Erg20** is fused to the truncated CroGES, separated by a glycine-serine linker (GS). ERG20**-GS-trunCroGES is a bi-functional enzyme with both GPPS and GES activity and catalyses conversion of IPP and DMAPP to GPP and conversion of GPP to geraniol. trunCroGES (SEQ ID NO: 65) is a truncated version of the GES native to Catharanthus roseus (CroGES, AFD64744.1) such that the chloroplast signal peptide is removed from said CroGES thus yielding trunCroGES. trunCroGES (SEQ ID NO: 65) catalyses formation of geraniol from geranyl diphosphate.

Functional variants of GES may be identified by expressing said enzyme in a host cell, purifying the enzyme and performing an enzyme assay to measure the conversion from GPP to geraniol using gas chromatography-mass spectometry as described by Denby et al. (2018).

The GES may be expressed in the microorganism by introducing a nucleic acid sequence as detailed further below, which encodes a GES. In particular, the nucleic acid sequence may be identical to or have at least 70% homology, similarity or identity to SEQ ID NO: 3 and/or SEQ ID NO: 4.

In preferred embodiments, the microorganism expresses trunCroGES (SEQ ID NO: 65) and/or ERG20**-GS-trunCroGES (SEQ ID NO: 66); RseSGD (SEQ ID NO: 82); and/or ERG20** (SEQ ID NO: 139), AgrGPPS2 (SEQ ID NO: 63) and/or GgaFPS*(N144W) (SEQ ID NO: 64); or functional variants thereof having at least 70% homology, similarity or identity to SEQ ID NO: 65, 66, 82, 139, 63 and/or 64, respectively.

The microorganism described in this section can be used in a method for production of geraniol.

### CPR and CYB5

The microorganism as described anywhere herein may further express a NADPH-cytochrome P450 reductase (CPR) and/or a Cytochrome b5 (CYB5), which are not natively present in the microorganism.

CPR has an EC number EC 1.6.2.4 and is required for electron transfer from NADPH to cytochrome P450. CYB5 has an EC number EC 1.6.2.2 and is a membrane bound hemoprotein which functions as an electron carrier. CPR can catalyse the following reaction:

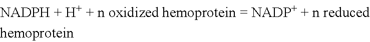

CYB5 can catalyse the following reaction:

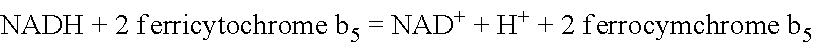

In preferred embodiments, the CPR is a CPR native to Catharanthus roseus or a functional variant thereof which retains the ability to transfer electrons from NADPH to cytochrome P450. Thus, in some embodiments, the CPR is CroCPR as set forth in SEQ ID NO: 68 or a functional variant thereof having at least 70% homology, similarity or identity to SEQ ID NO: 68.

In other embodiments, the CPR is a CPR native to or derived from Arabidopsis thaliana, Artemisia annua, Camptotheca acuminate, Olea europaea, Chrysomela populi, Aspergillus niger, Catharanthus longifolius, Rauvolfia serpentine and/or Amsonia hubrichtii or functional variants thereof. In some embodiments, the CPR is AthCPR (AAK96879.1) from Arabidopsis thaliana, AanCPR (ABC47946.1) from Artemisia annua, CacCPR (AJW67229.1) from Camptotheca acuminata, OeuCPR (XP_022867604.1) from Olea europaea, CpoCPR (QEG78946.1) from Chrysomela populi, AniCPR (Uniprot: Q00141) from Aspergillus niger, CloCPR (SEQ ID NO: 147) from Catharanthus longifolius, RseCPR (SEQ ID NO: 146) from Rauvolfia serpentine and/or AhuCPR (SEQ ID NO: 145) from Amsonia hubrichtii or functional variants thereof having at least 70% homology, similarity or identity to AthCPR, AanCPR, CacCPR, OeuCPR, CpoCPR, AniCPR, CloCPR (SEQ ID NO: 147), RseCPR (SEQ ID NO: 146), AhuCPR (SEQ ID NO: 145), respectively.

The CPR may be expressed in the microorganism by introducing a nucleic acid sequence as detailed further below, which encodes a CPR. In particular, the nucleic acid sequence may be identical to or have at least 70% homology, similarity or identity to SEQ ID NO: 6.

In preferred embodiments, the CYB5 is a CYB5 native to Catharanthus roseus or a functional variant thereof which retains the ability to function as an electron carrier.

Thus, in some embodiments, the CYB5 is CroCYB5 as set forth in SEQ ID NO: 69 or a functional variant thereof having at least 70% homology, similarity or identity to ID NO: 69.

The CYB5 may be expressed in the microorganism by introducing a nucleic acid sequence as detailed further below, which encodes a CYB5. In particular, the nucleic acid sequence may be identical to or have at least 70% homology, similarity or identity to SEQ ID NO: 7.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); and CroCPR (SEQ ID NO: 68); or functional variants thereof having at least 70% homology, similarity or identity to SEQ ID NO: 65, 66, 82, 139, 63, 64 and/or 68, respectively.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); and/or CroCPR (SEQ ID NO: 68) and/or CroCYB5 (SEQ ID NO: 69); or functional variants thereof having at least 70% homology, similarity or identity to SEQ ID NO: 65, 66, 82, 139, 63, 64, 68 and/or 69, respectively.

8-hydroxygeraniol

In addition to the above the microorganism may be further engineered so that it can produce 8-hydroxygeraniol. 8-hydroxygeraniol can be synthesised from geraniol. In some embodiments, the microorganism expresses one or more enzymes that together and/or individually can convert geraniol to 8-hydroxygeraniol.

In some embodiments, the microorganism expresses GES and SGD, and optionally at least one of GPPS, CPR or CYB5, and further expresses geraniol 8-hydroxylase (G8H), which is not natively present in the microorganism.

G8H has an EC number EC 1.14.14.83, is a cytochrome P450 enzyme and converts geraniol to 8-hydroxygeraniol. In other words, G8H catalyses the oxidation of geraniol to 8-hydroxygeraniol. The microorganism when expressing G8H, and optionally CPR and/or CYB5, is thus capable of converting geraniol to 8-hydroxygeraniol, thus producing 8-hydroxygeraniol.

In some embodiments, the microorganism expresses GES and SGD, and GPPS, CPR, CYB5 and G8H.

In some embodiments, the G8H is a G8H native to Catharanthus roseus or a functional variant thereof which retains the ability to convert geraniol to 8-hydroxygeraniol. Thus, in some embodiments, the G8H is CroG8H as set forth in SEQ ID NO: 67 or a functional variant thereof having at least 70% homology, similarity or identity to SEQ ID NO: 67. Functional variants of G8H or CroG8H may be identified by expressing said enzyme in a host cell and measure 8-hydroxygeraniol formation using gas or liquid chromatography-mass spectrometry. Functionality can also be inferred by purifying the enzyme and performing an in vitro enzyme assay to measure the conversion from geraniol to 8-hydroxygeraniol, again using gas chromatography-mass spectrometry (GCMS) as described in Davies et al. (2021).

The G8H may be expressed in the microorganism by introducing a nucleic acid sequence as detailed further below, which encodes a G8H. In particular, the nucleic acid sequence may be identical to or have at least 70% homology, similarity or identity to SEQ ID NO: 5.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); and/or CroG8H (SEQ ID NO: 67); or functional variants thereof having at least 70% homology, similarity or identity to SEQ ID NO: 65, 66, 82, 139, 63, 64, 68, 69 and/or 67, respectively.

The microorganism described in this section can be used in a method for production of 8-hydroxygeraniol.

8-oxogeranial

In addition to the above the microorganism may be further engineered so that it can produce 8-oxogeranial. 8-oxogeranial can be synthesised from 8-hydroxygeraniol. In some embodiments, the microorganism expresses an enzyme that can convert 8-hydroxygeraniol to 8-oxogeranial.

In some embodiments, the microorganism expresses GES and SGD, and optionally at least one of GPPS, CPR, CYB5 or G8H, and further expresses 8-hydroxygeraniol dehydrogenase (8HGO), which is not natively present in the microorganism.

8HGO has an EC number EC 1.1.1.324 and converts 8-hydroxygeraniol to 8-oxogeranial. In other words, 8HGO catalyses the conversion of 8-hydroxygeraniol to 8-oxogeranial. The microorganism when expressing 8HGO, is thus capable of converting 8-hydroxygeraniol to 8-oxogeranial, thus producing 8-hydroxygeraniol.

In some embodiments, the microorganism expresses GES and SGD, and GPPS, CPR, CYB5, G8H and 8HGO.

In some embodiments, the 8HGO is an 8HGO native to Catharanthus roseus such as Cro8HGO (AAQ55962.1, AHK60836.1) or a functional variant thereof having at least 70% homology, similarity or identity thereto. In preferred embodiments, the 8HGO is an 8HGO native to Vinca minor or a functional variant thereof which retains the ability to convert 8-hydroxygeraniol to 8-oxogeranial. Thus, in some embodiments, the 8HGO is Vmi8HGOA as set forth in SEQ ID NO: 70 or a functional variant thereof having at least 70% homology, similarity or identity to SEQ ID NO: 70. Functional variants of 8HGO, Cro8HGO or Vmi8HGOA may be identified by expressing said enzyme in a host cell, purifying the enzyme and performing an enzyme assay to measure the conversion from 8-hydroxygeraniol to 8-oxogeranial using gas chromatography-mass spectrometry (GC-MS) as described by Miettinen et al (2014).

The 8HGO may be expressed in the microorganism by introducing a nucleic acid sequence as detailed further below, which encodes an 8HGO. In particular, the nucleic acid sequence is identical to or has at least 70% homology, similarity or identity to SEQ ID NO: 8.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); and/or Vmi8HGOA (SEQ ID NO: 70); or functional variants thereof having at least 70% homology, similarity or identity to SEQ ID NO: 65, 66, 82, 139, 63, 64, 68, 69, 67 and/or 70, respectively.

The microorganism described in this section can be used in a method for production of 8-oxogeranial.

Cis-trans-nepetalactol

In addition to the above the microorganism may be further engineered so that it can produce 8-oxocitronellyl enol and/or cis-trans-nepetalactol. 8-oxocitronellyl enol and/or cis-trans-nepetalactol can be synthesised from 8-oxogeranial. In some embodiments, the microorganism expresses one or more enzymes that individually and/or together can convert 8-oxogeranial to 8-oxocitronellyl enol and/or cis-trans-nepetalactol. 8-oxocitronellyl enol may also be referred to as (S)-8-oxocitronellyl enol.

In some embodiments, the microorganism expresses GES and SGD, and optionally at least one of GPPS, CPR, CYB5, G8H or 8HGO, and further expresses iridoid synthase (ISY) and/or iridoid cyclase (CYC), which are not natively present in the microorganism.

ISY has an EC number EC 1.3.1.122 and converts 8-oxogeranial to 8-oxocitronellyl enol, which can be further converted to cis-trans-nepetalactol. ISY can catalyse the following reaction:

(S)-8-oxocitronellyl enol+NAD(P)⁺=(6E)-8-oxogeranial+NAD(P)H+H⁺

ISY can catalyse the first step in conversion of 8-oxogeranial to cis-trans-nepetalactol. The microorganism when expressing ISY, is thus capable of converting 8-oxogeranial to 8-oxocitronellyl enol which can be further converted to cis-trans-nepetalactol, thus producing 8-oxocitronellyl enol and/or cis-trans-nepetalactol.

In some embodiments, the microorganism expresses GES and SGD, and GPPS, CPR, CYB5, G8H, 8HGO and ISY. In other embodiments, the microorganism expresses GES and SGD, and GPPS, CPR, CYB5, G8H, 8HGO, ISY and CYC.

In some embodiments, the ISY is native to a plant. For example, the ISY is native to a plant belonging to one of the following genera Olea, Digitalis, Catharanthus, or Nepeta. In some embodiments, the ISY is an Olea europeae ISY, a Digitalis purpurea ISY, a Catharanthus roseus ISY, a Nepeta mussinii ISY, a Nepeta cataria ISY or functional variants thereof. In other embodiments, the ISY is an ISY native to Olea europeae such as OeulSY (KT954038.1), Digitalis purpurea such as DpulSY (ACZ66261.1), Catharanthus roseus such as CrolSY (AFW98981.1), Nepeta mussinii such as NmulSY2 (KY882236.1) or functional variants thereof having at least 70% homology, similarity or identity to OeulSY, DpulSY, CrolSY and/or NmulSY2, respectively.

In preferred embodiments, the ISY is an ISY native to Nepeta cataria or a functional variant thereof which retains the ability to convert 8-oxogeranial to 8-oxocitronellyl enol and/or cis-trans-nepetalactol. Thus, in preferred embodiments, the ISY is NcalSY as set forth in SEQ ID NO: 71 or a functional variant thereof having at least 70% homology, similarity or identity to SEQ ID NO: 71.

Functional variants of ISY or NcalSY may be modified enzymes which retain the capability to convert 8-oxogeranial to 8-oxocitronellyl enol. Functional variants of ISY or NcalSY may be identified by expressing said enzyme in a host cell, purifying the enzyme using standard techniques and measuring the conversion from 8-oxogeranial to 8-oxocitronellyl enol in an enzyme assay using gas chromatography-mass spectrometry as described in Lichman et al. (2020).

The ISY may be expressed in the microorganism by introducing a nucleic acid sequence as detailed further below, which encodes an ISY. In particular, the nucleic acid sequence may be identical to or have at least 70% homology, similarity or identity to SEQ ID NO: 9.

CYC has an EC number EC 5.5.1.34 and can convert 8-oxocitronellyl enol to cis-trans-nepetalactol. CYC can catalyse the following reaction:

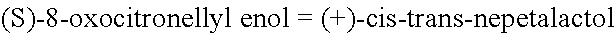

The microorganism when expressing CYC, is thus capable of converting 8-oxocitronellyl enol to cis-trans-nepetalactol, thus producing cis-trans-nepetalactol. CYC can catalyse the cyclization step in the conversion of 8-oxogeranial to cis-trans-nepetalactol. In some embodiments of the present invention, the CYC, when expressed in the microorganism together with ISY, is capable of converting 8-oxogeranial to cis-trans-nepetalactol. The microorganism when expressing ISY and CYC, is thus capable of converting 8-oxogeranial to cis-trans-nepetalactol, thus producing cis-trans-nepetalactol. The microorganism can be any of those described herein.

The CYC may be derived from a eukaryote or a prokaryote, as detailed below, preferably a eukaryotic cell such as a plant cell. CYCs (EC 5.5.1.34) are capable of performing a cyclisation reaction, and some of these enzymes can be characterised as cyclases, while others are progesterone reductases which are also able to perform a cyclisation reaction.

In some embodiments, the CYC is native to a plant belonging to one of the following genera: Olea, Nepeta or Catharanthus. In some embodiments, the CYC is an enzyme from one of the following species: Olea europeae, Nepeta mussinii, Nepeta cataria or Catharanthus roseus. In some embodiments, the CYC is a CYC native to Olea europeae such as OeulSYB (XP_022858441.1), Nepeta mussinii such as NmuMLPL (QKE59448.1) or NmuNEPS2 (AXF35972.1), Nepeta cataria such as NcaMLPLA (SEQ ID NO: 72) or NcaMLPLB (QKE59461.1), Catharanthus roseus such as CroP5bR4 (AIW09146.1) or a functional variant thereof. In other words, in some embodiments the CYC is derived from Olea europeae, Nepeta mussinii, Nepeta cataria, Catharanthus roseus or is a functional variant thereof.

In preferred embodiments, the CYC is a CYC native to Nepeta cataria or a functional variant thereof which retains the ability to convert 8-oxocitronellyl enol to cis-trans-nepetalactol and/or together with an ISY convert 8-oxogeranial to cis-trans-nepetalactol. Thus, in preferred embodiments, the CYC is NcaMLPLA as set forth in SEQ ID NO: 72 or a functional variant thereof having at least 70% homology, similarity or identity to SEQ ID NO: 72.

Functional variants of the CYC or NcaMLPLA may be modified enzymes which retain the capability to convert 8-oxocitronellyl enol to cis-trans-nepetalactol and/or together with ISY convert 8-oxogeranial to cis-trans-nepetalactol. Functional variants of CYC or NcaMLPLA may be identified by expressing said enzyme in a host cell, purifying the enzyme using standard techniques and measuring the conversion from 8-oxogeranial to cis-trans-nepetalactol in an enzyme assay using gas chromatography-mass spectrometry as described in Lichman et al. (2020), and/or the conversion from 8-oxocitronellyl enol to cis-trans-nepetalactol and/or together with ISY convert 8-oxogeranial using standard techniques.

The CYC may be expressed in the microorganism by introducing a nucleic acid sequence as detailed further below, which encodes a CYC. In particular, the nucleic acid sequence may be identical to or have at least 70% homology, similarity or identity to SEQ ID NO: 10.

The microorganism, ISY and/or CYC may be as described in detail in application WO2022/106638 entitled “Methods for production of cis-trans-nepetalactol and iridoids” filed 19 Nov. 2021 and assigned to the same applicant, particularly in the sections entitled “Yeast cell”, “Iridoid synthase (ISY)” and/or “Additional enzyme” therein.

In some embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71) and/or NcaMLPLA (SEQ ID NO: 72); or functional variants thereof having at least 70% homology, similarity or identity to SEQ ID NO: 65, 66, 82, 139, 63, 64, 68, 69, 67, 70, 71 and/or 72, respectively.

The microorganism described in this section can be used in a method for production of 8-oxocitronellyl enol and/or cis-trans-nepetalactol.

7-deoxyloganetic Acid

In addition to the above, the microorganism may be further engineered so that it can produce 7-deoxyloganetic acid. 7-deoxyloganetic acid can be synthesised from cis-trans-nepetalactol. In some embodiments, the microorganism expresses one or more enzymes that together and/or individually can convert cis-trans-nepetalactol to 7-deoxyloganetic acid.

In some embodiments, the microorganism expresses GES and SGD, and optionally at least one of GPPS, CPR, CYB5, G8H, 8HGO, ISY or CYC, and further expresses iridoid oxidase (IO) and/or CYP enzymes-assisting alcohol dehydrogenase (CYPADH), which are not natively present in the microorganism. IO may also sometimes be referred to as 7-deoxyloganetic acid synthase.

IO has an EC number EC 1.14.14.161. CYPADH has an EC number EC 1.1.1.-. Together, IO and CYPADH catalyse the conversion of cis-trans-nepetalactol to 7-deoxyloganetic acid. The microorganism when expressing IO and CYPADH, and optionally CPR and/or CYB5, is capable of converting cis-trans-nepetalactol to 7-deoxyloganetic acid, thus producing 7-deoxyloganetic acid.

In some embodiments, the microorganism expresses GES and SGD, and GPPS, CPR, CYB5, G8H, 8HGO, ISY, CYC, IO and CYPADH.

In some embodiments, the IO is an IO native to Catharanthus roseus or a functional variant thereof which retains the ability to convert cis-trans-nepetalactol to 7-deoxyloganetic acid together with CYPADH. Thus, in some embodiments, the IO is CroIO as set forth in SEQ ID NO: 73, or a functional variant thereof having at least 70% homology, similarity or identity to SEQ ID NO: 73. Functional variants of IO or CroIO may be identified by expressing said enzyme in a host cell, purifying the enzyme and performing an enzyme assay together with CYPADH to measure the conversion from cis-trans-nepetalactol to 7-deoxyloganetic acid using GC-MS described by Brown et al. (2015).

The IO may be expressed in the microorganism by introducing a nucleic acid sequence as detailed further below, which encodes an IO. In particular, the nucleic acid sequence may be identical to or have at least 70% homology, similarity or identity to SEQ ID NO: 11.

In some embodiments, the CYPADH is a CYPADH native to Catharanthus roseus or a functional variant thereof which retains the ability to convert cis-trans-nepetalactol to 7-deoxyloganetic acid together with IO. Thus, in some embodiments, the CYPADH is CroCYPADH as set forth in SEQ ID NO: 74, or a functional variant thereof having at least 70% homology, similarity or identity to SEQ ID NO: 74. Functional variants of CYPADH or CroCYPADH may be identified by expressing said enzyme in a host cell, purifying the enzyme and performing an enzyme assay together with IO to measure the conversion from cis-trans-nepetalactol to 7-deoxyloganetic acid using GC-MS described by Brown et al. (2015).

The CYPADH may be expressed in the microorganism by introducing a nucleic acid sequence as detailed further below. In particular, the nucleic acid sequence may be identical to or have at least 70% homology, similarity or identity to SEQ ID NO: 12.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73) and/or CroCYPADH (SEQ ID NO: 74); or functional variants thereof having at least 70% homology, similarity or identity to SEQ ID NO: 65, 66, 82, 139, 63, 64, 68, 69, 67, 70, 71, 72, 73 and/or 74, respectively.

The microorganism described in this section can be used in a method for production of 7-deoxyloganetic acid.

7-deoxyloganic Acid

In addition to the above, the microorganism may be further engineered so that it can produce 7-deoxyloganic acid. 7-deoxyloganic acid can be synthesised from 7-deoxyloganetic acid. In some embodiments, the microorganism expresses an enzyme that can convert 7-deoxyloganetic acid to 7-deoxyloganic acid.

In some embodiments, the microorganism expresses GES and SGD, and optionally at least one of GPPS, CPR, CYB5, G8H, 8HGO, ISY, CYC, IO or CYPADH, and further expresses 7-deoxyloganetic acid glucosyl transferase (7DLGT), which is not natively present in the microorganism.

7DLGT has an EC number EC 2.4.1.323. 7DLGT catalyses the conversion of 7-deoxyloganetic acid to 7-deoxyloganic acid. The microorganism when expressing 7DLGT is thus capable of converting 7-deoxyloganetic acid to 7-deoxyloganic acid, thus producing 7-deoxyloganic acid.

In some embodiments, the microorganism expresses GES and SGD, and GPPS, CPR, CYB5, G8H, 8HGO, ISY, CYC, IO, CYPADH and 7DLGT.

In some embodiments, the 7DLGT is a 7DLGT native to Catharanthus roseus or a functional variant thereof which retains the ability to convert 7-deoxyloganetic acid to 7-deoxyloganic acid. Thus, in some embodiments, the 7DLGT is Cro7DLGT as set forth in SEQ ID NO: 75 or a functional variant thereof having at least 70% homology, similarity or identity to SEQ ID NO: 75. Functional variants of 7DLGT or Cro7DLGT may be identified by expressing said enzyme in a host cell, purifying the enzyme and performing an enzyme assay to measure the conversion from 7-deoxyloganetic acid to 7-deoxyloganic acid using standard techniques.

The 7DLGT may be expressed in the microorganism by introducing a nucleic acid sequence as detailed further below, which encodes a 7DLGT. In particular, the nucleic acid sequence may be identical to or have at least 70% homology, similarity or identity to SEQ ID NO: 13.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); and/or Cro7DLGT (SEQ ID NO: 75); or functional variants thereof having at least 70% homology, similarity or identity to SEQ ID NO: 65, 66, 82, 139, 63, 64, 68, 69, 67, 70, 71, 72, 73, 74 and/or 75, respectively.

The microorganism described in this section can be used in a method for production of 7-deoxyloganic acid.

### Loganic Acid

In addition to the above the microorganism may be further engineered so that it can produce loganic acid. Loganic acid can be synthesised from 7-deoxyloganic acid. In some embodiments, the microorganism expresses one or more enzymes that together and/or individually can convert 7-deoxyloganic acid to loganic acid.

In some embodiments, the microorganism expresses GES and SGD, and optionally at least one of GPPS, CPR, CYB5, G8H, 8HGO, ISY, CYC, IO, CYPADH or 7DLGT, and further expresses 7-deoxyloganic acid hydroxylase (7DLH), which is not natively present in the microorganism.

7DLH has an EC number EC 1.14.14.85. 7DLH catalyses the conversion of 7-deoxyloganic acid to loganic acid. The microorganism expressing 7DLH, and optionally CPR and/or CYB5, is thus capable of converting 7-deoxyloganic acid to loganic acid, thus producing loganic acid.

In some embodiments, the microorganism expresses GES and SGD, and GPPS, CPR, CYB5, G8H, 8HGO, ISY, CYC, IO, CYPADH, 7DLGT and 7DLH.

In preferred embodiments, the 7DLH is a 7DLH native to Catharanthus roseus or a functional variant thereof which retains the ability to convert 7-deoxyloganic acid to loganic acid. Thus, in some embodiments, the 7DLH is Cro7DLH as set forth in SEQ ID NO: 76 or a functional variant thereof having at least 70% homology, similarity or identity to SEQ ID NO: 76. Functional variants of 7DLH or Cro7DLH may be identified by expressing said enzyme in a host cell, purifying the enzyme and performing an enzyme assay to measure the conversion from 7-deoxyloganic acid to loganic acid using liquid chromatography-mass spectrometry (LC-MS) as described in Brown et al. (2015).

The 7DLH may be expressed in the microorganism by introducing a nucleic acid sequence as detailed further below, which encodes a 7DLH. In particular, the nucleic acid sequence may be identical to or have at least 70% homology, similarity or identity to SEQ ID NO: 14.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); and/or Cro7DLH (SEQ ID NO: 76); or functional variants thereof having at least 70% homology, similarity or identity to SEQ ID NO: 65, 66, 82, 139, 63, 64, 68, 69, 67, 70, 71, 72, 73, 74, 75 and/or 76, respectively.

The microorganism described in this section can be used in a method for production of loganic acid.

### Loganin

In addition to the above the microorganism may be further engineered so that it can produce loganin. Loganin can be synthesised from loganic acid. In some embodiments, the microorganism expresses an enzyme that can convert loganic acid to loganin.

In some embodiments, the microorganism expresses GES and SGD, and optionally at least one of GPPS, CPR, CYB5, G8H, 8HGO, ISY, CYC, IO, CYPADH, 7DLGT or 7DLH, and further expresses loganic acid O-methyltransferase (LAMT), which is not natively present in the microorganism.

LAMT has an EC 2.1.1.50. LAMT catalyses the conversion of loganic acid to loganin. The microorganism expressing LAMT is thus capable of converting loganic acid to loganin, thus producing loganin.

In some embodiments, the microorganism expresses GES and SGD, and GPPS, CPR, CYB5, G8H, 8HGO, ISY, CYC, IO, CYPADH, 7DLGT, 7DLH and LAMT.

In some embodiments, the LAMT is a LAMT native to Catharanthus roseus or a functional variant thereof which retains the ability to convert loganic acid to loganin. Thus, in some embodiments, the LAMT is CroLAMT as set forth in SEQ ID NO: 77 or a functional variant thereof having at least 70% homology, similarity or identity to SEQ ID NO: 77. Functional variants of LAMT or CroLAMT may be identified by expressing said enzyme in a host cell, purifying the enzyme and performing an enzyme assay to measure the conversion from loganic acid to loganin using LC-MS as described in Brown et al. (2015).

The LAMT may be expressed in the microorganism by introducing a nucleic acid sequence as detailed further below, which encodes a LAMT. In particular, the nucleic acid sequence may be identical to or have at least 70% homology, similarity or identity to SEQ ID NO: 15.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); and/or CroLAMT (SEQ ID NO: 77); or functional variants thereof having at least 70% homology, similarity or identity to SEQ ID NO: 65, 66, 82, 139, 63, 64, 68, 69, 67, 70, 71, 72, 73, 74, 75, 76 and/or 77, respectively.

The microorganism described in this section can be used in a method for production of loganin.

### Secologanin

In addition to the above the microorganism may be further engineered so that it can produce secologanin. Secologanin can be synthesised from loganin. In some embodiments, the microorganism expresses one or more enzymes that together and/or individually can convert loganin to secologanin.

In some embodiments, the microorganism expresses GES and SGD, and optionally at least one of GPPS, CPR, CYB5, G8H, 8HGO, ISY, CYC, IO, CYPADH, 7DLGT, 7DLH or LAMT, and further expresses secologanin synthase (SLS), which is not natively present in the microorganism.

SLS has an EC number EC 1.14.19.62. SLS catalyses the conversion of loganin to secologanin. The microorganism when expressing SLS, and optionally CPR and/or CYB5, is thus capable of converting loganin to secologanin, thus producing secologanin.

In some embodiments, the microorganism expresses GES and SGD, and GPPS, CPR, CYB5, G8H, 8HGO, ISY, CYC, IO, CYPADH, 7DLGT, 7DLH, LAMT and SLS.

In some embodiments, the SLS is a SLS native to Catharanthus roseus or a functional variant thereof which retains the ability to convert loganin to secologanin. Thus, in some embodiments, the SLS is CroSLS as set forth in SEQ ID NO: 78 or a functional variant thereof having at least 70% homology, similarity or identity to SEQ ID NO: 78. Functional variants of SLS or CroSLS may be identified by expressing said enzyme in a host cell, purifying the enzyme and performing an enzyme assay to measure the conversion from loganin to secologanin using LC-MS as described in Brown et al. (2015).

The SLS may be expressed in the microorganism by introducing a nucleic acid sequence as detailed further below, which encodes a SLS. In particular, the nucleic acid sequence may be identical to or have at least 70% homology, similarity or identity to SEQ ID NO: 16.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); and/or CroSLS (SEQ ID NO: 78); or functional variants thereof having at least 70% homology, similarity or identity to SEQ ID NO: 65, 66, 82, 139, 63, 64, 68, 69, 67, 70, 71, 72, 73, 74, 75, 76, 77 and/or 78, respectively.

The microorganism described in this section can be used in a method for production of secologanin.

### Tryptophan Synthase

In addition to the above the microorganism may be further engineered so that it can produce halogenated tryptophan. Halogenated tryptophan can be synthesised from halogenated indole and serine and/or as described in the section “De novo halogenation” herein below. In some embodiments, the microorganism expresses one or more enzymes that together and/or individually can convert serine and halogenated indole to a corresponding halogenated tryptophan.

Halogenated indole may be provided to the microorganism, for example as part of the medium the microorganism is incubated in as also detailed herein, for example herein below. Tryptophan and/or halogenated tryptophan may also be provided to the microorganism, for example as part of the medium the microorganism is incubated in as also detailed herein, for example herein below. However, the microorganism may also be capable of synthesising tryptophan and/or halogenated tryptophan as detailed herein.

The halogenated indole and/or tryptophan may be as described anywhere else herein, in particular in the section “Products, substrates and compounds” herein below. For example, the halogenated indole and/or halogenated tryptophan may be 4-halogenated, 5-halogenated, 6-halogenated and/or 7-halogenated by a halogen selected from the group consisting of fluorine, chlorine and bromine, for example the halogenated indole may be 4-fluoroindole, 5-chloroindole, or 7-bromoindole. The halogenated indole and/or halogenated tryptophan may also be halogenated in more than one position, for example in two positions, by a halogen atom, each atom being independently selected from the group consisting of fluorine, chlorine and bromine. For example the halogenated tryptophan may be dichlorotryptophan such as 5,6-dichlorotryptophan, 5,7-dichlorotryptophan, 6,7-dichlorotryptophan and/or dibromotryptophan such as 5,6-dibromotryptophan, 5,7-dibromotryptophan, 6,7-dibromotryptophan. Dihalogenated indole may for example be fluoro-chloro-indole, dichloroindole, difluoroindole, and/or dibromoindole. In some embodiments, the dihalogenated indole is 7,6-difluoroindole, 7,5-difluoroindole, 7,4-difluoroindole, 6,4-difluoroindole, 6,5-difluoroindole and/or 5,4-difluoroindole. Dihalogenated indole may also be 7-bromo-5-fluoroindole, 6-bromo-4-fluoroindole and/or 6-chloro-5-fluoroindole. Trihalogenated indole may for example be trifluoroindole, trichloroindole or tribromoindole, such as for example, but not limited to 4,5,7-trifluoroindole, 5,6,7-trichloroindole and/or 4,6,7-tribromoindole.

In some embodiments, the microorganism expresses GES and SGD, and further expresses tryptophan synthase (TRP). In other embodiments, the microorganism expresses GES and SGD, and optionally at least one of GPPS, CPR, CYB5, G8H, 8HGO, ISY, CYC, IO, CYPADH, 7DLGT, 7DLH, LAMT or SLS, and further expresses TRP. In all cases the TRP may be natively expressed in the microorganism and/or not natively expressed in the microorganism. In other words, the TRP may be a native TRP and/or a heterologous TRP. In some embodiments, the microorganism may express one or more native and/or heterologous TRPs.

TRP has an EC number EC 4.2.1.20. TRP is known to catalyse the following reactions:

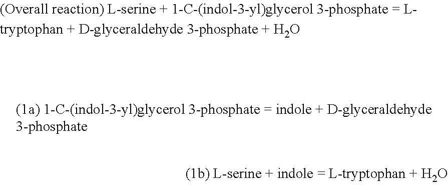

Many organisms, including microorganisms, natively express at least one TRP that can catalyse the above reactions. An example of this is Saccharomyces cerevisiae which for example expresses tryptophan synthase TRP5 (SEQ ID NO: 138).

TRP can also catalyse the conversion of serine and halogenated indole to a corresponding halogenated tryptophan. The microorganism when expressing TRP may thus be capable of converting serine and halogenated indole to a corresponding halogenated tryptophan, thus producing halogenated tryptophan. For example, TRP5 (SEQ ID NO: 138) native to Saccharomyces cerevisiae is capable of converting serine and a halogenated indole to halogenated tryptophan.

In some embodiments, the microorganism expresses GES and SGD, and GPPS, CPR, CYB5, G8H, 8HGO, ISY, CYC, IO, CYPADH, 7DLGT, 7DLH, LAMT, SLS and TRP.

In some embodiments, the TRP is a TRP native to the microorganism. In some embodiments the TRP is a TRP native to Saccharomyces cerevisiae or a functional variant thereof which retains the ability to convert halogenated indole to a corresponding halogenated tryptophan. Thus, in some embodiments, the TRP is TRP5 as set forth in SEQ ID NO: 138 or a functional variant thereof having at least 70% homology, similarity or identity to SEQ ID NO: 138. Functional variants of TRP or TRP5 may be identified by expressing said enzyme in a host cell, purifying the enzyme and performing an enzyme assay to measure the conversion from halogenated indole to a corresponding halogenated tryptophan using liquid chromatography-mass spectrometry as described by Glenn et al. (2011).

The TRP may be expressed in the microorganism by introducing a nucleic acid sequence as detailed further below, which encodes a TRP. In particular, the nucleic acid sequence may be identical to or have at least 70% homology, similarity or identity to SEQ ID NO: 127.

The microorganism described in this section can be used in a method for production of tryptophan, optionally said tryptophan is halogenated as described herein below.

### Tryptamine

In addition to the above the microorganism may be further engineered so that it can produce tryptamine and/or halogenated tryptamine. Tryptamine and/or halogenated tryptamine can be synthesised from tryptophan and/or halogenated tryptophan, respectively. In some embodiments, the microorganism expresses an enzyme that can convert tryptophan to tryptamine and/or halogenated tryptophan to halogenated tryptamine.

In some embodiments, the microorganism expresses GES and SGD, and further expresses tryptophan decarboxylase (TDC), which may not natively present in the microorganism. In other embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS or STR, and further expresses TDC. The microorganism may additionally also express TRP and/or a tryptophan halogenase and optionally a flavin reductase.

TDC has an EC number EC 4.1.1.28. TDC catalyses the conversion of tryptophan to tryptamine. The microorganism when expressing TDC is thus able to convert tryptophan to tryptamine and/or halogenated tryptophan to halogenated tryptamine, thus producing tryptamine and/or halogenated tryptamine, respectively.

In some embodiments, the microorganism expresses GES and SGD, and GPPS, G8H, CPR, CYB5, 8HGO, ISY, CYC, IO, CYPADH, 7DLGT, 7DLH, LAMT, SLS, STR and TDC.

In preferred embodiments, the TDC is a TDC native to Catharanthus roseus or Ruminococcus gnavus or functional variants thereof which retain the ability to convert tryptophan to tryptamine. Thus, in some embodiments, the TDC is CroTDC as set forth in SEQ ID NO: 79 or rgnTDC as set forth in SEQ ID NO: 80 or a functional variant thereof having at least 70% homology, similarity or identity to SEQ ID NO: 79 or SEQ ID NO: 80, respectively. In some embodiments, the TDC is CroTDC as set forth in SEQ ID NO: 79 or a functional variant thereof having at least 70% homology, similarity or identity to SEQ ID NO: 79. In other embodiments, the TDC is rgnTDC as set forth in SEQ ID NO: 80 or a functional variant thereof having at least 70% homology, similarity or identity to SEQ ID NO: 80. Functional variants of TDC, CroTDC, or rgnTDC may be identified by expressing said enzyme in a host cell, purifying the enzyme and performing an enzyme assay to measure the conversion from tryptophan to tryptamine and/or from halogenated tryptophan to halogenated tryptamine using standard high-performance liquid chromatography (LC) as described in Zhang et al. (2020) and/or GC-MS as described in Davies et al. (2021).

The TDC may be expressed in the microorganism by introducing a nucleic acid sequence as detailed further below, which encodes a TDC. In particular, the nucleic acid sequence may be identical to or have at least 70% homology, similarity or identity to SEQ ID NO: 17 or SEQ ID NO: 18, respectively.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); and/or CroTDC (SEQ ID NO: 79) and/or rgnTDC (SEQ ID NO: 80) or functional variants thereof having at least 70% homology, similarity or identity to SEQ ID NO: 65, 66, 82, 139, 63, 64, 68, 69, 67, 70, 71, 72, 73, 74, 75, 76, 77, 78, 79 and/or 80, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing tryptamine and/or halogenated tryptamine, may also produce tryptophan and/or halogenated tryptophan or the medium may comprise tryptophan or halogenated tryptophan as described in the section “Medium” herein below. The microorganism described in this section can be used in a method for production of tryptamine, optionally said tryptamine is halogenated as described herein below.

### Strictosidine

In addition to the above the microorganism may be further engineered so that it can produce strictosidine and/or halogenated strictosidine. Strictosidine can be synthesised from secologanin and tryptamine. Halogenated strictosidine can be synthesised from secologanin and halogenated tryptamine. In some embodiments, the microorganism expresses an enzyme that can convert secologanin and tryptamine to strictosidine, and/or secologanin and halogenated tryptamine to halogenated strictosidine.

It must be understood, that the tryptamine and/or strictosidine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

Tryptamine and/or halogenated tryptamine may be provided to the microorganism, for example as part of the medium the microorganism is incubated in. However, in some embodiments the microorganism may also be capable of synthesising tryptamine and/or halogenated tryptamine, for example the microorganism is further engineered to synthesise tryptamine and/or halogenated tryptamine as detailed herein above. In all cases, the halogenated tryptamine may be as described in section “Products, substrates and compounds” herein below. Secologanin may also be provided to the microorganism, for example as part of the medium the microorganism is incubated in as also detailed herein, for example herein below. However, the microorganism may also be capable of synthesising secologanin as detailed herein above.

In some embodiments, the microorganism expresses GES and SGD, and optionally at least one of GPPS, CPR, CYB5, G8H, 8HGO, ISY, CYC, IO, CYPADH, 7DLGT, 7DLH, LAMT, TDC or SLS, and further expresses strictosidine synthase (STR), which is not natively present in the microorganism. STR has an EC number EC 4.3.3.2. STR catalyses the conversion of secologanin and tryptamine to strictosidine and/or of secologanin and halogenated tryptamine to halogenated strictosidine. The microorganism expressing STR is thus capable of converting secologanin and tryptamine to strictosidine and/or secologanin and halogenated tryptamine to halogenated strictosidine, thus producing strictosidine and/or halogenated strictosidine, respectively.

In some embodiments, the microorganism expresses GES and SGD, and GPPS, CPR, CYB5, G8H, 8HGO, ISY, CYC, IO, CYPADH, 7DLGT, 7DLH, LAMT, SLS and STR. The microorganism may additionally also express TRP and/or a tryptophan halogenase and optionally a flavin reductase. Thus, in some embodiments, the microorganism expresses GES and SGD, and GPPS, CPR, CYB5, G8H, 8HGO, ISY, CYC, IO, CYPADH, 7DLGT, 7DLH, LAMT, SLS and STR, and further expresses TRP and TDC. In some embodiments, the microorganism expresses GES and SGD, and GPPS, CPR, CYB5, G8H, 8HGO, ISY, CYC, IO, CYPADH, 7DLGT, 7DLH, LAMT, SLS and STR, and further expresses TDC. In some embodiments, the microorganism expresses GES and SGD, and GPPS, CPR, CYB5, G8H, 8HGO, ISY, CYC, IO, CYPADH, 7DLGT, 7DLH, LAMT, SLS, TDC and STR, and further expresses tryptophan halogenase and optionally a flavin reductase.

In some embodiments, the STR is a STR native to Catharanthus roseus or a functional variant thereof which retains the ability to convert secologanin and tryptamine to strictosidine. Thus, in some embodiments, the STR is CroSTR as set forth in SEQ ID NO: 81 or a functional variant thereof having at least 70% homology, similarity or identity to SEQ ID NO: 81. Functional variants of STR or CroSTR may be identified by expressing said enzyme in a host cell, purifying the enzyme and performing an enzyme assay to measure the conversion from secologanin and tryptamine to strictosidine using LC-MS ss described in Brown et al. (2015).

The STR may be expressed in the microorganism by introducing a nucleic acid sequence as detailed further below, which encodes a STR. In particular, the nucleic acid sequence may be identical to or have at least 70% homology, similarity or identity to SEQ ID NO: 19.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68); CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); and/or CroSTR (SEQ ID NO: 81); or functional variants thereof having at least 70% homology, similarity or identity to SEQ ID NO: 65, 66, 82, 139, 63, 64, 68, 69, 67, 70, 71, 72, 73, 74, 75, 76, 77, 78 and/or 81, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In other embodiments, the microorganism expresses CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78) and CroSTR (SEQ ID NO: 81) or functional variants thereof having at least 70% homology, similarity or identity to SEQ ID NO: 68, 69, 67, 70, 71, 72, 73, 74, 75, 76, 77, 78, and/or 81, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In some embodiments, the microorganism expresses ERG20**-GS-trunCroGES (SEQ ID NO: 66), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79) and CroSTR (SEQ ID NO: 81) or functional variants thereof having at least 70% homology, similarity or identity to SEQ ID NO: 66, 63, 64, 68, 69, 67, 70, 71, 72, 73, 74, 75, 76, 77, 78, 79 and/or 81, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In further some embodiments, the microorganism expresses ERG20**-GS-trunCroGES (SEQ ID NO: 66), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), rgnTDC (SEQ ID NO: 80) and CroSTR (SEQ ID NO: 81) or functional variants thereof having at least 70% homology, similarity or identity to SEQ ID NO: 66, 63, 64, 68, 69, 67, 70, 71, 72, 73, 74, 75, 76, 77, 78, 80 and/or 81, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing strictosidine and/or halogenated strictosidine, also produces tryptamine and secologanin, and/or secologanin and halogenated tryptamine. The microorganism described in this section can be used in a method for production of strictosidine, optionally said strictosidine is halogenated as described herein below.

### Strictosidine Aglycone

In addition to the above the microorganism may be further engineered so that it can produce strictosidine aglycone and/or halogenated strictosidine aglycone. Strictosidine aglycone and/or halogenated strictosidine aglycone can be synthesised from strictosidine and/or halogenated strictosidine, respectively. In some embodiments, the microorganism expresses an enzyme that can convert strictosidine to strictosidine aglycone, and/or halogenated strictosidine to halogenated strictosidine aglycone.

It must be understood, that the strictosidine and/or strictosidine aglycone referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES, and optionally at least one of GPPS, CPR, CYB5, G8H, 8HGO, ISY, CYC, IO, CYPADH, 7DLGT, 7DLH, LAMT, TDC, SLS or STR, and further expresses strictosidine-O-β-D-glucosidase (SGD), which is not natively present in the microorganism. SGD has an EC number EC 3.2.1.105. SGD catalyses the conversion of strictosidine to strictosidine aglycone. In other words, SGD hydrolyses strictosidine to strictosidine aglycone. SGD may also catalyse the conversion of halogenated strictosidine to halogenated strictosidine aglycone. The microorganism expressing SGD is thus capable of converting strictosidine to strictosidine aglycone and/or halogenated strictosidine to halogenated strictosidine aglycone, thus producing strictosidine aglycone and/or halogenated strictosidine aglycone, respectively.

The microorganism may be modified as described in detail in application WO2020/229516 entitled “Methods for production of strictosidine aglycone and monoterpenoid indole alkaloids” filed on 13 May 2020 and assigned to the same applicant, particularly in the sections entitled “Strictosidine-O-beta-D-glucosidase (SGD)”, “Heterologous SGD or variants thereof” and “Mosaic SGD or variants thereof” therein. SGD may also sometimes be referred to as strictosidine-beta-glucosidase, strictosidine-β-D-glucosidase and/or strictosidine-beta-D-glucosidase.

In some embodiments, the microorganism expresses GES and SGD, and GPPS, CPR, CYB5, G8H, 8HGO, ISY, CYC, IO, CYPADH, 7DLGT, 7DLH, LAMT, SLS, STR and SGD. In other embodiments, the microorganism expresses GES and SGD, and GPPS, CPR, CYB5, G8H, 8HGO, ISY, CYC, IO, CYPADH, 7DLGT, 7DLH, LAMT, SLS, STR, and SGD, and further expresses TRP and TDC. In some embodiments, the microorganism expresses GES and SGD, and GPPS, CPR, CYB5, G8H, 8HGO, ISY, CYC, IO, CYPADH, 7DLGT, 7DLH, LAMT, SLS, STR and SGD, and further expresses TDC. The microorganism may additionally also express TRP and/or a tryptophan halogenase and optionally a flavin reductase.

In some embodiments, the SGD is a SGD native to Rauvolfia serpentina or a functional variant thereof which retains the ability to convert strictosidine to strictosidine aglycone and/or halogenated strictosidine to halogenated strictosidine aglycone. Thus, in some embodiments, the SGD is RseSGD as set forth in SEQ ID NO: 82 or a functional variant thereof having at least 70% homology, similarity or identity to SEQ ID NO: 82.

In other embodiments, the SGD is a SGD native to or derived from Vinca minor, Amsonia hubrichtii, Handroanthus impetiginosus, Sesamum indicum, Tabernaemontana elegans, Vigna unguiculata, Nyssa sinensis, Lomentospora prolificans, Actinidia chinensis, Heliocybe sulcate, Moniliophthora roreri, Rauvolfia serpentina, Pyricularia grisea, Ophiorrhiza pumila, Hydnomerulius pinastri, Helianthus annuus, Carapichea ipecacuanha, Lactuca sativa or a functional variant thereof. In some embodiments, the SGD is VmiSGD1 from Vinca minor(SEQ ID NO: 47 in WO2020/229516), AhuSGD from Amsonia hubrichtii (SEQ ID NO: 48 in WO2020/229516), TelSGD from Tabernaemontana elegans (SEQ ID NO: 51 in WO2020/229516), HimSGD2 (PIN06789.1), SinSGD (XP_011094151.1), VunSGD (XP_027910736.1), NsiSGD1 (KAA8549635.1), LprSGD (PKS11920.1), AchSGD1 (PSS10019.1), HsuSGD (TFK52902.1), MroSGD (ESK96275.1), RseRGD (AAF03675.1_1), PgrSGD (AAX07701.1), OpuSGD (BAP90523.1_1), HpiSGD (KIJ63193.1), HanSGD (XP_02201531.1), AchSGD2 (PSR88404.1), HimSGD1 (PIN07435.1), IpeSGD (BAH02544.1), LsaSGD1 (XP_023770227.1) or functional variants thereof having at least 70% homology, similarity or identity to VmiSGD1, AhuSGD, TelSGD, HimSGD1, SinSGD, VunSGD, NsiSGD1, LprSGD, AchSGD1, HsuSGD, MroSGD, RseRGD, PgrSGD, OpuSGD, HpiSGD, HanSGD1, AchSGD2, HimSGD1, IpeSGD, LsaSGD, respectively.

The SGD may be an engineered, new and active mosaic SGD capable of converting strictosidine to strictosidine aglycone. Design, engineering and/or characterisation of said mosaic SGD is described in detail in application WO2020/229516 entitled “Methods for production of strictosidine aglycone and monoterpenoid indole alkaloids” filed on 13 May 2020 and assigned to the same applicant, particularly in the sections entitled “Mosaic SGD or variants thereof” and/or “Example 3”, “Example 7”, “Example 8”, “Example 9” and/or “Example 10” therein. In some embodiments, the SGD is a mosaic SGD such as a hydrid SGD of RseSGD (SEQ ID NO: 82) and the Catharanthus roseus SGD CroSGD (ABW77570.1) such as hybrid SGD C1-R2-R3-C4 (SEQ ID NO: 108 in WO2020/229516), hybrid SGD R1-R2-R3-C4 (SEQ ID NO: 96 in WO2020/229516), hybrid SGD R1-C2-R3-C4 (SEQ ID NO: 97 in WO2020/229516), hybrid SGD C1-C2-R3-C4 (SEQ ID NO: 98 in WO2020/229516), hybrid SGD R1-C2-R3-R4 (SEQ ID NO: 95 in WO2020/229516), hybrid SGD C1-R2-R3-R4 (SEQ ID NO: 94 in WO2020/229516), hybrid SGD C1-C2-R3-R4 (SEQ ID NO: 93 in WO2020/229516) or functional variants thereof having at least 70% homology, similarity or identity to each of them respectively.

Functional variants of SGD or RseSGD may be identified by expressing said enzyme in a host cell, purifying the enzyme and performing an enzyme assay to measure the conversion from strictosidine to strictosidine aglycone using standard techniques. The conversion of strictosidine to strictosidine aglycone, may be measured directly by the amount of strictosidine aglycone as known in the art, or surrogate measure of the conversion of strictosidine to strictosidine aglycone may be measured as known in the art. Because strictosidine aglycone is highly reactive, indirect determination of strictosidine aglycone may be preferred. For example, colorimetric assays to follow strictosidine consumption as described in Geerlings et al., 2000, may be used. The disappearance of strictosidine may also be monitored by UV, as described in Guirimand et al., 2010, or the general β-glucosidase activity in the cells may be measured, e.g. by UV detection of a synthetic substrate such as 4-methylumbelliferyl-β-D-glucoside (Guirimand et al., 2010).

Thus, to determine whether a SGD is capable of converting strictosidine to strictosidine aglycone, the person skilled in the art could use any of said methods, or could use high-precision mass spectrometry to detect the accurate mass of strictosidine aglycone after cultivation of a strain expressing an SGD or an enzyme suspected of having SGD activity in a medium; the cell is either provided with strictosidine in the medium or it has been engineered and can synthesise strictosidine. The strictosidine aglycone can be detected directly in the medium or in a pellet, after centrifugation of the culture broth. Alternatively, the appearance of other products, downstream of strictosidine aglycone, for example tetrahydroalstonine, can be monitored; such products will only form in the presence of a functional SGD, strictosidine, and an enzyme capable of using strictosidine aglycone, as described in e.g. Stavrinides et al., 2015.

The SGD may be expressed in the microorganism by introducing a nucleic acid sequence as detailed further below, which encodes a SGD. In particular, the nucleic acid sequence may be identical to or have at least 70% homology, similarity or identity to SEQ ID NO: 20.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68); CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); and/or CroSTR (SEQ ID NO: 81); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80 and/or 81, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing strictosidine aglycone and/or halogenated strictosidine aglycone, also produces tryptamine, secologanin and strictosidine and/or secologanin, halogenated tryptamine and halogenated strictosidine. The microorganism described in this section can be used in a method for production of strictosidine aglycone, optionally said strictosidine aglycone is halogenated as described herein below.

Thus, also disclosed herein is a microorganism producing halogenated strictosidine aglycone and/or derivatives thereof, in the presence of GPP and halogenated tryptamine, said microorganism expressing a GES (EC 3.1.7.11) and a SGD (EC 3.2.1.105), optionally wherein:
the GES is a GES capable of converting GPP to geraniol, optionally said GES is a heterologous GES, preferably said GES is as set forth in SEQ ID NO: 65 and/or as set forth in SEQ ID NO: 66, or a functional variant thereof having at least 70% homology to SEQ ID NO: 65 and/or SEQ ID NO: 66; and/or
the SGD is a SGD capable of converting halogenated strictosidine to halogenated strictosidine aglycone, optionally said SGD is a heterologous SGD, preferably said SGD is RseSGD (SEQ ID NO: 82), or a functional variant thereof having at least 70% homology to SEQ ID NO: 82.

### Tetrahydroalstonine, Ajmalicine, Serpentine and Alstonine

In addition to the above the microorganism may be further engineered so that it can produce tetrahydroalstonine, ajmalicine, alstonine and/or serpentine, and/or halogenated tetrahydroalstonine, ajmalicine, alstonine and/or serpentine. In some embodiments, the microorganism expresses one or more enzymes that together and/or individually can convert strictosidine aglycone to tetrahydroalstonine, ajmalicine, alstonine and/or serpentine, and/or halogenated strictosidine aglycone to halogenated tetrahydroalstonine, ajmalicine, alstonine and/or serpentine.

Tetrahydroalstonine, ajmalicine, alstonine and/or serpentine can be synthesised from strictosidine aglycone. Halogenated tetrahydroalstonine, ajmalicine, alstonine and/or serpentine can be synthesised from halogenated strictosidine aglycone. The necessary substrates for each product may be provided to the microorganism as part of the medium used to incubate the microorganism. In preferred embodiments, the substrates for each of the above products may be synthesised by the microorganism itself. In all cases, the microorganism is capable of synthesising strictosidine aglycone and/or halogenated strictosidine aglycone.

In addition to the above, it must be understood, that the strictosidine aglycone and/or tetrahydroalstonine, ajmalicine, alstonine and/or serpentine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and further expresses tetrahydroalstonine synthase (THAS), which is not natively present in the microorganism. THAS has an EC number EC 1.-.-.- and converts strictosidine aglycone to tetrahydroalstonine. In other words, THAS catalyses conversion of strictosidine aglycone to tetrahydroalstonine. The microorganism when expressing THAS is thus able to convert strictosidine aglycone to tetrahydroalstonine, thus producing tetrahydroalstonine.

In preferred embodiments, the THAS is a THAS native to Catharanthus roseus or a functional variant thereof which retains the ability to convert strictosidine aglycone to tetrahydroalstonine. Thus, in some embodiments, the THAS is CroTHAS1 as set forth in SEQ ID NO: 83 or a functional variant thereof having at least 70% homology, similarity or identity to SEQ ID NO: 83. Functional variants of THAS or CroTHAS1 may be identified by expressing said enzyme in a host cell, purifying the enzyme and performing an enzyme assay to measure the conversion from strictosidine aglycone to tetrahydroalstonine using LC-MC as described in Liu et al. (2022). The microorganism may be modified as described in detail in application WO2020/229516 entitled “Methods for production of strictosidine aglycone and monoterpenoid indole alkaloids” filed on 13 May 2020, particularly in the section “Tetrahydroalstonine synthase, heteroyohimbine synthase”, “Sarpargan bridge enzyme (SBE)”.

The THAS may be expressed in the microorganism by introducing a nucleic acid sequence as detailed further below, which encodes a THAS. In particular, the nucleic acid sequence may be identical to or have at least 70% homology, similarity or identity to SEQ ID NO: 21.

In some embodiments, the microorganism expresses GES and SGD, and further expresses heteroyohimbine synthase (HYS), which is not natively present in the microorganism. HYS has an EC number EC 1.-.-.- and converts strictosidine aglycone to tetrahydroalstonine, ajmalicine and/or mayumbine. In other words, HYS catalyses conversion of strictosidine aglycone to tetrahydroalstonine, ajmalicine and/or mayumbine. The microorganism when expressing HYS is thus able to convert strictosidine aglycone to tetrahydroalstonine, ajmalicine and/or mayumbine, thus producing tetrahydroalstonine, ajmalicine and/or mayumbine.

In preferred embodiments, the HYS is a HYS native to Catharanthus roseus or a functional variant thereof which retains the ability to convert strictosidine aglycone to tetrahydroalstonine, ajmalicine and/or mayumbine. Thus, in some embodiments, the HYS is CroHYS as set forth in SEQ ID NO: 84 or a functional variant thereof having at least 70% homology, similarity or identity to SEQ ID NO: 84. Functional variants of HYS or CroHYS may be identified by expressing said enzyme in a host cell, purifying the enzyme and performing an enzyme assay to measure the conversion from strictosidine aglycone to tetrahydroalstonine, ajmalicine and/or mayumbine using LC-MS as described in Liu et al. (2022). The microorganism may be modified as described in detail in application WO2020/229516 entitled “Methods for production of strictosidine aglycone and monoterpenoid indole alkaloids” filed on 13 May 2020, particularly in the section entitled “Tetrahydroalstonine synthase, heteroyohimbine synthase”.

The HYS may be expressed in the microorganism by introducing a nucleic acid sequence as detailed further below, which encodes a HYS. In particular, the nucleic acid sequence may be identical to or have at least 70% homology, similarity or identity to SEQ ID NO: 22.

In some embodiments, the microorganism expresses GES and SGD, and further expresses THAS and/or a HYS, thus producing tetrahydroalstonine and/or ajmalicine. In some embodiments, the microorganism expresses CroHYS and/or CroTHAS1 or functional variants thereof having at least 70% homology, similarity or identity to SEQ ID NO: 84 and/or SEQ ID NO: 83, respectively.

In addition to the above, the microorganism may be further engineered so that it can produce a heteroyohimbine, in particular alstonine and/or serpentine and/or halogenated alstonine and/or serpentine. Heteroyohimbines are a prevalent subclass of the MIAs, which are found in many plant species, primarily from the Apocynaceae and Rubiaceae families.

In some embodiments, the microorganism expresses GES and SGD, and optionally at least one of CPR, CYB5, THAS or HYS, and further expresses serpentine synthase (SS), which is not natively present in the microorganism.

SS has an EC number EC 1.14.14.-. SS catalyses conversion of ajmalicine to serpentine and/or conversion of tetrahydroalstonine to alstonine. The microorganism when expressing SS, and optionally CPR and/or CYB5, is thus able to convert ajmalicine to serpentine and/or tetrahydroalstonine to alstonine, thus producing serpentine and/or alstonine, respectively.

The CPR and/or CYB5 may be the CPR and/or CYB5, respectively, as defined in section “CPR and CYB5” herein above.

In preferred embodiments, the SS is a SS native to Catharanthus roseus or a functional variant thereof which retains the ability to convert ajmalicine to serpentine and/or tetrahydroalstonine to alstonine, respectively. Thus, in some embodiments, the SS is CroSS as set forth in SEQ ID NO: 110 or a functional variant thereof having at 70% homology, similarity or identity to SEQ ID NO: 110. Functional variants of SS or CroSS may be identified by expressing said enzyme in a host cell, purifying the enzyme and performing an enzyme assay to measure the conversion from ajmalicine to serpentine and/or tetrahydroalstonine to alstonine using standard techniques.

The SS may be expressed in the microorganism by introducing a nucleic acid sequence as detailed further below, which encodes a SS. In particular, the nucleic acid sequence may be identical to or have at least 70% homology, similarity or identity to SEQ ID NO: 48.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66), RseSGD (SEQ ID NO: 82), ERG20** (SEQ ID NO: 139), AgrGPPS2 (SEQ ID NO: 63) and/or GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78) and/or CroTDC (SEQ ID NO: 79) and/or rgnTDC (SEQ ID NO: 80), CroSTR (SEQ ID NO: 81), CroSS (SEQ ID NO: 110), CroTHAS1 (SEQ ID NO: 83) and/or CroHYS (SEQ ID NO: 84) or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 110, 83 and/or 84, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In some embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79), CroSTR (SEQ ID NO: 81), CroSS (SEQ ID NO: 110) and CroHYS (SEQ ID NO: 84) or functional variants thereof having at least 70% homology, similarity or identity to SEQ ID NO: 66, 82, 63, 64, 68, 69, 67, 70, 71, 72, 73, 74, 75, 76, 77, 78, 79, 81, 110 and/or 84, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In other embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79), CroSTR (SEQ ID NO: 81), CroSS (SEQ ID NO: 110) and CroTHAS1 (SEQ ID NO: 83) or functional variants thereof having at least 70% homology, similarity or identity to SEQ ID NO: 66, 82, 63, 64, 68, 69, 67, 70, 71, 72, 73, 74, 75, 76, 77, 78, 79, 81, 110 and/or 83, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In other embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), rgnTDC (SEQ ID NO: 80), CroSTR (SEQ ID NO: 81), CroSS (SEQ ID NO: 110), CroTHAS1 (SEQ ID NO: 83), IaeRebH_N470S (SEQ ID NO: 111) and EcoSsuE (SEQ ID NO: 112) or functional variants thereof having at least 70% homology, similarity or identity to SEQ ID NO: 66, 82, 63, 64, 68, 69, 67, 70, 71, 72, 73, 74, 75, 76, 77, 78, 80, 81, 110, 83, 111 and/or 112, respectively. In addition, the microorganism may express TRP5 (SEQ ID NO: 138) or functional variants thereof having at least 70% homology, similarity or identity to SEQ ID NO: 138.

In other embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), rgnTDC (SEQ ID NO: 80), CroSTR (SEQ ID NO: 81), CroSS (SEQ ID NO: 110), CroHYS (SEQ ID NO: 84), IaeRebH_N470S (SEQ ID NO: 111) and EcoSsuE (SEQ ID NO: 112) or functional variants thereof having at least 70% homology, similarity or identity to SEQ ID NO: 66, 82, 63, 64, 68, 69, 67, 70, 71, 72, 73, 74, 75, 76, 77, 78, 80, 81, 110, 84, 111 and/or 112, respectively. In addition, the microorganism may express TRP5 (SEQ ID NO: 138) or functional variants thereof having at least 70% homology, similarity or identity to SEQ ID NO: 138.

The microorganism described in this section, when producing ajmalicine, serpentine, tetrahydroalstonine and/or alstonine, and/or halogenated ajmalicine, serpentine, tetrahydroalstonine and/or alstonine, also produces secologanin, tryptamine, strictosidine and strictosidine aglycone, and/or secologanin, halogenated tryptamine, halogenated strictosidine and halogenated strictosidine aglycone. The microorganism described in this section can be used in a method for production of ajmalicine, serpentine, tetrahydroalstonine and/or alstonine, optionally said ajmalicine, serpentine, tetrahydroalstonine and/or alstonine is halogenated as described herein below.

### 19E-Geissoschizine

In addition to the above the microorganism may be further engineered so that it can produce 19E-geissoschizine and/or halogenated 19E-geissoschizine. 19E-geissoschizine and/or halogenated 19E-geissoschizine can be synthesised from strictosidine aglycone and/or halogenated strictosidine aglycone, respectively. In some embodiments, the microorganism expresses an enzyme that can convert strictosidine aglycone to 19E-geissoschizine and/or halogenated strictosidine aglycone to halogenated 19E-geissoschizine. 19E-geissoschizine is also known as geissoschizine and may herein also be referred to as geissoschizine.

In addition to the above, it must be understood, that the strictosidine aglycone and/or 19E-geissoschizine referred to in this section may also be halogenated, such as mono, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally THAS, HYS or SS, and further expresses geissoschizine synthase (GS), which is not natively present in the microorganism.

GS has an EC number EC 1.3.1.36 and catalyses the reduction of strictosidine aglycone to 19E-geissoschizine. The microorganism when expressing GS is thus able to convert strictosidine aglycone to 19E-geissoschizine, thus producing 19E-geissoschizine.

In some embodiments, the microorganism expresses GES and SGD, and GS. In some embodiments, the microorganism expresses GES, SGD, THAS, SS and GS. In other embodiments, the microorganism expresses GES, SGD, HYS, SS and GS. In other embodiments, the microorganism expresses GES; SGD, THAS, HYS, SS and GS.

The CPR and/or CYB5 may be the CPR and/or CYB5, respectively, as defined in section “CPR and CYB5” herein above.

In preferred embodiments, the GS is a GS native to Catharanthus roseus or a functional variant thereof which retains the ability to catalyse the reduction of strictosidine aglycone to 19E-geissoschizine. Thus, in some embodiments, the GS is CroGS as set forth in SEQ ID NO: 85 or a functional variant thereof having at least 70% homology, similarity or identity to SEQ ID NO: 85. In other embodiments, the GS is CroADH13 as set forth in SEQ ID NO: 86 or a functional variant thereof having at least 70% homology, similarity or identity to SEQ ID NO: 86. Functional variants of GS, CroGS or CroADH13 may be identified by expressing said enzyme in a host cell, purifying the enzymes and performing an enzyme assay to measure the conversion from strictosidine aglycone to 19E-geissoschizine using LC-MS/MS as described by Dang et al. (2018).

The GS may be expressed in the microorganism by introducing a nucleic acid sequence as detailed further below, which encodes a GS. In particular, the nucleic acid sequence may be identical to or have at least 70% homology, similarity or identity to SEQ ID NO: 23 and/or SEQ ID NO: 24, respectively.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

Preferably, the GS is CroGS from Catharanthus roseus (SEQ ID NO: 85) or a functional variant thereof having at least 70% homology, similarity or identity to SEQ ID NO: 85.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68); CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); and/or CroGS (SEQ ID NO: 85) and/or CroADH13 (SEQ ID NO: 86) or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, and/or 86, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone and 19E-geissoschizine, and/or secologanin, halogenated tryptamine, halogenated strictosidine, halogenated strictosidine aglycone and halogenated 19E-geissoschizine. The microorganism described in this section can be used in a method for production of 19E-geissoschizine, optionally said 19E-geissoschizine is halogenated as described herein below.

### Stemmadenine

In addition to the above the microorganism may be further engineered so that it can produce stemmadenine and/or halogenated stemmadenine. Stemmadenine and/or halogenated stemmadenine can be synthesised from 19E-geissoschizine and/or halogenated 19E-geissoschizine, respectively. In some embodiments, the microorganism expresses one or more enzyme that together and/or individually can convert 19E-geissoschizine to stemmadenine and/or halogenated 19E-geissoschizine to halogenated stemmadenine.

In addition to the above, it must be understood, that the 19E-geissoschizine and/or stemmadenine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of CPR, CYB5, THAS, HYS, SS or GS, and further expresses geissoschizine oxidase (GO), protein Redox1 and/or protein Redox2, which are not natively present in the microorganism.

GO has an EC number EC 1.14.14.- and catalyses the oxidation of 19E-geissoschizine to produce a short-lived MIA unstable intermediate which can be oxidized either by protein Redox1 and protein Redox2 to produce stemmadenine. Protein Redox1 has a EC number EC 1.14.14.- and catalyses the first of two oxidation steps that convert the unstable product resulting from oxidation of 19E-geissoschizine by GO to stemmadenine. Protein Redox2 has an EC number EC 1.7.1.- and catalyses the second of two oxidation steps that convert the unstable product resulting from oxidation of 19E-geissoschizine by GO to stemmadenine. The microorganism when expressing GO, and optionally CPR and/or CYB5, protein Redox1 and protein Redox2 are thus able to convert 19E-geissoschizine to stemmadenine, thus producing stemmadenine. Stemmadenine is also known as 15a-stemmadenine and may herein be referred to as 15a-stemmadenine.

The CPR and/or CYB5 may be the CPR and/or CYB5, respectively, as defined in section “CPR and CYB5” herein above.

In some embodiments, the microorganism expresses GES and SGD, and GS, CPR, CYB5, GO, protein Redox1 and protein Redox2.

In some embodiments, the GO is a GO native to Catharanthus roseus, Tabernaemontana elegans, Vinca minor and/or Amsonia hubrichtii or a functional variant thereof which retains the ability to catalyse the oxidation of 19E-geissoschizine to produce a short-lived MIA unstable intermediate which can be oxidized either by protein Redox1 and protein Redox2 to produce stemmadenine. Thus, in some embodiments, the GO is CroGO as set forth in SEQ ID NO: 88 or a functional variant thereof having at least 70% homology, similarity or identity to SEQ ID NO: 88. In other embodiments, the GO is TelGO as set forth in SEQ ID NO: 89 or a functional variant thereof having at least 70% homology, similarity or identity to SEQ ID NO: 89. In other embodiments, the GO is VmiGO as set forth in SEQ ID NO: 90 or a functional variant thereof having at least 70% homology, similarity or identity to SEQ ID NO: 90. In other embodiments, the GO is AhuGO as set forth in SEQ ID NO: 91 or a functional variant thereof having at least 70% homology, similarity or identity to SEQ ID NO: 91. Functional variants of GO, CroGO, TelGO, VmiGO or AhuGO may be identified by expressing said enzyme in a host cell, purifying the enzyme and performing an enzyme assay to measure the conversion 19E-geissoschizine to a short-lived MIA unstable intermediate which may be oxidized by protein Redox1 and/or protein Redox2 to stemmadenine using standard techniques.

Preferably, the GO is AhuGO (SEQ ID NO: 91) or a functional variant having at least 70% homology, similarity or identity thereto.

The GO may be expressed in the microorganism by introducing a nucleic acid sequence as detailed further below, which encodes a GO. In particular, the nucleic acid sequence may be identical to or have at least 70% homology, similarity or identity to SEQ ID NO: 26, SEQ ID NO: 27, SEQ ID NO: 28, and/or SEQ ID NO: 29, respectively.

In preferred embodiments, the protein Redox1 is a protein Redox1 native to Catharanthus roseus or a functional variant thereof which retains the ability to catalyse the first of two oxidation steps that convert the unstable product resulting from oxidation of 19E-geissoschizine by GO to stemmadenine. Thus, in some embodiments, the protein Redox1 is CroRdx1 as set forth in SEQ ID NO: 92 or a functional variant thereof having at least 70% homology, similarity or identity to SEQ ID NO: 92. Functional variants of protein Redox1 or CroRdx1 may be identified by expressing said enzyme in a host cell, purifying the enzyme and performing an enzyme assay to measure the conversion the unstable product resulting from oxidation of 19E-geissoschizine by GO to stemmadenine using liquid chromatography with tandem mass spectrometry (LCMS/MS) as described by Qu et al. (2018).

The protein Redox1 may be expressed in the microorganism by introducing a nucleic acid sequence as detailed further below, which encodes a protein Redox1. In particular, the nucleic acid sequence may be identical to or have at least 70% homology, similarity or identity to SEQ ID NO: 30.

In preferred embodiments, the protein Redox2 is a protein Redox2 native to Catharanthus roseus or a functional variant thereof which retains the ability to catalyse the second of two oxidation steps that the converts the unstable product resulting from oxidation of 19E-geissoschizine by GO to stemmadenine. Thus, in some embodiments, the protein Redox2 is CroRdx2 as set forth in SEQ ID NO: 93 or a functional variant thereof having at least 70% homology, similarity or identity to SEQ ID NO: 93. Functional variants of protein Redox2 or CroRdx2 may be identified by expressing said enzyme in a host cell, purifying the enzyme and performing an enzyme assay to measure the conversion of the unstable product resulting from oxidation of 19E-geissoschizine by GO to stemmadenine using LC-MS/MS as described by Qu et al. (2018).

The protein Redox2 may be expressed in the microorganism by introducing a nucleic acid sequence as detailed further below, which encodes a protein Redox2. In particular, the nucleic acid sequence may be identical to or have at least 70% homology, similarity or identity to SEQ ID NO: 31.

Sometimes, protein Redox1 may be referred to as Redox1 and/or protein Redox2 may be referred to as Redox2.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92 and/or 93, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine and stemmadenine, and/or secologanin, halogenated tryptamine, halogenated strictosidine, halogenated strictosidine aglycone, halogenated 19E-geissoschizine and halogenated stemmadenine. The microorganism described in this section can be used in a method for production of stemmadenine, optionally said stemmadenine is halogenated as described herein below.

### Stemmadenine Acetate

In addition to the above the microorganism may be further engineered so that it can produce O-acetylstemmadenine and/or halogenated O-acetylstemmadenine, also known as stemmadenine acetate and/or halogenated stemmadenine acetate. O-acetylstemmadenine and/or halogenated O-acetylstemmadenine can be synthesised from stemmadenine and/or halogenated stemmadenine, respectively. In some embodiments, the microorganism expresses an enzyme that can convert stemmadenine to O-acetylstemmadenine and/or halogenated stemmadenine to halogenated O-acetylstemmadenine. O-acetylstemmadenine may also herein be referred to as stemmadenine acetate and vice versa. Halogenated O-acetylstemmadenine may also herein be referred to as halogenated stemmadenine acetate.

In addition to the above, it must be understood, that the stemmadenine and/or O-acetylstemmadenine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1 or protein Redox2, and further expresses stemmadenine O-acetyltransferase (SAT), which is not natively present in the microorganism.

SAT has an EC number EC 1.7.1.- and catalyses the acetylation of stemmadenine to O-acetylstemmadenine. The microorganism when expressing SAT is thus able to convert stemmadenine to O-acetylstemmadenine, thus producing O-acetylstemmadenine.

In some embodiments, the microorganism expresses GES and SGD, and GS, CPR, CYB5, GO, protein Redox1, protein Redox2 and SAT.

In preferred embodiments, the SAT is a SAT native to Catharanthus roseus or a functional variant thereof which retains the ability to convert stemmadenine to O-acetylstemmadenine. Thus, in some embodiments, the SAT is CroSAT as set forth in SEQ ID NO: 94 or a functional variant thereof having at least 70% homology, similarity or identity to SEQ ID NO: 94. Functional variants of SAT or CroSAT may be identified by expressing said enzyme in a host cell, purifying the enzyme and performing an enzyme assay to measure the conversion from stemmadenine to O-acetylstemmadenine using LC-MS/MS as described by Farrow et al. (2018).

The SAT may be expressed in the microorganism by introducing a nucleic acid sequence as detailed further below, which encodes a SAT. In particular, the nucleic acid sequence may be identical to or have at least 70% homology, similarity or identity to SEQ ID NO: 32.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); and/or CroSAT (SEQ ID NO: 94); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93 and/or 94, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In preferred embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79), CroSTR (SEQ ID NO: 81), CroGS (SEQ ID NO: 85), AhuGO (SEQ ID NO: 91), CroRdx1 (SEQ ID NO: 92), CroRdx2 (SEQ ID NO: 93) and CroSAT (SEQ ID NO: 94) or functional variants thereof having at least 70% homology, similarity or identity to SEQ ID NO: 66, 82, 63, 64, 68, 69, 67, 70, 71, 72, 73, 74, 75, 76, 77, 78, 79, 81, 85, 91, 92, 93 and/or 94, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine and stemmadenine acetate, and/or secologanin, halogenated tryptamine, halogenated strictosidine, halogenated strictosidine aglycone, halogenated 19E-geissoschizine, halogenated stemmadenine and halogenated stemmadenine acetate. The microorganism described in this section can be used in a method for production of stemmadenine acetate, optionally said stemmadenine acetate is halogenated as described herein below.

### Precondylcarpine Acetate

In addition to the above the microorganism may be further engineered so that it can produce precondylocarpine acetate and/or halogenated precondylocarpine acetate. Precondylocarpine acetate and/or halogenated precondylocarpine acetate can be synthesised from O-acetylstemmadenine and/or halogenated O-acetylstemmadenine, respectively. In some embodiments, the microorganism expresses an enzyme that can convert O-acetylstemmadenine to precondylocarpine acetate and/or halogenated O-acetylstemmadenine to halogenated precondylocarpine acetate.

In addition to the above, it must be understood, that the O-acetylstemmadenine and/or precondylocarpine acetate referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2 or SAT, and further expresses precondylocarpine acetate synthase/O-acetylstemmadenine oxidase (PAS/ASO), which is not natively present in the microorganism. Precondylocarpine acetate synthase (PAS) and O-acetylstemmadenine oxidase (ASO) are two different names for the same enzyme with an EC number EC 1.21.3.- and that converts O-acetylstemmadenine to precondylocarpine acetate. Therefore, in this disclosure precondylocarpine acetate synthase (PAS) and O-acetylstemmadenine oxidase (ASO) are referred to as precondylocarpine acetate synthase/O-acetylstemmadenine oxidase (PAS/ASO). However, sometimes only PAS may be used to refer to the enzyme and other times only ASO may be used to refer to the enzyme. The microorganism when expressing PAS/ASO is thus able to convert O-acetylstemmadenine to precondylocarpine acetate, thus producing precondylocarpine acetate.

In some embodiments, the microorganism expresses GES and SGD, and GS, CPR, CYB5, GO, protein Redox1, protein Redox2, SAT and PAS/ASO.

In preferred embodiments, the PAS/ASO is a PAS/ASO native to Catharanthus roseus, Amsonia hubrichtii, Tabernanthe iboga and/or Tabernaemontana elegans or functional variants thereof which retain the ability to convert O-acetylstemmadenine to precondylocarpine acetate. Thus, in some embodiments, the PAS/ASO is CroPAS as set forth in SEQ ID NO: 95 or a functional variant thereof having at least 70% homology, similarity or identity to SEQ ID NO: 95. In other embodiments, the PAS/ASO is AhuASO as set forth in SEQ ID NO: 96 or a functional variant thereof having at least 70% homology, similarity or identity to SEQ ID NO: 96. In other embodiments, the PAS/ASO is TibPAS2 as set forth in SEQ ID NO: 97 or a functional variant thereof having at least 70% homology, similarity or identity to SEQ ID NO: 97. In other embodiments, the PAS/ASO is TelASO as set forth in SEQ ID NO: 98 or a functional variant thereof having at least 70% homology, similarity or identity to SEQ ID NO: 98. Functional variants of PAS/ASO, CroPAS, AhuASO, TibPAS2 or TelASO may be identified by expressing said enzyme in a host cell, purifying the enzyme and performing an enzyme assay to measure the conversion from O-acetylstemmadenine to precondylocarpine acetate using LC-MS/MS as described by Farrow et al. (2018) or liquid chromatograph-high resolution mass spectrometry (LC-HRMS).

The PAS/ASO may be expressed in the microorganism by introducing a nucleic acid sequence as detailed further below, which encodes a PAS/ASO. In particular, the nucleic acid sequence may be identical to or have at least 70% homology, similarity or identity to SEQ ID NO: 33, SEQ ID NO: 34, SEQ ID NO: 35 and/or SEQ ID NO: 36, respectively.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the PAS/ASO is a Tabernanthe iboga PAS/ASO, preferably TibPAS2 as set forth in SEQ ID NO: 97 or a functional variant thereof having at least 70% homology, similarity or identity to thereto.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); and/or CroPAS (SEQ ID NO: 95), AhuASO (SEQ ID NO: 96), TibPAS2 (SEQ ID NO: 97) and/or TelASO (SEQ ID NO: 98); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 97 and/or 98, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate and precondylocarpine acetate, and/or secologanin, halogenated tryptamine, halogenated strictosidine, halogenated strictosidine aglycone, halogenated 19E-geissoschizine, halogenated stemmadenine, halogenated stemmadenine acetate and halogenated precondylocarpine acetate. The microorganism described in this section can be used in a method for production of precondylocarpine acetate, optionally said precondylocarpine acetate is halogenated as described herein below.

### Dihydroprecondylocarpine Acetate

In addition to the above the microorganism may be further engineered so that it can produce dihydroprecondylocarpine acetate and/or halogenated dihydroprecondylocarpine acetate. Dihydroprecondylocarpine acetate and/or halogenated dihydroprecondylocarpine acetate can be synthesised from precondylocarpine acetate and/or halogenated precondylocarpine acetate, respectively. In some embodiments, the microorganism expresses an enzyme that can convert precondylocarpine acetate to dihydroprecondylocarpine acetate and/or halogenated precondylocarpine acetate to halogenated dihydroprecondylocarpine acetate.

In addition to the above, it must be understood, that the precondylocarpine acetate and/or dihydroprecondylocarpine acetate referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT or PAS/ASO, and further expresses dihydroprecondylocarpine acetate synthase (DPAS), which is not natively present in the microorganism.

DPAS has an EC number EC 1.1.1.- and converts precondylocarpine acetate to dihydroprecondylocarpine acetate. The microorganism when expressing DPAS is thus able to convert precondylocarpine acetate to dihydroprecondylocarpine acetate, thus producing dihydroprecondylocarpine acetate.

In some embodiments, the microorganism expresses GES and SGD, and GS, CPR, CYB5, GO, protein Redox1, protein Redox2, SAT, PAS/ASO and DPAS.

In preferred embodiments, the DPAS is a DPAS native to Catharanthus roseus or Tabernanthe iboga or a functional variant thereof which retains the ability to convert precondylocarpine acetate to dihydroprecondylocarpine acetate. Thus, in some embodiments, the DPAS is CroDPAS as set forth in SEQ ID NO: 99 or a functional variant thereof having at least 70% homology, similarity or identity to SEQ ID NO: 99. In other embodiments, the DPAS is TibDPAS2 as set forth in SEQ ID NO: 100 or a functional variant thereof having at least 70% homology, similarity or identity to SEQ ID NO: 100.

Functional variants of DPAS, CroDPAS or TibDPAS2 may be identified by expressing said enzyme in a host cell, purifying the enzyme and performing an enzyme assay to measure the conversion from precondylocarpine acetate to dihydroprecondylocarpine acetate using LC-MS/MS described by Caputi et al. (2018).

The DPAS may be expressed in the microorganism by introducing a nucleic acid sequence as detailed further below, which encodes a DPAS. In particular, the nucleic acid sequence may be identical to or have at least 70% homology, similarity or identity to SEQ ID NO: 37 and/or SEQ ID NO: 38, respectively.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, DPAS is a Tabernanthe iboga DPAS, preferably TibDPAS2 as set forth in SEQ ID NO: 100 or a functional variant thereof having at least 70% homology, similarity or identity to thereto.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); and/or CroDPAS (SEQ ID NO: 99) and/or TibDPAS2 (SEQ ID NO: 100); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 97, 98, 99 and/or 100, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate and dihydroprecondylocarpine acetate, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, and halogenated dihydroprecondylocarpine acetate. The microorganism described in this section can be used in a method for production of dihydroprecondylocarpine acetate, optionally said dihydroprecondylocarpine acetate is halogenated as described herein below.

### Catharanthine

In addition to the above the microorganism may be further engineered so that it can produce catharanthine and/or halogenated catharanthine. Catharanthine and/or halogenated catharanthine can be synthesised from dihydroprecondylocarpine acetate and/or halogenated dihydroprecondylocarpine acetate, respectively. In some embodiments, the microorganism expresses an enzyme that can convert dihydroprecondylocarpine acetate to catharanthine and/or halogenated dihydroprecondylocarpine acetate to halogenated catharanthine.

In addition to the above, it must be understood, that the dihydroprecondylocarpine acetate and/or catharanthine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO or DPAS, and further expresses catharanthine synthase (CS), which is not natively present in the microorganism.

CS has an EC number EC 4.-.-.- and converts dihydroprecondylocarpine acetate to catharanthine. The microorganism when expressing CS is thus able to convert dihydroprecondylocarpine acetate to catharanthine, thus producing catharanthine.

In some embodiments, the microorganism expresses GES and SGD, and GS, CPR, CYB5, GO, protein Redox1, protein Redox2, SAT, PAS/ASO, DPAS and CS. Optionally the microorganism also expresses TS as described herein below.

In preferred embodiments, the CS is a CS native to Catharanthus roseus or a functional variant thereof which retains the ability to convert dihydroprecondylocarpine acetate to catharanthine. Thus, in some embodiments, the CS is CroCS as set forth in SEQ ID NO: 102 or a functional variant thereof having at least 70% homology, similarity or identity to SEQ ID NO: 102. Functional variants of CS or CroCS may be identified by expressing said enzyme in a host cell, purifying the enzyme and performing an enzyme assay to measure the conversion from dihydroprecondylocarpine acetate to catharanthine using LC-MS/MS described by Caputi et al. (2018).

The CS may be expressed in the microorganism by introducing a nucleic acid sequence as detailed further below, which encodes a CS. In particular, the nucleic acid sequence encoding CroCS may be identical to or have at least 70% homology, similarity or identity to SEQ ID NO: 40.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); and/or CroCS (SEQ ID NO: 102); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100 and/or 102, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate and catharanthine, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate and halogenated catharanthine. The microorganism described in this section can be used in a method for production of catharanthine, optionally said catharanthine is halogenated as described herein below.

### Tabersonine

In addition to the above the microorganism may be further engineered so that it can produce tabersonine and/or halogenated tabersonine. Tabersonine and/or halogenated tabersonine can be synthesised from dihydroprecondylocarpine acetate and/or halogenated dihydroprecondylocarpine acetate, respectively. In some embodiments, the microorganism expresses an enzyme that can convert dihydroprecondylocarpine acetate to tabersonine and/or halogenated dihydroprecondylocarpine acetate to halogenated tabersonine.

In addition to the above, it must be understood, that the dihydroprecondylocarpine acetate and/or tabersonine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO or DPAS, and further expresses tabersonine synthase (TS) which is not natively present in the microorganism.

TS has an EC number EC 4.-.-.- and converts dihydroprecondylocarpine acetate to tabersonine. The microorganism when expressing TS is thus able to convert dihydroprecondylocarpine acetate to tabersonine, thus producing tabersonine.

In some embodiments, the microorganism expresses GES and SGD, and GS, CPR, CYB5, GO, protein Redox1, protein Redox2, SAT, PAS/ASO, DPAS and TS. Optionally the microorganism also expresses CS which is described herein above.

In preferred embodiments, the TS is a TS native to Catharanthus roseus or a functional variant thereof which retains the ability to convert dihydroprecondylocarpine acetate to tabersonine. Thus, in some embodiments, the TS is CroTS as set forth in SEQ ID NO: 101 or a functional variant thereof having at least 70% homology, similarity or identity to SEQ ID NO: 101. Functional variants of TS or CroTS may be identified by expressing said enzyme in a host cell, purifying the enzyme and performing an enzyme assay to measure the conversion from dihydroprecondylocarpine acetate to tabersonine using LC-MS/MS described by Caputi et al. (2018).

The TS may be expressed in the microorganism by introducing a nucleic acid sequence as detailed further below, which encodes a TS. In particular, the nucleic acid sequence encoding CroTS may be identical to or have at least 70% homology, similarity or identity to SEQ ID NO: 39 and/or SEQ ID NO: 150.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); and/or CroTS (SEQ ID NO: 101); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100 and/or 101, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate and tabersonine, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate and halogenated tabersonine. The microorganism described in this section can be used in a method for production of tabersonine, optionally said tabersonine is halogenated as described herein below.

16-hydroxytabersonine

In addition to the above the microorganism may be further engineered so that it can produce 16-hydroxytabersonine and/or halogenated 16-hydroxytabersonine. 16-hydroxytabersonine and/or halogenated 16-hydroxytabersonine can be synthesised from tabersonine and/or halogenated tabersonine, respectively. In some embodiments, the microorganism expresses one or more enzymes that together and/or individually can convert tabersonine to 16-hydroxytabersonine and/or halogenated tabersonine to halogenated 16-hydroxytabersonine.

In addition to the above, it must be understood, that the tabersonine and/or 16-hydroxytabersonine referred to in this section may also be halogenated, such as mono, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS or TS, and further expresses tabersonine 16-hydroxylase (T16H), which is not natively present in the microorganism.

T16H has an EC number EC 1.14.14.103 and converts tabersonine to 16-hydroxytabersonine. In other words T16H catalyses 16-hydroxylation of tabersonine. The microorganism when expressing T16H, and optionally CPR and/or CYB5, is thus able to convert tabersonine to 16-hydroxytabersonine, thus producing 16-hydroxytabersonine.

In some embodiments, the microorganism expresses GES and SGD, and GS, CPR, CYB5, GO, protein Redox1, protein Redox2, SAT, PAS/ASO, DPAS, TS and T16H.

The CPR and/or CYB5 may be the CPR and/or CYB5, respectively, as defined in section “CPR and CYB5” herein above.

In preferred embodiments, the T16H is a T16H native to Catharanthus roseus or a functional variant thereof which retains the ability to convert tabersonine to 16-hydroxytabersonine. Thus, in some embodiments, the T16H is CroT16H2 as set forth in SEQ ID NO: 103 or a functional variant thereof having at least 70% homology, similarity or identity to SEQ ID NO: 103. Functional variants of T16H or CroT16H2 may be identified by expressing said enzyme in a host cell, purifying the enzyme and performing an enzyme assay to measure the conversion from tabersonine to 16-hydroxytabersonine using LC-MS/MS as described by Qu et al. (2015).

In other embodiments, the T16H is a T16H native to Catharanthus roseus such as CroT16H1 (ACM92061.1) or a functional variant thereof which retains the ability to convert tabersonine to 16-hydroxytabersonine.

The T16H may be expressed in the microorganism by introducing a nucleic acid sequence as detailed further below, which encodes a T16H. In particular, the nucleic acid sequence may be identical to or have at least 70% homology, similarity or identity to SEQ ID NO: 41.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); and/or CroT16H2 (SEQ ID NO: 103); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101 and/or 103, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine and 16-hydroxytabersonine, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine and halogenated 16-hydroxytabersonine. The microorganism described in this section can be used in a method for production of 16-hydroxytabersonine, optionally said 16-hydroxytabersonine is halogenated as described herein below.

16-methoxytabersonine

In addition to the above the microorganism may be further engineered so that it can produce 16-methoxytabersonine and/or halogenated 16-methoxytabersonine. 16-methoxytabersonine and/or halogenated 16-methoxytabersonine can be synthesised from 16-hydroxytabersonine and/or halogenated 16-hydroxytabersonine, respectively. In some embodiments, the microorganism expresses an enzyme that can convert 16-hydroxytabersonine to 16-methoxytabersonine and/or halogenated 16-hydroxytabersonine to halogenated 16-methoxytabersonine.

In addition to the above, it must be understood, that the 16-hydroxytabersonine and/or 16-methoxytabersonine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS or T16H, and further expresses tabersonine 16-O-methyltransferase (16OMT), which is not natively present in the microorganism.

16OMT has an EC number EC 2.1.1.94 and converts 16-hydroxytabersonine to 16-methoxytabersonine. In other words 16OMT catalyses O-methylation of 16-hydroxytabersonine. The microorganism when expressing 16OMT is thus able to convert 16-hydroxytabersonine to 16-methoxytabersonine, thus producing 16-methoxytabersonine.

In some embodiments, the microorganism expresses GES and SGD, and GS, CPR, CYB5, GO, protein Redox1, protein Redox2, SAT, PAS/ASO, DPAS, TS, T16H and 16OMT.

In preferred embodiments, the 16OMT is a 16OMT native to Catharanthus roseus or a functional variant thereof which retains the ability to convert 16-hydroxytabersonine to 16-methoxytabersonine. Thus, in some embodiments, the 16OMT is Cro16OMT as set forth in SEQ ID NO: 104 or a functional variant thereof having at least 70% homology, similarity or identity to SEQ ID NO: 104. Functional variants of 16OMT or Cro16OMT may be identified by expressing said enzyme in a host cell, purifying the enzyme and performing an enzyme assay to measure the conversion from 16-hydroxytabersonine to 16-methoxytabersonine using LC-MS/MS as described by Qu et al. (2015).

The 16OMT may be expressed in the microorganism by introducing a nucleic acid sequence as detailed further below, which encodes a 16OMT. In particular, the nucleic acid sequence may be identical to or have at least 70% homology, similarity or identity to SEQ ID NO: 42.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); CroT16H2 (SEQ ID NO: 103); and/or Cro16OMT (SEQ ID NO: 104); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101, 103 and/or 104, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine, 16-hydroxytabersonine and 16-methoxytabersonine, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine, halogenated 16-hydroxytabersonine and halogenated 16-methoxytabersonine. The microorganism described in this section can be used in a method for production of 16-methoxytabersonine, optionally said 16-methoxytabersonine is halogenated as described herein below.

3-hydroxy-16-methoxy-2,3-dihydrotabersonine

In addition to the above the microorganism may be further engineered so that it can produce 3-hydroxy-16-methoxy-2,3-dihydrotabersonine and/or halogenated 3-hydroxy-16-methoxy-2,3-dihydrotabersonine. 3-hydroxy-16-methoxy-2,3-dihydrotabersonine and/or halogenated 3-hydroxy-16-methoxy-2,3-dihydrotabersonine can be synthesised from 16-methoxytabersonine and/or halogenated 16-methoxytabersonine, respectively. In some embodiments, the microorganism expresses one or more enzymes that together and/or individually can convert 16-methoxytabersonine to 3-hydroxy-16-methoxy-2,3-dihydrotabersonine and/or halogenated 16-methoxytabersonine to halogenated 3-hydroxy-16-methoxy-2,3-dihydrotabersonine.

In addition to the above, it must be understood, that the 16-methoxytabersonine and/or 3-hydroxy-16-methoxy-2,3-dihydrotabersonine referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS, TS, T16H or 16OMT, and further expresses tabersonine 3-oxygenase (T3O) and/or 16-methoxy-2,3-dihydro-3-hydroxytabersonine synthase (T3R), which are not natively present in the microorganism.

T3O has an EC number EC 1.14.14.50 and catalyses the oxidation of 16-methoxytabersonine to produce an unstable imine alcohol which can be converted by T3R to 3-hydroxy-16-methoxy-2,3-dihydrotabersonine. T3R has an EC number EC 1.1.99.41 and converts the unstable imine alcohol resulting from oxidation of 16-methoxytabersonine by T3O to 3-hydroxy-16-methoxy-2,3-dihydrotabersonine. In other words, T3O is a cytochrome P450 that catalyses monooxygenation of 16-methoxytabersonine. The microorganism when expressing T3O, and optionally CPR and/or CYB5, and T3R is thus able to convert 16-methoxytabersonine to 3-hydroxy-16-methoxy-2,3-dihydrotabersonine, thus producing to 3-hydroxy-16-methoxy-2,3-dihydrotabersonine.

The CPR and/or CYB5 may be the CPR and/or CYB5, respectively, as defined in section “CPR and CYB5” herein above.

In some embodiments, the microorganism expresses GES and SGD, and GS, CPR, CYB5, GO, protein Redox1, protein Redox2, SAT, PAS/ASO, DPAS, TS, T16H, 16OMT, T3O and T3R.

In preferred embodiments, the T3O is a T3O native to Catharanthus roseus or a functional variant thereof which retains the ability to convert 16-methoxytabersonine to produce an unstable imine alcohol which can be converted by T3R to 3-hydroxy-16-methoxy-2,3-dihydrotabersonine. Thus, in some embodiments, the T3O is CroT3O as set forth in SEQ ID NO: 105 or a functional variant thereof having at least 70% homology, similarity or identity to SEQ ID NO: 105. Functional variants of T3O or CroT3O may be identified by expressing said enzyme in a host cell, purifying the enzyme and performing an enzyme assay to measure the conversion from 16-methoxytabersonine to produce an unstable imine alcohol which can be converted by T3R to 3-hydroxy-16-methoxy-2,3-dihydrotabersonine using LC-MS/MS as described by Qu et al. (2015).

The T3O may be expressed in the microorganism by introducing a nucleic acid sequence as detailed further below, which encodes a T3O. In particular, the nucleic acid sequence may be identical to or have at least 70% homology, similarity or identity to SEQ ID NO: 43.

In preferred embodiments, the T3R is a T3R native to Catharanthus roseus or a functional variant thereof which retains the ability to convert the unstable imine alcohol resulting from oxidation of 16-methoxytabersonine by T3O to 3-hydroxy-16-methoxy-2,3-dihydrotabersonine. Thus, in some embodiments, the T3R is CroT3R as set forth in SEQ ID NO: 106 or a functional variant thereof having at least 70% homology, similarity or identity to SEQ ID NO: 106. Functional variants of T3R or CroT3R may be identified by expressing said enzyme in a host cell, purifying the enzyme and performing an enzyme assay to measure the conversion from the unstable imine alcohol resulting from oxidation of 16-methoxytabersonine by T3O to 3-hydroxy-16-methoxy-2,3-dihydrotabersonine using standard techniques.

The T3R may be expressed in the microorganism by introducing a nucleic acid sequence as detailed further below, which encodes a T3R. In particular, the nucleic acid sequence may be identical to or have at least 70% homology, similarity or identity to SEQ ID NO: 44.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); CroT16H2 (SEQ ID NO: 103); Cro16OMT (SEQ ID NO: 104); and/or CroT3O (SEQ ID NO: 105) and/or CroT3R (SEQ ID NO: 106); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101, 103, 104, 105 and/or 106, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine, 16-hydroxytabersonine, 16-methoxytabersonine and 3-hydroxy-16-methoxy-2,3-dihydrotabersonine, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine, halogenated 16-hydroxytabersonine, halogenated 16-methoxytabersonine and halogenated 3-hydroxy-16-methoxy-2,3-dihydrotabersonine. The microorganism described in this section can be used in a method for production of 3-hydroxy-16-methoxy-2,3-dihydrotabersonine, optionally said 3-hydroxy-16-methoxy-2,3-dihydrotabersonine is halogenated as described herein below.

### Deacetoxyvindoline

In addition to the above the microorganism may be further engineered so that it can produce deacetoxyvindoline and/or halogenated deacetoxyvindoline. Deacetoxyvindoline and/or halogenated deacetoxyvindoline can be synthesised from 3-hydroxy-16-methoxy-2,3-dihydrotabersonine and/or halogenated 3-hydroxy-16-methoxy-2,3-dihydrotabersonine, respectively. In some embodiments, the microorganism expresses an enzyme that can convert 3-hydroxy-16-methoxy-2,3-dihydrotabersonine to deacetoxyvindoline and/or halogenated 3-hydroxy-16-methoxy-2,3-dihydrotabersonine to halogenated deacetoxyvindoline.

In addition to the above, it must be understood, that the 3-hydroxy-16-methoxy-2,3-dihydrotabersonine and/or deacetoxyvindoline referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS, TS, T16H, 16OMT, T3O or T3R, and further expresses 3-hydroxy-16-methoxy-2,3-dihydrotabersonine N-methyltransferase (NMT), which is not natively present in the microorganism.

NMT has an EC number EC 2.1.1.99 and converts 3-hydroxy-16-methoxy-2,3-dihydrotabersonine to deacetoxyvindoline. In other words NMT catalyses N-methylation of 3-hydroxy-16-methoxy-2,3-dihydrotabersonine. The microorganism when expressing NMT is thus able to convert 3-hydroxy-16-methoxy-2,3-dihydrotabersonine to deacetoxyvindoline, thus producing deacetoxyvindoline.

In some embodiments, the microorganism expresses GES and SGD, and GS, CPR, CYB5, GO, protein Redox1, protein Redox2, SAT, PAS/ASO, DPAS, TS, T16H, 16OMT, T3O, T3R and NMT.

In preferred embodiments, the NMT is a NMT native to Catharanthus roseus or a functional variant thereof which retains the ability to convert 3-hydroxy-16-methoxy-2,3-dihydrotabersonine to deacetoxyvindoline. Thus, in some embodiments, the NMT is CroNMT as set forth in SEQ ID NO: 107 or a functional variant thereof having at least 70% homology, similarity or identity to SEQ ID NO: 107. Functional variants of NMT or CroNMT may be identified by expressing said enzyme in a host cell, purifying the enzyme and performing an enzyme assay to measure the conversion from 3-hydroxy-16-methoxy-2,3-dihydrotabersonine to deacetoxyvindoline using LC-MS/MS as described by Qu et al. (2015).

The NMT may be expressed in the microorganism by introducing a nucleic acid sequence as detailed further below, which encodes a NMT. In particular, the nucleic acid sequence may be identical to or have at least 70% homology, similarity or identity to SEQ ID NO: 45.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); CroT16H2 (SEQ ID NO: 103); Cro16OMT (SEQ ID NO: 104); CroT3O (SEQ ID NO: 105); CroT3R (SEQ ID NO: 106); and/or CroNMT (SEQ ID NO: 107); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101, 103, 104, 105, 106 and/or 107, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine, 16-hydroxytabersonine, 16-methoxytabersonine, 3-hydroxy-16-methoxy-2,3-dihydrotabersonine and deacetoxyvindoline, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine, halogenated 16-hydroxytabersonine, halogenated 16-methoxytabersonine, halogenated 3-hydroxy-16-methoxy-2,3-dihydrotabersonine and halogenated deacetoxyvindoline. The microorganism described in this section can be used in a method for production of deacetoxyvindoline, optionally said deacetoxyvindoline is halogenated as described herein below.

### Deacetylvindoline

In addition to the above the microorganism may be further engineered so that it can produce deacetylvindoline and/or halogenated deacetylvindoline. Deacetylvindoline and/or halogenated deacetylvindoline can be synthesised from deacetoxyvindoline and/or halogenated deacetoxyvindoline, respectively. In some embodiments, the microorganism expresses an enzyme that can convert deacetoxyvindoline to deacetylvindoline and/or halogenated deacetoxyvindoline to halogenated deacetylvindoline.

In addition to the above, it must be understood, that the deacetoxyvindoline and/or deacetylvindoline referred to in this section may also be halogenated, such as mono-, di-, tri- or tetrahalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS, TS, T16H, 16OMT, T3O, T3R or NMT, and further expresses deacetoxyvindoline 4-hydroxylase (D4H), which is not natively present in the microorganism.

D4H has an EC number EC 1.14.11.20 and converts deacetoxyvindoline to deacetylvindoline. In other words D4H the C4-hydroxylation of deacetoxyvindoline. The microorganism when expressing D4H is thus able to convert deacetoxyvindoline to deacetylvindoline, thus producing deacetylvindoline.

In some embodiments, the microorganism expresses GES and SGD, and GS, CPR, CYB5, GO, protein Redox1, protein Redox2, SAT, PAS/ASO, DPAS, TS, T16H, 16OMT, T3O, T3R, NMT and D4H.

In preferred embodiments, the D4H is a D4H native to Catharanthus roseus or a functional variant thereof which retains the ability to convert deacetoxyvindoline to deacetylvindoline. Thus, in some embodiments, the D4H is CroD4H as set forth in SEQ ID NO: 108 or a functional variant thereof having at least 70% homology, similarity or identity to SEQ ID NO: 108. Functional variants of D4H or CroD4H may be identified by expressing said enzyme in a host cell, purifying the enzyme and performing an enzyme assay to measure the conversion from deacetoxyvindoline to deacetylvindoline using LC-MS/MS as described by Qu et al. (2015).

The D4H may be expressed in the microorganism by introducing a nucleic acid sequence as detailed further below, which encodes a D4H. In particular, the nucleic acid sequence may be identical to or have at least 70% homology, similarity or identity to SEQ ID NO: 46.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); CroT16H2 (SEQ ID NO: 103); Cro16OMT (SEQ ID NO: 104); CroT3O (SEQ ID NO: 105); CroT3R (SEQ ID NO: 106); CroNMT (SEQ ID NO: 107); and/or CroD4H (SEQ ID NO: 108); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101, 103, 104, 105, 106, 107 and/or 108, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine, 16-hydroxytabersonine, 16-methoxytabersonine, 3-hydroxy-16-methoxy-2,3-dihydrotabersonine, deacetoxyvindoline and deacetylvindoline, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine, halogenated 16-hydroxytabersonine, halogenated 16-methoxytabersonine, halogenated 3-hydroxy-16-methoxy-2,3-dihydrotabersonine, halogenated deacetoxyvindoline and halogenated deacetylvindoline. The microorganism described in this section can be used in a method for production of deacetylvindoline, optionally said deacetylvindoline is halogenated as described herein below.

### Vindoline

In addition to the above the microorganism may be further engineered so that it can produce vindoline and/or halogenated vindoline. Vindoline and/or halogenated vindoline can be synthesised from deacetylvindoline and/or halogenated deacetylvindoline, respectively. In some embodiments, the microorganism expresses an enzyme that can convert deacetylvindoline to vindoline and/or halogenated deacetylvindoline to halogenated vindoline.

In addition to the above, it must be understood, that the deacetylvindoline and/or vindoline referred to in this section may also be halogenated, such as mono-, di- or trihalogenated and may be as described in the section “Products, substrates and compounds” herein below. Thus, the polypeptides described herein this section may also be capable of converting a halogenated substrate to a corresponding halogenated product.

In some embodiments, the microorganism expresses GES and SGD, and optionally at least one of GS, CPR, CYB5, THAS, HYS, SS, GO, protein Redox1, protein Redox2, SAT, PAS/ASO, DPAS, TS, T16H, 16OMT, T3O, T3R, NMT or D4H, and further expresses deacetylvindoline O-acetyltransferase (DAT), which is not natively present in the microorganism.

DAT has an EC number EC 2.3.1.107 and converts deacetylvindoline to vindoline. The microorganism when expressing DAT is thus able to convert deacetylvindoline to vindoline, thus producing vindoline.

In some embodiments, the microorganism expresses GES and SGD, and GS, CPR, CYB5, GO, protein Redox1, protein Redox2, SAT, PAS/ASO, DPAS, TS, T16H, 16OMT, T3O, T3R, NMT, D4H and DAT.

In preferred embodiments, the DAT is a DAT native to Catharanthus roseus or a functional variant thereof which retains the ability to convert deacetylvindoline to vindoline.

Thus, in some embodiments, the DAT is CroDAT as set forth in SEQ ID NO: 109 or a functional variant thereof having at least 70% homology, similarity or identity to SEQ ID NO: 109. Functional variants of DAT or CroDAT may be identified by expressing said enzyme in a host cell, purifying the enzyme and performing an enzyme assay to measure the conversion from deacetylvindoline to vindoline using LC-MS/MS as described by Qu et al. (2015).

The DAT may be expressed in the microorganism by introducing a nucleic acid sequence as detailed further below, which encodes a DAT. In particular, the nucleic acid sequence may be identical to or have at least 70% homology, similarity or identity to SEQ ID NO: 47.

In preferred embodiments, the microorganism is capable of producing strictosidine aglycone as described in the sections herein above, said microorganism expressing at least GES and SGD, and GPPS, CPR, CYB5, G8H, 8HGO, ISY, CYC, IO, CYPADH, 7DLGT, 7DLH, LAMT, TDC, SLS and/or STR. The microorganism may additionally also express TRP and/or a tryptophan halogenase and optionally a flavin reductase.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); CroSTR (SEQ ID NO: 81); CroGS (SEQ ID NO: 85) and/or CroADH13 (SEQ ID NO: 86); CroGO (SEQ ID NO: 88), TelGO (SEQ ID NO: 89), VmiGO (SEQ ID NO: 90) and/or AhuGO (SEQ ID NO: 91); CroRdx1 (SEQ ID NO: 92); and/or CroRdx2 (SEQ ID NO: 93); CroSAT (SEQ ID NO: 94); CroPAS (SEQ ID NO: 95), AhuASO (SEQ ID NO: 96), TibPAS2 (SEQ ID NO: 97) and/or TelASO (SEQ ID NO: 98); CroDPAS (SEQ ID NO: 99) and/or TibDPAS2 (SEQ ID NO: 100); CroTS (SEQ ID NO: 101); CroT16H2 (SEQ ID NO: 103); Cro16OMT (SEQ ID NO: 104); CroT3O (SEQ ID NO: 105); CroT3R (SEQ ID NO: 106); CroNMT (SEQ ID NO: 107); CroD4H (SEQ ID NO: 108); and/or CroDAT (SEQ ID NO: 109); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 81, 85, 86, 88, 89, 90, 91, 92, 93, 94, 95, 96, 98, 99, 100, 101, 103, 104, 105, 106, 107, 108 and/or 109, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In some embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79), CroSTR (SEQ ID NO: 81), CroGS (SEQ ID NO: 85), CroGO (SEQ ID NO: 88), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99), CroTS (SEQ ID NO: 101), CroCS (SEQ ID NO: 102), CroT16H2 (SEQ ID NO: 103), Cro16OMT (SEQ ID NO: 104), CroT3O (SEQ ID NO: 105), CroT3R (SEQ ID NO: 106), CroNMT (SEQ ID NO: 107), CroD4H (SEQ ID NO: 108) and CroDAT (SEQ ID NO: 109) or functional variants thereof having at least 70% homology, similarity or identity to SEQ ID NO: 66, 82, 63, 64, 68, 69, 67, 70, 71, 72, 73, 74, 75, 76, 77, 78, 79, 81, 85, 88, 92, 93, 94, 95, 99, 101, 102, 103, 104, 105, 106, 107, 108 and/or 109, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

In some embodiments, the microorganism expresses ERG20**-GS-trunCroGES (SEQ ID NO: 66), RseSGD (SEQ ID NO: 82), AgrGPPS2 (SEQ ID NO: 63), GgaFPS*(N144W) (SEQ ID NO: 64), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78), CroTDC (SEQ ID NO: 79), CroSTR (SEQ ID NO: 81), CroGS (SEQ ID NO: 85), AhuGO (SEQ ID NO: 91), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99), CroTS (SEQ ID NO: 101), CroCS (SEQ ID NO: 102), CroT16H2 (SEQ ID NO: 103), Cro16OMT (SEQ ID NO: 104), CroT3O (SEQ ID NO: 105), CroT3R (SEQ ID NO: 106), CroNMT (SEQ ID NO: 107), CroD4H (SEQ ID NO: 108) and CroDAT (SEQ ID NO: 109) or functional variants thereof having at least 70% homology, similarity or identity to SEQ ID NO: 66, 82, 63, 64, 68, 69, 67, 70, 71, 72, 73, 74, 75, 76, 77, 78, 79, 81, 85, 91, 92, 93, 94, 95, 99, 101, 102, 103, 104, 105, 106, 107, 108 and/or 109, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section, when producing ajmalicine, serpentine, tetrahydroalstonine and/or alstonine and/or halogenated ajmalicine, serpentine, tetrahydroalstonine and/or alstonine, also produces secologanin, tryptamine, strictosidine, strictosidine aglycone, 19E-geissoschizine, stemmadenine, stemmadenine acetate, precondylocarpine acetate, dihydroprecondylocarpine acetate, tabersonine, 16-hydroxytabersonine, 16-methoxytabersonine, 3-hydroxy-16-methoxy-2,3-dihydrotabersonine, deacetoxyvindoline, deacetylvindoline and vindoline, and/or secologanin, halogenated tryptamine, halogenated strictosidine, halogenated strictosidine aglycone, halogenated 19E-geissoschizine, halogenated stemmadenine, halogenated stemmadenine acetate, halogenated precondylocarpine acetate, halogenated dihydroprecondylocarpine acetate, halogenated tabersonine, halogenated 16-hydroxytabersonine, halogenated 16-methoxytabersonine, halogenated 3-hydroxy-16-methoxy-2,3-dihydrotabersonine, halogenated deacetoxyvindoline, halogenated deacetylvindoline and halogenated vindoline. The microorganism described in this section can be used in a method for production of vindoline, optionally said vindoline is halogenated as described herein below.

### Other Modifications

Because the pathways disclosed herein requires geranyl diphosphate as a substrate, and without being bound by theory, it may be advantageous to modify the microorganism in such a manner that geranyl diphosphate metabolism is directed towards increased geranyl diphosphate synthesis and/or decreased geranyl diphosphate consumption by competing cellular pathways, thereby further increasing the titer of strictosidine aglycone, halogenated strictosidine aglycone, stemmadenine acetate, halogenated stemmadenine acetate and/or derivatives thereof. Competing cellular pathways may be defined as cellular pathways wherein geranyl diphosphate is not converted into geraniol and/or where geranyl diphosphate is converted into other metabolites than geraniol.

In some embodiments, the microorganism may further comprise one or more mutations resulting in increased availability of geranyl diphosphate. In some embodiments, the microorganism may further comprise one or more mutations resulting in loss of function of one or more of the polypeptides and/or proteins ROX1 (YPR065W), ATF1 (YOR377W), OYE2 (YHR179W), ADH6 (YMR318C), OYE3 (YPL171C) and/or ARI1 (YGL157W) or functional variants thereof having at least 70% homology, similarity or identity to ROX1 (YPR065W) ATF1 (YOR377W), OYE2 (YHR179W), ADH6 (YMR318C), OYE3 (YPL171C) and/or ARI1 (YGL157W), respectively, preferable the one or more mutations are mutations leading to loss of function of the corresponding polypeptide and/or protein, such as a deletion of said polypeptide and/or protein, such as deletion of the gene encoding said polypeptide. In preferred embodiments, the microorganism may be further modified to have a mutation leading to loss of function of the polypeptides and/or proteins ROX1 (YPR065W), ATF1 (YOR377W), OYE2 (YHR179W), ADH6 (YMR318C), OYE3 (YPL171C) and ARI1 (YGL157W) or functional variants thereof having at least 70% homology, similarity or identity to ROX1 (YPR065W), ATF1 (YOR377W), OYE2 (YHR179W), ADH6 (YMR318C), OYE3 (YPL171C) and/or ARI1 (YGL157W), respectively, wherein the mutation is a mutation leading to loss of function of the corresponding polypeptide and/or protein, such as a deletion of said polypeptide and/or protein.

In further embodiments, the microorganism may also be modified further by overexpressing one or more of the polypeptides and/or proteins IDI1 (SEQ ID NO: 136, YPL117C) and/or trunHMG1 (SEQ ID NO: 137) or functional variants thereof having at least 70% homology, similarity or identity to ID11 (SEQ ID NO: 136, YPL117C) and/or trunHMG1 (SEQ ID NO: 137), respectively.

In other embodiments, the microorganism may be further modified to have one or more mutations resulting in reduced function and/or activity of one or more of the proteins ERG20 (YJL167W) and/or ERG9 (YHR190W) or functional variants thereof having at least 70% homology, similarity or identity to ERG20 (YJL167W) and/or ERG9 (YHR190W), respectively, wherein said mutation is a mutation leading to reduced function of the corresponding protein, such as a down-regulation, said down-regulation might be due to reduced expression of the protein.

In preferred embodiments, the microorganism have one or more mutations resulting in loss of function of ATF1 (YOR377W), OYE2 (YHR179W), ADH6 (YMR318C), OYE3 (YPL171C) and ARI1 (YGL157W), and resulting in reduced function and/or activity of one or more of ERG20 (YJL167W) and/or ERG9 (YHR190W) or functional variants thereof having at least 70% homology, similarity or identity to ATF1 (YOR377W), OYE2 (YHR179W), ADH6 (YMR318C), OYE3 (YPL171C) and/or ARI1 (YGL157W), ERG20 (YJL167W) and/or ERG9 (YHR190W), respectively, and further be modified by overexpressing ID11 (SEQ ID NO: 136, YPL117C) and trunHMG1 (SEQ ID NO: 137) or functional variants thereof having at least 70% homology, similarity or identity to ID11 (SEQ ID NO: 136, YPL117C) and/or trunHMG1 (SEQ ID NO: 137), respectively.

In all the above sections, the tryptophan halogenase may be modified, such as tagged with a solubility tag to increase solubility, for example the tag may be the Trx solubility tag. Thus, IaeRebH may be replaced by Trx-IaeRebH (SEQ ID NO: 151) in any of the embodiments referring to said tryptophan halogenase herein.

In addition to, or alternatively to, any one or more of the above modifications, said microorganism may express a TRP5 (SEQ ID NO: 138), CroCPR (SEQ ID NO: 68), CroCYB5 (SEQ ID NO: 69), CroG8H (SEQ ID NO: 67), Vmi8HGOA (SEQ ID NO: 70), NcalSY (SEQ ID NO: 71), NcaMLPLA (SEQ ID NO: 72), CroIO (SEQ ID NO: 73), CroCYPADH (SEQ ID NO: 74), Cro7DLGT (SEQ ID NO: 75), Cro7DLH (SEQ ID NO: 76), CroLAMT (SEQ ID NO: 77), CroSLS (SEQ ID NO: 78) and CroSTR (SEQ ID NO: 81), optionally said microorganism further expresses AgrGPPS2 (SEQ ID NO: 63), GgaFPS*(N144W) (SEQ ID NO: 64), and ERG20**-GS-trunCroGES (SEQ ID NO: 66), and further optionally expresses:
A. rgnTDC (SEQ ID NO: 80), and further optionally expresses:
I. CroHYS (SEQ ID NO: 84) and CroSS (SEQ ID NO: 110);
II. CroTHAS1 (SEQ ID NO: 83), CroSS (SEQ ID NO: 110), IaeRebH_N470S (SEQ ID NO: 111) and EcoSsuE (SEQ ID NO: 112);
III. CroHYS (SEQ ID NO: 84), CroSS (SEQ ID NO: 110), IaeRebH_N470S (SEQ ID NO: 111) and EcoSsuE (SEQ ID NO: 112); or
IV. CroTHAS1 (SEQ ID NO: 83) and CroSS (SEQ ID NO: 110):
   i. EcoSsuE (SEQ ID NO: 112) and Trx-IaeRebH (SEQ ID NO: 151); or
B. CroTDC (SEQ ID NO: 79), and further optionally expresses:
   I. CroGS (SEQ ID NO: 85), CroGO (SEQ ID NO: 88), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99), CroTS (SEQ ID NO: 101), CroCS (SEQ ID NO: 102), CroT16H2 (SEQ ID NO: 103), Cro16OMT (SEQ ID NO: 104), CroT3O (SEQ ID NO: 105), CroT3R (SEQ ID NO: 106), CroNMT (SEQ ID NO: 107), CroD4H (SEQ ID NO: 108) and CroDAT (SEQ ID NO: 109);
   II. CroTHAS1 (SEQ ID NO: 83) and CroSS (SEQ ID NO: 110); or
   III. CroGS (SEQ ID NO: 85), CroGO (SEQ ID NO: 88), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99) and CroTS (SEQ ID NO: 101);
   IV. CroHYS (SEQ ID NO: 84) and CroSS (SEQ ID NO: 110)
      and further optionally expresses GES (SEQ ID NO: 65 and/or SEQ ID NO: 66), RseSGD (SEQ ID NO: 82),
      or functional variants thereof having at least 65% homology to any of the aforementioned sequences.

The microorganisms described in this section can be used in methods for production of one or more MIAs such as halogenated MIAs as described herein below.

### De Novo Halogenation

In addition to the above the microorganism of the present disclosure may be further engineered so that it can produce one or more halogenated tryptophans de novo. Halogenated tryptophan can be synthesised de novo from tryptophan and a halogen atom. Said halogen atom may also herein be referred to as halogen. Said halogen may be provided by a halogen source or halogen atom source, or in other words a halogen donor. In some embodiments, the microorganism expresses one or more enzymes that individually and/or together can convert tryptophan and a halogen to halogenated tryptophan. With respect to de novo halogenation of tryptophan, the halogen may preferably be selected from bromine and chlorine.

De novo halogenation of tryptophan requires that the microorganism expresses a tryptophan halogenase and optionally a flavin reductase such as FMN reductase (NADPH), whereby the microorganism is capable of converting tryptophan to halogenated tryptophan. Preferably the microorganism is a microorganism as described elsewhere herein, particularly in the section “Microorganism” herein above.

Depending on the nature of the halogen, said de novo halogenation may be referred to as chlorination and/or bromination. If the halogen atom is chlorine, the de novo halogenation may be referred to as chlorination, and the halogen source may be a chloride salt for example NaCl. If the halogen atom is bromine, the de novo halogenation may be referred to as bromination, and the halogen source may be a bromine salt for example KBr.

The halogenated tryptophan may be as described anywhere else herein, in particular in the section “Products, substrates and compounds” herein below.

### Tryptophan Halogenase

Tryptophan halogenase has an EC number EC 1.14.19.-. Tryptophan halogenase catalyses the conversion of tryptophan to halogenated tryptophan. The microorganism when expressing tryptophan halogenase is thus able to convert tryptophan to halogenated tryptophan, thus producing halogenated tryptophan in the presence of a halogen. With respect to the halogen as substrate for a tryptophan halogenase, the halogen may be selected from bromine and chlorine.

In some embodiments, the microorganism expresses GES and SGD, and further expresses tryptophan halogenase, which is not natively present in the microorganism, whereby the microorganism is further capable of producing halogenated tryptophan in the presence of tryptophan and a halogen atom, wherein the halogenated tryptophan is a tryptophan substituted with one, two, three or four halogen atoms, wherein each halogen atom is independently selected from the group consisting of chlorine and bromine. In other embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS, STR, or TDC, and further expresses tryptophan halogenase, which is not natively present in the microorganism.

In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC and tryptophan halogenase.

Depending on which type of halogenation is desired, different tryptophan halogenases can be used. If a 5-halogenated compound is desired, the tryptophan halogenase is a tryptophan-5-halogenase (EC 1.14.19.58). If a 6-halogenated compound is desired, the tryptophan halogenase is a tryptophan-6-halogenase (EC 1.14.19.59). If a 7-halogenated compound is desired, the tryptophan halogenase is a tryptophan-7-halogenase (EC 1.14.19.9). In some embodiments, the microorganism expresses at least two tryptophan halogenases independently selected from the group consisting of tryptophan-5-halogenase, tryptophan-6-halogenase and tryptophan-7-halogenase, whereby the microorganism is capable of converting tryptophan to a 5,6-dihalogenated, a 5,7-dihalogenated or a 6,7-dihalogenated tryptophan in the presence of a halogen such as bromine and/or chlorine.

The halogenated tryptophan may be as described anywhere else herein, in particular in the section “Products, substrates and compounds” herein below.

In some embodiments, the tryptophan halogenase is a tryptophan-5-halogenase. Tryptophan-5-halogenase has an EC number EC 1.14.19.58 and converts tryptophan and a halogen to a 5-halogenated tryptophan. Tryptophan-5-halogenase is known to catalyse the following reaction:

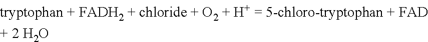

In other words, tryptophan-5-halogenase catalyses C5 halogenation of tryptophan, wherein the halogenation is selected from bromination and chlorination. Worded differently, tryptophan-5-halogenase catalyses a hydrogen-to-halogen substitution at carbon position 5 of tryptophan, said halogen being selected from bromine and chlorine, thus performing 5-halogenation. The microorganism when expressing tryptophan-5-halogenase is thus able to convert tryptophan to 5-halogenated tryptophan in the presence of a halogen, thus producing 5-halogenated tryptophan, wherein the halogen is selected from bromine and chlorine. In other embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS, STR, or TDC, and further expresses tryptophan-5-halogenase, which is not natively present in the microorganism.

In some embodiments, the tryptophan halogenase is a tryptophan-6-halogenase. Tryptophan-6-halogenase has an EC number EC 1.14.19.59 and converts tryptophan and a halogen to a 6-halogenated tryptophan. Tryptophan-6-halogenase is known to catalyse the following reaction:

tryptophan+FADH₂+chloride+O₂+H⁺=6-chloro-tryptophan+FAD+2 H₂O

In other words, tryptophan-6-halogenase catalyses C6 halogenation of tryptophan, wherein the halogenation is selected from bromination and chlorination. Worded differently, tryptophan-6-halogenase catalyses a hydrogen-to-halogen substitution at carbon position 6 of tryptophan, said halogen being selected from bromine and/or chlorine, thus performing 6-halogenation. The microorganism when expressing tryptophan-6-halogenase is thus able to convert tryptophan to 6-halogenated tryptophan in the presence of a halogen, thus producing 6-halogenated tryptophan, wherein the halogen is selected from bromine and chlorine. In some embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS, STR, or TDC, and further expresses tryptophan-6-halogenase, which is not natively present in the microorganism.

In some embodiments, the tryptophan halogenase is a tryptophan-7-halogenase. Tryptophan-7-halogenase has an EC number EC 1.14.19.9 and converts tryptophan and a halogen to a 7-halogenated tryptophan. Tryptophan-7-halogenase is known to catalyse the following reaction:

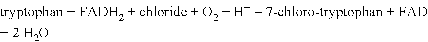

In other words, tryptophan-7-halogenase catalyses C7 halogenation of tryptophan, wherein the halogenation is selected from bromination and chlorination. Worded differently, tryptophan-7-halogenase catalyses a hydrogen-to-halogen substitution at carbon position 7 of tryptophan, said halogen being selected from bromine and/or chlorine, thus performing 7-halogenation. The microorganism when expressing tryptophan-7-halogenase is thus able to convert tryptophan to 7-halogenated tryptophan in the presence of a halogen, thus producing 7-halogenated tryptophan, wherein the halogen is selected from bromine and chlorine. In other embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS, STR, or TDC, and further expresses tryptophan-7-halogenase, which is not natively present in the microorganism.

In preferred embodiments, the tryptophan halogenase is a tryptophan-7-halogenase native to Lechevalieria aerocolonigenes such as Lechevalieria aerocolonigenes A TCC 39243 or a functional variant thereof which retains the ability to convert tryptophan to 7-halogenated tryptophan in the presence of a halogen. Thus, in some embodiments, the tryptophan-7-halogenase is IaeRebH_N470S as set forth in SEQ ID NO: 111, IaeRebH (SEQ ID NO: 153), or a functional variant thereof having at least 70% homology, similarity or identity to SEQ ID NO: 111 or SEQ ID NO: 153. Functional variants of tryptophan-7-halogenase such as IaeRebH_N470S may be identified by expressing said enzyme in a host cell, purifying the enzyme and performing an enzyme assay to measure the conversion from tryptophan to 7-halogenated tryptophan using LC-MS described by Glenn et al. (2011). In some embodiments, the microorganism expresses GES and SGD, and optionally at least one of GPPS, G8H, CPR, CYB5, 8HGO, ISY, CYC, IO, CYPADH, 7DLGT, 7DLH, LAMT, SLS, STR, or TDC, and further expresses IaeRebH (SEQ ID NO: 153) and/or IaeRebH_N470S (SEQ ID NO: 111).

Functional variants of tryptophan halogenase may be identified by expressing said enzyme in a host cell, purifying the enzyme and performing an enzyme assay to measure the conversion from tryptophan to halogenated tryptophan using LC-MS described by Glenn et al. (2011).

The tryptophan halogenase may be expressed in the microorganism by introducing a nucleic acid sequence as detailed further below, which encodes a tryptophan halogenase. In particular, the nucleic acid sequence may be identical to or have at least 70% homology, similarity or identity to SEQ ID NOs: 49, 152 and/or 154.

In preferred embodiments, the microorganism expresses trunCroGES (SEQ ID NO: 65) and/or ERG20**-GS-trunCroGES (SEQ ID NO: 66); RseSGD (SEQ ID NO: 82); ERG20** (SEQ ID NO: 139), AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); and/or IaeRebH (SEQ ID NO: 153) and/or IaeRebH_N470S (SEQ ID NO: 111); or functional variants thereof having at least 70% homology, similarity or identity to SEQ ID NO: 65, 66, 82, 139, 63, 64, 68, 69, 67, 70, 71, 72, 73, 74, 75, 76, 77, 78, 79, 80, 153 and/or 111, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The microorganism described in this section can be used in a method for production of halogenated tryptophan as described herein below.

### Flavin Reductase

In order to improve the production of halogenated tryptophan for example to increase the titer of halogenated tryptophan thereby aiding to increase the production and/or titer of halogenated MIAs such as halogenated stemmadenine acetate and derivatives thereof, it may be helpful to express in the microorganism a flavin reductase in addition to at least one of the tryptophan halogenases as described herein above. Flavin reductase herein as term covers both flavin reductases with EC numbers EC 1.5.1.36 and EC 1.5.1.30, and also FAD reductases such as FAD reductase (NADH) and/or FAD reductase (NADPH), and also FMN reductases such as FMN reductase (NADPH) and/or FMN reductase (NADH).

In some embodiments, the microorganism expresses GES and SGD, and further expresses tryptophan halogenase and flavin reductase, which may not be natively present in the microorganism, whereby the microorganism is further capable of producing halogenated tryptophan in the presence of tryptophan and a halogen atom, wherein the halogenated tryptophan is a tryptophan substituted with one, two, three or four halogen atoms, wherein each halogen atom is independently selected from the group consisting of chlorine and bromine. In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC, tryptophan halogenase and flavin reductase.

In some embodiments, the flavin reductase, which is not natively expressed in the microorganism. In other embodiments, the flavin reductase is a heterologous FMN reductase (NADPH). FMN reductase (NADPH) has an EC number EC 1.5.1.38. FMN reductase (NADPH) is known to catalyse the following reaction:

FMNH₂+NADP⁺=FMN+NADPH+H⁺

FMN is the preferred substrate, but FMN reductase (NADPH) can also use FAD (and reduce it to FADH₂) and riboflavin. In some embodiments, the flavin reductase is an FMN reductase (NADH) that can catalyse the same reaction as FMN reductase (NADPH) but may use NADH instead of NADPH as used by the FMN reductase (NADPH).

In preferred embodiments, the flavin reductase is a FMN reductase (NADPH) native to Escherichia coli such as Escherichia coli str. K-12 substr. MG1655 or a functional variant thereof which retains the ability to reduce FMN, FAD and/or riboflavin. Thus, in some embodiments, the FMN reductase (NADPH) is EcoSsuE as set forth in SEQ ID NO: 112 or a functional variant thereof having at least 70% homology, similarity or identity to SEQ ID NO: 112. Functional variants of FMN reductase (NADPH) or EcoSsuE may be identified by expressing said enzyme in a host cell, purifying the enzyme and performing an enzyme assay to measure the conversion from FMN, FAD, or riboflavin to FMNH₂, FADH₂ or reduced riboflavin, respectively, using standard techniques.

In other embodiments, the flavin reductase is a heterologous FAD reductase. FAD reductase has an EC number EC 1.5.1.45. FAD reductase is known to catalyse the following reaction:

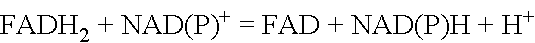

FAD is the preferred substrate, but FAD reductase can also use FMN (and reduce it to FMNH₂).

In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC and tryptophan halogenase and optionally flavin reductase such as FAD reductase, whereby the microorganism is further capable of halogenating tryptophan in the presence of tryptophan and a halogen atom, wherein the halogenated tryptophan is a tryptophan substituted with at least one, two, three or four halogen atoms, wherein each halogen atom is independently selected from the group consisting of chlorine and bromine. In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC, tryptophan halogenase and FAD reductase.

The flavin reductase such as FMN reductase (NADPH) may be expressed in the microorganism by introducing a nucleic acid sequence as detailed further below, which encodes a flavin reductase such as FMN reductase (NADPH). In particular, the nucleic acid sequence may be identical to or have at least 70% homology, similarity or identity to SEQ ID NO: 50.

In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC and tryptophan-5-halogenase and optionally flavin reductase such as FMN reductase (NADPH), whereby the microorganism is further capable of 5-halogenating tryptophan in the presence of tryptophan and a halogen atom, wherein the 5-halogenated tryptophan is a tryptophan substituted with at least one, two, three or four halogen atoms, wherein each halogen atom is independently selected from the group consisting of chlorine and bromine. In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC, tryptophan-5-halogenase and FMN reductase (NADPH).

In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC and tryptophan-6-halogenase and optionally flavin reductase such as FMN reductase (NADPH) whereby the microorganism is further capable of 6-halogenating tryptophan in the presence of tryptophan and a halogen atom, wherein the 6-halogenated tryptophan is a tryptophan substituted with at least one, two, three or four halogen atoms, wherein each halogen atom is independently selected from the group consisting of chlorine and bromine. In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC, tryptophan-6-halogenase and FMN reductase (NADPH).

In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC and tryptophan-7-halogenase and optionally flavin reductase such as FMN reductase (NADPH), whereby the microorganism is further capable of 7-halogenating tryptophan in the presence of tryptophan and a halogen atom, wherein the 7-halogenated tryptophan is a tryptophan substituted with at least one, two, three or four halogen atoms, wherein each halogen atom is independently selected from the group consisting of chlorine and bromine. In some embodiments, the microorganism expresses GES and SGD, and GPPS, G8H, CPR, CYB5, 8HGO, ISY, CYC, IO, CYPADH, 7DLGT, 7DLH, LAMT, SLS, STR, TDC, tryptophan-7-halogenase and FMN reductase (NADPH).

In preferred embodiments, the microorganism expresses trunCroGES (SEQ ID NO: 65) and/or ERG20**-GS-trunCroGES (SEQ ID NO: 66); RseSGD (SEQ ID NO: 82); AgrGPPS2 (SEQ ID NO: 63) and/or GgaFPS*(N144W) (SEQ ID NO: 64); CroCPR (SEQ ID NO: 68) and/or CroCYB5 (SEQ ID NO: 69); CroG8H (SEQ ID NO: 67); Vmi8HGOA (SEQ ID NO: 70); NcalSY (SEQ ID NO: 71); NcaMLPLA (SEQ ID NO: 72); CroIO (SEQ ID NO: 73); CroCYPADH (SEQ ID NO: 74); Cro7DLGT (SEQ ID NO: 75); Cro7DLH (SEQ ID NO: 76); CroLAMT (SEQ ID NO: 77); CroSLS (SEQ ID NO: 78); CroTDC (SEQ ID NO: 79) and/or rgnTDC (SEQ ID NO: 80); and/or IaeRebH (SEQ ID NO: 153) and/or IaeRebH_N470S (SEQ ID NO: 111) and/or EcoSsuE (SEQ ID NO: 112); or functional variants thereof having at least 70% homology, similarity or identity to SEQ ID NO: 65, 66, 82, 63, 64, 68, 69, 67, 70, 71, 72, 73, 74, 75, 76, 77, 78, 79, 80, 153, 111 and/or 112, respectively. In addition, the microorganism may express TRP5 (SEQ ID NO: 138) or a functional variant thereof having at least 70% homology, similarity or identity to SEQ ID NO: 138.

The microorganism described in this section can be used in a method for production of halogenated tryptophan as described herein below.

### Co-Localisation

Co-localisation of two or more proteins and/or polypeptides of an enzymatic pathway in the same cellular compartment and/or cellular membrane may be means to improve the titer of pathway products of interest such as the MIAs described herein produced by the microorganism disclosed herein. It may also be beneficial to localise two or more proteins of an enzymatic pathway in different cellular compartments and/or cellular membranes to avoid competing pathways, i.e. pathways with at least one protein utilizing the same substrate(s) as at least one protein of the pathway of interest. The improvement in titer may be a consequence of improved availability of substrates of said proteins and/or due to localisation in a more favourable conditions with regards to for example the catalytic activity, folding and/or stability/half-life of said proteins. Enzymes are catalytic proteins. In other words, the product of a first enzyme may be the substrate of a second enzyme and therefore the turnover of said second enzyme may benefit from being in close proximity of said first enzyme. Localisation of pathway enzymes in cellular compartments may also be referred to as compartmentalisation. Cellular compartments may also be referred to as subcellular compartments.

Thus, in addition to the above the microorganism may further be engineered so that optionally one or more of polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are tagged with a signal peptide.

A signal peptide may also be referred to as a signal sequence, localisation sequence, localisation signal, leader sequence, leader peptide targeting signal and/or a transit peptide. When a polypeptide is “tagged” with a signal peptide, said polypeptide comprises, at least transiently, said signal peptide. In other words, the sequence of said polypeptide tagged with a signal peptide may comprise the sequence of said signal peptide. Thus, the nucleic acid construct encoding said tagged polypeptide may comprise both the nucleic acid sequence encoding said polypeptide and the nucleic acid sequence encoding said signal peptide. The term “transiently” herein comprises that the signal peptide may be cleaved off the polypeptide for example by a peptidase. This implies that the polypeptide once comprising said signal peptide may upon cleavage not comprise the signal peptide anymore. In some embodiments, said signal peptide is a cleavable signal peptide such that said one or more polypeptides are at least transiently tagged with said signal peptide. In other embodiments, said signal peptide is not cleavable from the polypeptide which is tagged with said signal peptide. In other words, in some embodiments the signal peptide forms a stable part of the polypeptide such as the mature polypeptide. A signal peptide may be present in the N-terminus or C-terminus of a polypeptide. Signal peptides are short polypeptides, also known as peptides, which typically comprise less than 100 amino acids (AAs) such as 1-100 AAs, such as 10-90 AAs, such as 15-80 AAs, such as 20-70 AAs, such as 30-60 AAs.

Two or more polypeptides that are tagged with identical signal peptides that may target and/or direct the two or more polypeptides to the same cellular compartment and/or membrane may be localised in, such as being present in, the same cellular compartment and/or cellular membrane. This targeted and/or directed localisation of two or more polypeptides may also be referred to as co-localisation. Said cellular compartment may be the endoplasmic reticulum (ER) lumen, mitochondrial lumen (mitochondria), nucleus, peroxisomal lumen (peroxisome), vacuolar lumen (vacuole) and/or any other cellular compartment. Said cellular membrane might be the ER membrane. In some embodiments, the two or more polypeptides are targeted and/or directed to the cytoplasmic site of the ER membrane of a microorganism.

In other embodiments, said signal peptide is a signal peptide targeting a polypeptide to a given cellular compartment, said cellular compartment being a compartment of said microorganism. In other words, said signal peptide is a signal peptide for a subcellular compartment of said microorganism.

In some embodiments, the signal peptide is a mitochondrial signal peptide such as a mitochondrial lumen signal peptide for example a Neurospora crassa ATP9 signal peptide (ATP9sp) as set forth in SEQ ID NO: 140 or a functional variant thereof having at least 70% homology, similarity or identity thereto. ATP9sp is an N-terminal signal peptide capable of localizing in the mitochondrial lumen of a microorganism.

In other embodiments, the signal peptide is an endoplasmic reticulum (ER) signal peptide such as an ER lumen signal peptide for example a Saccharomyces cerevisiae calnexin homolog signal peptide also known as CNE1 signal peptide (CNE1sp) as set forth in SEQ ID NO: 141 or a functional variant thereof having at least 70% homology, similarity or identity thereto. CNE1sp is a C-terminal signal peptide capable of localizing in the ER lumen of a microorganism.

In some embodiments, the signal peptide is an endoplasmic reticulum (ER) signal peptide such as an ER membrane signal peptide for example Saccharomyces cerevisiae Cytochrome b5 signal peptide (CYB5sp) as set forth in SEQ ID NO: 142 or functional variants thereof having at least 70% homology, similarity or identity thereto. CYB5sp is a C-terminal signal peptide capable of localizing at the cytoplasmic site of the ER membrane of a microorganism. Thus, in some embodiments, the signal peptide is an ER membrane signal peptide for localizing a polypeptide at the cytoplasmic site of the ER membrane of a microorganism.

In other embodiments, the signal peptide is a peroxisomal signal peptide (a peroxisome signal peptide) such as a peroxisomal lumen signal peptide for example Saccharomyces cerevisiae enhanced peroxisomal targeting signal type 1 (PTS1), also known as ePTS1 signal peptide (ePTS1sp), as set forth in SEQ ID NO: 143 or functional variants thereof having at least 70% homology, similarity or identity thereto. ePTS1 is a C-terminal signal peptide capable of localizing in the peroxisomal lumen of a microorganism.

In some embodiments, the signal peptide is a vacuolar signal peptide such as a vacuolar signal peptide for example Saccharomyces cerevisiae carboxypeptidase Y signal peptide, also known as CPY signal peptide (CPYsp), as set forth in SEQ ID NO: 144 or functional variants thereof having at least 70% homology, similarity or identity thereto. CYPsp is an N-terminal signal peptide capable of localizing in the vacuole of a microorganism.

In other embodiments, the signal peptide is a nuclear signal peptide for example Simian virus 40 signal peptide, also known as SV40 signal peptide (SV40sp), as set forth in SEQ ID NO: 25 or functional variants thereof having at least 70% homology, similarity or identity thereto. SV40sp is an N-terminal signal peptide capable of localizing in the nucleus of a microorganism.

In some embodiments, two or more of the polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are localised in the same cellular compartment and/or cellular membrane such as in the ER lumen, ER membrane, the cytoplasmic site of the ER membrane, mitochondrial lumen, nucleus, peroxisomal lumen, vacuole and/or any other cellular compartment, optionally said one or more polypeptides are tagged with identical signal peptides that direct the polypeptides to the same cellular compartment and/or cellular membrane. In further some embodiments, wherein said two or more polypeptides are co-localised in the mitochondrial lumen, said microorganism further comprises a mitochondrial NADH kinase, for example an NADH kinase with EC 2.7.1.86. This can be helpful in overexpressing the polypeptides.

In some embodiments, the SGD and GS are localised in the same cellular compartment and/or cellular membrane of a microorganism expressing said SGD and GS. In other words, in some embodiments, the SGD and GS are co-localised in the same cellular compartment and/or cellular membrane of a microorganism expressing said SGD and GS. In some embodiments, the SGD and the GS are localised in, such as being present in, the same cellular compartment of a microorganism expressing said SGD and GS, such as in the ER lumen, the mitochondrial lumen and/or mitochondria, the nucleus, the peroxisomal lumen and/or peroxisome(s), the vacuole(s) and/or any other cellular compartment of the microorganism expressing said SGD and GS. In other embodiments, the SGD and GS are localised in the same cellular membrane of a microorganism expressing said SGD and GS, such as the ER membrane, in particular on the cytoplasmic site of the ER membrane. In preferred embodiments, the SGD and GS are localised in the ER lumen, the mitochondrial lumen and/or the on the cytoplasmic site of the ER membrane of a microorganism expressing said SGD and GS.

In some embodiments, said SGD is ATP9sp-RseSGD (SEQ ID NO: 113), RseSGD-CNE1sp (SEQ ID NO: 115), RseSGD-CYB5sp (SEQ ID NO: 117), RseSGD-ePTS1sp (SEQ ID NO: 119), RseSGD-CPYsp (SEQ ID NO: 121) and/or SV40sp-RseSGD (SEQ ID NO: 123) or functional variants thereof having at least 70% homology, similarity or identity to SEQ ID NO: 113, SEQ ID NO: 115, SEQ ID NO: 117, SEQ ID NO: 119, SEQ ID NO: 121 and/or SEQ ID NO: 123, respectively. In other embodiments, said GS is ATP9sp-CroGS (SEQ ID NO: 114), CroGS-CNE1sp (SEQ ID NO: 116) and/or CroGS-CYB5sp (SEQ ID NO: 118), CroGS-ePTS1sp (SEQ ID NO: 120), CroGS-CPYsp (SEQ ID NO: 122) and/or SV40sp-CroGS (SEQ ID NO: 124) or functional variants thereof having at least 70% homology, similarity or identity to SEQ ID NO: 114, SEQ ID NO: 116, SEQ ID NO: 118, SEQ ID NO: 120, SEQ ID NO: 122 and/or SEQ ID NO: 124, respectively.

In some embodiments, the SGD and GS are co-localised in the mitochondria and/or mitochondrial lumen of a microorganism expressing said SGD and GS, and optionally said SGD is ATP9sp-RseSGD (SEQ ID NO: 113) and/or said GS is ATP9sp-CroGS (SEQ ID NO: 114) or functional variants thereof having at least 70% homology, similarity or identity to SEQ ID NO: 113 and/or SEQ ID NO: 114, respectively, further optionally said microorganism comprises and/or overexpresses a mitochondrial NADH kinase such as POS5 (SEQ ID NO: 87) or functional variants thereof having at least 70% homology, similarity or identity to SEQ ID NO: 87.

In other embodiments, the SGD and GS are co-localised in the ER lumen of a microorganism expressing said SGD and GS, and optionally said SGD is RseSGD-CNE1sp (SEQ ID NO: 115) and/or said GS is CroGS-CNE1sp (SEQ ID NO: 116) or functional variants thereof having at least 70% homology, similarity or identity to SEQ ID NO: 115 and/or SEQ ID NO: 116, respectively.

In some embodiments, the SGD and GS are co-localised in or at the ER membrane of the microorganism expressing said SGD and GS. In preferred embodiments, the SGD and GS are co-localised at the cytoplasmic site of the ER membrane of a microorganism expressing said SGD and GS, and optionally said SGD is RseSGD-CYB5sp (SEQ ID NO: 117) and/or said GS is CroGS-CYB5sp (SEQ ID NO: 118) or functional variants thereof having at least 70% homology, similarity or identity to SEQ ID NO: 117 and/or SEQ ID NO: 118, respectively.

In other embodiments, the SGD and GS are co-localised in the peroxisome and/or peroxisomal lumen of a microorganism expressing said SGD and GS, and optionally said SGD is RseSGD-ePTS1sp (SEQ ID NO: 119) and/or said GS is CroGS-ePTS1sp (SEQ ID NO: 120) or functional variants thereof having at least 70% homology, similarity or identity to SEQ ID NO: 119 and/or SEQ ID NO: 120, respectively.

In other embodiments, the SGD and GS are co-localised in the vacuole and/or vacuolar lumen of a microorganism expressing said SGD and GS, and optionally said SGD is RseSGD-CPYsp (SEQ ID NO: 121) and/or said GS is CroGS-CPYsp (SEQ ID NO: 122) or functional variants thereof having at least 70% homology, similarity or identity to SEQ ID NO: 121 and/or SEQ ID NO: 122, respectively.

In other embodiments, the SGD and GS are co-localised in the nucleus and/or nuclear lumen of a microorganism expressing said SGD and GS, and optionally said SGD is SV40sp-RseSGD (SEQ ID NO: 123) and/or said GS is SV40sp-CroGS (SEQ ID NO: 124) or functional variants thereof having at least 70% homology, similarity or identity to SEQ ID NO: 123 and/or SEQ ID NO: 124, respectively.

In some embodiments, the SGD and GS are localised in the same cellular compartment and/or cellular membrane of a microorganism expressing said SGD and GS, said microorganism further expresses a NADH kinase specific to said cellular compartment and/or cellular membrane. In other embodiments, the SGD and GS are localised in the same cellular compartment and/or cellular membrane of a microorganism expressing said SGD and GS, said microorganism further expresses and/or overexpresses a NADH kinase in said same cellular compartment and/or cellular membrane.

Furthermore, in some embodiments the SGD and GS are co-localised the same cellular compartment and/or cellular membrane such as in the ER lumen, ER membrane, the cytoplasmic site of the ER membrane, mitochondrial lumen, nucleus, peroxisomal lumen, vacuole and/or any other cellular compartment, together with a NADH kinase such as POS5 as set forth in SEQ ID NO: 87 and/or NAD/NADH kinase YEF1 (YEL041W) or functional variants thereof having at least 70% homology, similarity or identity to SEQ ID NO: 87 and/or 149.

### Nucleic Acid Constructs

In addition to the above, herein disclosed is also nucleic acid constructs useful for expression in a microorganism.

The present nucleic acid constructs may be provided as one or more nucleic acid constructs or polynucleotides, for example they may be comprised in one or more vectors.

Said nucleic acid constructs, polynucleotides and/or vectors may be useful for expression in, engineering and/or modification of a microorganism. Provided herein is a microorganism comprising one or more nucleic acid constructs expressing the required polypeptides such as enzymes. Preferably, the microorganism is as defined herein above in “Microorganism” and/or elsewhere herein.

It will be understood that the terms nucleic acid construct, nucleic acid sequence, polynucleotide and/or vector herein refers to a nucleic acid such as a nucleic acid molecule capable of encoding one or more polypeptides, proteins, enzymes or fragments thereof and optionally other genetic and/or regulatory elements such as promoters, terminators etc. The nucleic acid construct and/or nucleic acid sequence may be comprised in another nucleic acid construct and/or nucleic acid sequence.

Thus, in addition to the above, the microorganism disclosed herein being capable of producing strictosidine aglycone, stemmadenine acetate, halogenated stemmadenine acetate and derivatives thereof may comprise one or more nucleic acid constructs.

In some embodiments, the present invention provides one or more nucleic acid constructs for modifying a microorganism, said one or more nucleic acid constructs comprising a nucleic acid sequence identical to or having at least 70% homology, similarity or identity to SEQ ID NO: 3, 4 and/or 20. In other embodiments, the one or more nucleic acid constructs further comprise a nucleic acid sequence identical to or having at least 70% homology, similarity or identity to SEQ ID NO: 23, 24, 6, 7, 26, 27, 28, 29, 30, 31 and/or 32. In further other embodiments, the one or more nucleic acid constructs further comprises a nucleic acid sequence identical to or having at least 70% homology, similarity or identity to SEQ ID NO: 1, 2 and/or 128. In some embodiments, the one or more nucleic acid constructs further comprises a nucleic acid sequence identical to or having at least 70% homology, similarity or identity to SEQ ID NO: 5, 6, 7, 8, 9, 10, 11, 12, 13, 14, 15, 16 and/or 19. In other embodiments, the one or more nucleic acid constructs further comprises a nucleic acid sequence identical to or having at least 70% homology, similarity or identity to SEQ ID NO: 17, 18, 127, 49, 152, 154 and/or 50. In some embodiments, the one or more nucleic acid constructs further comprises a nucleic acid sequence identical to or having at least 70% homology, similarity or identity to SEQ ID NO: 6, 7, 21, 22 and/or 48. In further other embodiments, the one or more nucleic acid constructs further comprises a nucleic acid sequence identical to or having at least 70% homology, similarity or identity to SEQ ID NO: 6, 7, 33, 34, 35, 36, 37, 38, 39 and/or 40. Furthermore, in some embodiments, the one or more nucleic acid constructs further comprises a nucleic acid sequence identical to or having at least 70% homology, similarity or identity to SEQ ID NO: 6, 7, 41, 42, 43, 44, 45, 46 and/or 47.

Additionally, in other embodiments, the one or more nucleic acid constructs further comprising a nucleic acid sequence identical to or having at least 70% homology, similarity or identity to SEQ ID NO: 129, 130, 131, 132, 133 and/or 134, preferably SEQ ID NO: 129, 130 and/or 131. In some embodiments, the one or more nucleic acid constructs further comprising a nucleic acid sequence identical to or having at least 70% homology, similarity or identity to SEQ ID NO: 51, 52, 53, 54, 55, 56, 57, 58, 59, 60, 61, 62 and/or 135, preferably SEQ ID NO: 51, 52, 53, 54, 55, 56, and/or 135. In addition to the above, in other embodiments, the one or more nucleic acid constructs further comprising a nucleic acid sequence identical to or having at least 70% homology, similarity or identity to SEQ ID NO: 125 and/or SEQ ID NO: 126.

Furthermore, in some embodiments, the one or more nucleic acid constructs and/or the one or more nucleic acid sequences are present in high copy number. In some embodiments, the one or more nucleic acid constructs and/or the one or more nucleic acid sequences are present in at least one copy, such as at least in two copies, such as at least in three copies, such as at least in four copies, or more. In some embodiments, the one or more nucleic acid constructs and/or the one or more nucleic acid sequences are present in low copy number. In some embodiments, the one or more nucleic acid constructs and/or the one or more nucleic acid sequences are under the control of an inducible promoter. In other embodiments, the one or more nucleic acid constructs and/or the one or more nucleic acid sequences are under the control of a constitutive promoter. In other embodiments, the one or more nucleic acid constructs and/or the one or more nucleic acid sequences are integrated into the genome of the microorganism. Methods for genomic integration of nucleic acid constructs and/or nucleic acid sequences are well known in the art.

The nucleic acid constructs may comprise features that can help improve the polypeptide encoded by the nucleic acid sequence comprised by the nucleic acid construct. Such modifications include, but are not limited to, introduction of signal peptides such as described in the section “Co-localisation” herein, gain-of-function and/or loss-of-function mutations, fusion of a polypeptide to a marker or a tag such as fluorescence tag, introduction of modifications conferring increased stability and/or half-life of said polypeptides and/or nucleic acid constructs.

In some embodiments, one or more nucleic acid sequences encoding the polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are comprised within a vector such as a plasmid, a high-copy vector, an episomal vector, an integrative vector and/or a replicative vector.

In some embodiments, one or more nucleic acid sequences encoding the polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are codon-optimised for the microorganism comprising said one or more nucleic acid constructs.

The nucleic acid constructs may be one or more PCR products or one or more synthetic nucleic acid molecules.

In some embodiments, the nucleic acid construct further comprises one or more promoters such as pTEF1, pPGK1, pMLS1, pFBA1, pTDH3, pICL1, pTEF2, pTPI1, pCCW12, pENO2, pYPR036W-A, pTDH2, pGAL1, pGAL10, pGAL2, pGAL7 or homologues thereof having at least 70% identity, homology or similarity thereto, respectively.

In other embodiments, the nucleic acid construct further comprises one or more glucose-repressible promoters. In other embodiments, the nucleic acid construct further comprises one or more constitutively active promoters.

Useful nucleic acid constructs is also described in detail in application WO2020/229516 entitled “Methods for production of strictosidine aglycone and monoterpenoid indole alkaloids” filed on 13 May 2020 and assigned to the same applicant, particularly in the section entitled “Nucleic acids, vectors and host cells” therein, and also in application WO2022/106638entitled “Methods for production of cis-trans-nepetalactol and iridoids” filed 19 Nov. 2021 and assigned to the same applicant, particularly in the section entitled “Nucleic acid constructs” therein.

### Vectors

In some embodiments, the nucleic acid construct is one or more vectors. Suitable vectors are known in the art and readily available to the skilled person.

Thus, also provided herein is a vector comprising one or more nucleic acid constructs or nucleic acid sequences as described herein. The nucleic acid constructs can be any of the nucleic acid constructs described herein above. The nucleic acid constructs can independently be comprised within one or several vectors.

In some embodiments, the vector is a high copy replicative vector. In other embodiments, the vector is a low copy replicative vector. In other embodiments, the vector is an episomal plasmid.

Also provided herein is a microorganism comprising one or more nucleic acid constructs and/or vectors as defined herein above. The microorganism may be any microorganism described herein, such as a prokaryote or a eukaryote. The host cell may be a yeast or a bacteria. In some embodiment, the host cell is Escherichia coli. In preferred embodiments, the host cell is Saccharomyces cerevisiae.

Also provided herein is the use of the nucleic acid constructs, the microorganisms and/or the vectors described herein for production of strictosidine, strictosidine aglycone, alstonine, tetrahydroalstonine, ajmalicine, serpentine, catharanthine, tabersonine, vindoline, stemmadenine acetate and/or derivatives thereof, including halogenated analogues thereof such as halogenated strictosidine, strictosidine aglycone, alstonine, tetrahydroalstonine, ajmalicine, serpentine, catharanthine, tabersonine, vindoline, stemmadenine acetate and/or derivatives thereof in a microorganism.

In some embodiments, the nucleic acid constructs, the microorganisms and/or the vectors described herein are used in a method for producing strictosidine, strictosidine aglycone, alstonine, tetrahydroalstonine, ajmalicine, serpentine, catharanthine, tabersonine, vindoline, stemmadenine acetate and/or derivatives thereof, including halogenated analogues thereof such as halogenated strictosidine, strictosidine aglycone, alstonine, tetrahydroalstonine, ajmalicine, serpentine, catharanthine, tabersonine, vindoline, stemmadenine acetate and/or derivatives thereof in a microorganism as described herein. Such vectors can comprise any of the nucleic acid constructs or sequences detailed herein.

In some embodiments, one or more of the vectors may be integrated in the genome of the microorganism. Each of the nucleic acid constructs or sequences comprised within the present vector(s) may be present in multiple copies in the microorganism.

The vectors and/or nucleic acid constructs may, in addition to the one or more nucleic acid constructs encoding the GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase, also comprise additional nucleic acid constructs useful for introducing additional modifications in the microorganism, to obtain microorganisms as disclosed in “Other modifications” above or anywhere herein.

Also provided herein is a kit of parts comprising a microorganism as described herein above, and/or nucleic acid constructs as described herein above, and/or a vector as described herein above.

### Methods for Producing MIAs

The microorganisms described herein, which can produce MIAs and derivatives thereof, where said MIAs and derivatives thereof can be halogenated as detailed herein elsewhere, can be used in methods for producing MIAs and derivatives thereof, which may be halogenated.

Herein is thus provided a method of producing strictosidine aglycone in a microorganism, said method comprising the steps of;
i. providing a microorganism, said microorganism expressing:
   a geraniol synthase (GES, EC 3.1.7.11) and
   a strictosidine-O-β-D-glucosidase (SGD, EC 3.2.1.105);
ii. incubating said microorganism in a medium comprising a substrate and optionally a halogen atom source, which can be converted to strictosidine aglycone and/or halogenated strictosidine aglycone by said microorganism in the presence of geranyl diphosphate and tryptamine and/or halogenated tryptamine;
iii. optionally recovering the strictosidine aglycone;
iv. optionally further converting the strictosidine aglycone to one or more monoterpene indole alkaloids (MIAs),
   wherein said GES is capable of converting geranyl diphosphate to geraniol, optionally said GES is a heterologous GES, preferably said GES is as set forth in SEQ ID NO: 65 and/or in SEQ ID NO: 66; and/or wherein said SGD is capable of converting strictosidine and/or halogenated strictosidine to strictosidine aglycone and/or halogenated strictosidine aglycone, respectively, option-ally said SGD is a heterologous SGD, preferably said SGD is RseSGD (SEQ ID NO: 82), or functional variants thereof having at least 70% homology, similarity or identity to SEQ ID NO: 65, SEQ ID NO: 66 and/or SEQ ID NO: 82, respectively.

In some embodiments the method comprises step iv. The strictisodine aglycone can thus be converted to other compounds by expressing the required enzymes as described in detail herein above. For example, a microorganism expressing a GES and an SGD and further expressing a GPPS, a G8H, a CPR, a CYB5, a 8HGO, an ISY, an CYC, an 10, a CYPADH, a 7DLGT, a 7DLH, a LAMT, a TDC, a SLS, a STR, a GS, a GO, a protein Redox1, a protein Redox2 and/or a SAT, can be used in a method to produce steammadenine acetate, which optionally can be halogenated as described herein above. These enzymes have been described in detail herein above. In some embodiments, said GPPS is ERG20** (SEQ ID NO: 139), AgrGPPS2 (SEQ ID NO: 63) and/or GgaFPS*(N144W) (SEQ ID NO: 64), said G8H is CroG8H (SEQ ID NO: 67), said CPR is CroCPR (SEQ ID NO: 68), said CYB5 is CroCYB5 (SEQ ID NO: 69), said 8HGO is Vmi8HGOA (SEQ ID NO: 70), said ISY is NcalSY (SEQ ID NO: 71), said CYC is NcaMLPLA (SEQ ID NO: 72), said IO is CroIO (SEQ ID NO: 73), said CYPADH is CroCYPADH (SEQ ID NO: 74), said 7DLGT is Cro7DLGT (SEQ ID NO: 75), said 7DLH is Cro7DLH (SEQ ID NO: 76), said LAMT is CroLAMT (SEQ ID NO: 77), said TDC is CroTDC (SEQ ID NO: 79) and/or rgnTDC (SEQ ID NO: 80), said SLS is CroSLS (SEQ ID NO: 78), said STR is CroSTR (SEQ ID NO: 81), said GS is CroGS (SEQ ID NO: 85) and/or CroADH13 (SEQ ID NO: 86), said GO is CroGO (SEQ ID NO: 88), TelGO (SEQ ID NO: 89), VmiGO (SEQ ID NO: 90), and/or AhuGO (SEQ ID NO: 91), said pro-tein Redox1 is CroRdx1 (SEQ ID NO: 92), said protein Redox2 is CroRdx2 (SEQ ID NO: 93) and/or said SAT is CroSAT (SEQ ID NO: 94) or functional variants thereof having at least 70% homology, similarity or identity to SEQ ID NO: 139, 63, 64, 67, 68, 69, 70, 71, 72, 73, 74, 75, 76, 77, 78, 81, 85, 86, 68, 69, 88, 89, 90, 91, 92, 93 and/or 94, respectively. In addition, the microorganism may express TRP5 (SEQ ID NO: 138), IaeRebH (SEQ ID NO: 153) and/or IaeRebH_N470S (SEQ ID NO: 111) and optionally EcoSsuE (SEQ ID NO: 112) or functional variants thereof having at least 70% homology, similarity or identity to SEQ ID NO: 138, 153, 111 and/or 112, respectively.

The compounds produced in the microorganism, for example strictosidine aglycone, stemmadenine acetate, halogenated strictosidine aglycone or halogenated stemmadenine acetate may in some embodiments of the methods be further converted into other MIAs and/or halogenated MIAs. The term “further conversion” herein simply means that the produced compound is transformed or converted into another compound which is a MIA. The conversion may happen in vivo, i.e. within the microorganism, which may be capable of catalysing further conversion of a produced compound into other compounds. The methods however may also comprise the steps of recovering any of the compounds such as products synthesised by the microorganism from the microorganism or from the medium by methods known in the art, and thereafter converting the compound into another compound, i.e. the further conversion may be an ex vivo conversion.

Furthermore, herein is provided a method of producing a halogenated MIA in a microorganism, said method comprising the steps of;
i. providing a microorganism, said microorganism expressing:

(SEQ ID NO: 138)
TRP5,
(SEQ ID NO: 68)
CroCPR,
(SEQ ID NO: 69)
CroCYB5,
(SEQ ID NO: 67)
CroG8H,
(SEQ ID NO: 70)
Vmi8HGOA,
(SEQ ID NO: 71)
NcaISY,
(SEQ ID NO: 72)
NcaMLPLA,
(SEQ ID NO: 73)
CroIO,
(SEQ ID NO: 74)
CroCYPADH,
(SEQ ID NO: 75)
Cro7DLGT,
(SEQ ID NO: 76)
Cro7DLH,
(SEQ ID NO: 77)
CroLAMT,
(SEQ ID NO: 78)
CroSLS,
and
(SEQ ID NO: 81)
CroSTR;,

ii. incubating said microorganism in a medium comprising a substrate and a halogen atom source, which can be converted to the halogenated MIA by the microorganism in the presence of GPP and halogenated tryptamine;
iii. optionally recovering the halogenated MIA;
iv. further optionally converting the halogenated MIA to one or more derivatives thereof,
   optionally wherein said microorganism further expresses AgrGPPS2 (SEQ ID NO: 63), GgaFPS*(N144W) (SEQ ID NO: 64), and ERG20**-GS-trunCroGES (SEQ ID NO: 66), and further optionally expresses:
   C. rgnTDC (SEQ ID NO: 80), further wherein the halogen atom source is 4-fluoroindole, 5-fluoroindole, 6-fluoroindole, 7-fluoroindole, 7-chloroindole, 7-bromoindole, 4,5-difluoroindole and/or 4,7-difluoroindole, wherein the one or more halogenated MIAs is 4-fluorostrictosidine, 5-fluorostrictosidine, 6-fluorostrictosidine, 7-fluorostrictosidine, 7-chlorostrictosidine, 7-bromostrictosidine, 4,5-difluorostrictosidine and/or 4,6-difluorostrictosidine, respectively, and further optionally expresses:
   I. CroHYS (SEQ ID NO: 84) and CroSS (SEQ ID NO: 110), further wherein the halogen atom source is 4-fluoroindole, 5-fluoroindole, 6-fluoroindole, 7-fluoroindole, 7-chloroindole, 7-bromoindole, 4,5-difluoroindole, 4,6-difluoroindole, 4,7-difluoroindole, 5,7-difluoroindole and/or 6,7-difluoroindole, wherein the one or more halogenated MIAs is 4-fluoroserpentine, 5-fluoroserpentine, 6-fluoroserpentine, 7-fluoroserpentine, 7-chloroserpentine, 7-bromoserpentine, 4,5-difluoroserpentine, 4,6-difluoroserpentine, 4,7-difluoroserpentine, 5,7-difluoroserpentine and/or 6,7-difluoroserpentine, respectively;
   II. CroTHAS1 (SEQ ID NO: 83), CroSS (SEQ ID NO: 110), IaeRebH_N470S (SEQ ID NO: 111) and EcoSsuE (SEQ ID NO: 112), wherein the halogen atom source is NaCl, wherein the one or more halogenated MIAs is 7-chloroalstonine;
   III. CroHYS (SEQ ID NO: 84), CroSS (SEQ ID NO: 110), IaeRebH_N470S (SEQ ID NO: 111) and EcoSsuE (SEQ ID NO: 112), further wherein the halogen atom source is NaCl, wherein the one or more halogenated MIAs is 7-chloroserpentine; or
   IV. CroTHAS1 (SEQ ID NO: 83) and CroSS (SEQ ID NO: 110), further wherein the halogen atom source is 4-fluoroindole, 5-fluoroindole, 6-fluoroindole, 7-fluoroindole, 7-chloroindole, 7-bromoindole, 5,6-difluoroindole and/or 6,7-difluoroindole, wherein the one or more halogenated MIAs is 4-fluoroalstonine, 5-fluoroalstonine, 6-fluoroalstonine, 7-fluoroalstonine, 7-chloroalstonine, 7-bromoalstonine, 5,6-difluoroalstonine and/or 6,7-difluoroalstonine, respectively,
      and further optionally expresses:
      i. EcoSsuE (SEQ ID NO: 112) and Trx-IaeRebH (SEQ ID NO: 151), further wherein the halogen atom source is NaCl, wherein the one or more halogenated MIAs is 7-chloroalstonine; or
   D. CroTDC (SEQ ID NO: 79), further wherein the halogen atom source is 4-fluoroindole, 5-fluoroindole, 6-fluoroindole, 7-fluoroindole, 4-chloroindole, 4,5-difluoroindole and/or 4,7-difluoroindole, the one or more halogenated MIAs is 4-fluorostrictosidine, 5-fluorostrictosidine, 6-fluorostrictosidine, 7-fluorostrictosidine, 4-chlorostrictosidine, 4,5-difluorostrictosidine and/or 4,7-difluorostrictosidine, respectively, and further optionally expresses:
   I. CroGS (SEQ ID NO: 85), CroGO (SEQ ID NO: 88), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99), CroTS (SEQ ID NO: 101), CroCS (SEQ ID NO: 102), CroT16H2 (SEQ ID NO: 103), Cro16OMT (SEQ ID NO: 104), CroT3O (SEQ ID NO: 105), CroT3R (SEQ ID NO: 106), CroNMT (SEQ ID NO: 107), CroD4H (SEQ ID NO: 108) and CroDAT (SEQ ID NO: 109), further wherein the halogen atom source is 6-fluoroindole, 7-fluoroindole, 6-fluoroindole and/or 7-fluoroindole, wherein the one or more halogenated MIAs is 6-fluorostemmadenine acetate, 7-fluorostemmadenine acetate, 6-fluorotabersonine and/or 7-fluorotabersonine, respectively;
   II. CroTHAS1 (SEQ ID NO: 83) and CroSS (SEQ ID NO: 110), further wherein the halogen atom source is 4-fluoroindole, 5-fluoroindole, 6-fluoroindole, 4-fluoroindole, 5-fluoroindole, 6-fluoroindole, 7-fluoroindole, 5-fluoroindole, 6-fluoroindole and/or 7-fluoroindole, wherein the one or more halogenated MIAs is 4-fluorostrictosidine aglycone, 5-fluorostrictosidine aglycone, 6-fluorostrictosidine aglycone, 4-fluorotetrahydroalstonine, 5-fluorotetrahydroalstonine, 6-fluorotetrahydroalstonine, 7-fluorotetrahydroalstonine, 5-fluoroalstonine, 6-fluoroalstonine and/or 7-fluoroalstonine, respectively; or
   III. CroGS (SEQ ID NO: 85), CroGO (SEQ ID NO: 88), CroRdx1 (SEQ ID NO: 92), CroRdx2 (SEQ ID NO: 93), CroSAT (SEQ ID NO: 94), CroPAS (SEQ ID NO: 95), CroDPAS (SEQ ID NO: 99) and CroTS (SEQ ID NO: 101), further wherein the halogen atom source is 6-fluoroindole, 7-fluoroindole, 6-fluoroindole and/or 7-fluoroindole, wherein the one or more halogenated MIAs is 6-fluorostemmadenine acetate, 7-fluorostemmadenine acetate, 6-fluorotabersonine and/or 7-fluorotabersonine, respectively,
      and further optionally expresses a GES (SEQ ID NO: 65 and/or SEQ ID NO: 66) and/or RseSGD (SEQ ID NO: 82),
      or functional variants thereof having at least 65% homology to any of the aforementioned sequences.

### Medium

For all of the above methods, i.e. for production of geranyl diphosphate, tryptamine, strictosidine aglycone, stemmadenine acetate, halogenated tryptamine, halogenated strictosidine aglycone, halogenated stemmadenine acetate and/or other MIAs and halogenated MIAs, the microorganism requires appropriate substrates.

The necessary substrates for each product may be provided to the cell as part of the medium used to grow the microorganisms. Alternatively, the substrates for each of the above products may be synthesised by the microorganism itself. In all cases, the microorganism is capable of synthesising the compounds and/or products as described above.

In some embodiments, the medium comprises geraniol. The microorganism can convert said geraniol to 8-hydroxygeraniol as described in detail herein above. In some embodiments, the medium comprises geraniol at a concentration of at least 0.05 mM, such as at least 0.1 mM, such as at least 0.5 mM, such as at least 1 mM.

In some embodiments, the medium may further comprise indole at a concentration of at least 0.05 mM, 0.1 mM, such as at least 0.5 mM, such as at least 1 mM, such as at least 2 mM or more. Alternatively, or in addition, the medium may comprise tryptamine, preferably at a concentration of at least 0.05 mM, such as at least 0.1 mM, such as at least 0.5 mM, such as at least 1 mM, such as at least 2 mM or more. Alternatively, or in addition, the medium may comprise halogenated tryptamine. In the presence of secologanin, the microorganism can convert said tryptamine and/or halogenated tryptamine to strictosidine and/or halogenated strictosidine, respectively, as described in detail herein above. The medium may comprise halogenated tryptamine, preferably at a concentration of at least 0.05 mM, such as at least 0.1 mM, such as at least 0.5 mM, such as at least 1 mM, such as at least 2 mM or more, wherein the halogenated tryptamine may be as described in the section “Products, substrates and compounds” herein. Alternatively, or in addition, the medium may comprise tryptophan, preferably at a concentration of at least 0.05 mM, such as at least 0.1 mM, such as at least 0.5 mM, such as at least 1 mM, such as at least 2 mM or more. The medium may comprise halogenated tryptophan, preferably at a concentration of at least 0.05 mM, such as at least 0.1 mM, such as at least 0.5 mM, such as at least 1 mM, such as at least 2 mM or more, wherein the halogenated tryptamine may be as described in the section “Products, substrates and compounds” herein. Secologanin may be synthesised by the microorganism itself as described herein above. Alternatively, or in addition, the medium may comprise secologanin, preferably at a concentration of at least 0.05 mM, such as at least 0.1 mM, such as at least 0.5 mM, such as at least 1 mM, such as at least 2 mM or more, or combinations thereof.

In some embodiments, where it is desired to obtain a halogenated compound, the medium may further comprise halogenated indole as described herein, preferably at a concentration of at least 0.05 mM. such as at least 0.1 mM. such as at least 0.5 mM, such as at least 1 mM, such as at least 2 mM or more. Alternatively, or in addition, the medium may comprise halogenated tryptamine, preferably at a concentration of at least 0.05 mM, such as at least 0.1 mM, such as at least 0.5 mM, such as at least 1 mM, such as at least 2 mM or more. Alternatively, or in addition, the medium may comprise a halogen atom source such as a salt, preferably NaCl, and/or KBr, preferably at a concentration of at least 0.05 M, such as at least 0.1 M, such as at least 0.5 M or more. Alternatively, or in addition, the medium may comprise halogenated tryptophan, preferably at a concentration of at least 0.05 mM, such as at least 0.1 mM, such as at least 0.5 mM, such as at least 1 mM, such as at least 2 mM or more. Alternatively, or in addition, the medium may comprise secologanin, preferably at a concentration of at least 0.05 mM, such as at least 0.1 mM, such as at least 0.5 mM, such as at least 1 mM, such as at least 2 mM or more, or combinations thereof.

### Titers

The microorganisms and methods disclosed herein can be used to produce different compounds at high titers. The titers can be determined by methods known in the art, for example by GC or LC-MS/MS.

In some embodiments, where the microorganism is capable of producing strictosidine, the microorganism produces strictosidine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated strictosidine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing strictosidine aglycone, the microorganism produces strictosidine aglycone with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated strictosidine aglycone as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing alstonine, the microorganism produces alstonine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated alstonine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing serpentine, the microorganism produces serpentine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated serpentine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing tetrahydroalstonine and/or ajmalicine, the microorganism produces ajmalicine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated tetrahydroalstonine and/or halogenated ajmalicine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing stemmadenine acetate, the microorganisms produces stemmadenine acetate with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated stemmadenine acetate as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing catharanthine, the microorganisms produces catharanthine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated catharanthine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing tabersonine, the microorganisms produces tabersonine with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated tabersonine as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

In some embodiments, where the microorganism is capable of producing vindoline, the microorganisms produces vindoline with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more. In some embodiments, the microorganism produces halogenated vindoline as described herein elsewhere with a titer of at least 1 μg/L, such as 2.5 μg/L, such as 5 μg/L, such as 10 μg/L, such as 25 μg/L, such as 50 μg/L, such as 100 μg/L, such as 250 μg/L, such as 500 μg/L, such as 1 mg/L, such as 2.5 mg/L, such as 5 mg/L, such as 10 mg/L, or more.

The halogenated strictosidine, strictosidine aglycone, ajmalicine, serpentine, tetrahydroalstonine, alstonine, stemmadenine acetate, catharanthine, tabersonine and/or vindoline may be halogenated as described in the section “Products, substrates and compounds” herein.

### Recovery

The present methods may comprise a further step of recovering any of the compounds described herein and/or derivatives thereof such as strictosidine, strictosidine aglycone, alstonine, tetrahydroalstonine, ajmalicine, serpentine, catharanthine, tabersonine, vindoline, stemmadenine acetate and/or derivatives thereof, including halogenated analogues thereof such as halogenated strictosidine, strictosidine aglycone, alstonine, tetrahydroalstonine, ajmalicine, serpentine, catharanthine, tabersonine, vindoline, stemmadenine acetate and/or derivatives thereof, obtained by the methods and/or microorganisms disclosed anywhere herein.

Methods for recovering the compounds and/or derivatives thereof obtained by the present invention are known in the art, for example solid-phase extraction (SPE) or liquidliquid extraction (LLE).

For example, the step of recovering any of the compounds described herein may comprise separating the cell culture in a solid phase to obtain a cell phase and in a liquid phase to obtain a supernatant. The compounds may be present in the cell phase and/or the supernatant. The compounds may also be volatile. The cell phase is also known as cell pellet.

### Products, Substrates and Compounds

Provided herein is strictosidine, strictosidine aglycone, alstonine, tetrahydroalstonine, ajmalicine, serpentine, catharanthine, tabersonine, vindoline, stemmadenine acetate and/or derivatives thereof, including halogenated analogues and/or derivatives thereof obtainable by the methods disclosed herein. Provided herein is also halogenated strictosidine, strictosidine aglycone, alstonine, tetrahydroalstonine, ajmalicine, serpentine, catharanthine, tabersonine, vindoline, stemmadenine acetate and/or analogues and/or derivatives thereof obtainable by the methods disclosed herein. Thus, disclosed herein is strictosidine, strictosidine aglycone, alstonine, tetrahydroalstonine, ajmalicine, serpentine, catharanthine, tabersonine, vindoline, stemmadenine acetate and/or derivatives hereof, including halogenated analogues thereof, obtainable by the methods described herein.

In particular, provided herein is stemmadenine acetate and/or derivatives thereof obtainable by the methods disclosed herein. Also provided is halogenated stemmadenine acetate and/or derivatives thereof obtainable by the methods disclosed herein.

### Halogenated Indole

Halogenated indole may be provided to the microorganism disclosed herein, for example as part of the cultivation medium that the microorganism is incubated in. When halogenated indole is provided said microorganism, the microorganism may be capable of converting serine and said halogenated indole into a corresponding halogenated tryptophan as described herein for example in the section “Tryptophan” herein above. The microorganism may be able to synthesise serine natively and/or by overexpression of heterologous enzymes that enable the microorganism to synthesis serine. Serine may also be supplemented the microorganism as part of the cultivation medium that the microorganism is incubated in. Halogenated indole may be referred herein to as halogen atom source.

With respect to the halogenated indole that may be provided the microorganism, said halogenated indole may be halogenated in more than one position, for example in two positions, by a halogen atom, wherein each atom is independently selected from the group consisting of fluorine, chlorine and bromine.

Thus, in some embodiments the halogenated indole is 4-halogenated, 5-halogenated, 6-halogenated and/or 7-halogenated by a halogen selected from the group consisting of fluorine, chlorine and bromine. In other embodiments, the halogenated indole is selected from the group consisting of fluoroindole, chloroindole and bromoindole. The halogenated indole may be 4-fluoroindole. The halogenated indole may be 5-fluoroindole. The halogenated indole may be 6-fluoroindole. The halogenated indole may be 7-fluoroindole. The halogenated indole may be 4-chloroindole. The halogenated indole may be 5-chloroindole. The halogenated indole may be 6-chloroindole. The halogenated indole may be 7-chloroindole. The halogenated indole may be 4-bromoindole. The halogenated indole may be 5-bromoindole. The halogenated indole may be 6-bromoindole. The halogenated indole may be 7-bromoindole.

The halogenated indole may also be dihalogenated. Thus, the halogenated indole may be difluoroindole. The halogenated indole may be dichloroindole. The halogenated indole may be dibromoindole. The position of the halogen on the indole may be at carbon 4, 5, 6 and/or 7. In some embodiments, the halogenated indole is 4,5-dihalogenated, 4,6-dihalogenated, 4,7-dihalogenated, 5,6-dihalogenated, 5,7-dihalogenated and/or 6,7-dihalogenated by two halogen atoms, each halogen atom independently selected from the group consisting of fluorine, bromine and chlorine. For example, and not bound by theory, the halogenated indole may be 4-chloro-7-fluoroindole, 5-bromo-6-chloroindole, or 5,5-dibromoindole. Dihalogenated indole may for example be fluorochloro-indole, dichloroindole, difluoroindole, and/or dibromoindole. In some embodiments, the dihalogenated indole is 7,6-difluoroindole, 7,5-difluoroindole, 7,4-difluoroindole, 6,4-difluoroindole, 6,5-difluoroindole and/or 5,4-difluoroindole. Dihalogenated indole may also be 7-bromo-5-fluoroindole, 6-bromo-4-fluoroindole and/or 6-chloro-5-fluoroindole. In some embodiment, the dihalogenated indole is 4,5-difluoroindole. In some other embodiments, the dihalogenated indole is 4,6-difluoroindole. In other embodiments, the dihalogenated indole is 4,7-difluoroindole. In further some embodiments, the dihalogenated indole is 5,6-difluoroindole. In another some embodiments, the dihalogenated indole is 5,7-difluoroindole. In some other embodiments, the dihalogenated indole is 6,7-difluoroindole. Trihalogenated indole may for example be trifluoroindole, trichloroindole or tribromoindole, such as for example, but not limited to 4,5,7-trifluoroindole, 5,6,7-trichloroindole and/or 4,6,7-tribromoindole.

The halogenated indole may also be trihalogenated. Thus, the halogenated indole may be trifluoroindole, trichloroindole and/or tribromoindole. In some embodiments, the halogenated indole is 4,5,6-trihalogenated, 4,5,7-trihalogenated, 4,6,7-trihalogenated and/or 5,6,7-trihalogenated by three halogen atoms, each halogen atom independently selected from the group consisting of fluorine, bromine and chlorine. As an example, and not bound by theory, the halogenated indole may be 4-chloro-5-bromo-6-bromoindole, 5-fluoro-6-chloro-7-bromoindole or 4-bromo-5-fluoro-7-chloroindole.

The halogenated indole may also be tetrahalogenated. Thus, the halogenated indole may be tetrafluoroindole, tetrachloroindole and/or tetrabromoindole. In some embodiments, the halogenated indole is 4,5,6,7-tetrahalogenated by four halogen atoms, each halogen atom independently selected from the group consisting of fluorine, bromine and chlorine. For example, and not bound by theory, the halogenated indole may be 4-fluoro-5-chloro-6-bromo-7-bromoindole.

The halogenation pattern of the halogenated indole which is provided a microorganism capable of converting said halogenated indole and serine into a halogenated tryptophan will be reflected in the halogenation pattern of the halogenated tryptophan and any other compound derived from the halogenated indole and/or tryptophan. Any compound derived from the halogenated indole by the action of one or more enzymes may contain the same halogen atom(s) in the same position(s) of the indole ring as the halogenated indole that it is derived from. For example, dichloroindole may be converted to dichlorotryptophan by the action of TRP such as TRP5 disclosed herein. Said dichlorotryptophan may in turn by the action of TDC such as rgnTDC be converted to dichlorotryptamine.

Different halogenation patterns of the di-, tri-, and/or tetrahalogenated compounds may also be obtained by combining different haloindoles and tryptophan halogenases. For example, halogenated indole, such as 4-fluoroindole, may be provided to a microorganism expressing at least one tryptophan halogenase, such as tryptophan-7-halogenase, and TRP, in the presence of chlorine, whereby the microorganism may be capable of producing 4-fluorotryptophan, 7-chlorotryptophan and/or 4-fluoro-7-chlorotryptophan.

### Halogenated Compounds

In some embodiments, the halogenated compounds, i.e. the halogenated tryptophan, tryptamine, strictosidine, strictosidine aglycone, alstonine, tetrahydroalstonine, ajmalicine, serpentine, tetrahydroalstonine, ajmalicine, geissoschizine, stemmadenine, precondylocarpine acetate, dihydroprecondylocarpine acetate, 16-hydroxytabersonine, 16-methoxytabersonine, 3-hydroxy-16-methoxy-2,3-dihydrotabersonine, deacetoxyvindoline, deacetylvindoline, catharanthine, tabersonine, stemmadenine acetate and/or derivatives thereof is substituted with at least one, such as at least two, such as at least three, such as at least four halogen atoms, each halogen atom independently selected from the group consisting of fluorine, bromine and chlorine, preferably chlorine or bromine.

In other embodiments, the halogenated tryptophan, tryptamine, strictosidine, strictosidine aglycone, alstonine, tetrahydroalstonine, ajmalicine, serpentine, tetrahydroalstonine, ajmalicine, geissoschizine, stemmadenine, precondylocarpine acetate, dihydroprecondylocarpine acetate, 16-hydroxytabersonine, 16-methoxytabersonine, 3-hydroxy-16-methoxy-2,3-dihydrotabersonine, deacetoxyvindoline, deacetylvindoline, catharanthine, tabersonine, stemmadenine acetate and/or derivatives thereof is 4-halogenated, 5-halogenated, 6-halogenated and/or 7-halogenated by a halogen atom selected from the group consisting of fluorine, bromine and chlorine, preferably chlorine or bromine. In some embodiments, the halogenated vindoline and/or derivatives thereof is 4-halogenated, 5-halogenated and/or 7-halogenated by a halogen atom selected from the group consisting of fluorine, bromine and chlorine.

In other embodiments, the halogenated tryptophan, tryptamine, strictosidine, strictosidine aglycone, alstonine, tetrahydroalstonine, ajmalicine, serpentine, tetrahydroalstonine, ajmalicine, geissoschizine, stemmadenine, precondylocarpine acetate, dihydroprecondylocarpine acetate, 16-hydroxytabersonine, 16-methoxytabersonine, 3-hydroxy-16-methoxy-2,3-dihydrotabersonine, deacetoxyvindoline, deacetylvindoline, catharanthine, tabersonine, stemmadenine acetate and/or derivatives thereof is 4,5-dihalogenated, 4,6-dihalogenated, 4,7-dihalogenated, 5,6-dihalogenated, 5,7-dihalogenated and/or 6,7-dihalogenated by two halogen atoms, each halogen atom independently selected from the group consisting of fluorine, bromine and chlorine, preferably chlorine or bromine. In other embodiments, the halogenated vindoline and/or derivatives thereof is 4,5-dihalogenated, 4,7-dihalogenated and/or 5,7-dihalogenated by two halogen atoms, each halogen atom independently selected from the group consisting of fluorine, bromine and chlorine. For example the halogenated tryptophan may be dichlorotryptophan such as 5,6-dichlorotryptophan, 5,7-dichlorotryptophan, 6,7-dichlorotryptophan and/or dibromotryptophan such as 5,6-dibromotryptophan, 5,7-dibromotryptophan, 6,7-dibromotryptophan.

In some embodiments, the halogenated strictosidine and/or derivatives thereof is 4-fluorostrictosidine, 5-fluorostrictosidine, 6-fluorostrictosidine, 7-fluorostrictosidine, 4-chlorostrictosidine, 7-chlorostrictosidine, 7-bromostrictosidine, 4,5-difluorostrictosidine, 4,6-difluorostrictosidine, 4,7-difluorostrictosidine, 5,6-difluorostrictosidine, 5,7-difluorostrictosidine, 6,7-difluorostrictosidine and/or derivatives thereof. In some other embodiments, the halogenated strictosidine aglycone is 7-chlorostrictosidine aglycone, 7-bromostrictosidine aglycone, 4-fluorostrictosidine aglycone, 5-fluorostrictosidine aglycone, 6-fluorostrictosidine aglycone, 7-fluorostrictosidine aglycone, 5,6-difluorostrictosidine aglycone, 6,7-difluorostrictosidine aglycone, 4,5-difluorostrictosidine aglycone, 4,6-difluorostrictosidine aglycone, 4,7-difluorostrictosidine aglycone, 5,7-difluorostrictosidine aglycone and/or derivatives thereof. In some other embodiments, the halogenated ajmalicine and/or derivatives is 7-chloroajmalicine, 4-fluoroajmalicine, 5-fluoroajmalicine, 6-fluoroajmalicine, 7-fluoroajmalicine, 7-bromoajmalicine, 4,5-difluoroajmalicine, 4,6-difluoroajmalicine, 4,7-difluoroajmalicine, 5,6-difluoroajmalicine, 5,7-difluoroajmalicine, 6,7-difluoroajmalicine, and/or derivatives thereof. In further some embodiments, the halogenated serpentine and/or derivatives is 7-chloroserpentine, 4-fluoroserpentine, 5-fluoroserpentine, 6-fluoroserpentine, 7-fluoroserpentine, 7-bromoserpentine, 4,5-difluoroserpentine, 4,6-difluoroserpentine, 4,7-difluoroserpentine, 5,6-difluoroserpentine, 5,7-difluoroserpentine, 6,7-difluoroserpentine, and/or derivatives thereof. In further other embodiments, the halogenated alstonine and/or derivatives thereof is 7-chloroalstonine, 7-bromoalstonine, 4-fluoroalstonine, 5-fluoroalstonine, 6-fluoroalstonine, 7-fluoroalstonine, 5,6-difluoroalstonine, 6,7-difluoroalstonine, and/or derivatives thereof. In other embodiments, the halogenated tetrahydroalstonine, and/or derivatives thereof is 7-chlorotetrahydroalstonine, 7-bromotetrahydroalstonine, 4-fluorotetrahydroalstonine, 5-fluorotetrahydroalstonine, 6-fluorotetrahydroalstonine, 7-fluorotetrahydroalstonine, 5,6-difluorotetrahydroalstonine, 6,7-difluorotetrahydroalstonine, and/or derivatives thereof. In some further embodiments, the halogenated geissoschizine and/or derivatives thereof is fluorinated geissoschizine and/or derivatives thereof such as 4-fluorogeissoschizine and/or 5-fluorogeissoschizine, preferably 6-fluorogeissoschizine and/or 7-fluorogeissoschizine, and/or derivatives thereof. In other embodiments, the halogenated stemmadenine acetate and/or derivatives thereof is fluorinated stemmadenine acetate and/or derivatives thereof such as 4-fluorostemmadenine acetate and/or 5-fluorostemmadenine acetate, preferably 6-fluorostemmadenine acetate and/or 7-fluorostemmadenine acetate, and/or derivatives thereof. In some other embodiments, the halogenated tabersonine and/or derivatives thereof is fluorinated tabersonine and/or derivatives thereof such as 4-fluorotabersonine and/or 5-fluorotabersonine, preferably 6-fluorotabersonine and/or 7-fluorotabersonine, and/or derivatives thereof.

In some embodiments, the tryptophan, tryptamine, strictosidine, strictosidine aglycone, alstonine, serpentine, tetrahydroalstonine, ajmalicine, geissoschizine, stemmadenine, precondylocarpine acetate, dihydroprecondylocarpine acetate, 16-hydroxytabersonine, 16-methoxytabersonine, 3-hydroxy-16-methoxy-2,3-dihydrotabersonine, deacetoxyvindoline, deacetylvindoline, catharanthine, tabersonine, stemmadenine acetate and/or derivatives thereof is 4,5,6-trihalogenated, 4,5,7-trihalogenated, 4,6,7-trihalogenated and/or 5,6,7-trihalogenated by three halogen atoms, each halogen atom independently selected from the group consisting of fluorine, bromine and chlorine, preferably chlorine or bromine. In other embodiments, the halogenated vindoline and/or derivatives thereof is 4,5,7-trihalogenated by three halogen atoms, each halogen atom independently selected from the group consisting of fluorine, bromine and chlorine.

In other embodiments, the tryptophan, tryptamine, strictosidine, strictosidine aglycone, alstonine, serpentine, tetrahydroalstonine, ajmalicine, geissoschizine, stemmadenine, precondylocarpine acetate, dihydroprecondylocarpine acetate, 16-hydroxytabersonine, 16-methoxytabersonine, 3-hydroxy-16-methoxy-2,3-dihydrotabersonine, deacetoxyvindoline, deacetylvindoline, catharanthine, tabersonine, stemmadenine acetate, vindoline, and/or derivatives thereof is 4,5,6,7-tetrahalogenated by at least four halogen atoms, each halogen atom independently selected from the group consisting of fluorine, bromine and chlorine, preferably chlorine or bromine.

Compositions, fermentation liquid, cell cultures and pharmaceutical compounds Provided herein is also a composition comprising any of or a combination of strictosidine, strictosidine aglycone, alstonine, tetrahydroalstonine, ajmalicine, serpentine, catharanthine, tabersonine, vindoline, stemmadenine acetate and/or derivatives thereof, including halogenated analogues thereof such as halogenated strictosidine, strictosidine aglycone, alstonine, tetrahydroalstonine, ajmalicine, serpentine, catharanthine, tabersonine, vindoline, stemmadenine acetate and/or derivatives thereof, obtainable by the methods disclosed herein. The halogen atom with respect to halogenated strictosidine, strictosidine aglycone, alstonine, tetrahydroalstonine, ajmalicine, serpentine, catharanthine, tabersonine, vindoline, stemmadenine acetate and/or derivatives thereof may be selected from the group consisting of fluorine, bromine, and chlorine. In any case the halogenated compounds may be as described in section “Products, substrates and compounds” herein. The composition may also contain the fermentation liquid and/or one or more agents, additives and/or excipients.

Further provided is a cell culture, obtained by the methods described herein, for example in the section “Methods for producing MIAs”, and/or comprising a microorganism described herein, for example in the section “Microorganism”, and optionally a medium, such as a medium described in the section “Medium”.

Provided herein is also a fermentation liquid comprising one of more derivatives of halogenated strictosidine aglycone, such as one or more MIAs, halogenated MIAs, halogenated MIAs of interest, and/or derivatives thereof, obtained by the methods described herein, for example in the section “Methods for producing MIAs”, or comprised in a cell culture as described elsewhere herein, for example in the present section. In some embodiments, the fermentation liquid comprises at least 50% disrupted cells. In further some embodiments, at least 50% of solid cellular material is separated from the liquid of the fermentation liquid.

The compounds obtainable by the present methods may be useful for manufacturing pharmaceutical compounds. Thus, the methods may further comprise a step of producing a pharmaceutical compound from any of the compounds, in particular MIA such as halogenated MIAs, produced by the microorganism of the present invention.

In some embodiments, the fermentation liquid and/or composition have been processed into a semi-dry or dry solid form, such as in form of a powder, tablet, capsule, chewable and/or gum. In other embodiments, the fermentation liquid and/or composition is in a liquid form and/or gel form, optionally in a stabilized liquid form and/or gel form.

Therefore, also provided are methods of treating a disorder such as a cancer, arrhythmia, malaria, fibrosis, pain, anxiety, Parkinson's disease, schizophrenia, bipolar disorder, psychotic diseases, hypertension, depression, Alzheimer's disease, addiction and/or neuronal diseases, comprising administration of a therapeutic sufficient amount of a MIA and/or a pharmaceutical compound obtained by the methods described anywhere herein.

### EXAMPLES

### Example 1: Construction of Yeast Strains

All strains were constructed using the CRISPR-Cas9 method described in Jakočiūnas T. et al. 2015.

The yeast strain MIA-CH-A2 was used as a base strain from which all other strains were derived. MIA-CH-A2 was constructed by integrating all genes necessary for conversion of geraniol and tryptamine to strictosidine into the wild-type yeast strain CEN.PK2-1C. To reduce side-product formation, five native yeast genes were knocked out by complete deletion of the open reading frame of each gene. The genotype of MIA-CH-A2 is summarized in Table 1.

TABLE 1

| Strain | Integrated | Deleted native | Substrate -> |
|---|---|---|---|
| name | heterologous genes | genes | product |
| MIA- | CroCPR, CroCYB5, CroG8H, | atf1Δ oye2Δ | Geraniol + |
| CH-A2 | Vmi8HGOA, NcaISY, | adh6Δ oye3Δ | tryptamine -> |
| | NcaMLPLA, CroIO, | ari1Δ | strictosidine |
| | CroCYPADH, Cro7DLGT, | | |
| | Cro7DLH, CroLAMT, | | |
| | CroSLS, CroSTR | | |

### Example 2: De Novo Production of Strictosidine in Yeast

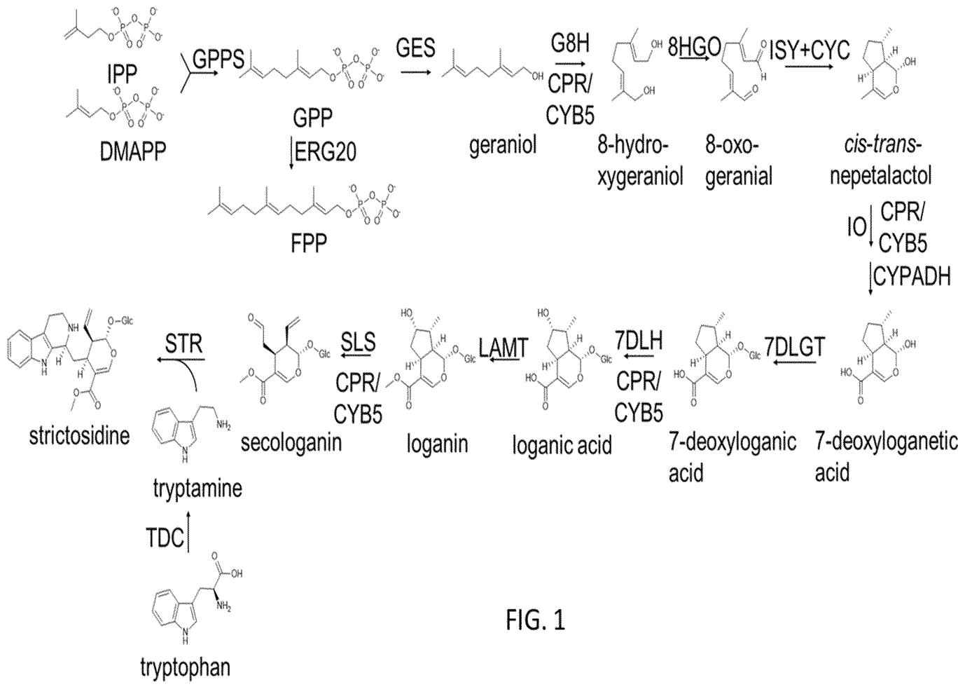

To enable de novo strictosidine production in yeast, we combined the genes for biosynthesis of the precursors geraniol and tryptamine with an optimized strictosidine pathway-design integrated into strain MIA-CH-A2. The full pathway is shown in FIG. 1.

Yeast can be engineered to produce geraniol de novo from the native metabolite geranyl diphosphate (GPP). The reaction can be catalyzed by geraniol synthase, for example from Catharanthus roseus, which can be functionally expressed in yeast cytosol by truncating the enzyme to remove the chloroplast signal peptide (trunCroGES). Conversion of GPP to geraniol can be improved by fusing trunCroGES to an enzyme with GPP synthase activity such as farnesyl pyrophosphate synthase variant F96W-N127W from Saccharomyces cerevisiae (ERG20**) with a short flexible peptide linker (Gly-Gly-Gly-Ser (SEQ ID NO: 148) yielding the enzyme ERG20**-GS-trunCroGES (SEQ ID NO: 66).

We considered different strategies to improve the intracellular concentration of geraniol precursor GPP: 1) overexpression of enzymes with GPP synthase activity such as farnesyl pyrophosphate synthase variant N144W from Gallus gallus (GgaFPS*(N144W)), or geranyl diphosphate synthase from Abies grandis (AgrGPPS2), 2) over-expression of native genes from the mevalonate pathway such as Isopentenyl diphosphate:dimethylallyl diphosphate isomerase (ID11) or truncated HMG-CoA reductase (trunHMG1), and 3) reducing competition for GPP from yeast ergosterol biosynthesis by reducing expression of native genes ERG20 and ERG9. This can be achieved by replacing the native promoters (pERG20 and pERG9) with pHTX1 and pHTX3 promoters, which are repressed upon glucose depletion.

Tryptamine is synthesized by decarboxylation of tryptophan by the enzyme tryptophan decarboxylase from Catharanthus roseus (CroTDC). To enable de novo production of strictosidine in yeast, we combined all strategies in the MIA-CH-A2 base strain, yielding strains MIA-CM-3 and MIA-CM-5:

$$MIA - CH - A 2 : CroCPR + CroCYB 5 + CroG 8 H + Vmi 8 HGOA + NcaISY + NcaMLPLA + CroIO + CroCYPADH + Cro 7 DLGT + Cro 7 DLLH + CroLAMT + CroSLS + CroSTR$$

$$MIA - CM - 3 : MIA - CH - A 2 + pERG 9 \text{::} pHXT 1 + pERG 20 \text{::} pHXT 3 + pMLS 1 - AgrGPPS 2 + {GgaFPS}^{*} ( N 144 W ) + IDI 1 + trunHMG 1 + CroTDC + pICL 1 - ERG {20}^{**} - GS - trunCroGES$$

$$MIA - CM - 5 : MIA - CH - A 2 + pERG 9 \text{::} pHXT 1 + pERG 20 \text{::} pHXT 3 + pTEF 2 - AgrGPPS 2 + {GgaFPS}^{*} ( N 144 W ) + IDI 1 + trunHMG 1 + CroTDC + pCCW 12 - ERG {20}^{**} - GS - trun - CroGES$$

### Verification of De Novo Strictosidine Production in Yeast

MIA-CM-3, MIA-CM-5 and MIA-CH-A2 were grown on YPD agar plates for 3 days at 30° C. to obtain individual colonies. From each strain, four individual colonies were picked and used to inoculate a pre-culture of 100 μl YP+2% glucose in a 96-well microtiter plate. The cultures were incubated at 30° C., 300 rpm for 24 h. From these precultures, 10 μL were used to inoculate 500 μL of YP+2% glucose. The cultures were incubated in a 96-deepwell plate for 9 days at 30° C., 300 rpm. Strictosidine, loganin, secologanin and tryptamine were analyzed by targeted LC-MS on an advance UHPLC system (Bruker Daltonics, Fremont, CA, USA) coupled to an EVOQ Elite triple quadrupole mass spectrometer (Bruker Daltonics, Fremont, CA, USA).

### Results:

The titers obtained in the cultivation are shown in table 2.

TABLE 2
Titers obtained by de novo production of strictosidine, tryptamine,
loganin and secologanin in yeast. Yeast strains MIA-CM-5 and MIA-CM-3
both produced strictosidine de novo while no strictosidine or strictosidine
precursors were produced by the parent strain MIA-CH-A2.

| | MIA-CM-3 | | MIA-CM-5 | | MIA-CH-A2 | |
|---|---|---|---|---|---|---|
| | Mean | Standard | Mean | Standard | mean | Standard |
| MIA | titer | deviation | titer | deviation | titer | deviation |
| Compound | (mg/L) | (mg/L) | (mg/L) | (mg/L) | (mg/L) | (mg/L) |
| Loganin | 4.4 | 1.2 | 1.6 | 0.1 | 0.0 | 0.0 |
| Secologanin | 0.1 | 0.0 | 0.1 | 0.0 | 0.0 | 0.0 |
| Strictosidine | 25.2 | 3.1 | 8.7 | 0.5 | 0.0 | 0.0 |
| Tryptamine | 13.7 | 4.1 | 6.8 | 0.3 | 0.0 | 0.0 |

MIA-CM-3 and MIA-CM-5 both produced strictosidine (25.2±4.1 mg/L and 6.8±0.3 mg/L, respectively) and the intermediates loganin, secologanin, and tryptamine without supplementation of geraniol or tryptamine. The parent strain MIA-CH-A2 did not produce any MIA compounds when supplemented with glucose and tryptophan alone. This demonstrates de novo production of strictosidine by a yeast strain with optimized strictosidine pathway design.

### Example 3: De Novo Production of Tetrahydroalstonine, Ajmalicine, Alstonine and Serpentine in Yeast

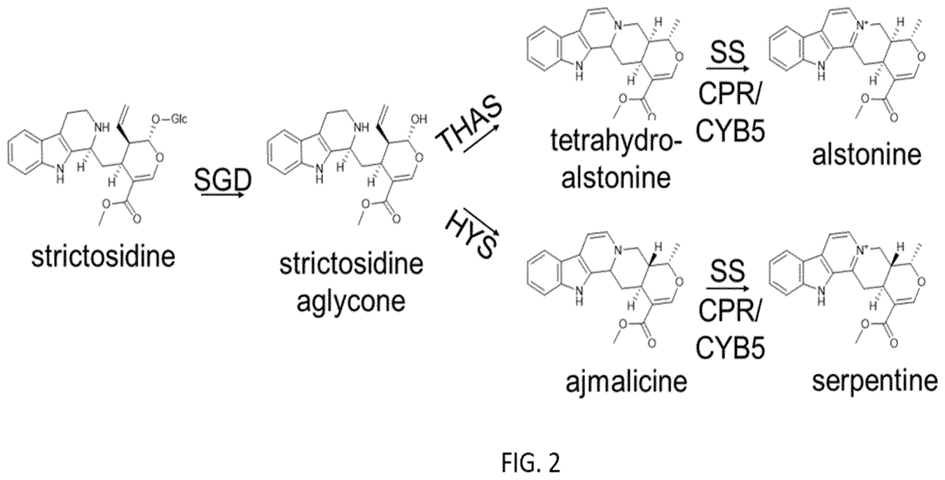

Alstonine and serpentine are natural MIAs derived from strictosidine. The biosynthetic pathway is shown in FIG. 2. First strictosidine is hydrolyzed by strictosidine-O-β-D-glucosidase to strictosidine aglycone. In yeast this reaction can be functionalized by expression of strictosidine-O-β-D-glucosidase from Rauvolfia serpentina (RseSGD). Strictosidine aglycone can be reduced by heteroyohimbine synthase from Catharanthus roseus (CroHYS) or tetrahydroalstonine synthase from Catharanthus roseus (CroTHAS1). CroTHAS1 produces tetrahydroalstonine and CroHYS produces primarily ajmalicine and small amounts of tetrahydroalstonine and mayumbine. Serpentine synthase from Catharanthus roseus (CroSS) catalyzes the oxidation of ajmalicine and tetrahydroalstonine to form serpentine and alstonine, respectively.

Yeast strains Sc85 and Sc112 were constructed to produce serpentine and alstonine from strictosidine.

$$Sc 85 : MIA - CM - 5 + RseSGD + CroHYS + CroSS$$

$$Sc 112 : MIA - CM - 5 + RseSGD + CroTHAS 1 + CroSS$$

### Verification of De Novo Production of Tetrahydroalstonine, Ajmalicine, Alstonine and Serpentine.

Yeast strains Sc85 and Sc112 were grown on YPD agar plates for 3 days at 30° C. to obtain individual colonies. Three colonies of each strain were inoculated in 150 μL SC+2% glucose and incubated overnight at 30° C. and 300 rpm in a 96-well microtiter plate. After 16 h, 10 μL of each culture was transferred into 500 μL of fresh 3×SC+2% glucose+3 mM tryptophan in a 96-deepwell plate and incubated for 144 h at 30° C. and 300 rpm. MIA metabolites in culture broth were analyzed by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA).

### Results:

The titers obtained in the cultivation are shown in table 3.

TABLE 3
Titers obtained by de novo production of ajmalicine, serpentine,
tetrahydroalstonine and alstonine. Strain Sc85 produced
1270 ± 130 μg/L serpentine and strain Sc112 produced
1034 ± 94 μg/L alstonine. This shows that de novo
production of heteroyohimbine MIAs is possible in yeast.

| | | Mean titer | Standard deviation |
|---|---|---|---|
| Strain | Compound | (μg/L) | (μg/L) |
| Sc85 | Ajmalicine | 906 | 100 |
| | Serpentine | 1270 | 130 |
| Sc112 | Tetrahydroalstonine | 166 | 35 |
| | Alstonine | 1034 | 94 |

This result shows that strictosidine produced de novo in yeast can be further converted into heteroyohimbine type MIAs. Strain Sc112 produced 166 μg/L tetrahydroalstonine and 1034 μg/L alstonine, and strain Sc85 produced 906 μg/L ajmalicine and 1270 μg/L serpentine.

### Example 4: De Novo Production of Stemmadenine Acetate

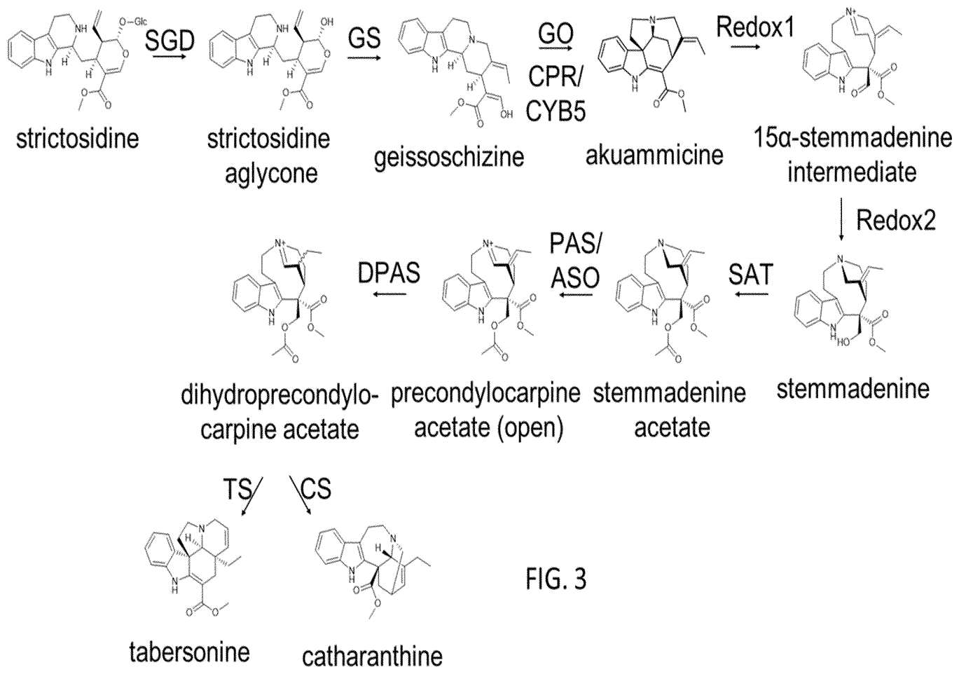

Stemmadenine acetate or its direct precursor stemmadenine are precursors for many bio-active MIAs e.g. tabersonine and catharanthine. In nature strictosidine is converted to stemmadenine acetate in six enzyme-catalyzed steps (FIG. 3): First, strictosidine is hydrolyzed by strictosidine-O-β-D-glucosidase (SGD) to strictosidine aglycone, which is then converted to stemmadenine by geissoschizine synthase (GS), geissoschizine oxidase (GO), protein Redox 1 (Redox1), and protein Redox 2 (Redox2). Finally, stemmadenine is acetylated by stemmadenine-O-acetyltransferase (SAT) to form the more stable compound stemmadenine acetate.

Stemmadenine acetate can be converted into catharanthine and tabersonine in three enzymatic steps (FIG. 3): First, stemmadenine acetate is oxidized by the enzyme O-acetylstemmadenine oxidase/precondylocarpine acetate synthase to form precondylocarpine acetate (PAS/ASO), which is reduced by dihydroprecondylocarpine acetate synthase (DPAS) to form dihydroprecondylocarpine acetate. The last step is cyclization by either tabersonine synthase (TS) to form tabersonine or by catharanthine synthase (CS) to form catharanthine.

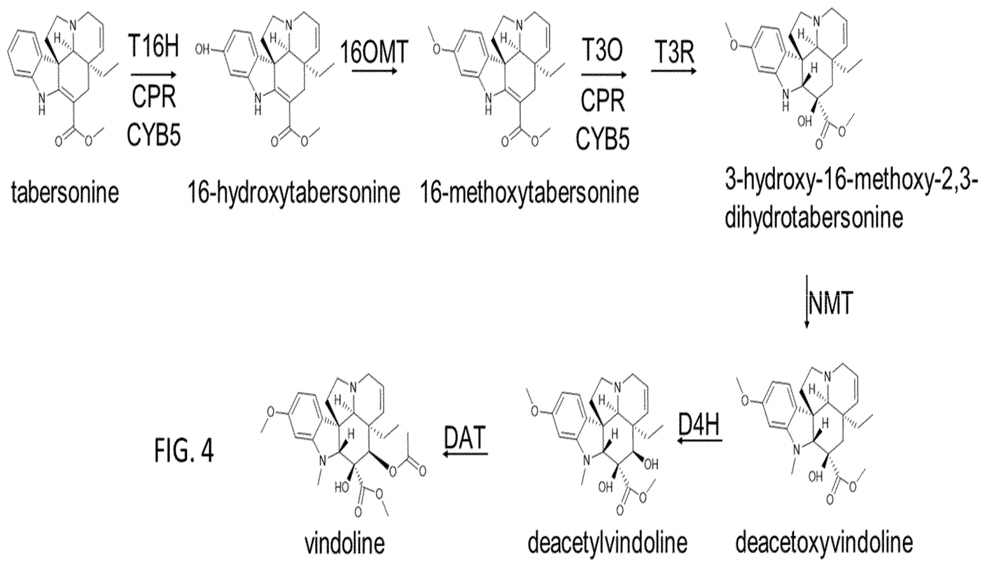

Tabersonine can be converted into vindoline (FIG. 4). The seven enzymes required for this conversion are: tabersonine 16-hydroxylase (T16H), tabersonine 16-O-methyltransferase (16OMT), tabersonine 3-oxygenase (T3O), 16-methoxy-2,3-dihydro-3-hydroxytabersonine synthase (T3R), 3-hydroxy-16-methoxy-2,3-dihydrotabersonine N-methyltransferase (NMT), deacetoxyvindoline 4-hydroxylase (D4H), and deacetylvindoline O-acetyltransferase (DAT).

To construct a strain for de novo production of stemmadenine acetate, tabersonine, vindoline and catharanthine all the required genes were integrated into MIA-CM-3 resulting in the yeast strain MIA-EM-2. Selected bottleneck genes were integrated in two or three copies to improve flux through the pathway. CroCS was expressed under the control of a galactose-inducible promotor (pGAL1). MIA-EM-2 expressed one copy of the variant of CroTS as set forth in SEQ ID NO: 39 and one copy of the variant of CroTS as set forth in SEQ ID NO: 150.

$$MIA - EM - 2 : MIA - CM - 3 + CroCPR + ERG {20}^{*} - GS - trunCroGES + AgrGPPS 2 + CroSTR + RseSGD + 3 x CroGS + 3 x CroGO + CroRdx 1 + CroRdx 2 + 2 x CroSAT + 2 x CroPAS + 2 x CroDPAS + 2 x CroTS + CroCS + CroT 16 H 2 + Cro 16 OMT + CroT 3 O + CroT 3 R + CroNMT + CroD 4 H + CroDAT$$

### Verification of De Novo Production of Stemmadenine Acetate

To demonstrate de novo production of stemmadenine acetate in yeast we cultivated yeast strains MIA-CM-5 and MIA-EM-2 on YPD agar plates for 3 days at 30° C. to obtain individual colonies. From each strain three individual colonies were picked and used to inoculate a pre-culture of 100 μL 3×SC+2% glucose+3 mM tryptophan in a 96-well microtiter plate. The cultures were incubated at 30° C., 300 rpm for 24 hours. From these precultures 10 μl were used to inoculate 500 μL 3×SC+2% glucose+3 mM tryptophan in a 96-deepwell plate and incubated for 6 days at 30° C., 300 rpm. Cells were precipitated by centrifugation and spent medium was harvested for metabolite analysis. MIA metabolites produced during cultivation were analyzed by untargeted high-resolution LC-MS on an Orbitrap Fusion Mass Spectrometer (Thermo Fisher Scientific, San Jose, CA). No commercial standard of stemmadenine acetate being available, we identified stemmadenine acetate based on exact mass (monoisotopic m/Z 397.2127) and the most abundant MS/MS fragment ions reported for a semi-synthetic stemmadenine acetate standard (Farrow et al. 2019).

### Results:

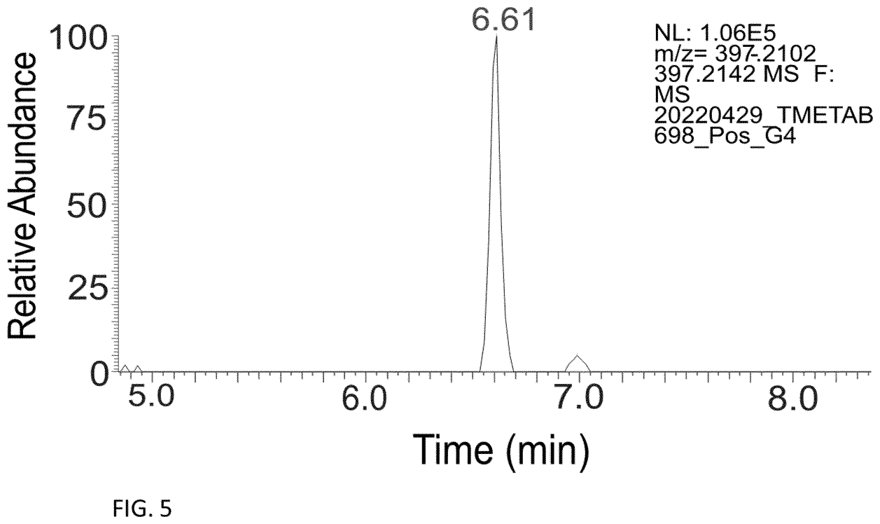

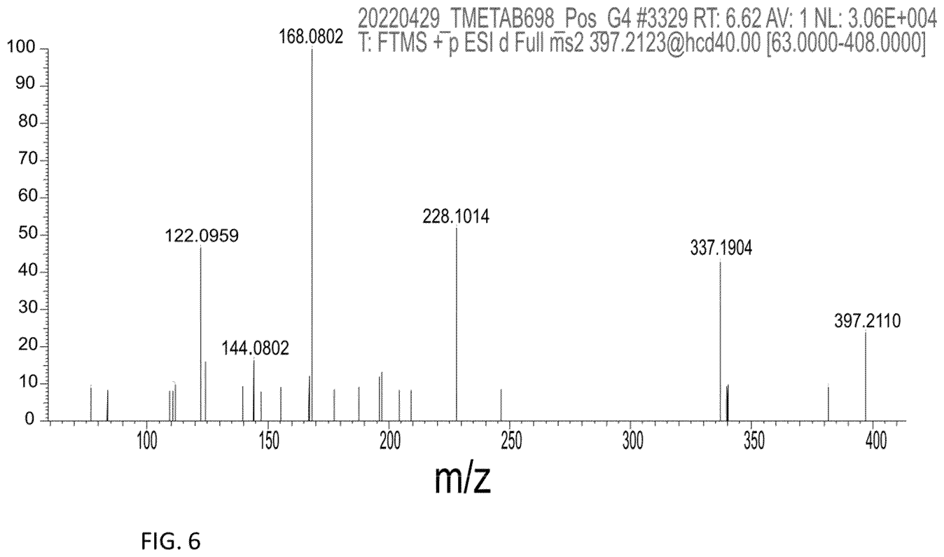

In spent medium from MIA-EM-2 we detected a compound with the expected mass of stemmadenine acetate eluding at 6.61 min (FIG. 5). The peak was not detected in the spent medium from MIA-CM-5. In the MS/MS spectrum of this peak, the four most abundant ions observed have m/Z of 168.08, 228.10, 122.09, and 337.19 (FIG. 6), which matches the most abundant MS/MS fragments of semi-synthetic stemmadenine acetate reported by Farrow et al. (2019). Altogether, this shows that stemmadenine acetate can be produced de novo by yeast strain MIA-EM-2.

### Example 5: De Novo Production of Catharanthine, Tabersonine and Vindoline in Yeast

De novo production of catharanthine, tabersonine and vindoline was tested in a fed-batch process cultivation with continuous exponential feeding of glucose and galactose induction of CroCS.

### Verification of De Novo Catharanthine and Vindoline Production

Yeast strain MIA-EM-2 was cultivated on YPD agar plates for 3 days at 30° C. to obtain individual colonies. Small-scale fed-batch cultivations were performed in BioLector Pro systems using flower-shaped plates. The fermentation started with inoculating 1 mL 3×SC+2% glucose+3 mM tryptophan to an initial optical density (OD_{600 nm}) of 0.5. Continuous exponential feeding of the same medium except containing 36% glucose started 20 hours after the inoculation. Temperature was kept at 30° C. throughout the whole cultivation. The pH was controlled at 5.5 using 10% NH₄OH solution. The plate was shaken at speed 1,000 rpm. The relative humidity in the growth chamber was maintained at 85% using distilled water to minimize evaporation of the media. On day 5, 10 g/L galactose was added to induce the expression of CroCS for de novo catharanthine production. Cultivation was stopped on day 7 and MIA compounds were analyzed by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA).

### Results:

The titers obtained in the cultivation are shown in table 4. Yeast strain MIA-EM-2 produced tabersonine (0.153 mg/L), vindoline (0.005 mg/L) and catharanthine (0.069 mg/L) under the tested cultivation conditions. This result shows that catharanthine, tabersonine and vindoline derived from stemmadenine acetate can be simultaneously produced de novo in yeast.

TABLE 4
Titers obtained of loganic acid, loganin, secologanin, strictosidine,
tabersonine, catharanthine and vindoline by de novo production
in yest. MIA-EM-2 produced tabersonine (0.153 mg/L), vindoline
(0.005 mg/L) and catharanthine (0.069 mg/L).

| | metabolite concentration (mg/L) | | | | |
|---|---|---|---|---|---|
| | replicate | replicate | replicate | | Standard |
| MIA-EM-2 | 1 | 2 | 3 | Mean | deviation |
| loganic acid | | 0.317 | 0.553 | 0.435 | 0.167 |
| loganin | 3.352 | 4.354 | 13.1 | 6.94 | 5.37 |
| secologanin | | | 0.133 | 0.133 | |
| strictosidine | 0.041 | 0.031 | 0.046 | 0.039 | 0.008 |
| tabersonine | 0.12 | 0.129 | 0.209 | 0.153 | 0.049 |
| vindoline | 0.003 | 0.004 | 0.009 | 0.005 | 0.003 |
| catharanthine | 0.042 | 0.045 | 0.121 | 0.069 | 0.045 |

### Example 6: De Novo Production of Tabersonine in Yeast

Some natural homologues of plant enzymes may express or fold better in a microbial cell. To identify more homologs of GS, GO, PAS/ASO and DPAS we searched NCBI with BLAST using CroGS, CroGO, CroPAS and CroDPAS as queries. All newly identified homologs are summarized in table 5 together with those already identified and tested from Catharanthus roseus.

TABLE 5
All identified homologs of GS, GO, PAS/ASO and DPAS

| Name | Species of origin | Function |
|---|---|---|
| CroGO | Catharanthus roseus | geissoschizine synthase |
| CroADH13 | Catharanthus roseus | geissoschizine synthase |
| CroGO | Catharanthus roseus | geissoschizine oxidase |
| AhuGO | Amsonia hubrichtii | geissoschizine oxidase |
| TelGO | Tabernaemontana | geissoschizine oxidase |
| | elegans | |
| VmiGO | Vinca minor | geissoschizine oxidase |
| CroPAS | Catharanthus roseus | O-acetylstemmadenine oxidase/ |
| | | precondylocarpine acetate synthase |
| TibPAS2 | Tabernanthe iboga | O-acetylstemmadenine oxidase/ |
| | | precondylocarpine acetate synthase |
| AhuASO | Amsonia hubrichtii | O-acetylstemmadenine oxidase/ |
| | | precondylocarpine acetate synthase |
| TelASO | Tabernaemontana | O-acetylstemmadenine oxidase/ |
| | elegans | precondylocarpine acetate synthase |
| CroDPAS | Catharanthus roseus | dihydroprecondylocarpine acetate |
| | | synthase |
| TibDPAS2 | Tabernanthe iboga | dihydroprecondylocarpine acetate |
| | | synthase |

All homologs (except for TelASO) were tested for tabersonine production in yeast.

By integrating additional genes in strain MIA-CM-5 we constructed several yeast strains for de novo production of tabersonine. In each strain we integrated one GS homolog (CroGS or CroADH13), one GO homolog (CroGO or AhuGO or TelGO or VmiGO), one PAS homolog (CroPAS or TibPAS2 or AhuASO), and one DPAS homolog (CroDPAS or TibDPAS2) together with RseSGD, CroRdx1, CroRdx2, CroSAT and CroTS. The gene homologs were integrated in different combinations to test if a specific combination was required for tabersonine production. MIA-CV expressed the variant of CroTS as set forth in SEQ ID NO: 39, and MIA-CT-A-04, MIA-CT-A-12, MIA-CTA-16, MIA-CT-A-18, MIA-CT-A-34, and MIA-CT-A-36 expressed the variant of CroTS as set forth in SEQ ID NO: 150.

$$MIA - CT - A - 04 : MIA - CM - 5 + CroADH 13 + TelGO + AhuASO + CroDPAS + RseSGD + CroRdx 1 + CroRdx 2 + CroTS + CroSAT$$

$$MIA - CT - A - 12 : MIA - CM - 5 + CroADH 13 + VmiGO + TibPAS 2 + CroDPAS + RseSGD + CroRdx 1 + CroRdx 2 + CroTS + CroSAT$$

$$MIA - CT - A - 16 : MIA - CM - 5 + CroADH 13 + AhuGO + AhuASO + CroDPAS + RseSGD + CroRdx 1 + CroRdx 2 + CroTS + CroSAT$$

$$MIA - CT - A - 18 : MIA - CM - 5 + CroADH 13 + AhuGO + TibPAS 2 + CroDPAS + RseSGD + CroRdx 1 + CroRdx 2 + CroTS + CroSAT$$

$$MIA - CT - A - 34 : MIA - CM - 5 + CroADH 13 + AhuGO + AhuASO + TibPAS 2 + RseSGD + CroRdx 1 + CroRdx 2 + CroTS + CroSAT$$

$$MIA - CT - A - 36 : MIA - CM - 5 + CroADH 13 + AhuGO + TibPAS 2 + TibDPAS + RseSGD + CroRdx 1 + CroRdx 2 + CroTS + CroSAT$$

$$MIA - CV : MIA - CM - 5 + CroGS + CroGO + CroPAS + CroDPAS + RseSGD + CroRdx 1 + CroRdx 2 + CroTS + CroSAT$$

### Verification of De Novo Tabersonine Production in Yeast

MIA-CM-5, all MIA-CT-A strains, and MIA-CV strain were grown on YPD agar plates for 3 days at 30° C. to obtain individual colonies. From each strain, three individual colonies were picked and used to inoculate a pre-culture of 100 μL YP+2% glucose in a 96-well microtiter plate. The cultures were incubated at 30° C., 300 rpm for 24 hours. From these precultures, 10 μL were used to inoculate 500 μL YP+2% glucose in a 96-deepwell plate and incubated for 6 days at 30° C., 300 rpm. Tabersonine production was quantified by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA).

### Results

Tabersonine titers are shown in table 6.

TABLE 6
Tabersonine titers obtained by de novo production in yeast. The integrated homolog
of GS, GO, PAS/ASO and DPAS is indicated for each constructed strain. All strains
constructed for tabersonine production could produce at least 193.7 ±
4.5 μg/L tabersonine. This result indicates that all tested GS, GO, PAS/ASO
and DPAS homologs are functional and can function in any combination.

| | | | | | Mean | |
|---|---|---|---|---|---|---|
| | | | | | tabersonine | Standard |
| | GS | GO | PAS | DPAS | titer | deviation |
| Strain | homolog | homolog | homolog | homolog | (μg/L) | (μg/L) |
| MIA-CT-A-04 | CroADH13 | TelGO | AhuASO | CroDPAS | 201.3 | 0.0 |
| MIA-CT-A-12 | CroADH13 | VmiGO | TibPAS2 | CroDPAS | 263.0 | 0.2 |
| MIA-CT-A-16 | CroADH13 | AhuGO | AhuASO | CroDPAS | 201.0 | 9.9 |
| MIA-CT-A-18 | CroADH13 | AhuGO | TibPAS2 | CroDPAS | 285.5 | 13.5 |
| MIA-CT-A-34 | CroADH13 | AhuGO | AhuASO | TibDPAS2 | 193.7 | 4.5 |
| MIA-CT-A-36 | CroADH13 | AhuGO | TibPAS2 | TibDPAS2 | 295.1 | 1.5 |
| MIA-CV | CroGS | CroGO | CroPAS | CroDPAS | 240.4 | 0.0 |
| MIA-CM-5 | | | | | 0.0 | 0.0 |

As expected, MIA-CM-5 did not produce tabersonine, while all yeast strains engineered to express RseSGD, CroRdx1, CroRdx2, CroSAT, CroTS and a homolog of GS, GO, PAS/ASO, and DPAS produced at least 193.7±4.5 μg/L tabersonine under the tested cultivation conditions. This demonstrates that all identified and tested homologs of GS, GO, PAS/ASO and DPAS are functional for the production of tabersonine in yeast.

### Example 7: Production of Halogenated Strictosidine

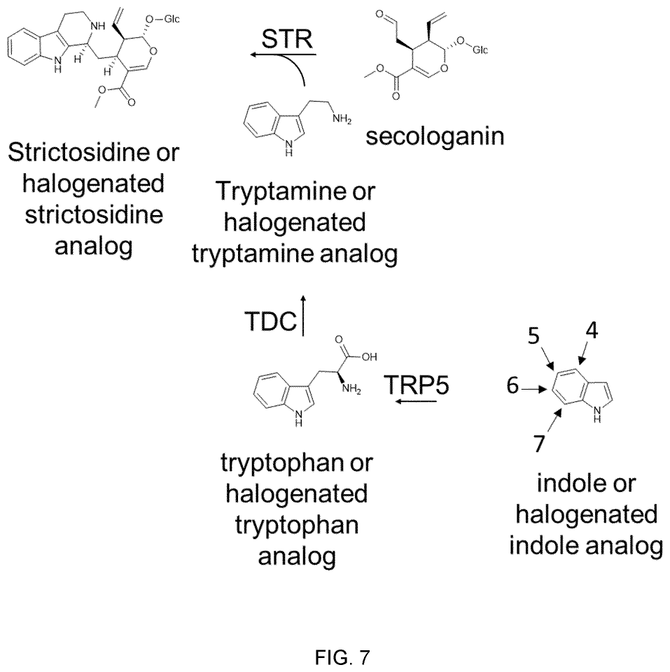

The native yeast enzyme tryptophan synthase (TRP5) catalyzes the conversion of indole and serine to tryptophan. We exploited this activity for the production of halogenated strictosidine analogues by feeding fluoro-, chloro-, bromo- and difluoro-indole analogues to de novo strictosidine strains. First, a fed halogenated indole is converted to halogenated tryptophan by TRP5, which is then decarboxylated to form halogenated tryptamine by tryptophan carboxylase (TDC) (FIG. 7). Finally, halogenated tryptamine is coupled with secologanin by strictosidine synthase (STR) to form halogenated strictosidine. Tryptophan decarboxylase from Catharanthus roseus is known to have low activity for chloro- and bromotryptophan. For efficient production of chloro- and bromostrictosidine, we constructed a new de novo strictosidine strain (MIA-CM-10) harboring a promiscuous tryptophan decarboxylase from the bacterium Ruminococcus gnavus (rgnTDC) expressed under an inducible pGAL1 promoter:

$$MIA - CM - 10 : MIA - CH - A 2 + pERG 9 \text{::} pHXT 1 + pERG 20 \text{::} pHXT 3 + pTEF 2 - AgrGPPS 2 + {GgaFPS}^{*} ( N 144 W ) + IDI 1 + trunHMG 1 + pGAL 1 - rgnTDC + pCCW 12 - ERG {20}^{**} - GS - trunCroGES$$

### Example 7a: Verification of Production of Halogenated Strictosidine

To test production of halogenated strictosidine from feeding halogenated indole analogues three colonies of yeast strains MIA-CM-3 and MIA-CM-10 were used for inoculation of three cultures of 7.5 mL cultures SC+2% glucose and incubated overnight 30° C., 300 rpm. Each culture was centrifuged at 3,000 rpm for 5 min. The supernatant was discarded, and the pellet resuspended in 7.5 mL SC+2% galactose supplemented with 0.25 mM secologanin. 450 μL of the cell suspension was transferred to a deep-well plate, 50 μL of 1 g/L 4-fluoroindole, 5-fluoroindole, 6-fluoroindole, 7-fluoroindole, 4-chloroindole, 5-chloroindole, 6-chloroindole, 7-chloroindole, 4-bromoindole, 5-bromoindole, 6-bromoindole or 7-bromoindole dissolved in 20% DMSO or 50 μL 20% DMSO control was added to the well. The plate was incubated for 144 h at 30° C. and 300 rpm. Samples were analyzed by untargeted high-resolution LC-MS on an Orbitrap Fusion Mass Spectrometer (Thermo Fisher Scientific, San Jose, CA). Halogenated tryptamine analogues, halogenated tryptophan analogues and halogenated strictosidine analogues were identified based on the exact mass, elongated retention time compared with natural analogues and a mass shift in MS/MS spectra as expected from hydrogen-to-halogen substitution. Titers were quantified as total peak area.

### Results:

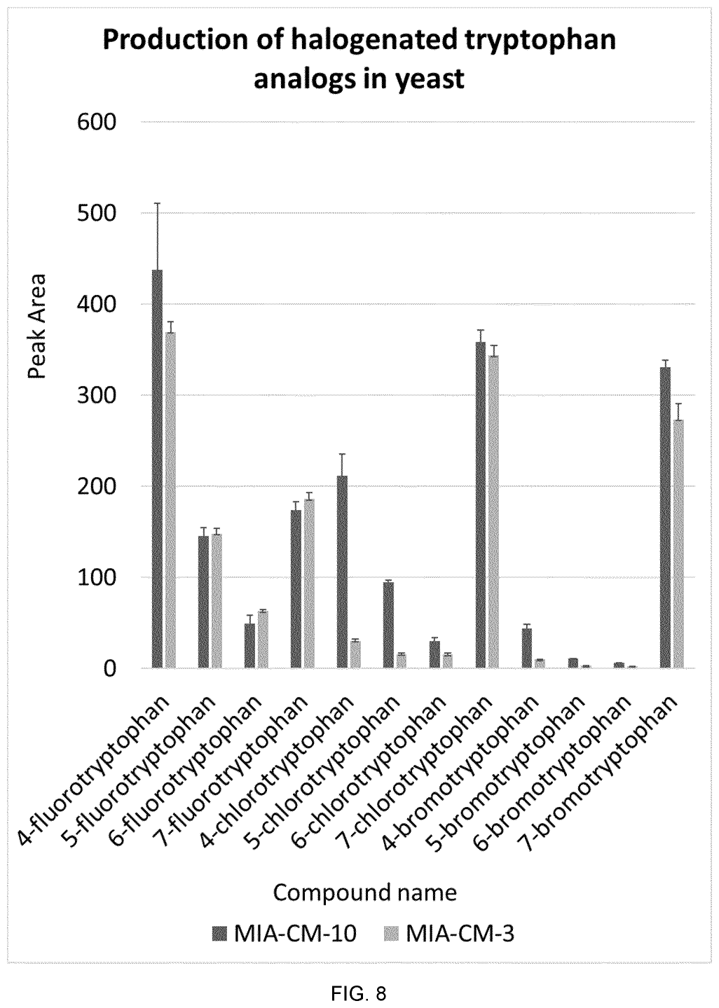

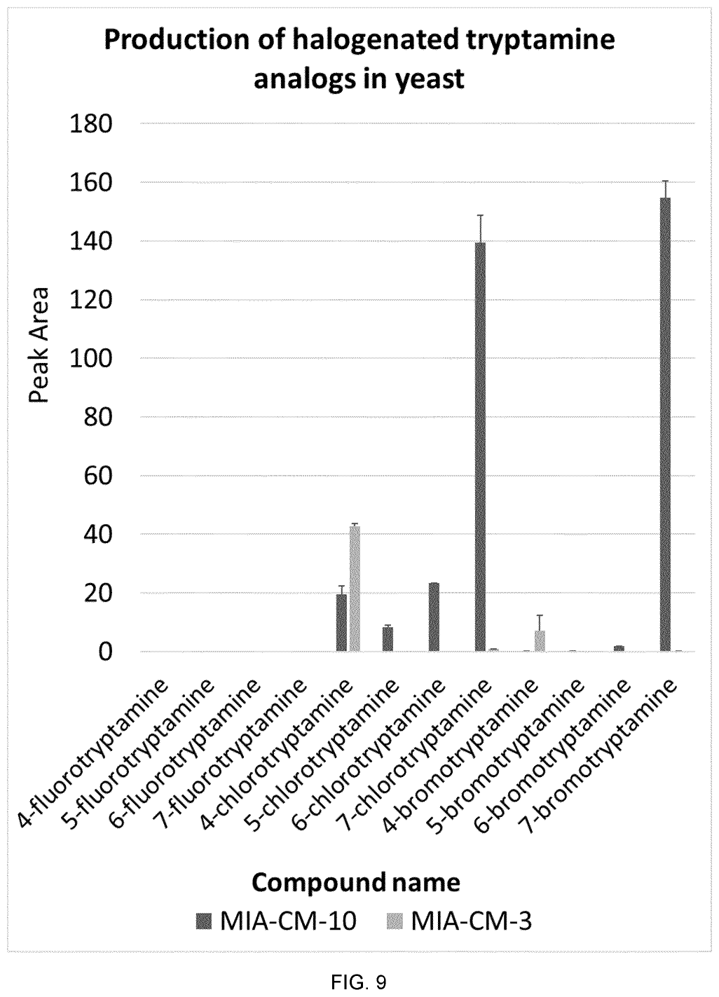

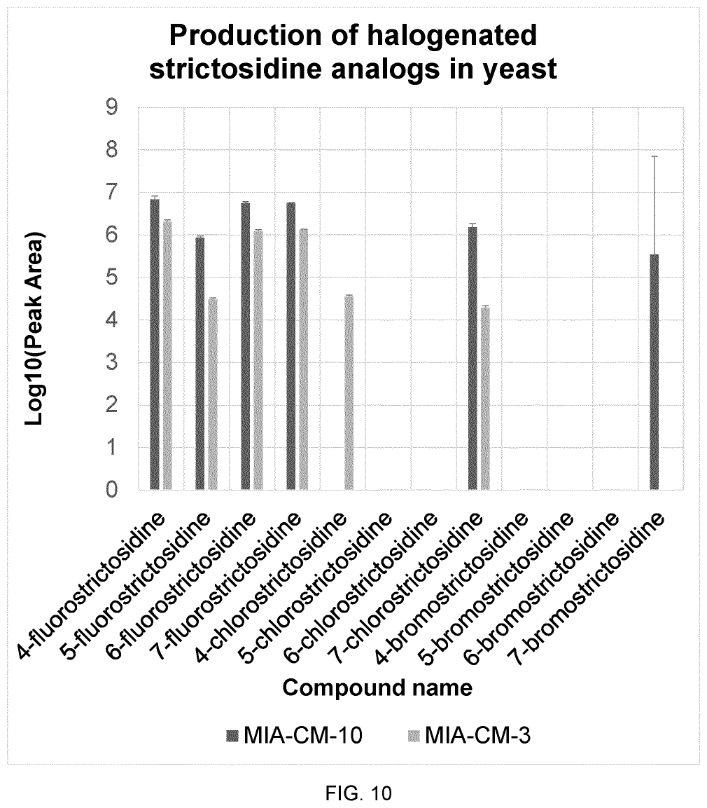

Results for halogenated tryptophan are shown in FIG. 8. Strains MIA-CM-3 and MIA-CM-10 were able to produce both fluoro-, chloro-, and bromotryptophan with the halogen atom on all four tested positions. MIA-CM-10 could produce 4-, 5-, 6-, and 7-chlorotryptamine and 6- and 7-bromotryptamine (FIG. 9). MIA-CM-3 only produced 4- and 7-chlororotryptamine and 4-bromotryptamine (FIG. 9). The more limited product profile of MIA-CM-3 shows that a promiscuous TDC such as rgnTDC allows for more bulky halogens at position 5 and 6. No fluorotryptamine analogues were detected, however the detection of 4-, 5-, 6-, and 7-fluorostrictosidine produced by both MIA-CM-3 and MIA-CM-10 indicates that fluorotryptamine must have been produced (FIG. 10). MIA-CM-3 also produced 4- and 7-chlorostrictosidine. MIA-CM-10 produced 7-chlorostrictosidine and 7-bromostrictosidine. This demonstrated that halogenated strictosidine analogues can be produced in yeast from halo-indole feeding.

This demonstrated that halogenated strictosidine analogues can be produced in yeast from fed halogenated-indole analogues. CroSTR accepted tryptamine fluorinated at all four tested positions, while chlorination is possible at least at positions 4 and 7 and bromination at least at position 7.

### Example 7b

To test production of halogenated strictosidine from feeding halogenated indole analogues three colonies of yeast strains MIA-CM-3 and MIA-CM-10 were used for inoculation of three cultures of 22.5 mL cultures 3×SC+2% glucose+20 g/L peptone and incubated for 72 hours at 30° C., 300 rpm. Each culture was centrifuged at 3,000 rpm for 5 min. The supernatant was discarded, and the pellet resuspended in 7.5 mL SC-TRP+2% galactose supplemented with 0.25 mM secologanin. The OD₆₀₀ was corrected to 80. 270 μL of the cell suspension was transferred to a deep-well plate, 30 μL of 1 g/L indole, 4-fluoroindole, 5-fluoroindole, 6-fluoroindole, 7-fluoroindole, 4-chloroindole, 5-chloroindole, 6-chloroindole, 7-chloroindole, 4-bromoindole, 5-bromoindole, 6-bromoindole, 7-bromoindole, 4,5-difluoroindole, 4,6-difluoroindole, 4,7-difluoroindole, 5,6-difluoroindole, 5,7-difluoroindole or 6,7-difluoroindole dissolved in 3×SC 5% acetone. The plate was incubated for 144 h at 30° C. and 300 rpm. Samples were analyzed by untargeted high-resolution LC-MS on an Orbitrap Fusion Mass Spectrometer (Thermo Fisher Scientific, San Jose, CA). Fluorinated and chlorinated tryptophan analogues, halogenated tryptamine analogues and halogenated strictosidine analogues were identified based on the exact mass, elongated retention time compared with natural analogues and a mass shift in MS/MS spectra as expected from hydrogen-to-halogen substitution(s). MS/MS data for brominated tryptophan analogs was inconclusive and the compounds were identified by exact mass and confirmed by the presence of derivatized brominated tryptamine. Results were quantified as total peak area.

### Results:

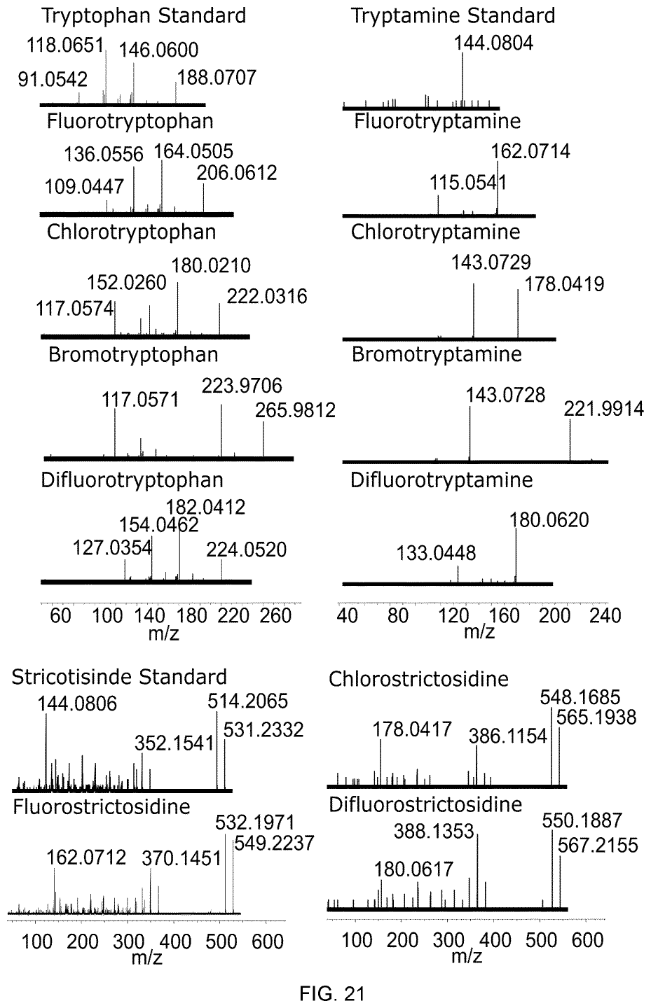

FIG. 21 shows representative MS/MS spectra obtained for standard and halogenated tryptophan, tryptamine and strictosidine. Halogenation changes the m/z of fragments containing the indole moiety according to the molecular mass of the substituting halogen. A change consistent with the mass of the halogen atom minus that of the removed hydrogen atom is indicative of such a substitution and is important in the identification of halogenated analogues. For example, the major fragments in the MS/MS spectrum of tryptophan have observed m/z of 188.0707, 146.0600, 118.0651 and 91.0542. Fluorine has an atomic mass of 18.998 g/mol and the major peak of the MS/MS spectrum of fluorotryptophan are 206.0612, 164.0505, 136.0556 and 109.0447, demonstrating a mass shift consistent with the expected hydrogen to fluorine substitution. Difluorination substitutions increase the mass by 37.996, a chlorination substitution increases the mass by 34.9689 (isotope C135) or 36.9659 (isotope C137) and bromination increases the mass by 78.9183 (isotope Br79) or 80.9163 (isotope Br81). The expected mass shift is observed for all fragments of detected fluoro-, difluoro-, chloro- and bromotryptophan, fluoro-, difluoro-, chloro- and bromotryptamine and all fluoro-, difluoro- and chlorostrictosidine (FIG. 21). No reliable MS/MS spectra could be obtained for bromostrictosidine. Peak areas for detected halogenated tryptophan are shown in table 7. Strains MIA-CM-3 and MIA-CM-10 were able to produce both fluoro-, chloro-, difluoro-, and bromotryptophan with the halogen atom(s) on all four tested positions. MIA-CM-10 could produce 4-, 5-, 6- and 7-fluorotryptamine, 4-, 5-, 6-, and 7-chlorotryptamine and 4-, 5-, 6- and 7-bromotryptamine and 4,5-, 4,6-, 4,7-, 5,6-, 5,7-, and 6,7-difluorotryptamine (table 7). MIA-CM-3 could only produce 4-, 5-, 6- and 7-fluorotryptamine, 4-, and 7-chlorotryptamine and 4,5-, 4,6-, 4,7-, 5,6-, 5,7-, and 6,7-difluorotryptamine (table 7). The more limited product profile of MIA-CM-3 shows that a promiscuous TDC such as rgnTDC allows for more bulky halogens at position 5 and 6.

MIA-CM-3 also produced 4-, 5-, 6- and 7-fluorostrictosidine, 4-chlorostrictosidine and 4,5-, 4,7-difluorostrictosidine (table 7). MIA-CM-10 also produced 4-, 5-, 6- and 7-fluorostrictosidine, 7-chlorostrictosidine and 4,5-, 4,6-difluorostrictosidine (table 7). This demonstrates that halogenated strictosidine analogues can be produced in yeast from halo-indole feeding. CroSTR accepted tryptamine fluorinated at all four tested positions, while chlorination is possible at least at positions 4 and 7 and bromination is possible at least at position 7. Difluorination of strictosidine was possible at least at position 4 in combination with position 5, 6, or 7.

TABLE 7
Halogenated tryptophan, tryptamine and strictosidine
retention times and peak areas. NF: not found

| | | MIA-CM-3 | MIA-CM-10 |
|---|---|---|---|
| Compound | RT | Peak Area | Peak Area |
| 4-fluorotryptophan | 5.3 | 251881803 | 53064275 |
| 5-fluorotryptophan | 5.3 | 153824617 | 35680950 |
| 6-fluorotryptophan | 5.3 | 64382490 | 9693963 |
| 7-fluorotryptophan | 5.3 | 74864908 | 24092525 |
| 4-chlorotryptophan | 5.8 | 180289445 | 159055893 |
| 5-chlorotryptophan | 5.8 | 168638434 | 93625116 |
| 6-chlorotryptophan | 5.9 | 50095222 | 10269537 |
| 7-chlorotryptophan | 5.8 | 109822946 | 27975039 |
| 4-bromotryptophan | 5.86 | 34900421.87 | 60418721.15 |
| 5-bromotryptophan | 5.95 | 6410171.06 | 7281603.03 |
| 6-bromotryptophan | 5.91 | 111739207.28 | 29364778.81 |
| 7-bromotryptophan | 5.89 | 111345376.40 | 25581204.63 |
| 4,5-difluorotryptophan | 5.6 | 242324893 | 109013098 |
| 4,6-difluorotryptophan | 5.6 | 217133772 | 57975702 |
| 4,7-difluorotryptophan | 5.5 | 189664946 | 129767463 |
| 5,6-difluorotryptophan | 5.6 | 154550975 | 37133848 |
| 5,7-difluorotryptophan | 5.6 | 145684798 | 105019674 |
| 6,7-difluorotryptophan | 5.6 | 137530278 | 41785307 |
| 4-fluorotryptamine | 5.7 | 877127450 | 577895283 |
| 5-fluorotryptamine | 5.6 | 18512807 | 219266877 |
| 6-fluorotryptamine | 5.6 | 29927141 | 177038860 |
| 7-fluorotryptamine | 5.6 | 103850542 | 111612016 |
| 4-chlorotryptamine | 6.1 | 490598968 | 235597855 |
| 5-chlorotryptamine | 6.2 | 491431 | 99316199 |
| 6-chlorotryptamine | 6.2 | 627417 | 37505497 |
| 7-chlorotryptamine | 6.1 | 4974073 | 120987890 |
| 4-bromotryptamine | 6.1 | NF | 5758959 |
| 5-bromotryptamine | 6.3 | NF | 1849232 |
| 6-bromotryptamine | 6.2 | NF | 219402437 |
| 7-bromotryptamine | 6.2 | NF | 185462237 |
| 4,5-difluorotryptamine | 5.9 | 344537067 | 825156793 |
| 4,6-difluorotryptamine | 5.9 | 272149875 | 372112061 |
| 4,7-difluorotryptamine | 5.9 | 607145130 | 547659094 |
| 5,6-difluorotryptamine | 5.9 | 3024435 | 142211132 |
| 5,7-difluorotryptamine | 5.9 | 2639439 | 194715357 |
| 6,7-difluorotryptamine | 5.9 | 8013412 | 127345820 |
| 4-fluorostrictosidine | 6.5 | 19831032 | 13895659 |
| 5-fluorostrictosidine | 6.5 | 763445 | 3278350 |
| 6-fluorostrictosidine | 6.5 | 3890630 | 3970515 |
| 7-fluorostrictosidine | 6.5 | 9432612 | 4721842 |
| 4-chlorostrictosidine | 6.7 | 541461.00 | NF |
| 7-chlorostrictosidine | 6.6 | NF | 642452.00 |
| 7-bromostrictosidine | 6.7 | NF | 129289.00 |
| 4,5-difluorostrictosidine | 6.6 | 214944.00 | 564502.00 |
| 4,6-difluorostrictosidine | 6.6 | NF | 319632.00 |
| 4,7-difluorostrictosidine | 6.6 | 850050.00 | NF |
| 5,6-difluorostrictosidine | 6.6 | NF | NF |
| 5,7-difluorostrictosidine | 6.5 | NF | NF |
| 6,7-difluorostrictosidine | 6.5 | NF | NF |

### Example 8: Production of Halogenated, Incl. Fluorinated, MIAs from Halogenated Indole Analogues, Such as Fluoroindole Analogues

In example 7, we demonstrated that halogenated strictosine, such as fluorostrictosidine, can be produced in yeast by feeding halogenated indole analogues, such as fluoroindole analogues, and secologanin to a yeast strain engineered for de novo production of strictosidine. To test if yeast-produced halogenated strictosidine, such as fluorostrictosidine analogues, can be further converted into other halogenated MIA analogues, such as other fluorinated MIA analogues, we fed 0.25 mM secologanin together with either 4-, 5-, 6-, or 7-fluoroindole, 4-, 5-, 6- or 7-chloroindole, 4-, 5-, 6-, or 7-bromoindole or 4,5-, 4,6-, 4,7-, 5,6-, 5,7-, or 6,7-fluoroindole to five yeast strains —MIA-CM-5, Sc112, Sc161, SAB125, and MIA-EM-2—engineered for de novo production of strictosidine, alstonine, alstonine, serpentine, and vindoline/catharanthine, respectively.

In example 7 we showed that a more promiscuous TDC such as rgnTDC is more permissible for bulky substitutions at position 5 and 6. To leverage this for production of halogenated MIAs, we constructed two new strains based on strain MIA-CM-10 expressing rgnTDC. Strain SAB125 was constructed for de novo production of serpentine and strain Sc161 was constructed for de novo production of alstonine. To improve alstonine production, the native yeast gene ROX1 was deleted in strain Sc161 by integration of a kanMX antibiotics resistance selection cassette into the ROX1 open reading frame. ROX1 is a negative regulator of heme synthesis. Heme is required as a cofactor for the five cytochrome P450 enzymes—CroG8H, CroIO, Cro7DLH, CroSLS, and CroSS—in the alstonine pathway.

$$SAB 125 : MIA - CM - 10 + RseSGD + CroHYS + CroSS$$

$$Sc 161 : MIA - CM - 10 + RseSGD + 2 x CroTHAS 1 + CroSS + rox Δ \text{::} kanMX$$

Verification of Production of MIAs Derived from Fluorostrictosidine

The strains MIA-CM-5, Sc112, and MIA-EM-2 strains were streaked on YPD agar plates. The plates were incubated for 3 days at 30° C. to obtain individual colonies. Four colonies were picked from each strain and inoculated into 2 mL 3×SC+2% glucose and incubated at 30° C., 300 rpm to obtain cells for bioconversion of secologanin and fluoroindole analogues. After 72 hours, the cells were harvested and washed with 2 mL 3×SC-HULT. The washed cells were resuspended in 1250 μL SC-HULT+2% glucose+0.25 mM secologanin and spilt into five wells in a 96-deepwell plate, each well containing 250 μl culture. For the four colonies of each strain, tryptophan was added to the first well to a final concentration of 0.150 g/L, to the second well 4-fluoroindole was added to a final concentration of 0.100 g/L, to the third well 5-fluoroindole was added to a final concentration of 0.100 g/L, to the fourth well 6-fluoroindole was added to a final concentration of 0.100 g/L and to the fifth well 7-fluoroindole was added to a final concentration of 0.100 g/L. The cultures were incubated for 72 hours at 30° C., 300 rpm.

The strains Sc161 and SAB125 were streaked on YPD agar plates. The plates were incubated for 3 days at 30° C. to obtain individual colonies. Three colonies were picked from each strain and inoculated into 22.5 mL 3×SC+2% glucose and incubated at 30° C., 300 rpm, to obtain cells for bioconversion of secologanin and haloindole analogues. After 72 hours, the cells were harvested and washed with 10 ml PBS+4% ethanol. The washed cells were resuspended in 1250 μL SC-TRP+2% glucose+0.25 mM secologanin and the OD₆₀₀ corrected to 80. For each strain, 270 μl each replicate was put into 19 separate wells in 96-deepwell plate, for a total of 57 wells. For each replicate, indole was added to the first well to a final concentration of 0.150 g/L, to the second well 4-fluoroindole was added to a final concentration of 0.100 g/L, to the third well 5-fluoroindole was added to a final concentration of 0.100 g/L, to the fourth well 6-fluoroindole was added to a final concentration of 0.100 g/L and to the fifth well 7-fluoroindole was added to a final concentration of 0.100 g/L, to the sixth well 4-chloroindole was added to a final concentration of 0.100 g/L, to the seventh well 5-chloroindole was added to a final concentration of 0.100 g/L, to the eighth well 6-chloroindole was added to a final concentration of 0.100 g/L and to the ninth well 7-chloroindole was added to a final concentration of 0.100 g/Lt, o the tenth well 4-bromoindole was added to a final concentration of 0.100 g/L, to the twelfth well 5-bromoindole was added to a final concentration of 0.100 g/L, to the thirteenth well 6-bromoindole was added to a final concentration of 0.100 g/L and to the fourteenth well 7-bromoindole was added to a final concentration of 0.100 g/L to the fifteenth well 4,5-difluoroindole was added to a final concentration of 0.100 g/L, to the sixteenth well 4,6-difluoroindole was added to a final concentration of 0.100 g/L, to the seventeenth well 4,7-difluoroindole was added to a final concentration of 0.100 g/L and to the eighteenth well 5,7-difluoroindole was added to a final concentration of 0.100 g/L, to the nineteenth well 6,7-difluoroindole was added to a final concentration of 0.100 g/L. The cultures were incubated for 144 hours at 30° C., 300 rpm.

Production of halogenated MIAs, such as fluorinated MIAs, derived from halogenated strictosidine, such as fluorostrictosidine, were analyzed by Orbitrap Fusion Mass Spectrometer (Thermo Fisher Scientific, San Jose, CA). Halogenated MIA analogues, including fluorinated MIA analogues, were identified based on the exact mass, elongated retention time compared with natural analogue and a mass shift in MS/MS spectra as expected from hydrogen-to-halogen substitution. If concentration was too low to obtain the MS/MS data, the samples were reanalyzed by targeted LC-MS/MS on an advanced UHPLC system (Bruker Daltonics, Fremont, CA, USA) coupled to an EVOQ Elite triple quadrupole mass spectrometer (Bruker Daltonics, Fremont, CA, USA).

### Results:

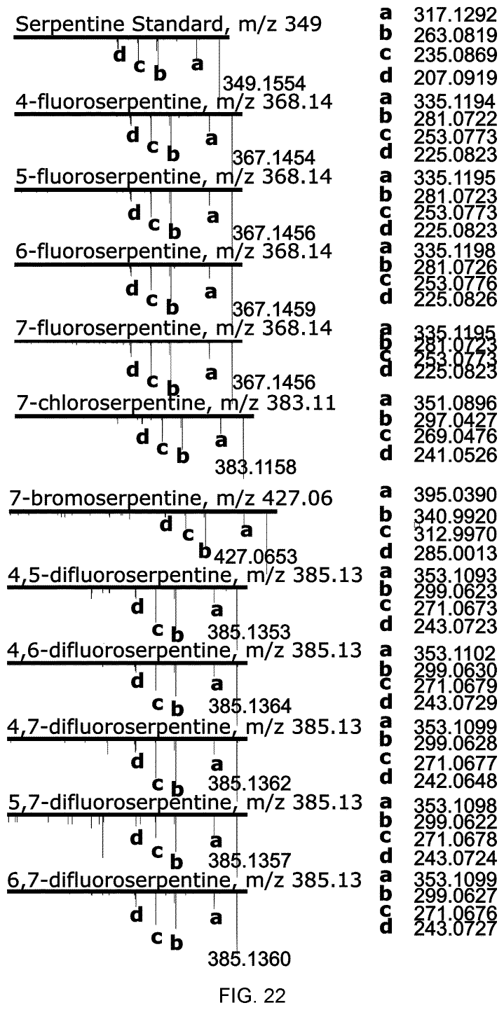

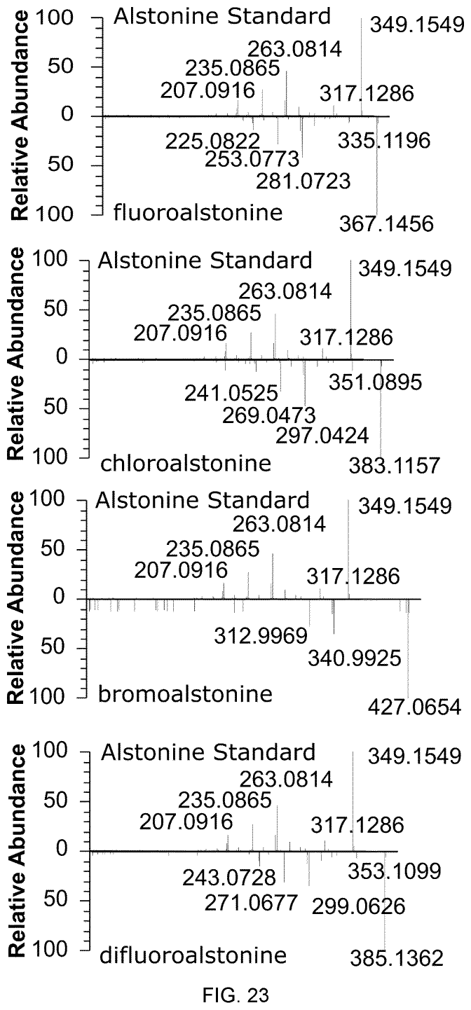

FIG. 22 shows representative MS/MS spectra for all detected haloserpentine analogs produced by strain SAB125. All spectra have four major fragments labelled a-d. All fragments show the expected mass shift caused by hydrogen-to-halogen substitution(s). The MS/MS spectra for 4-, 5-, 6-, and 7-fluoroserpentine are close to identical, which shows that the position of substitution does not affect MS/MS fragmentation. FIG. 23 shows representative MS/MS spectra from haloalstonine produced by strain Sc161. All fragments show the expected mass shift e.g. the fragment (m/z 349.1549) in the alstonine standard shows the expected mass shift upon fluorination (m/z 367.1456), chlorination (m/z 383.1157), bromination (m/z 427.0654), or difluorination (m/z385.1362).

Table 8 summarizes detected peak areas for fluorostrictosidine aglycones, fluorotetrahydroalstonines, halogenated serpentines, and halogenated alstonines, including fluoroalstonines, fluorostemmadenine acetate and fluorotabersonine.

Strain Sc112 produced 4-, 5-, 6-, and 7-fluorotetrahydroalstonine, and 5-, 6-, and 7-fluoroalstonine. Strain MIA-EM-2 produced 4-, 5-, 6-, and 7-fluorostrictosidine aglycone. Strain Sc161 produced 4-, 5-, 6-, and 7-fluoroalstonine, 7-chloroalstonine, 7-bromoalstonine, 6,7-difluoroalstonine and 5,6-difluoroalstonine. Strain SAB125 produced 4-, 5-, 6-, and 7-fluoroserpentine, 7-chloroserpentine, 7-bromoserpentine, 6,7-difluoroserpentine, 5,7-difluoroserpentine, 4,7-difluoroserpentine, 4,6-difluroserpentine and 4,5-difluoroserpentine.

With these data we have shown that it is possible to use yeast strains engineered for production of MIAs to produce alstonine or serpentine fluorinated at position 4, 5, 6, or 7, chlorinated or brominated at position 7, and difluorinated at position 4+5, 4+6, 4+7, 5+7, 5+6, or 6+7.

TABLE 8
Production of halogenated, such as fluorinated, MIA analogues in yeast quantified as
total peak area. Flourination at position 4, 5, 6, and 7 was demonstrated for fluorostrictosidine
aglycone, fluorotetrahydroalstonine, fluoroserpentine and fluoroalstonine. Fluorination
was also demonstrated at position 6 and 7 for fluorotabersonine and fluorostemmadenine
acetate. Difluorination was demonstrated at position 4 + 5, 4 + 6, 4 +
7, 5 + 7, 5 + 6, and 6 + 7 for difluoroalstonine and/or difluoroserpentine.
Chlorination was demonstrated at position 7 for chloroalstonine and chloroserpentine.
Bromination was demonstrated at position 7 for bromoalstonine and bromoserpentine.

| | | MIA-CM-5 | Sc112 | MIA-EM-2 | Sc161 | SAB125 |
|---|---|---|---|---|---|---|
| | RT | Peak | Peak | Peak | Peak | Peak |
| | (min) | Area | Area | Area | Area | Area |
| 4- | 6.51 | NF | 13486584 | 23925202 | NA | NA |
| fluorostrictosidine | | | | | | |
| aglycone | | | | | | |
| 5- | 6.45 | NF | 551517 | 87612 | NA | NA |
| fluorostrictosidine | | | | | | |
| aglycone | | | | | | |
| 6- | 6.49 | NF | 360283 | 1350222 | NA | NA |
| fluorostrictosidine | | | | | | |
| aglycone | | | | | | |
| 7- | 6.48 | NF | NF | 2160041 | NA | NA |
| fluorostrictosidine | | | | | | |
| aglycone | | | | | | |
| 4- | 6.86 | NF | 298073 | NA | NA | NA |
| fluorotetrahydroalstonine | | | | | | |
| 5- | 6.83 | NF | 72213 | NA | NA | NA |
| fluorotetrahydroalstonine | | | | | | |
| 6- | 6.81 | NF | 644990 | NA | NA | NA |
| fluorotetrahydroalstonine | | | | | | |
| 7- | 6.79 | NF | 30693 | NA | NA | NA |
| fluorotetrahydroalstonine | | | | | | |
| 4- | NF | NF | NF | NA | 1242696 | NA |
| fluoroalstonine | | | | | | |
| 5- | 6.77 | NF | 5561216 | NA | 10316543 | NA |
| fluoroalstonine | | | | | | |
| 6- | 6.87 | NF | 6107129 | NA | 30918995 | NA |
| fluoroalstonine | | | | | | |
| 7- | 6.85 | NF | 69744 | NA | 50065048 | NA |
| fluoroalstonine | | | | | | |
| 7- | 7.0 | NF | NF | NA | 2296077 | NA |
| chloroalstonine | | | | | | |
| 7- | 7.2 | NF | NF | NA | 354799 | NA |
| bromoalstonine | | | | | | |
| 5,6- | 7.1 | NA | NA | NA | 694340 | NA |
| difluoroalstonine | | | | | | |
| 6,7- | 7.0 | NA | NA | NA | 931119 | NA |
| difluoroalstonine | | | | | | |
| 4- | 6.9 | NA | NA | NA | NA | 193134322 |
| fluoroserpentine | | | | | | |
| 5- | 6.9 | NA | NA | NA | NA | 30065496 |
| fluoroserpentine | | | | | | |
| 6- | 6.9 | NA | NA | NA | NA | 475978110 |
| fluoroserpentine | | | | | | |
| 7- | 6.8 | NA | NA | NA | NA | 749683965 |
| fluoroserpentine | | | | | | |
| 7- | 7.1 | NA | NA | NA | NA | 8340211 |
| chloroserpentine | | | | | | |
| 7- | 7.2 | NA | NA | NA | NA | 869668 |
| bromoserpentine | | | | | | |
| 4,5- | 7.0 | NA | NA | NA | NA | 4744126 |
| difluoroserpentine | | | | | | |
| 4,6- | 7.0 | NA | NA | NA | NA | 4484543 |
| difluoroserpentine | | | | | | |
| 4,7- | 6.9 | NA | NA | NA | NA | 2014736 |
| difluoroserpentine | | | | | | |
| 5,7- | 6.9 | NA | NA | NA | NA | 869989 |
| difluoroserpentine | | | | | | |
| 6,7- | 7.0 | NA | NA | NA | NA | 3891944 |
| difluoroserpentine | | | | | | |
| 6- | 6.8 | NF | NA | 12981 | NA | NA |
| fluorostemmadenine | | | | | | |
| acetate | | | | | | |
| 7- | 6.8 | NF | NA | 12930 | NA | NA |
| fluorostemmadenine | | | | | | |
| acetate | | | | | | |
| 6- | 7.0 | NF | NA | 13089 | NA | NA |
| fluorotabersonine | | | | | | |
| 7- | 7.0 | NF | NA | 2633 | NA | NA |
| fluorotabersonine | | | | | | |

NF = not found, NA = not analyzed.

### Fluorostemmadenine Acetate

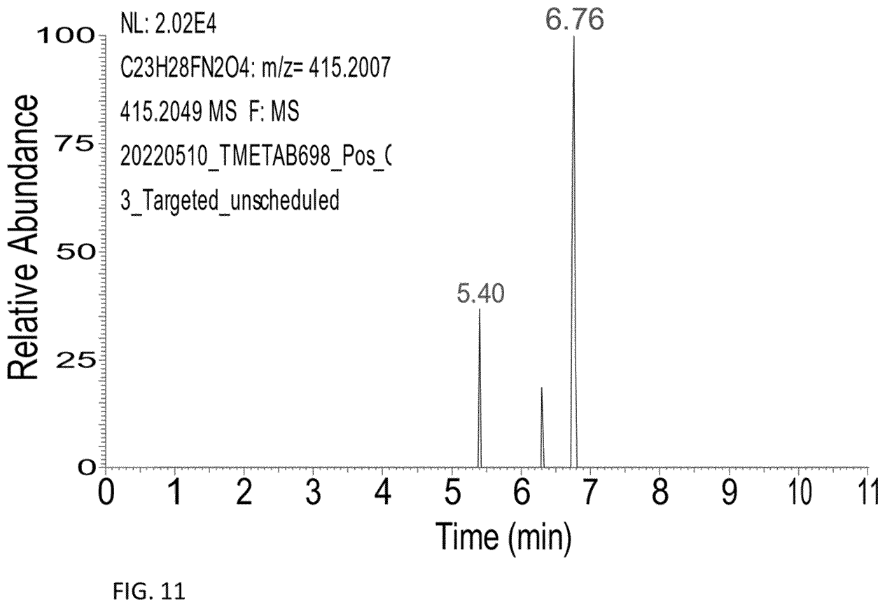

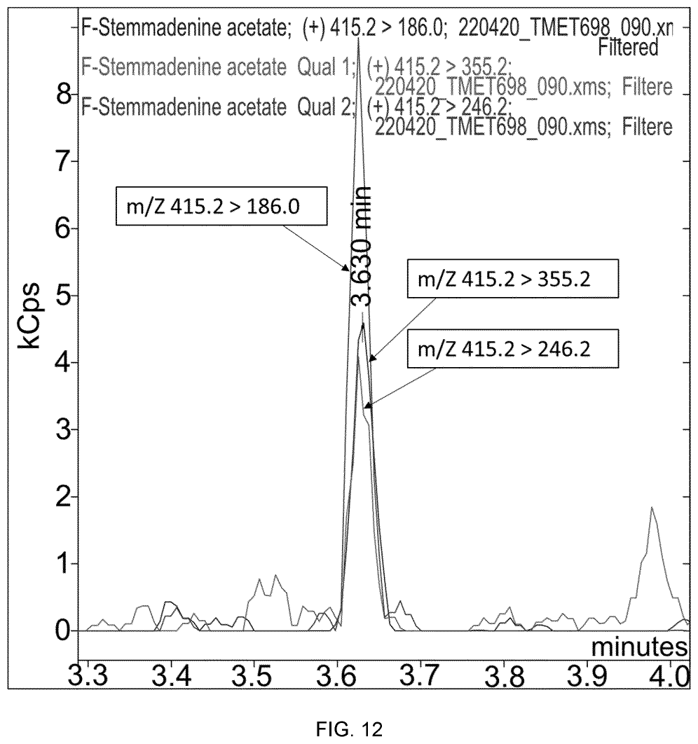

A peak with the expected mass of fluorostemmadenine acetate (m/Z 415.20) was detected at retention time 6.76 min in samples of MIA-EM-2 fed with secologanin and 6-fluoroindole or 7-fluoroindole (FIG. 11). This peak was not detected for strains unable to produce stemmadenine acetate, such as MIA-CM-5. FIG. 12 shows EIC of three fragment ions from fluorostemmadenine acetate with the expected mass shift from hydrogen-to-fluorine substitution (m/Z 415>186.0, 415.2>355.2, 415.2>246.2). All three fragments co-eluded, which indicates they are derived from the same compound. Retention time differs from FIG. 11 due to a different chromatographic method. Accurate mass and fragment ion mass shift together confirms that the detected compound was fluorostemmadenine acetate. Peak areas for 6- and 7-fluorostemmadenine acetate are summarized in table 8. With these data we have demonstrated production of 6- and 7-fluorostemmadenine acetate in yeast.

### Fluorotabersonine

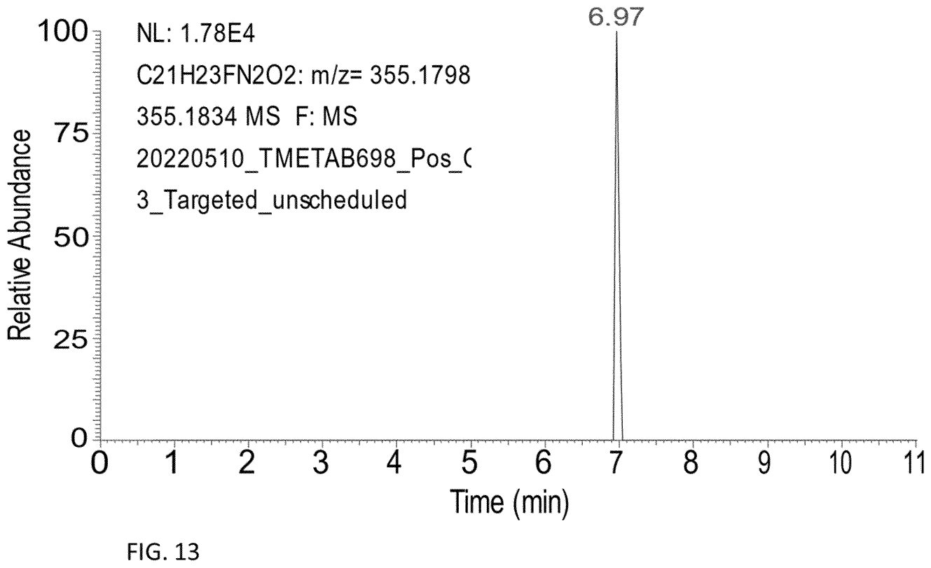

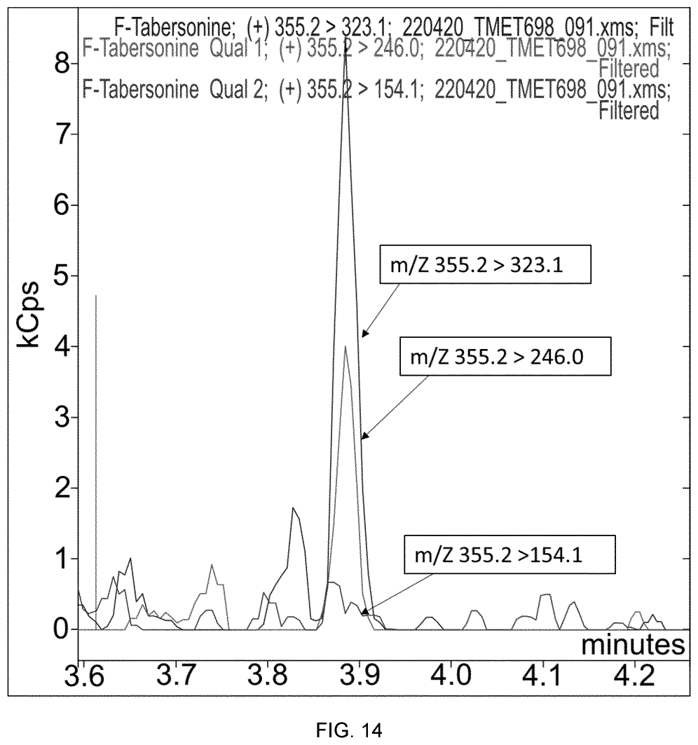

A peak with the expected mass of fluorotabersonine (m/Z 355.18) was detected at retention time 6.97 min in broth of MIA-EM-2 fed with secologanin and 7-fluoroindole or 6-fluoroindole (FIG. 13). The peak was not observed in strains that are unable to produce tabersonine, such as MIA-CM-5. FIG. 14 shows EIC of three fragment ions of fluorotabersonine with expected mass shift from hydrogen-to-fluorine substitution (m/Z 355.2>323.1, 355.2>246.0, 355.2>154.1). All three fragments co-eluded, which confirms they originated from the same compound. Accurate mass and fragment ions with expected mass shift together confirms that the detected compound was fluorotabersonine. Peak areas for 6- and 7-fluorotabersonine are summarized in table 8. These results demonstrate the production of 6- and 7-fluorotabersonine in engineered yeast.

We have demonstrated that halogenated MIA analogues, including fluorinated MIA analogues, can be produced in yeast from feeding halogenated indole analogues, such as fluoroindole analogues. With wild-type enzymes tetrahydroalstonine fluorination was possible at positions 4, 5, 6 and 7. For stemmadenine acetate and tabersonine fluorination was possible at least at positions 6 and 7.

### Example 9: Conservation of Fluorine-Carbon Bond in Yeast Produced Fluorotryptophan

The indole ring position of hydrogen-to-halogen substitution is likely to be important for bioactivity of halogenated MIA analogues. Since no standards of halogenated MIA are commercially available, we could not compare retention time of yeast-produced halogenated MIA analogue to an authentic standard to confirm the halogen position. When halogenated MIAs were produced from feeding indole analogues it was assumed that the position of the halogen is conserved due to the stability of covalent fluorine-carbon, chlorine-carbon, or bromine-carbon bonds.

Authentic standards of fluorotryptophan analogues are commercially available. To demonstrate that the fluorine-carbon bond in a fluoroindole is conserved when converted to fluorotryptophan by yeast, we compared the retention time of yeast-produced 4-fluorotryptophan and 6-fluorotryptophan to the corresponding authentic standards.

### Verification of Fluorine Position of Fluorotryptophan Produced by Fluoroindole Feeding.

The strain MIA-CM-5 was grown on YPD agar plate to obtain individual colonies. One colony was used to inoculate 2 mL 3×SC+2% glucose+1 mM tryptophan and incubated at 30° C., 300 rpm to obtain cells for bioconversion of secologanin and fluoroindole. After 72 hours, the cells were harvested and washed with 2 mL 3×SC-HULT. The washed cells were resuspended in 1 mL SC-HULT+2% glucose+0.25 mM secologanin and spilt into 4 wells in a 96-deepwell plate, each well containing 250 μL culture. To two wells tryptophan was added to a final concentration of 0.150 g/L and to two other wells either 4-fluoroindole or 6-fluoroindole was added to a final concentration of 0.100 g/L. The cultures were incubated for 72 hours at 30° C., 300 rpm after which the cells were precipitated by centrifugation. 50 μg/L of authentic standards of 4-flurotryotophan (Sigma-Aldrich) or 6-fluorotryptophan (Sigma-Aldrich) was added to samples from MIA-CM-5 fed tryptophan and secologanin for matrix matching. Samples were analyzed by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA). Fluorotryptophan isomers were separated on an PFP narrow pore LC column (InfinityLab Poroshell 120 PFP, 2.1×150 mm, 1.9 μm).

### Results:

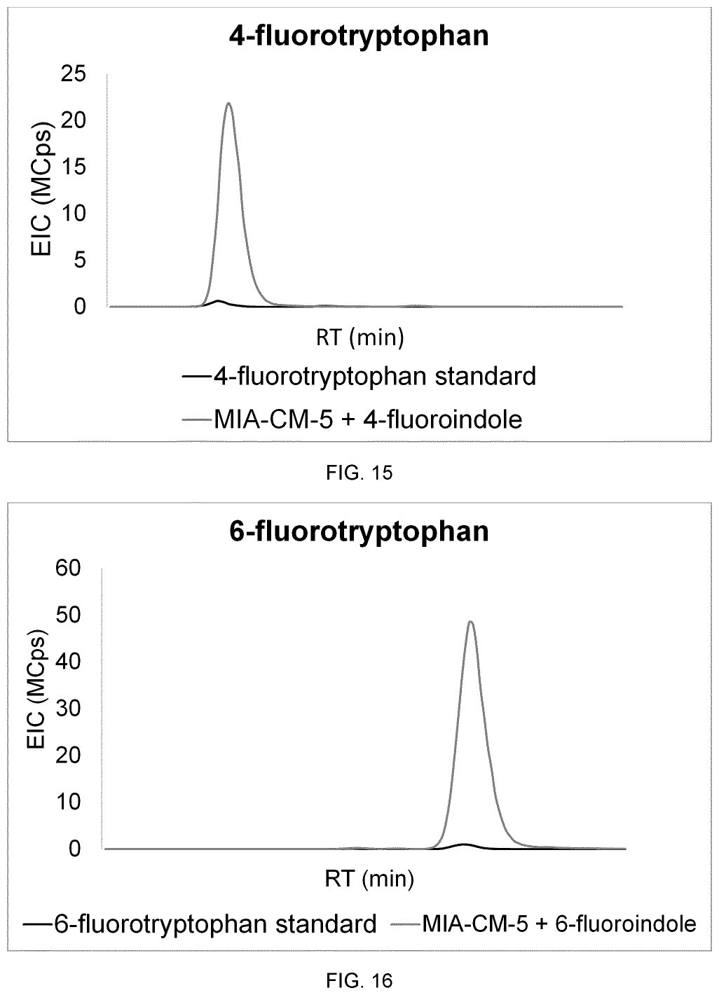

FIG. 15 and FIG. 16 show extracted ion chromatograms (EIC) of fluorotryptophan fragment ion m/Z 223.3>205.9. Retention time of fluorotryptophan produced by strain MIA-CM-5 from 4-fluoroindole matches perfectly with the 4-fluorotryptophan standard (Signa-Aldrich). Likewise, retention time for fluorotryptophan produced from 6-fluoroindole matches the 6-fluorotryptophan standard (Sigma-Aldrich). This demonstrates that the fluoro-carbon bonds are stable in yeast and that the position of the indole halogen is conserved during biosynthesis of halogenated MIAs.

### Example 10: De Novo Production of Chlorinated MIAs in Yeast

De novo chlorination and bromination of tryptamine is possible by co-expression of a mutant variant (N470S) of the flavin-dependent halogenase LaeRebH from Lechevalieria aerocolonigenes ATCC 39243 (IaeRebH_N470S), which has been previously shown to have enhanced activity towards C7 of tryptamine halogenation and the FMN reductase SsuE from Escherichia coli str. K-12 substr. MG1655 (EcoSsuE).

To construct strains for de novo production of chlorinated MIAs, we integrated IaeRebH_N470S and EcoSsuE in yeast strain MIA-CM-10. Subsequently, yeast strains Sc113 and Sc114 were created by integration of the genes for synthesis of alstonine or serpentine, respectively. Alternatively, IaeRebH can be fused with an N-terminal Trx tag and coexpressed with EcoSsuE in Sc161 to produce de novo chloroalstonine, making ScH135. The Trx tag increases the solubility of the IaeRebH protein in the yeast cytosol which in turn increases halogenation activity.

$$Sc 113 : MIA - CM - 10 + RseSGD + CroTHAS 1 + CroSS + IaeRebH\_N470S + EcoSsuE$$

$$Sc 114 : MIA - CM - 10 + RseSGD + CroHYS + CroSS + IaeRebH\_N470S + EcoSsuE$$

$$ScH 135 : Sc 161 + Trx - IaeRebH + EcoSsuE$$

### Verification of De Novo Production of Chloroalstonine and Chloroserpentine

To test the de novo production of halogenated alstonine or serpentine, strains Sc112, Sc85, Sc113 and Sc114 were used to inoculate 7.5 mL cultures of SC+2% glucose and incubated overnight at 30° C. and 300 rpm. Each culture was centrifuged at 3,000 rpm for 5 min, the supernatant discarded, and the pellet resuspended in 7.5 mL SC+2% galactose supplemented with 0.25 mM secologanin, 100 mg/L tryptophan and 300 mM NaCl or KBr. The plate was incubated for 144 h at 30° C. and 300 rpm, and sampled for targeted LC-HRMS analysis using an Orbitrap Fusion Mass Spectrometer (Thermo Fisher Scientific, San Jose, CA). To demonstrate that the strain ScH135 is able to produce chloroalstonine without the need for secologanin feeding, ScH135 and Sc161 were used to inoculate two cultures of 7.5 ml 3×SC 2% glucose 20 g/L peptone and incubated at 25° C. for 72 hours. The cultures were centrifuged at 3000 rpm for 5 min, the supernatant discarded, and each pellet resuspended in 2.5 mL 3×SC+2% galactose supplemented with 100 mg/L tryptophan and 300 mM NaCl. The plate was incubated for 144 h at 25° C. and 300 rpm, and sampled for targeted LC-HRMS analysis using an Orbitrap Fusion Mass Spectrometer (Thermo Fisher Scientific, San Jose, CA).

### Results:

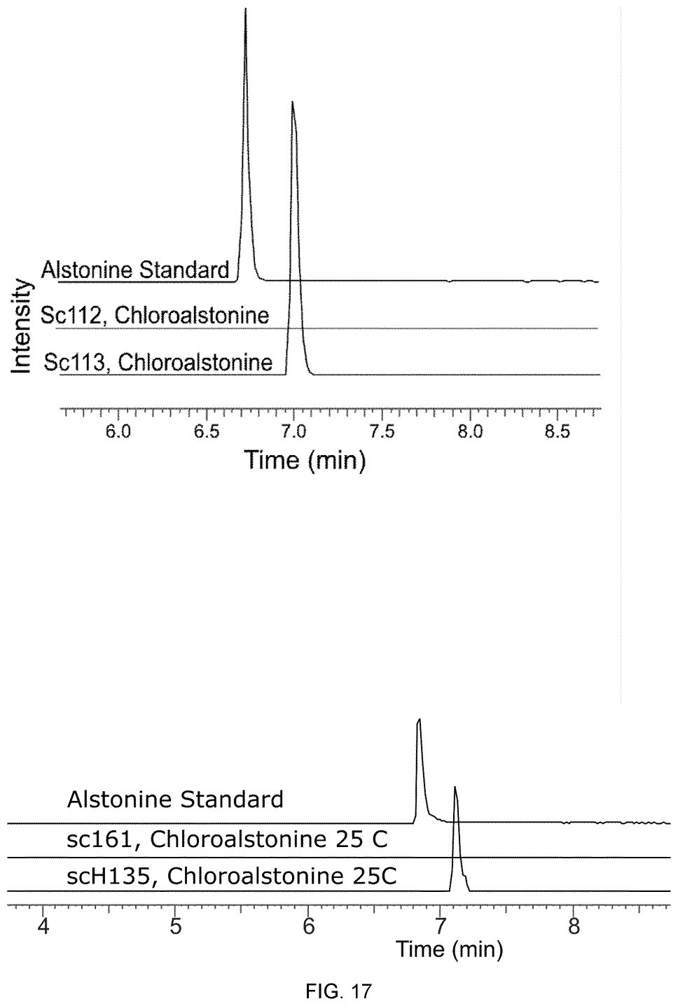

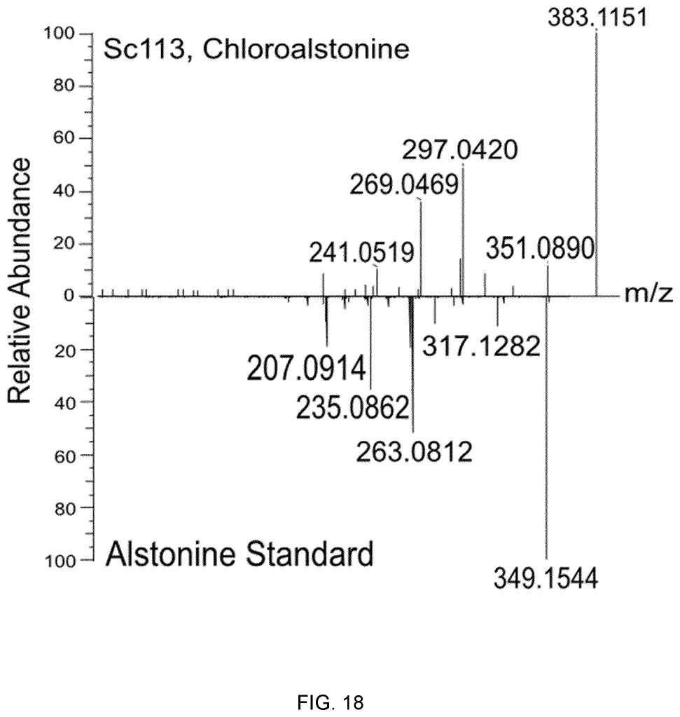

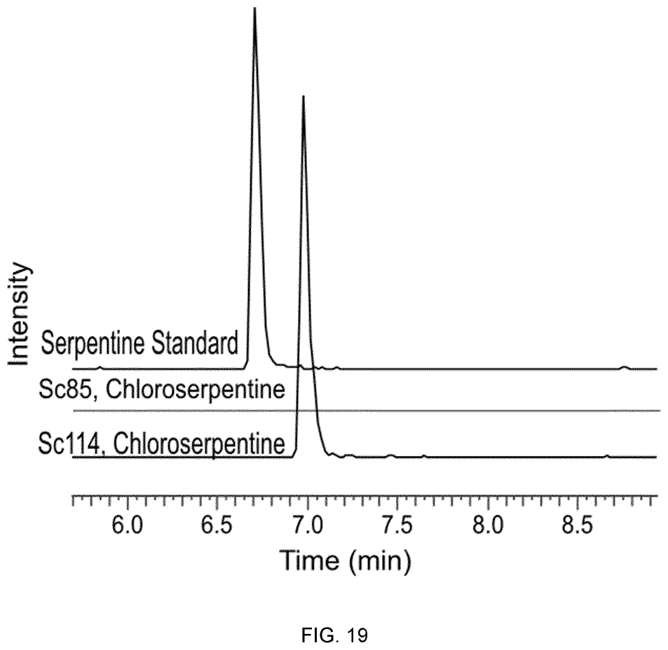

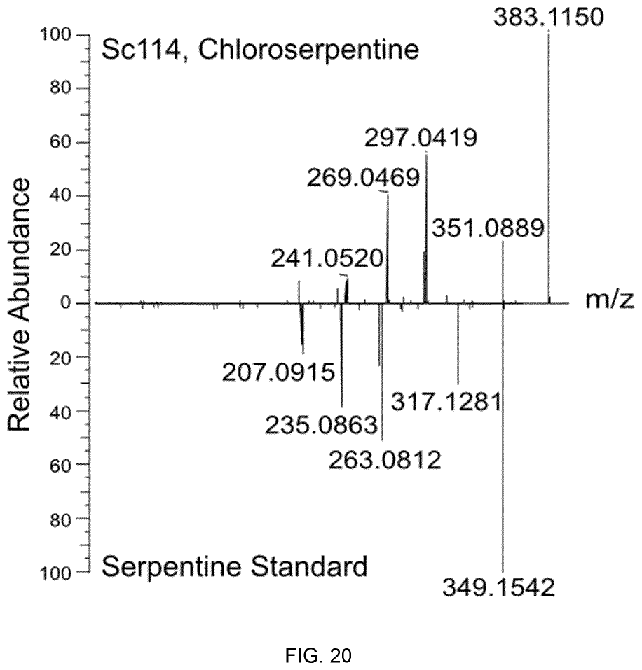

7-bromotryptamine was produced from KBr feeding by both Sc113 and Sc114, however 7-bromostrictosidine was not detected in any of the tested strains. 7-chloroalstonine was detected in broth of Sc113 fed with NaCl. Identification of 7-chloroalstonine is confirmed by exact mass, shift in retention time (FIG. 17), and mass shift in MS/MS spectra (FIG. 18, table 9). 7-chloroalstonine was similarly detected in the broth of the ScH135 cultivation (FIG. 17, FIG. 18, table 9). Only natural alstonine was detected in broth from the control strain Sc161 (FIG. 17, table 9). 7-chloroserpentine was identified in broth of SdA14 fed with NaCl. Identification of 7-chloroserpentine was supported by exact mass (FIG. 19, table 9) and MS/MS spectra (FIG. 20, table 9). The control strain Sc85 does not express a halogenase and produces serpentine, but no chloroserpentine (FIG. 19, table 9).

These results demonstrate that 7-chlorinated MIAs can be produced de novo in yeast as well as by feeding 7-chloroindole. Improving halogenase solubility by N-terminal fusion of IaeRebH with the Trx tag allowed for production of chloroalstonine without secologanin supplementation.

TABLE 9
Production of halogenated, such as chlorinated, MIA analogues in yeast quantified as
total peak area. De novo biosynthesis at of MIAs chlorinated at position 7 was demonstrated
for chlorotryptophan, chlorotryptamine, chloroalstonine and chloroserpentine.

| | | Sc85 | Sc114 | Sc112 | Sc113 | Sc161 | ScH135 |
|---|---|---|---|---|---|---|---|
| | RT | Peak | Peak | Peak | Peak | Peak | Peak |
| Compound | (min) | Area | Area | Area | Area | Area | Area |
| Tryptophan | 4.80 | 168797167.22 | 290627300.43 | 129348926.65 | 320923433.59 | 1348309392.49 | 1155069575.93 |
| Tryptamine | 5.20 | 95234600.49 | 69429256.85 | 98805215.13 | 25419578.04 | 54580051.46 | 101015207.09 |
| Alstonine | 6.84 | NA | NA | 81693551.60 | 89975995.31 | 774459695.03 | 818123864.67 |
| Serpentine | 6.98 | 559359849.47 | 1195985794.25 | NA | NA | NA | NA |
| 7- | 5.73 | NF | 19927380.86 | NF | 21859785.67 | NF | 4793857.90 |
| chlorotryptophan | | | | | | | |
| 7- | 6.00 | NF | 153074579.19 | NF | 74773620.87 | NF | 18538656.21 |
| chlorotryptamine | | | | | | | |
| 7- | 7.12 | NA | NA | NF | 785056.05 | NF | 1240233.67 |
| chloroalstonine | | | | | | | |
| 7- | 6.92 | NF | 4359385.56 | NA | NA | NA | NA |
| chloroserpentine | | | | | | | |
| 7- | 5.86 | NF | 1534031.34 | NF | 4287268.65 | NA | NA |
| bromotryptophan | | | | | | | |

NF = not found, NA = not analyzed.

### Example 11: Spatial Engineering of SGD and GS

To increase tabersonine and catharanthine titers, spatial enzyme engineering was applied to localize enzymes RseSGD and CroGS in various yeast compartments.

Strains were constructed with either localized SGD, localized GS or co-localized SGD and GS. Previously tested protein targeting sequences were genetically fused to RseSGD and CroGS to modify their subcellular localization: Peptide tag ATP9sp localizes to mitochondrial lumen, peptide tag ePTS1sp localizes to peroxisome, peptide tag CPYsp localizes to vacuole, peptide tag SV40sp localizes to nucleus, the peptide tag CNE1sp localizes to endoplasmic reticulum (ER) lumen and the peptide tag CYB5sp localizes to the ER membrane facing cytoplasm.

To test the effect of both single enzyme localization and co-localization, the tagged enzyme variants were introduced in one of three background strains, capable of producing tabersonine and catharanthine from secologanin and tryptamine feeding when supplemented with a functional GS, SGD or GS and SGD respectively. Strain MIA-DC contains the full pathway from secologanin and tryptamine to tabersonine and catharanthine. All strains expressed the variant of CroTS as set forth in SEQ ID NO: 39.

$$MIA - DC : CroCPR + CroCYB 5 + CroSTR + CroGS + CroGO + CroPAS + CroDPAS + RseSGD + CroRdx 1 + CroRdx 2 + CroTS + CroSAT + CroCS$$

$$y\_dmb \_ 38 ( no SGD ) : CroCPR + CroCYB 5 + CroSTR + CroGS + CroGO + CroPAS + CroDPAS + CroRdx 1 + CroRdx 2 + CroTS + CroSAT + CroCS$$

$$y\_dmb \_ 39 ( no GS ) : CroCPR + CroCYB 5 + CroSTR + CroGO + CroPAS + CroDPAS + RseSGD + CroRdx 1 + CroRdx 2 + CroTS + CroSAT + CroCS$$

$$y\_dmb \_ 132 ( no SGD + GS ) : CroCPR + CroCYB 5 + CroSTR + CroGO + CroPAS + CroDPAS + CroRdx 1 + CroRdx 2 + CroTS + CroSAT + CroCS$$

### Strains for Localization of SGD:

$$Y\_dmb \_ 139 : y\_dmb \_ 38 + RseSGD$$

$$y\_dmb \_ 140 : y\_dmb \_ 38 + ATP 9 sp - RseSGD$$

$$y\_dmb \_ 141 : y\_dmb \_ 38 + RseSGD - ePTS 1 sp$$

$$y\_dmb \_ 142 : y\_dmb \_ 38 + RseSGD - CYB 5 sp$$

$$y\_dmb \_ 143 : y\_dmb \_ 38 + RseSGD - CNE 1 sp$$

$$y\_dmb \_ 144 : y\_dmb \_ 38 + RseSGD - CPYsp$$

$$Y\_dmb \_ 44 : y\_dmb \_ 38 + SV 40 sp - RseSGD$$

### Strains for Localization of GS:

$$y\_dmb \_ 187 : y\_dmb \_ 39 + CroGS$$

$$y\_dmb \_ 188 : y\_dmb \_ 39 + ATP 9 sp - CroGS$$

$$y\_dmb \_ 189 : y\_dmb \_ 39 + CroGS - ePTS 1 sp$$

$$y\_dmb \_ 190 : y\_dmb \_ 39 + CroGS - CYB 5 sp$$

$$y\_dmb \_ 191 : y\_dmb \_ 39 + CroGS - CNE 1 sp$$

$$y\_dmb \_ 192 : y\_dmb \_ 39 + CroGS - CPYsp$$

$$Y\_NJM \_ 45 : y\_dmb \_ 39 + SV 40 sp - CroGS$$

### Strains for Co-Localization of SGD+GS:

$$y\_NJM \_ 39 : y\_dmb \_ 132 + RseSGD + CroGS$$

$$y\_NJM \_ 40 : y\_dmb \_ 132 + ATP 9 sp - RseSGD + ATP 9 sp - CroGS$$

$$y\_NJM \_ 41 : y\_dmb \_ 132 + RseSGD - ePTS 1 sp + CroGS - ePTS 1 sp$$

$$y\_NJM \_ 42 : y\_dmb \_ 132 + RseSGD - CYB 5 sp + CroGS - CYB 5 sp$$

$$y\_NJM \_ 43 : y\_dmb \_ 132 + RseSGD - CNE 1 sp + CroGS - CNE 1 sp$$

$$y\_NJM \_ 47 : y\_dmb \_ 132 + SV 40 sp - RseSGD + SV 40 sp - CroGS$$

$$y\_NJM \_ 48 : y\_dmb \_ 132 + RseSGD - CPYsp + CroGS - CPYsp$$

### Verification of Tabersonine Production by Localized or Co-Localized SGD and GS:

The resulting strains were cultivated at 30° C., 300 rpm for five days in 3×SC+2% glucose+0.25 mM secologanin+1 mM tryptamine. Cells were precipitated by centrifugation and 100 μL spent medium was harvested. Tabersonine production was quantified by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA). Tabersonine titers obtained in the cultivation are shown in table 10.

RseSGD was functional when localized to either mitochondria, peroxisome, ER lumen, or on ER membrane. CroGS was functional when localized to mitochondrial lumen, peroxisome, vacuole, nucleus, ER lumen, or on ER membrane. Localization of RseSGD or CroGS alone to one of the compartments did not improve the tabersonine titer. Colocalization of RseSGD and CroGS was functional in mitochondrial lumen, peroxisome, ER lumen, and on ER membrane, with ER lumen and on ER membrane producing the highest tabersonine titer of 23.42±2.11 μg/L and 17.08±0.82 μg/L, respectively. These titers represent a significant improvement compared with the WT RseSGD+CroGS control strain y_NJM_39. This demonstrates that spatial enzyme engineering can improve MIA production in yeast.

TABLE 10
Tabersonine titers obtained for strains with localized or co-localized
GS and SGD. The subcellular localization of GS or SGD is indicated
under strain description. Co-localization of RseSGD and CroGS in
ER lumen or on ER membrane resulted in the highest tabersonine titers
of 23.42 ± 2.11 μg/L and 17.08 ± 0.82 μg/L, respectively.

| | Mean | Standard | |
|---|---|---|---|
| | Tabersonine | deviation | |
| Strain | (μg/L) | (μg/L) | Strain description |
| MIA-DC | 5.04 | 0.50 | Control |
| Y_dmb_132 | 0 | 0 | Control |
| Y_dmb_38 | 0.86 | 0.07 | Control |
| Y_dmb_39 | 0 | 0 | Control |
| Y_dmb_139 | 9.23 | 0.66 | WT SGD |
| y_dmb_140 | 5.15 | 1.35 | SGD in Mitochondrial lumen |
| y_dmb_141 | 5.91 | 0.28 | SGD in Peroxisome |
| y_dmb_142 | 8.33 | 0.84 | SGD on ER membrane |
| y_dmb_143 | 6.02 | 0.90 | SGD in ER lumen |
| y_dmb_144 | 0.83 | 0.10 | SGD in Vacuole |
| Y_NJM_44 | 0 | 0 | SGD in Nucleus |
| y_dmb_187 | 4.59 | 0.58 | WT GS |
| y_dmb_188 | 0.84 | 0.14 | GS in Mitochondrial lumen |
| y_dmb_189 | 1.64 | 0.03 | GS in Peroxisome |
| y_dmb_190 | 2.53 | 0.36 | GS on ER membrane |
| y_dmb_191 | 1.05 | 0.11 | GS in ER lumen |
| y_dmb_192 | 0.82 | 0.13 | GS in Vacuole |
| Y_NJM_45 | 0.78 | 0.12 | GS in Nucleus |
| y_NJM_39 | 7.08 | 0.03 | WT SGD + GS |
| y_NJM_40 | 7.87 | 0.40 | SGD + GS in Mitochondrial |
| | | | lumen |
| y_NJM_41 | 0.88 | 0.06 | SGD + GS in Peroxisome |
| y_NJM_42 | 17.08 | 0.82 | SGD + GS on ER membrane |
| y_NJM_43 | 23.42 | 2.11 | SGD + GS in ER lumen |
| y_NJM_47 | 0 | 0 | SGD + GS in Vacuole |
| y_NJM_48 | 0 | 0 | SGD + GS in Nucleus |

### Example 12: Overexpression of NAD Kinase POS5

Several reactions in the MIA pathway require NADPH as co-factor. One example is the reduction of strictosidine aglycone to geissoschizine catalyzed by GS which requires one molecule of NADPH. In yeast NADPH can be synthesized by reduction of NADP+ or by phosphorylation of NADH. To improve NADPH availability for MIA synthesis we overexpressed NAD kinase (POS5) from Saccharomyces cerevisiae. POS5 catalyzes the phosphorylation of NADH to NADPH, and phosphorylation of NAD+ to NADP+ with lower specificity. POS5 localizes naturally to the mitochondrial lumen. Over-expression was achieved by integrating an additional copy of POS5 under a strong promoter into the genome of yeast stains y_NJM_39 and y_NJM_40 to create yeast strains y_NJM_64 and y_NJM_66, respectively. Yeast strain y_NJM_40 expresses ATP9sp-RseSGD and ATP9sp-CroGS which are co-localized in mitochondrial lumen. Yeast strain y_NJM_39 expresses WT RseSGD and CroGS. Thus, in yeast strain y_NJM_66, POS5, ATP9sp-RseSGD, and ATP9sp-CroGS are all co-localized in the mitochondrial lumen.

All strains were designed to produce tabersonine and catharanthine when fed with tryptamine and secologanin. All strains expressed the variant of CroTS as set forth in SEQ ID NO: 39.

Strain with Overexpression of POS5

$$Y\_NJM \_ 64 : y\_NJM \_ 39 + POS 5$$

Strain with Co-Localization of SGD+GS and Overexpression of POS5

$$Y\_NJM \_ 66 : y\_NJM \_ 40 + POS 5$$

Verification of Tabersonine Production by Strains with Overexpressed POS5:

Yeast strains y_NJM_39, y_NJM_40, y_NJM_64, and y_NJM_66 were cultivated at 30° C., 300 rpm for five days in 3×SC+2% glucose+0.25 mM secologanin+1 mM tryptamine. Cells were precipitated by centrifugation and 100 μL spend medium was harvested. Tabersonine production was quantified by targeted LC-MS on an advanced UHPLC system (Bruker Daltonics, Fremont, CA, USA) coupled to an EVOQ Elite triple quadrupole mass spectrometer (Bruker Daltonics, Fremont, CA, USA).

### Results:

Tabersonine titers obtained in the cultivation are shown in table 11. Strain y_NJM_64 overexpressing POS5 and expressing WT RseSGD and CroGS produced 11.88±11.47 μg/L tabersonine. Strain y_NJM_66 overexpressing POS5 and mitochondrial overexpressing RseSGD and CroGS in the mitochondrial lumenproduced 16.77±2.2 μg/L tabersonine. The results present a significant improvement in tabersonine production by overexpression of POS5.

This result demonstrates that improving synthesis of the cofactor NADPH can improve MIA production in yeast.

TABLE 11
Tabersonine titers obtained for strains with overexpressed
POS5 in strains with co-localized GS and SGD. The overexpression
of POS5 and intracellular localization of GS or SGD is
indicated under strain description. WT: wild type.

| | Mean | Standard | |
|---|---|---|---|
| | Tabersonine | deviation | |
| Strain | (μg/L) | (μg/L) | Strain description |
| y_NJM_39 | 7.08 | 0.03 | Control: WT SGD + GS |
| y_NJM_40 | 7.87 | 0.40 | Control: SGD + GS in |
| | | | Mitochondrial lumen |
| y_NJM_64 | 11.88 | 11.47 | WT SGD + WT GS + POS5 |
| y_NJM_66 | 16.77 | 2.21 | SGD in Mitochondrial lumen + |
| | | | GS in Mitochondrial lumen + |
| | | | POS5 |

### Sequence Overview

All DNA sequences are codon-optimised or modified variants of the native DNA sequence, except SEQ ID NO: 48, 125, 126, 127, 130, 131, 132, 133, 135 and 150, which are the native DNA sequences.

| SEQ | | | | Accession | Nature |
|---|---|---|---|---|---|
| ID | | | | Number or | of |
| NO: | Short Name | Organism | Name | reference | sequence |
| 1 | AgrGPPS2 | Abies grandis | geranyl diphosphate | AAN01134.1 | DNA |
| | | | synthase | | |
| 2 | GgaFPS* | Gallus gallus | farnesyl pyrophosphate | P08836.2 | DNA |
| | (N144W) | | synthase variant N144W | | |
| 3 | trunCroGES | Catharanthus | geraniol synthase | | DNA |
| | | roseus | | | |
| 4 | ERG20**-GS- | synthetic | | | DNA |
| | trunCroGES | construct | | | |
| 5 | CroG8H | Catharanthus | geraniol 8-hydroxylase | CAC80883.1 | DNA |
| | | roseus | | | |
| 6 | CroCPR | Catharanthus | NADPH-cytochrome | CAA49446.1 | DNA |
| | | roseus | P450 reductase | | |
| 7 | CroCYB5 | Catharanthus | cytochrome b5 | A0A0C5DKP2 | DNA |
| | | roseus | | | |
| 8 | Vmi8HGOA | Vinca minor | 8-hydroxygeraniol | Stander et al., 2020 | DNA |
| | | | oxidoreductase, | | |
| | | | type A (NAD+-dependent) | | |
| 9 | NcaISY | Nepeta cataria | iridoid synthase | KY882234.1 | DNA |
| 10 | NcaMLPLA | Nepeta cataria | iridoid cyclase | QKE59462.1 | DNA |
| 11 | CroIO | Catharanthus | iridoid oxidase | AHK60833.1 | DNA |
| | | roseus | | | |
| 12 | CroCYPADH | Catharanthus | CYP enzymes assisting | KP411012.1 | DNA |
| | | roseus | alcohol dehydrogenase | | |
| 13 | Cro7DLGT | Catharanthus | 7-deoxyloganetic acid | AHK60835.1 | DNA |
| | | roseus | glucosyl transferase | | |
| 14 | Cro7DLH | Catharanthus | 7-deoxyloganic acid | AHK60834.1 | DNA |
| | | roseus | hydroxylase | | |
| 15 | CroLAMT | Catharanthus | loganic acid O-methyl- | ABW38009.1 | DNA |
| | | roseus | transferase | | |
| 16 | CroSLS | Catharanthus | secologanin synthase | AKF02529.1 | DNA |
| | | roseus | | | |
| 17 | CroTDC | Catharanthus | tryptophan | CAA47898.1 | DNA |
| | | roseus | decarboxylase | | |
| 18 | rgnTDC | Ruminococcus | tryptophan | EDN78222.1 | DNA |
| | | gnavus | decarboxylase | | |
| 19 | CroSTR | Catharanthus | strictosidine synthase | CAA43936.1 | DNA |
| | | roseus | | | |
| 20 | RseSGD | Rauvolfia | strictosidine- | AAF03675.1_1 | DNA |
| | | serpentina | O-β-D-glucosidase | | |
| 21 | CroTHAS1 | Catharanthus | tetrahydroalstonine | KM524258 | DNA |
| | | roseus | synthase | | |
| 22 | CroHYS | Catharanthus | heteroyohimbine | KU865325.1 | DNA |
| | | roseus | synthase | | |
| 23 | CroGS | Catharanthus | geissoschizine synthase | AHK60846.1 | DNA |
| | | roseus | | | |
| 24 | CroADH13 | Catharanthus | geissoschizine synthase | KF302078.1 | DNA |
| | | roseus | | | |
| 25 | SV40sp | Simian virus 40 | simian virus | P03070 | protein |
| | | | 40 signal peptide | | |
| 26 | CroGO | Catharanthus | geissoschizine oxidase | AHK60839.1 | DNA |
| | | roseus | | | |
| 27 | TelGO | Tabernaemontana | geissoschizine oxidase | AIR93636.1 | DNA |
| | | elegans | | | |
| 28 | VmiGO | Vinca minor | CYP71AY2 | AIR93637.1 | DNA |
| | | | (geissoschizine | | |
| | | | oxidase)-like protein | | |
| 29 | AhuGO | Amsonia | geissoschizine oxidase | AIR93639.1 | DNA |
| | | hubrichtii | | | |
| 30 | CroRdx1 | Catharanthus | protein Redox 1 | AVM85917.1 | DNA |
| | | roseus | | | |
| 31 | CroRdx2 | Catharanthus | protein Redox 2 | AVM85918.1 | DNA |
| | | roseus | | | |
| 32 | CroSAT | Catharanthus | stemmadenine-O- | AVM85919.1 | DNA |
| | | roseus | acetyltransferase | | |
| 33 | CroPAS | Catharanthus | O-acetyl-stemmadenine | AYE56095.1 | DNA |
| | | roseus | oxidase/ | | |
| | | | precondylocarpine | | |
| | | | acetate synthase | | |
| 34 | AhuASO | Amsonia | O-acetyl-stemmadenine | AYE56103.1 | DNA |
| | | hubrichtii | oxidase/ | | |
| | | | precondylocarpine | | |
| | | | acetate synthase | | |
| 35 | TibPAS2 | Tabernanthe | O-acetyl-stemmadenine | QED20590.1 | DNA |
| | | iboga | oxidase/ | | |
| | | | precondylocarpine | | |
| | | | acetate synthase | | |
| 36 | TelASO | Tabernaemontana | O-acetyl-stemmadenine | AYE56102.1 | DNA |
| | | elegans | oxidase/ | | |
| | | | precondylocarpine | | |
| | | | acetate synthase | | |
| 37 | CroDPAS | Catharanthus | dihydroprecondylocarpine | ANQ45231.1 | DNA |
| | | roseus | acetate synthase | | |
| 38 | TibDPAS2 | Tabernanthe | dihydroprecondylocarpine | QED20595.1 | DNA |
| | | iboga | acetate synthase | | |
| 39 | CroTS | Catharanthus | tabersonine synthase | AVM85921.1 | DNA |
| | | roseus | | | |
| 40 | CroCS | Catharanthus | catharanthine synthase | AVM85920.1 | DNA |
| | | roseus | | | |
| 41 | CroT16H2 | Catharanthus | tabersonine | AEB69788.1 | DNA |
| | | roseus | 16-hydroxylase 2 | | |
| 42 | Cro16OMT | Catharanthus | tabersonine | ABR20103.1 | DNA |
| | | roseus | 16-O-methyltransferase | | |
| 43 | CroT3O | Catharanthus | tabersonine | AEX07771.1 | DNA |
| | | roseus | 3-oxygenase | | |
| 44 | CroT3R | Catharanthus | 16-methoxy-2,3-dihydro- | AKV60044.1 | DNA |
| | | roseus | 3-hydroxytabersonine | | |
| | | | synthase | | |
| 45 | CroNMT | Catharanthus | 3-hydroxy-16-methoxy- | AHH33092.1 | DNA |
| | | roseus | 2,3-dihydrotabersonine | | |
| | | | N-methyl-transferase | | |
| 46 | CroD4H | Catharanthus | deacetoxyvindoline | AAB97311.1 | DNA |
| | | roseus | 4-hydroxylase | | |
| 47 | CroDAT | Catharanthus | deacetylvindoline | AAC99311.1 | DNA |
| | | roseus | O-acetyltransferase | | |
| 48 | CroSS | Catharanthus | Serpentine synthase | MT829151 | DNA |
| | | roseus | | | |
| 49 | laeRebH_N470S | synthetic | Flavin-dependent | | DNA |
| | | construct | tryptophan halogenase | | |
| 50 | EcoSsuE | Escherichia | FMN reductase (NADPH) | NP_415457.1 | DNA |
| | | coli str. K-12 | | | |
| | | substr. | | | |
| | | MG1655 | | | |
| 51 | ATP9sp- | synthetic | mitochondrial | | DNA |
| | RseSGD | construct | strictosidine-O- | | |
| | | | β-D-glucosidase | | |
| 52 | ATP9sp- | synthetic | mitochondrial | | DNA |
| | CroGS | construct | geissoschizine synthase | | |
| 52 | ATP9sp- | synthetic | mitochondrial | | DNA |
| | CroGS | construct | geissoschizine synthase | | |
| 54 | CroGS- | synthetic | ER luminal | | DNA |
| | CNE1sp | construct | geissoschizine synthase | | |
| 55 | RseSGD- | synthetic | ER membrane bound | | DNA |
| | CYB5sp | construct | strictosidine- | | |
| | | | O-β-D-glucosidase | | |
| 56 | CroGS- | synthetic | ER membrane bound | | DNA |
| | CYB5sp | construct | geissoschizine synthase | | |
| 57 | RseSGD- | synthetic | peroxisomal | | DNA |
| | ePTS1sp | construct | strictosidine- | | |
| | | | O-β-D-glucosidase | | |
| 58 | CroGS- | synthetic | peroxisomal | | DNA |
| | ePTS1sp | construct | geissoschizine synthase | | |
| 59 | RseSGD- | synthetic | Vacuolar strictosidine- | | DNA |
| | CPYsp | construct | O-β-D-glucosidase | | |
| 60 | CroGS- | synthetic | Vacuolar geissoschizine | | DNA |
| | CPYsp | construct | synthase | | |
| 61 | SV40sp- | synthetic | Nuclear strictosidine- | | DNA |
| | RseSGD | construct | O-β-D-glucosidase | | |
| 62 | SV40sp- | synthetic | Nuclear geissoschizine | | DNA |
| | CroGS | construct | synthase | | |
| 63 | AgrGPPS2 | Abies grandis | geranyl diphosphate | AAN01134.1 | protein |
| | | | synthase | | |
| 64 | GgaFPS* | Gallus gallus | farnesyl pyrophosphate | P08836.2 | protein |
| | (N144W) | | synthase variant N144W | | |
| 65 | trunCroGES | Catharanthus | geraniol synthase | | protein |
| | | roseus | | | |
| 66 | ERG20**-GS- | synthetic | | | protein |
| | trunCroGES | construct | | | |
| 67 | CroG8H | Catharanthus | geraniol 8-hydroxylase | CAC80883.1 | protein |
| | | roseus | | | |
| 68 | CroCPR | Catharanthus | NADPH-cytochrome | CAA49446.1 | protein |
| | | roseus | P450 reductase | | |
| 69 | CroCYB5 | Catharanthus | cytochrome b5 | A0A0C5DKP2 | protein |
| | | roseus | | | |
| 70 | Vmi8HGOA | Vinca minor | 8-hydroxygeraniol | Stander et al., 2020 | protein |
| | | | oxidoreductase, type A | | |
| | | | (NAD+-dependent) | | |
| 71 | NcaISY | Nepeta cataria | iridoid synthase | KY882234.1 | protein |
| 72 | NcaMLPLA | Nepeta cataria | iridoid cyclase | QKE59462.1 | protein |
| 73 | CroIO | Catharanthus | iridoid oxidase | AHK60833.1 | protein |
| | | roseus | | | |
| 74 | CroCYPADH | Catharanthus | CYP enzymes assisting | KP411012.1 | protein |
| | | roseus | alcohol dehydrogenase | | |
| 75 | Cro7DLGT | Catharanthus | 7-deoxyloganetic acid | AHK60835.1 | protein |
| | | roseus | glucosyltransferase | | |
| 76 | Cro7DLH | Catharanthus | 7-deoxyloganic acid | AHK60834.1 | protein |
| | | roseus | hydroxylase | | |
| 77 | CroLAMT | Catharanthus | loganic acid | ABW38009.1 | protein |
| | | roseus | O-methyltransferase | | |
| 78 | CroSLS | Catharanthus | secologanin synthase | AKF02529.1 | protein |
| | | roseus | | | |
| 79 | CroTDC | Catharanthus | tryptophan | CAA47898.1 | protein |
| | | roseus | decarboxylase | | |
| 80 | rgnTDC | Ruminococcus | tryptophan | EDN78222.1 | protein |
| | | gnavus | decarboxylase | | |
| 81 | CroSTR | Catharanthus | strictosidine synthase | CAA43936.1 | protein |
| | | roseus | | | |
| 82 | RseSGD | Rauvolfia | strictosidine- | AAF03675.1_1 | protein |
| | | serpentina | O-β-D-glucosidase | | |
| 83 | CroTHAS1 | Catharanthus | tetrahydroalstonine | KM524258 | protein |
| | | roseus | synthase | | |
| 84 | CroHYS | Catharanthus | heteroyohimbine | KU865325.1 | protein |
| | | roseus | synthase | | |
| 85 | CroGS | Catharanthus | geissoschizine synthase | AHK60846.1 | protein |
| | | roseus | | | |
| 86 | CroADH13 | Catharanthus | geissoschizine synthase | KF302078.1 | protein |
| | | roseus | | | |
| 87 | POS5 | Saccharomyces | NADH kinase POS5, | CAA59017.1 | protein |
| | | cerevisiae | mitochondrial | | |
| 88 | CroGO | Catharanthus | geissoschizine oxidase | AHK60839.1 | protein |
| | | roseus | | | |
| 89 | TelGO | Tabernaemontana | geissoschizine oxidase | AIR93636.1 | protein |
| | | elegans | | | |
| 90 | VmiGO | Vinca minor | CYP71AY2 | AIR93637.1 | protein |
| | | | (geissoschizine | | |
| | | | oxidase)-like protein | | |
| 91 | AhuGO | Amsonia | geissoschizine oxidase | AIR93639.1 | protein |
| | | hubrichtii | | | |
| 92 | CroRdx1 | Catharanthus | protein Redox 1 | AVM85917.1 | protein |
| | | roseus | | | |
| 93 | CroRdx2 | Catharanthus | protein Redox 2 | AVM85918.1 | protein |
| | | roseus | | | |
| 94 | CroSAT | Catharanthus | stemmadenine-O- | AVM85919.1 | protein |
| | | roseus | acetyltransferase | | |
| 95 | CroPAS | Catharanthus | O-acetyl- | AYE56095.1 | protein |
| | | roseus | stemmadenine oxidase/ | | |
| | | | precondylocarpine | | |
| | | | acetate synthase | | |
| 96 | AhuASO | Amsonia | O-acetyl- | AYE56103.1 | protein |
| | | hubrichtii | stemmadenine oxidase/ | | |
| | | | precondylocarpine | | |
| | | | acetate synthase | | |
| 97 | TibPAS2 | Tabernanthe | O-acetyl- | QED20590.1 | protein |
| | | iboga | stemmadenine oxidase/ | | |
| | | | precondylocarpine | | |
| | | | acetate synthase | | |
| 98 | TelASO | Tabernaemontana | O-acetyl- | AYE56102.1 | protein |
| | | elegans | stemmadenine oxidase/ | | |
| | | | precondylocarpine | | |
| | | | acetate synthase | | |
| 99 | CroDPAS | Catharanthus | dihydroprecondylocarpine | ANQ45231.1 | protein |
| | | roseus | acetate synthase | | |
| 100 | TibDPAS2 | Tabernanthe | dihydroprecondylocarpine | QED20595.1 | protein |
| | | iboga | acetate synthase | | |
| 101 | CroTS | Catharanthus | tabersonine synthase | AVM85921.1 | protein |
| | | roseus | | | |
| 102 | CroCS | Catharanthus | catharanthine synthase | AVM85920.1 | protein |
| | | roseus | | | |
| 103 | CroT16H2 | Catharanthus | tabersonine 16- | AEB69788.1 | protein |
| | | roseus | hydroxylase 2 | | |
| 104 | Cro16OMT | Catharanthus | tabersonine 16-O- | ABR20103.1 | protein |
| | | roseus | methyltransferase | | |
| 105 | CroT3O | Catharanthus | tabersonine | AEX07771.1 | protein |
| | | roseus | 3-oxygenase | | |
| 106 | CroT3R | Catharanthus | 16-methoxy-2,3-dihydro- | AKV60044.1 | protein |
| | | roseus | 3-hydroxytabersonine | | |
| | | | synthase | | |
| 107 | CroNMT | Catharanthus | 3-hydroxy-16-methoxy- | AHH33092.1 | protein |
| | | roseus | 2,3-dihydrotabersonine | | |
| | | | N-methyltransferase | | |
| 108 | CroD4H | Catharanthus | deacetoxyvindoline | AAB97311.1 | protein |
| | | roseus | 4-hydroxylase | | |
| 109 | CroDAT | Catharanthus | deacetylvindoline | AAC99311.1 | protein |
| | | roseus | O-acetyltransferase | | |
| 110 | CroSS | Catharanthus | Serpentine synthase | MT829151 | protein |
| | | roseus | | | |
| 111 | laeRebH_N470S | synthetic | Flavin-dependent | | protein |
| | | construct | tryptophan halogenase | | |
| 112 | EcoSsuE | Escherichia | FMN reductase (NADPH) | NP_415457.1 | protein |
| | | coli str. K-12 | | | |
| | | substr. | | | |
| | | MG1655 | | | |
| 113 | ATP9sp- | synthetic | mitochondrial | | protein |
| | RseSGD | construct | strictosidine-O- | | |
| | | | β-D-glucosidase | | |
| 114 | ATP9sp- | synthetic | mitochondrial | | protein |
| | CroGS | construct | geissoschizine synthase | | |
| 115 | RseSGD- | synthetic | ER luminal | | protein |
| | CNE1sp | construct | strictosidine- | | |
| | | | O-β-D-glucosidase | | |
| 116 | CroGS- | synthetic | ER luminal | | protein |
| | CNE1sp | construct | geissoschizine synthase | | |
| 117 | RseSGD- | synthetic | ER membrane bound | | protein |
| | CYB5sp | construct | strictosidine- | | |
| | | | O-β-D-glucosidase | | |
| 118 | CroGS- | synthetic | ER membrane bound | | protein |
| | CYB5sp | construct | geissoschizine synthase | | |
| 119 | RseSGD- | synthetic | peroxisomal | | protein |
| | ePTS1sp | construct | strictosidine- | | |
| | | | O-β-D-glucosidase | | |
| 120 | CroGS- | synthetic | peroxisomal | | protein |
| | ePTS1sp | construct | geissoschizine synthase | | |
| 121 | RseSGD- | synthetic | Vacuolar strictosidine- | | protein |
| | CPYsp | construct | O-β-D-glucosidase | | |
| 122 | CroGS- | synthetic | Vacuolar | | protein |
| | CPYsp | construct | geissoschizine synthase | | |
| 123 | SV40sp- | synthetic | Nuclear strictosidine- | | protein |
| | RseSGD | construct | O-β-D-glucosidase | | |
| 124 | SV40sp- | synthetic | Nuclear | | protein |
| | CroGS | construct | geissoschizine synthase | | |
| 125 | IDI1 | Saccharomyces | Isopentenyl | J05090 | DNA |
| | | cerevisiae | diphosphate:dimethylallyl | | |
| | | | diphosphate isomerase | | |
| 126 | trunHMG1 | Saccharomyces | HMG-COA reductase | AAA34676.1 | DNA |
| | | cerevisiae | | | |
| 127 | TRP5 | Saccharomyces | tryptophan synthase | NM_001180891.1 | DNA |
| | | cerevisiae | | | |
| 128 | ERG20** | Saccharomyces | farnesyl pyrophosphate | Ignea et al. 2014 | DNA |
| | | cerevisiae | synthase variant | | |
| | | | F96W-N127W | | |
| 129 | ATP9sp | Neurospora | ATP9 signal peptide | CAA24230 | DNA |
| | | crassa | | | |
| 130 | CNE1sp | Saccharomyces | Calnexin homolog | P27825 | DNA |
| | | cerevisiae | signal peptide | | |
| 131 | CYB5sp | Saccharomyces | Cytochrome b5 | P40312 | DNA |
| | | cerevisiae | signal peptide | | |
| 132 | ePTS1sp | Saccharomyces | enhanced peroxisomal | | DNA |
| | | cerevisiae | targeting signal | | |
| | | | type 1 (PTS1) | | |
| 133 | CPYsp | Saccharomyces | carboxypeptidase Y | P00729 | DNA |
| | | cerevisiae | signal peptide | | |
| 134 | SV40sp | Simian virus 40 | simian virus | P03070 | DNA |
| | | | 40 signal peptide | | |
| 135 | POS5 | Saccharomyces | NADH kinase POS5, | CAA59017.1 | DNA |
| | | cerevisiae | mitochondrial | | |
| 136 | IDI1 | Saccharomyces | Isopentenyl | J05090 | protein |
| | | cerevisiae | diphosphate:dimethylallyl | | |
| | | | diphosphate isomerase | | |
| 137 | trunHMG1 | Saccharomyces | HMG-COA reductase | AAA34676.1 | protein |
| | | cerevisiae | | | |
| 138 | TRP5 | Saccharomyces | tryptophan synthase | NM_001180891.1 | protein |
| | | cerevisiae | | | |
| 139 | ERG20** | Saccharomyces | farnesyl pyrophosphate | | protein |
| | | cerevisiae | synthase variant | | |
| | | | F96W-N127W | | |
| 140 | ATP9sp | Neurospora | ATP9 signal peptide | CAA24230 | protein |
| | | crassa | | | |
| 141 | CNE1sp | Saccharomyces | Calnexin homolog | P27825 | protein |
| | | cerevisiae | signal peptide | | |
| 142 | CYB5sp | Saccharomyces | Cytochrome b5 | P40312 | protein |
| | | cerevisiae | signal peptide | | |
| 143 | ePTS1sp | Saccharomyces | enhanced peroxisomal | | protein |
| | | cerevisiae | targeting signal | | |
| | | | type 1 (PTS1) | | |
| 144 | CPYsp | Saccharomyces | carboxypeptidase Y | P00729 | protein |
| | | cerevisiae | signal peptide | | |
| 145 | AhuCPR | Amsonia | NADPH-cytochrome | MAGPIE:ahu_{—} | protein |
| | | hubrichtii | P450 reductase | AHUWL1VD_Velvet-- | |
| | | | | Singlet39043 | |
| 146 | RseCPR | Rauvolfia | NADPH-cytochrome | MAGPIE:rse_{—} | protein |
| | | serpentina | P450 reductase | RSERTVD_Trinity-- | |
| | | | | comp50814_c0_seq1 | |
| 147 | CloCPR | Catharanthus | NADPH-cytochrome | MAGPIE:clo_{—} | protein |
| | | longifolius | P450 reductase | CLOLF1VD_Trinity-- | |
| | | | | comp713_c0_seq1 | |
| 148 | Linker | synthetic | | | protein |
| | | construct | | | |
| 149 | YEF1 | Saccharomyces | NAD/NADH kinase | YEL041W | protein |
| | | cerevisiae | | | |
| 150 | CroTS | Catharanthus | tabersonine synthase | AVM85921.1 | DNA |
| | | roseus | | | |
| 151 | Trx- | synthetic | Flavin-dependent | | Protein |
| | laeRebH | construct | tryptophan halogenase | | |
| | | | with solubility tag | | |
| 152 | Trx- | synthetic | Flavin-dependent | | DNA |
| | laeRebH | construct | tryptophan halogenase | | |
| | | | with solubility tag | | |
| 153 | laeRebH | Lechevalieria | Flavin-dependent | CAC93722.1 | Protein |
| | | aerocolonigenes | tryptophan halogenase | | |
| | | ATCC 39243 | | | |
| 154 | laeRebH | synthetic | Flavin-dependent | | DNA |
| | | construct | tryptophan halogenase | | |

### REFERENCES

hftp://www.chem.qmul.ac.uk/iubmb/misc/naseq.html; NC-IUB, Eur J Biochem (1985)
Chhonker et al. 2018. Simultaneous Quantitation of Isoprenoid Pyrophosphates in Plasma and Cancer Cells Using LC-MS/MS. Molecules. 2018, 23(12): 3275.
Miettinen et al 2014. The seco-iridoid pathway from Catharanthus roseus. Nat Comm. 7; 5: 3606.
Lichman et al. 2020. The evolutionary origins of the cat attractant nepetalactone in catnip. Sci. Adv. 13: eaba0721.
Davies et al. 2021. Engineering yeast for de novo synthesis of the insect repellent nepetalactone. ACS Synth Biol. 2021, 10, 2896-2903
Liu et al. 2022. Construction of ajmalicine and sanguinarine de novo biosynthetic pathways using stable integration sites in yeast. Biotechnol Bioeng. https://doi.org/10.1002/bit.28040
Dang et al. 2018. Sarpagan bridge enzyme has substrate-controlled cyclization and aromatization modes. Nat Chem Biol. 14, 760-763
Brown et al. 2015. De novo production of the plant-derived alkaloid strictosidine in yeast. PNAS. https://doi.org/10.1073/pnas.1423555112
Qu et al. 2015 Completion of the seven-step pathway from tabersonine to the anticancer drug precursor vindoline and its assembly in yeast.
PNAS. https://doi.org/10.1073/pnas.1501821112
Denby et al. 2018. Industrial brewing yeast engineered for the production of primary flavor determinants in hopped beer. Nat Comm. 9, Article number: 965 (2018)
Zhang & Petersen et al. 2020. Combining mechanistic and machine learning models for predictive engineering and optimization of tryptophan metabolism
Nat Comm. 11, Article number: 4880 (2020)
WO2022/106638. Methods for production of cis-trans-nepetalactol and iridoids. Filed on 19 Nov. 2021.
WO2020/229516. Methods for production of strictosidine aglycone and monoterpenoid indole alkaloids. Filed on 13 May 2020.
Stavrinides et al., 2015. Unlocking the Diversity of Alkaloids in Catharanthus roseus: Nuclear Localization Suggests Metabolic Channeling in Secondary Metabolism. Chemistry & Biology 22, 336-341, Mar. 19, 2015
Qu et al. 2018. Solution of the multistep pathway for assembly of corynanthean, strychnos, iboga, and aspidosperma monoterpenoid indole alkaloids from 19E-geissoschizine. PNAS. 2018 Mar. 20; 115(12):3180-3185. doi: 10.1073/pnas.1719979115.
Caputi et al. 2018 Missing enzymes in the biosynthesis of the anticancer drug vinblastine in Madagascar periwinkle. Science. 2018 Jun. 15; 360(6394):1235-1239. doi: 10.1126/science.aat4100.
Glenn et al. 2011. Reengineering a Tryptophan Halogenase To Preferentially Chlorinate a Direct Alkaloid Precursor. Journal of the American Chemical Society—2011, Volume 133, Issue 48, pp. 19346-19349
Jakočiūnas et al. 2015 CasEMBLR: Cas9-Facilitated Multiloci Genomic Integration of in Vivo Assembled DNA Parts in Saccharomyces cerevisiae. ACS Synthetic Biology 2015 4 (11), 1226-1234. DOI: 10.1021/acssynbio.5b00007
Thodey et al. 2014. A microbial biomanufacturing platform for natural and semisynthetic opioids. Nat. Chem. Biol. 10, 837-844.
Billingsley et al. 2017. Engineering the biocatalytic selectivity of iridoid production in Saccharomyces cerevisiae. Metab. Eng. 44, 117-125.
Li et al. (2018). Complete biosynthesis of noscapine and halogenated alkaloids in yeast. Proc Natl Acad Sci USA 115, E3922-E3931.
Misa et al. (2022). Engineered production of strictosidine and analogues in yeast. ACS Synth. Biol. (2022) doi:10.1021/acssynbio.2c00037.
Kulagina et al. (2021). Enhanced bioproduction of anticancer precursor vindoline by yeast cell factories. Microb. Biotechnol. 14, 2693-2699.
Runguphan et al. (2010). Integrating carbon-halogen bond formation into medicinal plant metabolism. Nature 468, 461-464.
Ignea et al. (2014). Engineering monoterpene production in yeast using a synthetic dominant negative geranyl diphosphate synthase. ACS Synth Biol. 2014 May 16; 3(5):298-306. doi: 10.1021/sb400115e.
Stander et al. (2020). Identifying Genes Involved in alkaloid Biosynthesis in Vinca minor Through Transcriptomics and Gene Co-Expression Analysis. Biomolecules. 2020 Nov. 24; 10(12):1595. doi: 10.3390/biom10121595.

### Items 1

1. A microorganism capable of producing strictosidine aglycone and/or halogenated strictosidine aglycone and/or derivatives thereof, in the presence of geranyl diphosphate and tryptamine, and/or geranyl diphosphate and halogenated tryptamine, respectively, said microorganism expressing a geraniol synthase (GES, EC 3.1.7.11) and a strictosidine-O-β-D-glucosidase (SGD, EC 3.2.1.105).
2. The microorganism according to item 1, further expressing;
   a geissoschizine synthase (GS, EC 1.3.1.36);
   a NADPH-cytochrome P450 reductase (CPR, EC 1.6.2.4);
   a cytochrome b5 (CYB5, EC 1.6.2.2);
   a geissoschizine oxidase (GO, EC 1.14.14.-);
   a protein Redox1 (EC 1.14.14.-);
   a protein Redox2 (EC 1.7.1.-); and/or
   a stemmadenine-O-acetyltransferase (SAT, EC 1.7.1.-),
   whereby the microorganism is further capable of producing stemmadenine acetate and/or halogenated stemmadenine acetate and/or derivatives thereof,
   wherein preferably said GS is CroGS (SEQ ID NO: 85) and/or CroADH13 (SEQ ID NO: 86), said CPR is CroCPR (SEQ ID NO: 68), said CYB5 is CroCYB5 (SEQ ID NO: 69), said GO is CroGO (SEQ ID NO: 88), TelGO (SEQ ID NO: 89), VmiGO (SEQ ID NO: 90), and/or AhuGO (SEQ ID NO: 91), said protein Redox1 is CroRdx1 (SEQ ID NO: 92), said protein Redox2 is CroRdx2 (SEQ ID NO: 93) and/or said SAT is CroSAT (SEQ ID NO: 94) or functional variants thereof having at least 70% homology, similarity or identity to SEQ ID NO: 85, SEQ ID NO: 86, SEQ ID NO: 68, SEQ ID NO: 69, SEQ ID NO: 88, SEQ ID NO: 89, SEQ ID NO: 90, SEQ ID NO: 91, SEQ ID NO: 92, SEQ ID NO: 93 and/or SEQ ID NO: 94, respectively.
3. The microorganism according to any one of the preceding items, wherein;
   i) the geraniol synthase (GES, EC 3.1.7.11) is a GES capable of converting geranyl diphosphate to geraniol, optionally said GES is a heterologous GES, preferably said GES is as set forth in SEQ ID NO: 65 and/or as set forth in SEQ ID NO: 66 or a functional variant thereof having at least 70% homology, similarity or identity to SEQ ID NO: 65 and/or SEQ ID NO: 66, respectively; and/or
   ii) the strictosidine-O-β-D-glucosidase (SGD, EC 3.2.1.105) is a SGD capable of converting strictosidine and/or halogenated strictosidine to strictosidine aglycone and/or halogenated strictosidine aglycone, respectively, optionally said SGD is a heterologous SGD, preferably said SGD is RseSGD as set forth in SEQ ID NO: 82 or a functional variant thereof having at least 70% homology, similarity or identity to SEQ ID NO: 82.
4. The microorganism according to any one of the preceding items, further expressing a geranyl diphosphate synthase (GPPS, EC 2.5.1.1) capable of converting isopentenyl diphosphate (IPP) and dimethylallyl diphosphate (DMAPP) to geranyl diphosphate, optionally said GPPS is a heterologous GPPS, preferably said GPPS is AgrGPPS2 as set forth in SEQ ID NO: 63, GgaFPS*(N144w) as set forth in SEQ ID NO: 64 or ERG20** as set forth in SEQ ID NO: 139 or a functional variant thereof having at least 70% homology, similarity or identity to SEQ ID NO: 63, SEQ ID NO: 64 or SEQ ID NO: 139, respectively.
5. The microorganism according to any one of the preceding items, further expressing;
   a geraniol-8-hydroxylase (G8H, EC 1.14.14.83);
   a NADPH-cytochrome P450 reductase (CPR, EC 1.6.2.4);
   a cytochrome b5 (CYB5, EC 1.6.2.2);
   a 8-hydroxygeraniol oxidoreductase (8HGO, EC 1.1.1.324);
   an iridoid synthase (ISY, EC 1.3.1.122);
   an iridoid cyclase (CYC, EC 5.5.1.34);
   an iridoid oxidase (10, EC 1.14.14.161);
   a CYP enzymes assisting alcohol dehydrogenase (CYPADH, EC 1.1.1.-);
   a 7-deoxyloganetic acid glucosyl transferase (7DLGT, EC 2.4.1.323);
   a 7-deoxyloganic acid hydroxylase (7DLH, EC 1.14.14.85);
   a loganic acid O-methyltransferase (LAMT, EC 2.1.1.50);
   a secologanin synthase (SLS, EC 1.14.19.62); and/or
   a strictosidine synthase (STR, EC 4.3.3.2),
   whereby the microorganism is further capable of producing strictosidine and/or halogenated strictosidine and/or derivatives thereof,
wherein preferably said G8H is CroG8H (SEQ ID NO: 67), said CPR is CroCPR (SEQ ID NO: 68), said CYB5 is CroCYB5 (SEQ ID NO: 69), said 8HGO is Vmi8HGOA (SEQ ID NO: 70), said ISY is NcalSY (SEQ ID NO: 71), said CYC is NcaMLPLA (SEQ ID NO: 72), said IO is CroIO (SEQ ID NO: 73), said CYPADH is CroCYPADH (SEQ ID NO: 74), said 7DLGT is Cro7DLGT (SEQ ID NO: 75), said 7DLH is Cro7DLH (SEQ ID NO: 76), said LAMT is CroLAMT (SEQ ID NO: 77), said SLS is CroSLS (SEQ ID NO: 78) and/or said STR is CroSTR (SEQ ID NO: 81) or functional variants thereof having at least 70% homology, similarity or identity to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78 and/or SEQ ID NO: 81, respectively.
6. The microorganism according to any one of the preceding items, further expressing a tryptophan decarboxylase (TDC, EC 4.1.1.28) capable of converting tryptophan to tryptamine and/or halogenated tryptophan to halogenated tryptamine, whereby the microorganism is further capable of producing tryptamine and/or halogenated tryptamine, respectively, optionally said TDC is a heterologous TDC, preferably said TDC is CroTDC as set forth in SEQ ID NO: 79 or rgnTDC as set forth in SEQ ID NO: 80 or a functional variant thereof having at least 70% homology, similarity or identity to SEQ ID NO: 79 or SEQ ID NO: 80, respectively.
7. The microorganism according to any one of the preceding items, further expressing a tryptophan synthase (TRP, EC 4.2.1.20) such as TRP5 as set forth in SEQ ID NO: 138 or a functional variant thereof having at least 70% homology, similarity or identity to SEQ ID NO: 138, and/or a heterologous tryptophan synthase, wherein the tryptophan synthase is capable of converting serine and indole, optionally halogenated indole, to tryptophan, optionally halogenated tryptophan, whereby the microorganism is further capable of producing tryptophan in the presence of serine and indole, optionally said microorganism is further capable of producing halogenated tryptophan in the presence of serine and halogenated indole.
8. The microorganism according to any one of the preceding items, further expressing:
i) a tryptophan halogenase (EC 1.14.19.-), preferably a heterologous tryptophan halogenase such as IaeRebH_N470S as set forth in SEQ ID NO: 111, Trx-IaeRebH (SEQ ID NO: 151), IaeRebH (SEQ ID NO: 153), or a functional variant thereof having at least 70% homology, similarity or identity to SEQ ID NO: 111; and
ii) optionally a flavin reductase such as a FMN reductase (NADPH) (EC 1.5.1.38), preferably a heterologous FMN reductase (NADPH) such as EcoSsuE as set forth in SEQ ID NO: 112 or a functional variant thereof having at least 70% homology, similarity or identity to SEQ ID NO: 112,
wherein the tryptophan halogenase is capable of converting tryptophan to a halogenated tryptophan,
whereby the microorganism is further capable of producing halogenated tryptophan in the presence of tryptophan and a halogen atom, wherein the halogenated tryptophan is a tryptophan substituted with one, two, three or four halogen atoms,
wherein each halogen atom is independently selected from the group consisting of chlorine and bromine.
9. The microorganism according to any one of the preceding items, wherein the tryptophan halogenase is IaeRebH_N470S as set forth in SEQ ID NO: 111 or a functional variant thereof having at least 70% homology, similarity or identity to SEQ ID NO: 111, and wherein the halogen atom is independently selected from chlorine and/or bromine.
10. The microorganism according to any one of the preceding items, wherein the tryptophan halogenase (EC 1.14.19.-) is a tryptophan-5-halogenase (EC 1.14.19.58), a tryptophan-6-halogenase (EC 1.14.19.59), and/or a tryptophan-7-halogenase (EC 1.14.19.9).
11. The microorganism according to any one of the preceding items, further expressing;
   a NADPH-cytochrome P450 reductase (CPR, EC 1.6.2.4);
   a cytochrome b5 (CYB5, EC 1.6.2.2);
   a tetrahydroalstonine synthase (THAS, EC 1.-.--);
   a heteroyohimbine synthase (HYS, EC 1.-.-.-); and/or
   a serpentine synthase (SS),
   whereby the microorganism is further capable of producing alstonine, halogenated alstonine, serpentine and/or halogenated serpentine and/or derivatives thereof,
wherein preferably said CPR is CroCPR (SEQ ID NO: 68), said CYB5 is CroCYB5 (SEQ ID NO: 69), said THAS is CroTHAS1 (SEQ ID NO: 83), said HYS is CroHYS (SEQ ID NO: 84) and/or said SS is CroSS (SEQ ID NO: 110) or functional variants thereof having at least 70% homology, similarity or identity to SEQ ID NO: 68, SEQ ID NO: 69, SEQ ID NO: 83, SEQ ID NO: 84 and/or SEQ ID NO: 110, respectively.
12. The microorganism according to any one of the preceding items, further expressing;
   a NADPH-cytochrome P450 reductase (CPR, EC 1.6.2.4);
   a cytochrome b5 (CYB5, EC 1.6.2.2);
   a precondylocarpine acetate synthase/O-acetylstemmadenine oxidase (PAS/ASO, EC 1.21.3.-);
   a dihydroprecondylocarpine acetate synthase (DPAS, EC 1.1.1.-);
   a tabersonine synthase (TS, EC 4.-.-.-); and/or
   a catharanthine synthase (CS, EC 4.-.-.-),
   whereby the microorganism is further capable of producing tabersonine, halogenated tabersonine, catharanthine and/or halogenated catharanthine and/or derivatives thereof,
wherein preferably said CPR is CroCPR (SEQ ID NO: 68), said CYB5 is CroCYB5 (SEQ ID NO: 69), said PAS/ASO is CroPAS (SEQ ID NO: 95), AhuASO (SEQ ID NO: 96), TibPAS2 (SEQ ID NO: 97) and/or TelASO (SEQ ID NO: 98), said DPAS is CroDPAS (SEQ ID NO: 99) and/or TibDPAS2 (SEQ ID NO: 100), said TS is CroTS (SEQ ID NO: 101) and/or said CS is CroCS (SEQ ID NO: 102) or functional variants thereof having at least 70% homology, similarity or identity to SEQ ID NO: 68, SEQ ID NO: 69, SEQ ID NO: 95, SEQ ID NO: 96, SEQ ID NO: 97, SEQ ID NO: 98, SEQ ID NO: 99, SEQ ID NO: 100, SEQ ID NO: 101 and/or SEQ ID NO: 102, respectively.
13. The microorganism according to any one of the preceding items, further expressing:
   a NADPH-cytochrome P450 reductase (CPR, EC 1.6.2.4);
   a cytochrome b5 (CYB5, EC 1.6.2.2);
   a tabersonine 16-hydroxylase (T16H, EC 1.14.14.103);
   a tabersonine 16-O-methyltransferase (16OMT, EC 2.1.1.94);
   a tabersonine 3-oxygenase (T3O, EC 1.14.14.50);
   a 16-methoxy-2,3-dihydro-3-hydroxytabersonine synthase (T3R, EC 1.1.99.41);
   a 3-hydroxy-16-methoxy-2,3-dihydrotabersonine N-methyltransferase (NMT, EC 2.1.1.99);
   a deacetoxyvindoline 4-hydroxylase (D4H, EC 1.14.11.20); and/or
   a deacetylvindoline O-acetyltransferase (DAT, EC 2.3.1.107),
   whereby the microorganism is further capable of producing vindoline and/or halogenated vindoline and/or derivatives thereof,
wherein preferably said CPR is CroCPR (SEQ ID NO: 68), said CYB5 is CroCYB5 (SEQ ID NO: 69), said TH16 is CroT16H2 (SEQ ID NO: 103), said 16OMT is Cro16OMT (SEQ ID NO: 104), said T3O is CroT3O (SEQ ID NO: 105), said T3R is CroT3R (SEQ ID NO: 106), said NMT is CroNMT (SEQ ID NO: 107), said D4H is CroD4H (SEQ ID NO: 108) and/or said DAT is CroDAT (SEQ ID NO: 109) or functional variants thereof having at least 70% homology, similarity or identity to SEQ ID NO: 68, SEQ ID NO: 69, SEQ ID NO: 103, SEQ ID NO: 104, SEQ ID NO: 105, SEQ ID NO: 106, SEQ ID NO: 107, SEQ ID NO: 108 and/or SEQ ID NO: 109, respectively.
14. The microorganism according to any one of the preceding items, wherein one or more of the polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are tagged with a signal peptide, optionally said signal peptide is a cleavable signal peptide such that said one or more said polypeptides is at least transiently tagged with said signal peptide, preferably said signal peptide is a mitochondrial lumen signal peptide, an endoplasmic reticulum (ER) luminal signal peptide, an ER membrane signal peptide, a nuclear signal peptide, a peroxisomal lumen signal peptide, a vacuolar signal peptide and/or a signal peptide for any other cellular compartment.
15. The microorganism according to any one of the preceding items, wherein the signal peptide is:
   a mitochondrial lumen signal peptide such as ATP9 signal peptide (ATP9sp) as set forth in SEQ ID NO: 140;
   an ER lumen signal peptide such as CNE1 signal peptide (CNE1sp) as set forth in SEQ ID NO: 141;
   an ER membrane signal peptide such as CYB5 signal peptide (CYB5sp) as set forth in SEQ ID NO: 142;
   a peroxisomal lumen signal peptide such as ePTS1 signal peptide (ePTS1sp) as set forth in SEQ ID NO: 143;
   a vacuolar signal peptide such as CPY signal peptide (CPYsp) as set forth in SEQ ID NO: 144; and/or
   a nuclear signal peptide such as SV40 signal peptide (SV40sp) as set forth in SEQ ID NO: 25,
   or functional variants thereof having at least 70% homology, similarity or identity to SEQ ID NO: 140, SEQ ID NO: 141, SEQ ID NO: 142, SEQ ID NO: 143, SEQ ID NO: 144 and/or SEQ ID NO: 25, respectively.
16. The microorganism according to any one of the preceding items, wherein two or more of the polypeptides GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase are localised in the same cellular compartment and/or cellular membrane such as in the ER lumen, ER membrane, the cytoplasmic site of the ER membrane, mitochondrial lumen, nucleus, peroxisomal lumen, vacuole and/or any other cellular compartment, optionally said one or more polypeptides are tagged with identical signal peptides that direct the polypeptides to the same cellular compartment and/or cellular membrane.
17. The microorganism according to any one of the preceding items, wherein the SGD and the GS are localised in the same cellular compartment and/or cellular membrane such as in the ER lumen, ER membrane, the cytoplasmic site of the ER membrane, mitochondrial lumen, nucleus, peroxisomal lumen, vacuole and/or any other cellular compartment, preferably in the ER lumen, the cytoplasmic site of the ER membrane and/or the mitochondrial lumen.
18. The microorganism according to any one of the preceding items, wherein said SGD is ATP9sp-RseSGD (SEQ ID NO: 113), RseSGD-CNE1sp (SEQ ID NO: 115), RseSGD-CYB5sp (SEQ ID NO: 117), RseSGD-ePTS1sp (SEQ ID NO: 119), RseSGD-CPYsp (SEQ ID NO: 121) and/or SV40sp-RseSGD (SEQ ID NO: 123), and/or said GS is ATP9sp-CroGS (SEQ ID NO: 114), CroGS-CNE1sp (SEQ ID NO: 116) and/or CroGS-CYB5sp (SEQ ID NO: 118), CroGS-ePTS1sp (SEQ ID NO: 120), CroGS-CPYsp (SEQ ID NO: 122) and/or SV40sp-CroGS (SEQ ID NO: 124) or functional variants thereof having at least 70% homology, similarity or identity to SEQ ID NO: 113, SEQ ID NO: 115, SEQ ID NO: 117, SEQ ID NO: 119, SEQ ID NO: 121, SEQ ID NO: 123, SEQ ID NO: 114, SEQ ID NO: 116, SEQ ID NO: 118, SEQ ID NO: 120, SEQ ID NO: 122 and/or SEQ ID NO: 124, respectively,
   preferably said SGD is ATP9sp-RseSGD (SEQ ID NO: 113), RseSGD-CNE1sp (SEQ ID NO: 115) and/or RseSGD-CYB5sp (SEQ ID NO: 117), and/or said GS is ATP9sp-CroGS (SEQ ID NO: 114), CroGS-CNE1sp (SEQ ID NO: 116) and/or CroGS-CYB5sp (SEQ ID NO: 118) or functional variants thereof having at least 70% homology, similarity or identity to SEQ ID NO: 113, SEQ ID NO: 115, SEQ ID NO: 117, SEQ ID NO: 114, SEQ ID NO: 116 and/or SEQ ID NO: 118, respectively.
19. The microorganism according to any one of the preceding items, further overexpressing an NADH kinase specific to said cellular compartment, preferably a mitochondrial NADH kinase such as POS5 as set forth in SEQ ID NO: 87 or a functional variant thereof having at least 70% homology, similarity or identity thereto.
20. The microorganism according to any one of the preceding items, further expressing ATP9sp-RseSGD (SEQ ID NO: 113), GS is ATP9sp-CroGS (SEQ ID NO: 114) and overexpressing POS5 (SEQ ID NO: 87) or functional variants thereof having at least 70% homology, similarity or identity to SEQ ID NO: 113, 114 and/or 87.
21. The microorganism according to any one of the preceding items, wherein the halogenated indole is substituted with at least one, such as at least two, such as at least three, such as at least four halogen atoms, each halogen atom independently selected from the group consisting of fluorine, bromine and chlorine.
22. The microorganism according to any one of the preceding items, wherein the halogenated indole is 4-halogenated, 5-halogenated, 6-halogenated or 7-halogenated by a halogen selected from the group consisting of fluorine, chlorine and bromine.
23. The microorganism according to any one of the preceding items, wherein the halogenated indole is selected from the group consisting of fluoroindole, chloroindole and bromoindole.
24. The microorganism according to any one of the preceding items, wherein the halogenated indole is selected from the group consisting of 4-fluoroindole, 5-fluoroindole, 6-fluoroindole, 7-fluoroindole, 4-chloroindole, 5-chloroindole, 6-chloroindole, 7-chloroindole, 4-bromoindole, 5-bromoindole, 6-bromoindole and 7-bromoindole.
25. The microorganism according to any one of the preceding items, wherein the halogenated indole is 4,5-dihalogenated, 4,6-dihalogenated, 4,7-dihalogenated, 5,6-dihalogenated, 5,7-dihalogenated and/or 6,7-dihalogenated by two halogen atoms, each halogen atom independently selected from the group consisting of fluorine, bromine and chlorine.
26. The microorganism according to any one of the preceding items, wherein the halogenated indole is 4,5,6-trihalogenated, 4,5,7-trihalogenated, 4,6,7-trihalogenated and/or 5,6,7-trihalogenated by three halogen atoms, each halogen atom independently selected from the group consisting of fluorine, bromine and chlorine.
27. The microorganism according to any one of the preceding items, wherein the halogenated indole is 4,5,6,7-tetrahalogenated by four halogen atoms, each halogen atom independently selected from the group consisting of fluorine, bromine and chlorine.
28. The microorganism according to any one of the preceding items, wherein the halogenated strictosidine, strictosidine aglycone, alstonine, serpentine, catharanthine, tabersonine, vindoline, stemmadenine acetate and/or derivatives thereof is a tryptophan, tryptamine, strictosidine, strictosidine aglycone, alstonine, serpentine, catharanthine, tabersonine, vindoline, stemmadenine acetate and/or derivatives thereof is substituted with at least one, such as at least two, such as at least three, such as at least four halogen atoms, each halogen atom independently selected from the group consisting of fluorine, bromine and chlorine.
29. The microorganism according to any one of the preceding items, wherein the halogenated strictosidine, strictosidine aglycone, alstonine, serpentine, catharanthine, tabersonine, stemmadenine acetate and/or derivatives thereof is 4-halogenated, 5-halogenated, 6-halogenated and/or 7-halogenated by a halogen atom selected from the group consisting of fluorine, bromine and chlorine, and/or the halogenated vindoline and/or derivatives thereof is 4-halogenated, 5-halogenated and/or 7-halogenated by a halogen atom selected from the group consisting of fluorine, bromine and chlorine, preferably with a titer of at least 1 μg/L, or more.
30. The microorganism according to any one of the preceding items, wherein the halogenated strictosidine, strictosidine aglycone, alstonine, serpentine, catharanthine, tabersonine, stemmadenine acetate and/or derivatives thereof is 4,5-dihalogenated, 4,6-dihalogenated, 4,7-dihalogenated, 5,6-dihalogenated, 5,7-dihalogenated and/or 6,7-dihalogenated by two halogen atoms, each halogen atom independently selected from the group consisting of fluorine, bromine and chlorine, and/or the halogenated vindoline and/or derivatives thereof is 4,5-dihalogenated, 4,7-dihalogenated and/or 5,7-dihalogenated by two halogen atoms, each halogen atom independently selected from the group consisting of fluorine, bromine and chlorine, preferably with a titer of at least 1 μg/L, or more.
31. The microorganism according to any one of the preceding items, wherein the halogenated strictosidine, strictosidine aglycone, alstonine, serpentine, catharanthine, tabersonine, stemmadenine acetate and/or derivatives thereof is 4,5,6-trihalogenated, 4,5,7-trihalogenated, 4,6,7-trihalogenated and/or 5,6,7-trihalogenated by three halogen atoms, each halogen atom independently selected from the group consisting of fluorine, bromine and chlorine, and/or the halogenated vindoline and/or derivatives thereof is 4,5,7-trihalogenated by three halogen atoms, each halogen atom independently selected from the group consisting of fluorine, bromine and chlorine, preferably with a titer of at least 1 μg/L, or more.
32. The microorganism according to any one of the preceding items, wherein the halogenated strictosidine, strictosidine aglycone, alstonine, serpentine, catharanthine, tabersonine, stemmadenine acetate and/or derivatives thereof is 4,5,6,7-tetrahalogenated by at least four halogen atoms, each halogen atom independently selected from the group consisting of fluorine, bromine and chlorine, preferably with a titer of at least 1 μg/L, or more.
33. The microorganism according to any one of the preceding items, wherein the halogen atom is independently selected from the group consisting of fluorine, bromine, and chlorine, preferably bromine and chlorine.
34. The microorganism according to any one of the preceding items, further comprising one or more mutations, preferably said one or more mutations result in increased availability of geranyl diphosphate for said microorganism.
35. The microorganism according to any one of the preceding items, wherein the one or more mutations are mutations resulting in loss of function of one or more of:
   ATF1 (YOR377W);
   OYE2 (YHR179W);
   ADH6 (YMR318C);
   OYE3 (YPL171C);
   ROX1 (YPR065W);
   ARI1 (YGL157W),
or functional variants thereof having at least 70% homology, similarity or identity to ATF1 (YOR377W), OYE2 (YHR179W), ADH6 (YMR318C), OYE3 (YPL171C), ROX1 (YPR065W) and/or ARI1 (YGL157W), respectively, preferably wherein the one or more mutations are mutations of the genes encoding said proteins, such as one or more deletions.
36. The microorganism according to any one of the preceding items, further overexpressing one or more of:
   ID11 (SEQ ID NO: 136, YPL117C);
   trunHMG1 (SEQ ID NO: 137),
or functional variants thereof having at least 70% homology, similarity or identity to ID11 (SEQ ID NO: 136) and/or trunHMG1 (SEQ ID NO: 137), respectively.
37. The microorganism according to any one of the preceding items, wherein the one or more mutations is a mutation leading to reduced function, such as downregulation, of one or more of:
   ERG20 (YJL167W);
   ERG9 (YHR190W),
or functional variants thereof having at least 70% homology, similarity or identity to ERG20 (YJL167W) and/or ERG9 (YHR190W), respectively,
preferably wherein the one or more mutations are mutations of the genes encoding said proteins, such as one or more deletions, said down-regulation might be due to reduced expression.
38. The microorganism according to any of the preceding items, wherein the microorganism is selected from the group consisting of yeasts, bacteria, archaea, fungi, protozoa, algae, and viruses, preferably the microorganism is a yeast or a bacterium.
39. The microorganism according to any one of the preceding items, wherein the microorganism is a yeast.
40. The microorganism according to item 39, wherein the genus of said yeast is selected from the group consisting of Saccharomyces, Pichia, Komagataella, Yarrowia, Kluyveromyces, Candida, Rhodotorula, Rhodosporidium, Cryptococcus, Trichosporon and Lipomyces.
41. The microorganism according to any one of items 39 to 40, wherein the yeast is selected from the group consisting of Saccharomyces cerevisiae, Saccharomyces boulardii, Komagataella phaffi (Pichia pastoris), Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica.
42. The microorganism according to any one of items 39 to 41, wherein the yeast is Saccharomyces cerevisiae.
43. The microorganism according to item 38, wherein the microorganism is a bacterium.
44. The microorganism according to item 43, wherein the genus of said bacterium is selected from the groups consisting of Escherichia, Corynebacterium, Pseudomonas, Bacillus, Streptomyces, Lactococcus, Lactobacillus, Halomonas, Bifidobacterium and Enterococcus.
45. The microorganism according to any one of items 43 to 44, wherein the bacterium is selected from the group consisting of Escherichia coli, Corynebacterium glutamicum, Pseudomonas putida, Bacillus subtilis, Streptomyces albus, Lactococcus bacillus, Halomonas elongate, Bifidobacterium infantis and Enterococcus faecal, preferably an Escherichia coli.
46. The microorganism according to any one of the preceding items comprising one or more nucleic acid sequences encoding the GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase, wherein said one or more nucleic acids are codon-optimised for the microorganism.
47. The microorganism according to any one of the preceding items comprising one or more nucleic acid sequences encoding the GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase, wherein said one or more nucleic acids are;
   i. present in high copy number, in low copy number and/or combinations thereof;
   ii. under the control of an inducible promoter, a constitutive promoter and/or combinations thereof;
   iii. comprised in one or more vectors; and/or
   iv. integrated into the genome of the microorganism according to any one of the items 1 to 46.
48. A nucleic acid construct comprising a nucleic acid sequence identical to or having at least 70% homology, similarity or identity to SEQ ID NO: 3, 4 and/or 20.
49. The nucleic acid construct according to item 48, wherein the nucleic acid construct further comprises a nucleic acid sequence identical to or having at least 70% homology, similarity or identity to SEQ ID NO: 23, 24, 6, 7, 26, 27, 28, 29, 30, 31 and/or 32.
50. The nucleic acid construct according to any one of the items 48 to 49, wherein the nucleic acid construct further comprises a nucleic acid sequence identical to or having at least 70% homology, similarity or identity to SEQ ID NO: 1, 2 and/or 128.
51. The nucleic acid construct according to any one of the items 48 to 50, wherein the nucleic acid construct further comprises a nucleic acid sequence identical to or having at least 70% homology, similarity or identity to SEQ ID NO: 5, 6, 7, 8, 9, 10, 11, 12, 13, 14, 15, 16 and/or 19.
52. The nucleic acid construct according to any one of the items 48 to 51, wherein the nucleic acid construct further comprises a nucleic acid sequence identical to or having at least 70% homology, similarity or identity to SEQ ID NO: 17, 18, 127, 49, 152, 154 and/or 50.
53. The nucleic acid construct according to any one of the items 48 to 52, wherein the nucleic acid construct further comprises a nucleic acid sequence identical to or having at least 70% homology, similarity or identity to SEQ ID NO: 6, 7, 21, 22 and/or 48.
54. The nucleic acid construct according to any one of the items 48 to 53, wherein the nucleic acid construct further comprises a nucleic acid sequence identical to or having at least 70% homology, similarity or identity to SEQ ID NO: 6, 7, 33, 34, 35, 36, 37, 38, 39, 150, and/or 40.
55. The nucleic acid construct according to any one of the items 48 to 54, wherein the nucleic acid construct further comprises a nucleic acid sequence identical to or having at least 70% homology, similarity or identity to SEQ ID NO: 6, 7, 41, 42, 43, 44, 45, 46 and/or 47.
56. The nucleic acid construct according to any one of the items 48 to 55, further comprising a nucleic acid sequence identical to or having at least 70% homology, similarity or identity to SEQ ID NO: 129, 130, 131, 132, 133 and/or 134, preferably SEQ ID NO: 129, 130 and/or 131.
57. The nucleic acid construct according to any one of the items 48 to 56, further comprising a nucleic acid sequence identical to or having at least 70% homology, similarity or identity to SEQ ID NO: 51, 52, 53, 54, 55, 56, 57, 58, 59, 60, 61, 62 and/or 135, preferably SEQ ID NO: 51, 52, 53, 54, 55, 56, and/or 135.
58. The nucleic acid construct according to any one of the items 48 to 57, further comprising a nucleic acid sequence identical to or having at least 70% homology, similarity or identity to SEQ ID NO: 125 and/or SEQ ID NO: 126.
59. The nucleic acid construct according to any one of the items 48 to 58, wherein one or more of the nucleic acid constructs and/or nucleic acid sequences are;
   i. present in high copy number, in low copy number and/or combinations thereof;
   ii. under the control of an inducible promoter, a constitutive promoter and/or a combination thereof; and/or
   iii. integrated into the genome of the microorganism according to any one of the items 1 to 47.
60. The nucleic acid construct according to any one of the items 48 to 59, wherein the nucleic acid construct is a vector such as a plasmid, a high-copy vector, an episomal vector, an integrative vector and/or a replicative vector.
61. A vector comprising a nucleic acid construct according to any one of items 48 to 59.
62. A method of producing strictosidine aglycone, such as halogenated strictosidine aglycone, and optionally derivatives thereof in a microorganism, said method comprising the steps of;
   i. providing a microorganism, said microorganism expressing:
      a geraniol synthase (GES, EC 3.1.7.11) and
      a strictosidine-O-β-D-glucosidase (SGD, EC 3.2.1.105);
   ii. incubating said microorganism in a medium comprising a substrate and optionally a halogen atom source, which can be converted to strictosidine aglycone and/or halogenated strictosidine aglycone by said microorganism in the presence of geranyl diphosphate and tryptamine and/or halogenated tryptamine;
   iii. optionally recovering the strictosidine aglycone and/or halogenated strictosidine aglycone;
   iv. optionally further converting the strictosidine aglycone and/or halogenated strictosidine aglycone to one or more monoterpene indole alkaloids (MIAs) and/or halogenated MIAs,
wherein said GES is capable of converting geranyl diphosphate to geraniol, optionally said GES is a heterologous GES, preferably said GES is as set forth in SEQ ID NO: 65 and/or in SEQ ID NO: 66; and/or
wherein said SGD is capable of converting strictosidine and/or halogenated strictosidine to strictosidine aglycone and/or halogenated strictosidine aglycone, respectively, optionally said SGD is a heterologous SGD, preferably said SGD is RseSGD (SEQ ID NO: 82), or functional variants thereof having at least 70% homology, similarity or identity to SEQ ID NO: 65, SEQ ID NO: 66 and/or SEQ ID NO: 82, respectively.
63. The method according to item 62, wherein the method comprises step iv. and wherein said microorganism is as defined in any one of items 2 to 47.
64. The method according to any one of items 62 to 63, wherein the method comprises step iv. and wherein the one of more MIAs comprise stemmadenine acetate and/or halogenated stemmadenine acetate, said microorganism further expressing:
   a GPPS, a G8H, a CPR, a CYB5, a 8HGO, an ISY, an CYC, an 10, a CYPADH, a 7DLGT, a 7DLH, a LAMT, a TDC, a SLS, a STR, a GS, a GO, a protein Redox1, a protein Redox2 and/or a SAT,
wherein preferably said GPPS is AgrGPPS2 (SEQ ID NO: 63) and/or GgaFPS*(N144W) (SEQ ID NO: 64), said G8H is CroG8H (SEQ ID NO: 67), said CPR is CroCPR (SEQ ID NO: 68), said CYB5 is CroCYB5 (SEQ ID NO: 69), said 8HGO is Vmi8HGOA (SEQ ID NO: 70), said ISY is NcalSY (SEQ ID NO: 71), said CYC is NcaMLPLA (SEQ ID NO: 72), said IO is CroIO (SEQ ID NO: 73), said CYPADH is CroCYPADH (SEQ ID NO: 74), said 7DLGT is Cro7DLGT (SEQ ID NO: 75), said 7DLH is Cro7DLH (SEQ ID NO: 76), said LAMT is CroLAMT (SEQ ID NO: 77), said TDC is CroTDC (SEQ ID NO: 79) and/or rgnTDC (SEQ ID NO: 80), said SLS is CroSLS (SEQ ID NO: 78), said STR is CroSTR (SEQ ID NO: 81), said GS is CroGS (SEQ ID NO: 85) and/or CroADH13 (SEQ ID NO: 86), said GO is CroGO (SEQ ID NO: 88), TelGO (SEQ ID NO: 89), VmiGO (SEQ ID NO: 90), and/or AhuGO (SEQ ID NO: 91), said protein Redox1 is CroRdx1 (SEQ ID NO: 92), said protein Redox2 is CroRdx2 (SEQ ID NO: 93) and/or said SAT is CroSAT (SEQ ID NO: 94) or functional variants thereof having at least 70% homology, similarity or identity to SEQ ID NO: 63, 64, 67, 68, 69, 70, 71, 72, 73, 74, 75, 76, 77, 78, 81, 85, 86, 68, 69, 88, 89, 90, 91, 92, 93 and/or 94, respectively.
65. The method according to any one of items 62 to 64, wherein the microorganism further expresses a tryptophan halogenase (EC 1.14.19.-) and optionally a flavin reductase such as FMN reductase (NADPH) (EC 1.5.1.38), wherein the tryptophan halogenase is capable of converting tryptophan to a halogenated tryptophan,
wherein preferably said tryptophan halogenase is IaeRebH_N470S (SEQ ID NO: 111), IaeRebH (SEQ ID NO: 153) or Trx-IaeRebH (SEQ ID NO: 151), and/or said FMN reductase (NADPH) is EcoSsuE (SEQ ID NO: 112), or functional variants thereof having at least 70% homology, similarity or identity to SEQ ID NO: 111 and/or SEQ ID NO: 112, respectively, whereby the microorganism is further capable of producing halogenated tryptophan in the presence of tryptophan and at least one halogen atom, wherein the halogenated tryptophan is a tryptophan substituted with one, two, three or four halogen atoms, wherein each halogen atom is independently selected from the group consisting of fluorine, chlorine and bromine.
66. The method according to any one of items 64 to 65, wherein the halogenated stemmadenine acetate is 4-fluorostemmadenine acetate, 5-fluorostemmadenine acetate, 6-fluorostemmadenine acetate, 7-fluorostemmadenine acetate, 4-bromostemmadenine acetate, 5-bromostemmadenine acetate, 6-bromostemmadenine acetate, 7-bromostemmadenine acetate, 4-chlorostemmadenine acetate, 5-chlorostemmadenine acetate, 6-chlorostemmadenine acetate, 7-chlorostemmadenine acetate and/or derivatives thereof, preferably said halogenated stemmadenine acetate and/or derivatives thereof is 4-fluorostemmadenine acetate, 6-fluorostemmadenine acetate, 7-fluorostemmadenine acetate and/or derivatives thereof.
67. The method according to any one of items 62 to 66, wherein the medium further comprises at least:
   i. halogenated indole as defined in any one of items 21 to 24, preferably at a concentration of at least 0.05 mM, such as at least 0.1 mM, such as at least 0.5 mM, such as at least 1 mM, such as at least 2 mM or more;
   ii. tryptamine and/or halogenated tryptamine, preferably at a concentration of at least 0.05 mM, such as at least 0.1 mM, such as at least 0.5 mM, such as at least 1 mM, such as at least 2 mM or more;
   iii. a halogen atom source such as a salt, preferably NaCl and/or KBr, preferably at a concentration of at least 0.05 M, such as at least 0.1 M, such as at least 0.5 M or more;
   iv. tryptophan and/or halogenated tryptophan, preferably at a concentration of at least 0.05 mM, such as at least 0.1 mM, such as at least 0.5 mM, such as at least 1 mM, such as at least 2 mM or more;
   v. secologanin, preferably at a concentration of at least 0.05 mM, such as at least 0.1 mM, such as at least 0.5 mM, such as at least 1 mM, such as at least 2 mM or more,
or combinations thereof.
68. The method according to any one of items 62 to 67, further comprising one or more of the steps of:
   i. recovering the MIA and/or derivatives thereof;
   ii. converting said MIA and/or derivatives thereof to derivatives thereof and/or downstream products thereof, respectively; and/or
   iii. formulating said MIA and/or derivatives thereof and/or downstream products thereof in a composition such as a pharmaceutical composition.
69. The method according to any one of items 62 to 68, wherein the microorganism is as defined in item 11, and wherein the one or more MIAs comprise alstonine, halogenated alstonine, serpentine and/or halogenated serpentine and/or derivatives thereof.
70. The method according to any one of items 62 to 69, wherein the microorganism is as defined in item 12, and wherein the one or more MIAs comprise tabersonine, halogenated tabersonine, catharanthine and/or halogenated catharanthine and/or derivatives thereof.
71. The method according to any one of items 62 to 70, wherein the microorganism is as defined in item 13, and wherein the one or more MIAs comprise vindoline and/or halogenated vindoline and/or derivatives thereof.
72. A composition comprising one of more derivatives of strictosidine aglycone and/or halogenated strictosidine aglycone obtained by the method according to any one of items 62 to 71.
73. The composition according to item 72, wherein the one of more derivatives comprise stemmadenine acetate and/or halogenated stemmadenine acetate obtained by the method according to any one of items 62 to 71.
74. Strictosidine aglycone, stemmadenine acetate, alstonine, serpentine, catharanthine, tabersonine, vindoline, halogenated strictosidine aglycone, halogenated stemmadenine acetate, halogenated alstonine, halogenated serpentine, halogenated catharanthine, halogenated tabersonine, halogenated vindoline and/or derivatives thereof obtained by the method according to any one of items 62 to 71.
75. Strictosidine, halogenated strictosidine and/or derivatives thereof obtained by the method according to any one of items 62 to 71, preferably wherein said halogenated strictosidine and/or derivatives thereof is 4-fluorostrictosidine, 5-fluorostrictosidine, 6-fluorostrictosidine, 7-fluorostrictosidine, 4-chlorostrictosidine, 5-chlorostrictosidine, 6-chlorostrictosidine, 7-chlorostrictosidine, 4-bromostrictosidine, 5-bromostrictosidine, 6-bromostrictosidine, 7-bromostrictosidine and/or derivatives thereof, preferably said halogenated strictosidine and/or derivatives thereof is 4-fluorostrictosidine, 5-fluorostrictosidine, 6-fluorostrictosidine, 7-fluorostrictosidine, 7-chlorostrictosidine, 7-bromostrictosidine and/or derivatives thereof.
76. Ajmalicine, serpentine, alstonine and/or derivatives thereof and/or halogenated ajmalicine, serpentine, alstonine and/or derivatives thereof, obtained by the method according to any one of items 62 to 71, preferably wherein said halogenated tetrahydroalstonine, ajmalicine, serpentine, alstonine and/or derivatives thereof is 7-chloroajmalicine, 7-chloroserpentine, 7-chloroalstonine and/or derivatives thereof, respectively.
77. Tetrahydroalstonine and/or derivatives thereof and/or halogenated tetrahydroalstonine and/or derivatives thereof obtained by the method according to any one of items 62 to 71, preferably wherein said halogenated tetrahydroalstonine and/or derivatives thereof is 7-chlorotetrahydroalstonine, 4-fluorotetrahydroalstonine, 5-fluorotetrahydroalstonine, 6-fluorotetrahydroalstonine, 7-fluoro tetrahydroalstonine and/or derivatives thereof.
78. Geissoschizine and/or halogenated geissoschizine and/or derivatives thereof obtained by the method according to any one of items 62 to 71, preferably wherein said halogenated geissoschizine and/or derivatives thereof is fluorinated geissoschizine and/or derivatives thereof such as 4-fluorogeissoschizine, 5-fluorogeissoschizine, 6-fluorogeissoschizine and/or 7-fluorogeissoschizine and/or derivatives thereof, preferably said halogenated geissoschizine and/or derivatives thereof is 4-fluorogeissoschizine, 6-fluorogeissoschizine and/or 7-fluorogeissoschizine and/or derivatives thereof.
79. Stemmadenine acetate and/or halogenated stemmadenine acetate and/or derivatives thereof obtained by the method according to any one of items 62 to 71, preferably wherein said halogenated stemmadenine acetate and/or derivatives thereof is fluorinated stemmadenine acetate and/or derivatives thereof such as 4-fluorostemmadenine acetate, 5-fluorostemmadenine acetate, 6-fluorostemmadenine acetate, 7-fluorostemmadenine acetate and/or derivatives thereof, preferably said halogenated stemmadenine acetate and/or derivatives thereof is 4-fluorostemmadenine acetate, 6-fluorostemmadenine acetate, 7-fluorostemmadenine acetate and/or derivatives thereof.
80. Tabersonine and/or halogenated tabersonine and/or derivatives thereof obtained by the method according to any one of items 62 to 71, preferably wherein said halogenated tabersonine and/or derivatives thereof is fluorinated tabersonine and/or derivatives thereof such as 4-fluorotabersonine, 5-fluorotabersonine, 6-fluorotabersonine, 7-fluorotabersonine and/or derivatives thereof, preferably said halogenated tabersonine and/or derivatives thereof is 6-fluorotabersonine, 7-fluorotabersonine and/or derivatives thereof.
81. A microorganism comprising a nucleic acid construct according to any one of items 48 to 60 or the vector according to item 61, preferably wherein the microorganism is a bacterium such as Escherichia coli or a yeast such as Saccharomyces cerevisiae.
82. A method for manufacturing a monoterpene indole alkaloid (MIA) and/or a halogenated MIA and/or derivatives thereof of interest, said method comprising the steps of:
   iii. providing a MIA and/or a halogenated MIA and/or derivatives thereof; and
   iv. optionally converting said MIA and/or halogenated MIA and/or derivatives thereof to the MIA and/or halogenated MIA and/or derivatives thereof of interest.
83. A kit of parts comprising a microorganism according to any one of items 1 to 47, and/or at least one nucleic acid construct according to any one of items 48 to 60 and/or the vector according to item 61, and instructions for use.
84. Use of the nucleic acid construct according to any one of items 48 to 60, the microorganism according to any one of items 1 to 47, the vector according to item 61, or the microorganism according to item 81, for the production of strictosidine aglycone, stemmadenine acetate, tetrahydroalstonine, alstonine, serpentine, catharanthine, tabersonine and/or vindoline and/or derivatives thereof in a microorganism, optionally said strictosidine aglycone, stemmadenine acetate, tetrahydroalstonine, alstonine, serpentine, catharanthine, tabersonine and/or vindoline and/or derivatives thereof are halogenated.
85. A method of treating a disorder such as a cancer, arrhythmia, malaria, fibrosis, pain, anxiety, Parkinson's disease, schizophrenia, bipolar disorder, psychotic diseases or disorders, hypertension, depression, Alzheimer's disease, addiction neuronal diseases, and/or withdrawal symptoms, comprising administration of a therapeutic sufficient amount of a MIA, a halogenated MIA and/or a pharmaceutical compound obtained by the method according to any one of items 62 to 71.

### Items 2

1. A microorganism capable of producing strictosidine aglycone and/or halogenated strictosidine aglycone and/or derivatives thereof, in the presence of geranyl diphosphate and tryptamine, and/or geranyl diphosphate and halogenated tryptamine, respectively, said microorganism expressing a geraniol synthase (GES, EC 3.1.7.11) and a strictosidine-O-β-D-glucosidase (SGD, EC 3.2.1.105),
optionally wherein:
   i) the geraniol synthase (GES, EC 3.1.7.11) is a GES capable of converting geranyl diphosphate to geraniol, optionally said GES is a heterologous GES, preferably said GES is as set forth in SEQ ID NO: 65 and/or as set forth in SEQ ID NO: 66 or a functional variant thereof having at least 70% homology, similarity or identity to SEQ ID NO: 65 and/or SEQ ID NO: 66, respectively; and/or
   ii) the strictosidine-O-β-D-glucosidase (SGD, EC 3.2.1.105) is a SGD capable of converting strictosidine and/or halogenated strictosidine to strictosidine aglycone and/or halogenated strictosidine aglycone, respectively, optionally said SGD is a heterologous SGD, preferably said SGD is RseSGD as set forth in SEQ ID NO: 82 or a functional variant thereof having at least 70% homology, similarity or identity to SEQ ID NO: 82,
   preferably wherein the microorganism further expresses:
      a geissoschizine synthase (GS, EC 1.3.1.36);
      a NADPH-cytochrome P450 reductase (CPR, EC 1.6.2.4);
      a cytochrome b5 (CYB5, EC 1.6.2.2);
      a geissoschizine oxidase (GO, EC 1.14.14.-);
      a protein Redox1 (EC 1.14.14.-);
      a protein Redox2 (EC 1.7.1.-); and/or
      a stemmadenine-O-acetyltransferase (SAT, EC 1.7.1.-),
      whereby the microorganism is further capable of producing stemmadenine acetate and/or halogenated stemmadenine acetate and/or derivatives thereof,
   wherein preferably said GS is CroGS (SEQ ID NO: 85) and/or CroADH13 (SEQ ID NO: 86), said CPR is CroCPR (SEQ ID NO: 68), said CYB5 is CroCYB5 (SEQ ID NO: 69), said GO is CroGO (SEQ ID NO: 88), TelGO (SEQ ID NO: 89), VmiGO (SEQ ID NO: 90), and/or AhuGO (SEQ ID NO: 91), said protein Redox1 is CroRdx1 (SEQ ID NO: 92), said protein Redox2 is CroRdx2 (SEQ ID NO: 93) and/or said SAT is CroSAT (SEQ ID NO: 94) or functional variants thereof having at least 70% homology, similarity or identity to SEQ ID NO: 85, SEQ ID NO: 86, SEQ ID NO: 68, SEQ ID NO: 69, SEQ ID NO: 88, SEQ ID NO: 89, SEQ ID NO: 90, SEQ ID NO: 91, SEQ ID NO: 92, SEQ ID NO: 93 and/or SEQ ID NO: 94, respectively.
2. The microorganism according to any one of the preceding items, further expressing a geranyl diphosphate synthase (GPPS, EC 2.5.1.1) capable of converting isopentenyl diphosphate (IPP) and dimethylallyl diphosphate (DMAPP) to geranyl diphosphate, optionally said GPPS is a heterologous GPPS, preferably said GPPS is AgrGPPS2 as set forth in SEQ ID NO: 63, GgaFPS*(N144w) as set forth in SEQ ID NO: 64 or ERG20** as set forth in SEQ ID NO: 139 or a functional variant thereof having at least 70% homology, similarity or identity to SEQ ID NO: 63, SEQ ID NO: 64 or SEQ ID NO: 139, respectively;
and/or further expressing:
   a geraniol-8-hydroxylase (G8H, EC 1.14.14.83);
   a NADPH-cytochrome P450 reductase (CPR, EC 1.6.2.4);
   a cytochrome b5 (CYB5, EC 1.6.2.2);
   a 8-hydroxygeraniol oxidoreductase (8HGO, EC 1.1.1.324);
   an iridoid synthase (ISY, EC 1.3.1.122);
   an iridoid cyclase (CYC, EC 5.5.1.34);
   an iridoid oxidase (10, EC 1.14.14.161);
   a CYP enzymes assisting alcohol dehydrogenase (CYPADH, EC 1.1.1.-);
   a 7-deoxyloganetic acid glucosyl transferase (7DLGT, EC 2.4.1.323);
   a 7-deoxyloganic acid hydroxylase (7DLH, EC 1.14.14.85);
   a loganic acid O-methyltransferase (LAMT, EC 2.1.1.50);
   a secologanin synthase (SLS, EC 1.14.19.62); and/or
   a strictosidine synthase (STR, EC 4.3.3.2),
   whereby the microorganism is further capable of producing strictosidine and/or halogenated strictosidine and/or derivatives thereof,
wherein preferably said G8H is CroG8H (SEQ ID NO: 67), said CPR is CroCPR (SEQ ID NO: 68), said CYB5 is CroCYB5 (SEQ ID NO: 69), said 8HGO is Vmi8HGOA (SEQ ID NO: 70), said ISY is NcalSY (SEQ ID NO: 71), said CYC is NcaMLPLA (SEQ ID NO: 72), said IO is CroIO (SEQ ID NO: 73), said CYPADH is CroCYPADH (SEQ ID NO: 74), said 7DLGT is Cro7DLGT (SEQ ID NO: 75), said 7DLH is Cro7DLH (SEQ ID NO: 76), said LAMT is CroLAMT (SEQ ID NO: 77), said SLS is CroSLS (SEQ ID NO: 78) and/or said STR is CroSTR (SEQ ID NO: 81) or functional variants thereof having at least 70% homology, similarity or identity to SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78 and/or SEQ ID NO: 81, respectively.
3. The microorganism according to any one of the preceding items, further expressing:
   a) a tryptophan decarboxylase (TDC, EC 4.1.1.28) capable of converting tryptophan to tryptamine and/or halogenated tryptophan to halogenated tryptamine, whereby the microorganism is further capable of producing tryptamine and/or halogenated tryptamine, respectively, optionally said TDC is a heterologous TDC, preferably said TDC is CroTDC as set forth in SEQ ID NO: 79 or rgnTDC as set forth in SEQ ID NO: 80 or a functional variant thereof having at least 70% homology, similarity or identity to SEQ ID NO: 79 or SEQ ID NO: 80, respectively; and/or
   b) a tryptophan synthase (TRP, EC 4.2.1.20) such as TRP5 as set forth in SEQ ID NO: 138 or a functional variant thereof having at least 70% homology, similarity or identity to SEQ ID NO: 138, and/or a heterologous tryptophan synthase, wherein the tryptophan synthase is capable of converting serine and indole, optionally halogenated indole, to tryptophan, optionally halogenated tryptophan, whereby the microorganism is further capable of producing tryptophan in the presence of serine and indole, optionally said microorganism is further capable of producing halogenated tryptophan in the presence of serine and halogenated indole; and/or
   c) a tryptophan halogenase (EC 1.14.19.-), preferably a heterologous tryptophan halogenase such as IaeRebH_N470S as set forth in SEQ ID NO: 111 or a functional variant thereof having at least 70% homology, similarity or identity to SEQ ID NO: 111, wherein the tryptophan halogenase is capable of converting tryptophan to a halogenated tryptophan; and optionally a flavin reductase such as a FMN reductase (NADPH) (EC 1.5.1.38), preferably a heterologous FMN reductase (NADPH) such as EcoSsuE as set forth in SEQ ID NO: 112 or a functional variant thereof having at least 70% homology, similarity or identity to SEQ ID NO: 112,
whereby the microorganism is further capable of producing halogenated tryptophan in the presence of tryptophan and a halogen atom, wherein the halogenated tryptophan is a tryptophan substituted with one, two, three or four halogen atoms, wherein each halogen atom is independently selected from the group consisting of chlorine and bromine; and/or
   d) a NADPH-cytochrome P450 reductase (CPR, EC 1.6.2.4);
      a cytochrome b5 (CYB5, EC 1.6.2.2);
      a tetrahydroalstonine synthase (THAS, EC 1.-.--);
      a heteroyohimbine synthase (HYS, EC 1.-.-.-); and/or
      a serpentine synthase (SS),
      whereby the microorganism is further capable of producing alstonine, halogenated alstonine, serpentine and/or halogenated serpentine and/or derivatives thereof,
wherein preferably said CPR is CroCPR (SEQ ID NO: 68), said CYB5 is CroCYB5 (SEQ ID NO: 69), said THAS is CroTHAS1 (SEQ ID NO: 83), said HYS is CroHYS (SEQ ID NO: 84) and/or said SS is CroSS (SEQ ID NO: 110) or functional variants thereof having at least 70% homology, similarity or identity to SEQ ID NO: 68, SEQ ID NO: 69, SEQ ID NO: 83, SEQ ID NO: 84 and/or SEQ ID NO: 110, respectively.
4. The microorganism according to any one of the preceding items, further expressing:
   a) a NADPH-cytochrome P450 reductase (CPR, EC 1.6.2.4);
      a cytochrome b5 (CYB5, EC 1.6.2.2);
      a precondylocarpine acetate synthase/O-acetylstemmadenine oxidase (PAS/ASO, EC 1.21.3.-);
      a dihydroprecondylocarpine acetate synthase (DPAS, EC 1.1.1.-);
      a tabersonine synthase (TS, EC 4.-.-.-); and/or
      a catharanthine synthase (CS, EC 4.-.-.-),
   whereby the microorganism is further capable of producing tabersonine, halogenated tabersonine, catharanthine and/or halogenated catharanthine and/or derivatives thereof,
wherein preferably said CPR is CroCPR (SEQ ID NO: 68), said CYB5 is CroCYB5 (SEQ ID NO: 69), said PAS/ASO is CroPAS (SEQ ID NO: 95), AhuASO (SEQ ID NO: 96), TibPAS2 (SEQ ID NO: 97) and/or TelASO (SEQ ID NO: 98), said DPAS is CroDPAS (SEQ ID NO: 99) and/or TibDPAS2 (SEQ ID NO: 100), said TS is CroTS (SEQ ID NO: 101) and/or said CS is CroCS (SEQ ID NO: 102) or functional variants thereof having at least 70% homology, similarity or identity to SEQ ID NO: 68, SEQ ID NO: 69, SEQ ID NO: 95, SEQ ID NO: 96, SEQ ID NO: 97, SEQ ID NO: 98, SEQ ID NO: 99, SEQ ID NO: 100, SEQ ID NO: 101 and/or SEQ ID NO: 102, respectively, and/or
   b) a NADPH-cytochrome P450 reductase (CPR, EC 1.6.2.4);
      a cytochrome b5 (CYB5, EC 1.6.2.2);
      a tabersonine 16-hydroxylase (T16H, EC 1.14.14.103);
      a tabersonine 16-O-methyltransferase (16OMT, EC 2.1.1.94);
      a tabersonine 3-oxygenase (T3O, EC 1.14.14.50);
      a 16-methoxy-2,3-dihydro-3-hydroxytabersonine synthase (T3R, EC 1.1.99.41);
      a 3-hydroxy-16-methoxy-2,3-dihydrotabersonine N-methyltransferase (NMT, EC 2.1.1.99);
      a deacetoxyvindoline 4-hydroxylase (D4H, EC 1.14.11.20); and/or
      a deacetylvindoline O-acetyltransferase (DAT, EC 2.3.1.107),
   whereby the microorganism is further capable of producing vindoline and/or halogenated vindoline and/or derivatives thereof,
wherein preferably said CPR is CroCPR (SEQ ID NO: 68), said CYB5 is CroCYB5 (SEQ ID NO: 69), said TH16 is CroT16H2 (SEQ ID NO: 103), said 16OMT is Cro16OMT (SEQ ID NO: 104), said T3O is CroT3O (SEQ ID NO: 105), said T3R is CroT3R (SEQ ID NO: 106), said NMT is CroNMT (SEQ ID NO: 107), said D4H is CroD4H (SEQ ID NO: 108) and/or said DAT is CroDAT (SEQ ID NO: 109) or functional variants thereof having at least 70% homology, similarity or identity to SEQ ID NO: 68, SEQ ID NO: 69, SEQ ID NO: 103, SEQ ID NO: 104, SEQ ID NO: 105, SEQ ID NO: 106, SEQ ID NO: 107, SEQ ID NO: 108 and/or SEQ ID NO: 109, respectively.
5. The microorganism according to any one of the preceding items, wherein the SGD and the GS are localised in the same cellular compartment and/or cellular membrane such as in the ER lumen, ER membrane, the cytoplasmic site of the ER membrane, mitochondrial lumen, nucleus, peroxisomal lumen, vacuole and/or any other cellular compartment, preferably in the ER lumen, the cytoplasmic site of the ER membrane and/or the mitochondrial lumen,
wherein preferably said SGD is ATP9sp-RseSGD (SEQ ID NO: 113), RseSGD-CNE1sp (SEQ ID NO: 115), RseSGD-CYB5sp (SEQ ID NO: 117), RseSGD-ePTS1sp (SEQ ID NO: 119), RseSGD-CPYsp (SEQ ID NO: 121) and/or SV40sp-RseSGD (SEQ ID NO: 123), and/or said GS is ATP9sp-CroGS (SEQ ID NO: 114), CroGS-CNE1sp (SEQ ID NO: 116) and/or CroGS-CYB5sp (SEQ ID NO: 118), CroGS-ePTS1sp (SEQ ID NO: 120), CroGS-CPYsp (SEQ ID NO: 122) and/or SV40sp-CroGS (SEQ ID NO: 124) or functional variants thereof having at least 70% homology, similarity or identity to SEQ ID NO: 113, SEQ ID NO: 115, SEQ ID NO: 117, SEQ ID NO: 119, SEQ ID NO: 121, SEQ ID NO: 123, SEQ ID NO: 114, SEQ ID NO: 116, SEQ ID NO: 118, SEQ ID NO: 120, SEQ ID NO: 122 and/or SEQ ID NO: 124, respectively,
preferably said SGD is ATP9sp-RseSGD (SEQ ID NO: 113), RseSGD-CNE1sp (SEQ ID NO: 115) and/or RseSGD-CYB5sp (SEQ ID NO: 117), and/or said GS is ATP9sp-CroGS (SEQ ID NO: 114), CroGS-CNE1sp (SEQ ID NO: 116) and/or CroGS-CYB5sp (SEQ ID NO: 118) or functional variants thereof having at least 70% homology, similarity or identity to SEQ ID NO: 113, SEQ ID NO: 115, SEQ ID NO: 117, SEQ ID NO: 114, SEQ ID NO: 116 and/or SEQ ID NO: 118, respectively,
and/or wherein the microorganism further overexpresses an NADH kinase specific to said cellular compartment, preferably a mitochondrial NADH kinase such as POS5 as set forth in SEQ ID NO: 87 or a functional variant thereof having at least 70% homology, similarity or identity thereto.
6. The microorganism according to any one of the preceding items, wherein the halogenated indole is substituted with at least one, such as at least two, such as at least three, such as at least four halogen atoms, each halogen atom independently selected from the group consisting of fluorine, bromine and chlorine, and/or wherein the halogenated indole is 4-halogenated, 5-halogenated, 6-halogenated or 7-halogenated by a halogen selected from the group consisting of fluorine, chlorine and bromine,
preferably wherein the halogenated indole is selected from the group consisting of fluoroindole, chloroindole and bromoindole,
and/or preferably
   wherein the halogenated indole is selected from the group consisting of 4-fluoroindole, 5-fluoroindole, 6-fluoroindole, 7-fluoroindole, 4-chloroindole, 5-chloroindole, 6-chloroindole, 7-chloroindole, 4-bromoindole, 5-bromoindole, 6-bromoindole and 7-bromoindole,
   or
   wherein the halogenated indole is 4,5-dihalogenated, 4,6-dihalogenated, 4,7-dihalogenated, 5,6-dihalogenated, 5,7-dihalogenated and/or 6,7-dihalogenated by two halogen atoms, each halogen atom independently selected from the group consisting of fluorine, bromine and chlorine,
   or
   wherein the halogenated indole is 4,5,6-trihalogenated, 4,5,7-trihalogenated, 4,6,7-trihalogenated and/or 5,6,7-trihalogenated by three halogen atoms, each halogen atom independently selected from the group consisting of fluorine, bromine and chlorine,
   or
   wherein the halogenated indole is 4,5,6,7-tetrahalogenated by four halogen atoms, each halogen atom independently selected from the group consisting of fluorine, bromine and chlorine.
7. The microorganism according to any one of the preceding items, wherein the halogenated strictosidine, strictosidine aglycone, alstonine, serpentine, catharanthine, tabersonine, vindoline, stemmadenine acetate and/or derivatives thereof such as tryptophan, tryptamine, strictosidine, strictosidine aglycone, alstonine, serpentine, catharanthine, tabersonine, vindoline, stemmadenine acetate is substituted with at least one, such as at least two, such as at least three, such as at least four halogen atoms, each halogen atom independently selected from the group consisting of fluorine, bromine and chlorine,
preferably wherein the halogenated strictosidine, strictosidine aglycone, alstonine, serpentine, catharanthine, tabersonine, stemmadenine acetate and/or derivatives thereof is:
   a) 4-halogenated, 5-halogenated, 6-halogenated and/or 7-halogenated by a halogen atom selected from the group consisting of fluorine, bromine and chlorine, and/or the halogenated vindoline and/or derivatives thereof is 4-halogenated, 5-halogenated and/or 7-halogenated by a halogen atom selected from the group consisting of fluorine, bromine and chlorine,
   b) 4,5-dihalogenated, 4,6-dihalogenated, 4,7-dihalogenated, 5,6-dihalogenated, 5,7-dihalogenated and/or 6,7-dihalogenated by two halogen atoms, and/or the halogenated vindoline and/or derivatives thereof is 4,5-dihalogenated, 4,7-dihalogenated and/or 5,7-dihalogenated by two halogen atoms, each halogen atom independently selected from the group consisting of fluorine, bromine and chlorine,
   c) 4,5,6-trihalogenated, 4,5,7-trihalogenated, 4,6,7-trihalogenated and/or 5,6,7-trihalogenated by three halogen atoms, and/or the halogenated vindoline and/or derivatives thereof is 4,5,7-trihalogenated by three halogen atoms, each halogen atom independently selected from the group consisting of fluorine, bromine and chlorine, and/or
   d) 4,5,6,7-tetrahalogenated by at least four halogen atoms, each halogen atom independently selected from the group consisting of fluorine, bromine and chlorine,
preferably with a titer of at least 1 μg/L, or more.
8. The microorganism according to any one of the preceding items, wherein the microorganism is a yeast, preferably wherein the genus of said yeast is selected from the group consisting of Saccharomyces, Pichia, Komagataella, Yarrowia, Kluyveromyces, Candida, Rhodotorula, Rhodosporidium, Cryptococcus, Trichosporon and Lipomyces, such as a yeast selected from the group consisting of Saccharomyces cerevisiae, Saccharomyces boulardii, Komagataella phaffi (Pichia pastoris), Kluyveromyces marxianus, Cryptococcus albidus, Lipomyces lipofera, Lipomyces starkeyi, Rhodosporidium toruloides, Rhodotorula glutinis, Trichosporon pullulan and Yarrowia lipolytica, preferably the yeast is Saccharomyces cerevisiae,
or wherein the microorganism is a bacterium, preferably wherein the genus of said bacterium is selected from the group consisting of Escherichia, Corynebacterium, Pseudomonas, Bacillus, Streptomyces, Lactococcus, Lactobacillus, Halomonas, Bifidobacterium and Enterococcus, such as selected from the group consisting of Escherichia coli, Corynebacterium glutamicum, Pseudomonas putida, Bacillus subtilis, Streptomyces albus, Lactococcus bacillus, Halomonas elongate, Bifidobacterium infantis and Enterococcus faecal, preferably the bacterium is Escherichia coli.
9. The microorganism according to any one of the preceding items comprising one or more nucleic acid sequences encoding the GPPS, GES, G8H, CPR, CYB5, 8HGO, ISY, CYC, IO, CYPADH, 7DLGT, 7DLH, LAMT, SLS, TDC, STR, SGD, THAS, HYS, SS, GS, GO, protein Redox1, protein Redox2, SAT, PAS/ASO, DPAS, TS, CS, T16H, 16OMT, T3O, T3R, NMT, D4H, DAT, TRP, tryptophan halogenase and/or flavin reductase,
wherein said one or more nucleic acids are codon-optimised for the microorganism,
and/or wherein said one or more nucleic acids are:
   i. present in high copy number, in low copy number and/or combinations thereof;
   ii. under the control of an inducible promoter, a constitutive promoter and/or combinations thereof;
   iii. comprised in one or more vectors; and/or
   iv. integrated into the genome of the microorganism according to any one of the items 1 to 8.
10. A method of producing strictosidine aglycone, such as halogenated strictosidine aglycone, and optionally derivatives thereof in a microorganism, said method comprising the steps of;
   i. providing a microorganism, said microorganism expressing:
      a geraniol synthase (GES, EC 3.1.7.11) and
      a strictosidine-O-β-D-glucosidase (SGD, EC 3.2.1.105);
   ii. incubating said microorganism in a medium comprising a substrate and optionally a halogen atom source, which can be converted to strictosidine aglycone and/or halogenated strictosidine aglycone by said microorganism in the presence of geranyl diphosphate and tryptamine and/or halogenated tryptamine;
   iii. optionally recovering the strictosidine aglycone;
   iv. optionally further converting the strictosidine aglycone to one or more monoterpene indole alkaloids (MIAs),
      wherein said GES is capable of converting geranyl diphosphate to geraniol, optionally said GES is a heterologous GES, preferably said GES is as set forth in SEQ ID NO: 65 and/or in SEQ ID NO: 66; and/or
      wherein said SGD is capable of converting strictosidine and/or halogenated strictosidine to strictosidine aglycone and/or halogenated strictosidine aglycone, respectively, optionally said SGD is a heterologous SGD, preferably said SGD is RseSGD (SEQ ID NO: 82), or functional variants thereof having at least 70% homology, similarity or identity to SEQ ID NO: 65, SEQ ID NO: 66 and/or SEQ ID NO: 82, respectively,
      optionally wherein the method comprises step iv. and wherein said microorganism is as defined in any one of items 1 to 9.
11. The method according to item 10, wherein the method comprises step iv. and wherein the one of more MIAs comprise stemmadenine acetate and/or halogenated stemmadenine acetate, said microorganism further expressing:
   a GPPS, a G8H, a CPR, a CYB5, a 8HGO, an ISY, an CYC, an 10, a CYPADH, a 7DLGT, a 7DLH, a LAMT, a TDC, a SLS, a STR, a GS, a GO, a protein Redox1, a protein Redox2 and/or a SAT,
wherein preferably said GPPS is AgrGPPS2 (SEQ ID NO: 63) and/or GgaFPS*(N144W) (SEQ ID NO: 64), said G8H is CroG8H (SEQ ID NO: 67), said CPR is CroCPR (SEQ ID NO: 68), said CYB5 is CroCYB5 (SEQ ID NO: 69), said 8HGO is Vmi8HGOA (SEQ ID NO: 70), said ISY is NcalSY (SEQ ID NO: 71), said CYC is NcaMLPLA (SEQ ID NO: 72), said IO is CroIO (SEQ ID NO: 73), said CYPADH is CroCYPADH (SEQ ID NO: 74), said 7DLGT is Cro7DLGT (SEQ ID NO: 75), said 7DLH is Cro7DLH (SEQ ID NO: 76), said LAMT is CroLAMT (SEQ ID NO: 77), said TDC is CroTDC (SEQ ID NO: 79) and/or rgnTDC (SEQ ID NO: 80), said SLS is CroSLS (SEQ ID NO: 78), said STR is CroSTR (SEQ ID NO: 81), said GS is CroGS (SEQ ID NO: 85) and/or CroADH13 (SEQ ID NO: 86), said GO is CroGO (SEQ ID NO: 88), TelGO (SEQ ID NO: 89), VmiGO (SEQ ID NO: 90), and/or AhuGO (SEQ ID NO: 91), said protein Redox1 is CroRdx1 (SEQ ID NO: 92), said protein Redox2 is CroRdx2 (SEQ ID NO: 93) and/or said SAT is CroSAT (SEQ ID NO: 94) or functional variants thereof having at least 70% homology, similarity or identity to SEQ ID NO: 63, 64, 67, 68, 69, 70, 71, 72, 73, 74, 75, 76, 77, 78, 81, 85, 86, 68, 69, 88, 89, 90, 91, 92, 93 and/or 94, respectively.
12. The method according to item 11, wherein the microorganism further expresses a tryptophan halogenase (EC 1.14.19.-) and optionally a flavin reductases such as FMN reductase (NADPH) (EC 1.5.1.38), wherein the tryptophan halogenase is capable of converting tryptophan to a halogenated tryptophan,
wherein preferably said tryptophan halogenase is IaeRebH_N470S (SEQ ID NO: 111) and/or said FMN reductase (NADPH) is EcoSsuE (SEQ ID NO: 112) or functional variants thereof having at least 70% homology, similarity or identity to SEQ ID NO: 111 and/or SEQ ID NO: 112, respectively, whereby the microorganism is further capable of producing halogenated tryptophan in the presence of tryptophan and at least one halogen atom, wherein the halogenated tryptophan is a tryptophan substituted with one, two, three or four halogen atoms, wherein each halogen atom is independently selected from the group consisting of fluorine, chlorine and bromine,
and/or wherein the halogenated stemmadenine acetate is 4-fluorostemmadenine acetate, 5-fluorostemmadenine acetate, 6-fluorostemmadenine acetate, 7-fluorostemmadenine acetate, 4-bromostemmadenine acetate, 5-bromostemmadenine acetate, 6-bromostemmadenine acetate, 7-bromostemmadenine acetate, 4-chlorostemmadenine acetate, 5-chlorostemmadenine acetate, 6-chlorostemmadenine acetate, 7-chlorostemmadenine acetate and/or derivatives thereof, preferably said halogenated stemmadenine acetate and/or derivatives thereof is 4-fluorostemmadenine acetate, 6-fluorostemmadenine acetate, 7-fluorostemmadenine acetate and/or derivatives thereof
and/or wherein the medium further comprises at least:
   i. halogenated indole as defined in item 6, preferably at a concentration of at least 0.05 mM, such as at least 0.1 mM, such as at least 0.5 mM, such as at least 1 mM, such as at least 2 mM or more;
   ii. tryptamine and/or halogenated tryptamine, preferably at a concentration of at least 0.05 mM, such as at least 0.1 mM, such as at least 0.5 mM, such as at least 1 mM, such as at least 2 mM or more;
   iii. a halogen atom source such as a salt, preferably NaCl and/or KBr, preferably at a concentration of at least 0.05 M, such as at least 0.1 M, such as at least 0.5 M or more;
   iv. tryptophan and/or halogenated tryptophan, preferably at a concentration of at least 0.05 mM, such as at least 0.1 mM, such as at least 0.5 mM, such as at least 1 mM, such as at least 2 mM or more;
   v. secologanin, preferably at a concentration of at least 0.05 mM, such as at least 0.1 mM, such as at least 0.5 mM, such as at least 1 mM such as at least 2 mM or more,
or combinations thereof.
13. The method according to any one of items 10 to 12, further comprising one or more of the steps of:
   i. recovering the MIA and/or derivatives thereof;
   ii. converting said MIA and/or derivatives thereof to derivatives thereof and/or downstream products thereof, respectively; and/or
   iii. formulating said MIA and/or derivatives thereof and/or downstream products thereof in a composition such as a pharmaceutical composition,
optionally
wherein the microorganism is as defined in item 3 d), and wherein the one or more MIAs comprise alstonine, halogenated alstonine, serpentine and/or halogenated serpentine and/or derivatives thereof,
and/or wherein the microorganism is as defined in item 4 a), and wherein the one or more MIAs comprise tabersonine, halogenated tabersonine, catharanthine and/or halogenated catharanthine and/or derivatives thereof,
and/or wherein the microorganism is as defined in item 4 b), and wherein the one or more MIAs comprise vindoline and/or halogenated vindoline and/or derivatives thereof.
14. A method for manufacturing a monoterpene indole alkaloid (MIA) and/or a halogenated MIA and/or derivatives thereof of interest, said method comprising the steps of:
   i. providing a MIA and/or a halogenated MIA and/or derivatives thereof; and
   ii. optionally converting said MIA and/or halogenated MIA and/or derivatives thereof to the MIA and/or halogenated MIA and/or derivatives thereof of interest.
15. A method of treating a disorder such as a cancer, arrhythmia, malaria, fibrosis, pain, anxiety, Parkinson's disease, schizophrenia, bipolar disorder, psychotic diseases or disorders, hypertension, depression, Alzheimer's disease, addiction neuronal diseases, and/or withdrawal symptoms, comprising administration of a therapeutic sufficient amount of a MIA, a halogenated MIA and/or a pharmaceutical compound obtained by the method according to any one of items 10 to 13.

1-54. (canceled)

55. A microorganism producing halogenated strictosidine aglycone or derivatives thereof in the presence of geranyl diphosphate (GPP) and halogenated tryptamine, said microorganism expressing a geraniol synthase (GES, EC 3.1.7.11) and a strictosidine-O-β-D-glucosidase (SGD, EC 3.2.1.105), wherein the GES is a GES capable of converting GPP to geraniol and the SGD is capable of converting halogenated strictosidine to halogenated strictosidine aglycone.

56. The microorganism according to claim 55, wherein said microorganism expresses a tetrahydroalstonine synthase and a serpentine synthase (SS).

57. The microorganism according to claim 56, wherein said microorganism produces one or more of the following halogenated alstonines:
a) alstonine that is 6-halogenated, 4-halogenated, 5-halogenated, or 7-halogenated, by one halogen atom, or
b) alstonine that is 4,5-dihalogenated, 4,6-dihalogenated, 4,7-dihalogenated, 5,6-dihalogenated, 5,7-dihalogenated or 6,7-dihalogenated, by two halogen atoms, or
c) alstonine that is 4,5,6-trihalogenated, 4,5,7-trihalogenated, 4,6,7-trihalogenated or 5,6,7-trihalogenated by three halogen atoms, or
d) alstonine that is 4,5,6,7-tetrahalogenated by at least four halogen atoms, or
e) 6-fluoroalstonine, 5-fluoroalstonine, 4-fluoroaltsonine, 7-fluoroalstonine, 5,6-difluoroalstonine, 6,7-difluoroalstonine, 7-chloroalstonine or 7-bromoalstonine,
wherein each of said halogen atoms of i.-iv. is independently selected from fluorine, bromine and chlorine.

58. The microorganism according to claim 56, further expressing a NADPH-cytochrome P450 reductase (CPR, EC 1.6.2.4), a cytochrome b5 (CYB5, EC 1.6.2.2), or a CPR and a CYB5.

59. The microorganism according to claim 55, wherein:
a) the GES is a heterologous GES, a GES as set forth in SEQ ID NO: 65, or a GES as set forth in SEQ ID NO: 66, or a functional variant of SEQ ID NOs: 65 or 66 having at least 70% homology thereto; or
b) the SGD is a heterologous SGD, RseSGD as set forth in SEQ ID NO: 82, ATP9sp-RseSGD as set forth in SEQ ID NO: 113, RseSGD-CNE1sp as set forth in SEQ ID NO: 115, RseSGD-CYB5sp as set forth in SEQ ID NO: 117, RseSGD-ePTS1sp as set forth in SEQ ID NO: 119, RseSGD-CPYsp as set forth in SEQ ID NO: 121, SV40sp-RseSGD as set forth in SEQ ID NO: 123, or a functional variant of SEQ ID NOs: 82, 113, 115, 117, 119, 121 or 123 having at least 70% homology thereto.

60. The microorganism according to claim 56, wherein:
a) the THAS is CroTHAS1 as set forth in SEQ ID NO: 83, or a functional variant thereof having at least 70% homology to SEQ ID NO: 83; or
b) the SS is CroSS as set forth in SEQ ID NO: 110 or a functional variant thereof having at 70% homology to SEQ ID NO: 110.

61. The microorganism according to claim 55, further expressing;
a geraniol-8-hydroxylase (G8H, EC 1.14.14.83); or
a NADPH-cytochrome P450 reductase (CPR, EC 1.6.2.4); or
a cytochrome b5 (CYB5, EC 1.6.2.2); or
a 8-hydroxygeraniol oxidoreductase (8HGO, EC 1.1.1.324); or
an iridoid synthase (ISY, EC 1.3.1.122); or
an iridoid cyclase (CYC, EC 5.5.1.34); or
an iridoid oxidase (IO, EC 1.14.14.161); or
a CYP enzyme assisting alcohol dehydrogenase (CYPADH, EC 1.1.1.-); or
a 7-deoxyloganetic acid glucosyl transferase (7DLGT, EC 2.4.1.323); or
a 7-deoxyloganic acid hydroxylase (7DLH, EC 1.14.14.85); or
a loganic acid O-methyltransferase (LAMT, EC 2.1.1.50); or
a secologanin synthase (SLS, EC 1.14.19.62); or
a strictosidine synthase (STR, EC 4.3.3.2).

62. The microorganism according to claim 55, wherein the microorganism is a yeast, bacterium, archaeon, fungus, protozoan, alga, or virus, or wherein the microorganism is a yeast of the genus Saccharomyces, Pichia, Komagataella, Yarrowia, Kluyveromyces, Candida, Rhodotorula, Rhodosporidium, Cryptococcus, Trichosporon or Lipomyces.

63. The microorganism according to claim 55, wherein the halogenated tryptamine is 6-fluorotryptamine, 5-fluorotryptamine, 4-fluorotryptamine, 7-fluorotryptamine, 5,6-difluorotryptamine, 6,7-difluorotryptamine, 7-chlorotryptamine or 7-bromotryptamine.

64. A method of producing halogenated strictosidine aglycone or halogenated strictosidine aglycone and derivatives thereof in a microorganism, said method comprising the steps of:
a) providing a microorganism as defined in claim 55;
b) incubating said microorganism in a medium comprising a substrate and a halogen atom source, which can be converted to halogenated strictosidine aglycone by said microorganism in the presence of GPP and halogenated tryptamine.

65. The method according to claim 64, wherein the halogenated tryptamine is 6-fluorotryptamine, 5-fluorotryptamine, 4-fluorotryptamine, 7-fluorotryptamine, 5,6-difluorotryptamine, 6,7-difluorotryptamine, 7-chlorotryptamine or 7-bromotryptamine.

66. The method according to claim 64, wherein the microorganism expresses
a) a THAS and a SS; or
b) a CPR and a CYB5; or
c) a THAS, a SS, a CPR and a CYB5.

67. A fermentation liquid comprising one of more derivatives of halogenated strictosidine aglycone, wherein said fermentation liquid is obtained by a method according to claim 64.

68. A fermentation liquid comprising one of more derivatives of halogenated strictosidine aglycone, wherein said fermentation liquid comprised within or secreted from the microorganism as defined in claim 55.

69. A composition comprising one of more derivatives of halogenated strictosidine aglycone.

70. The composition according to claim 69, wherein said one or more derivatives are one or more of the following halogenated alstonines:
a) alstonine that is 6-halogenated, 4-halogenated, 5-halogenated, or 7-halogenated, by one halogen atom, or
b) alstonine that is 4,5-dihalogenated, 4,6-dihalogenated, 4,7-dihalogenated, 5,6-dihalogenated, 5,7-dihalogenated or 6,7-dihalogenated, by two halogen atoms, or
c) alstonine that is 4,5,6-trihalogenated, 4,5,7-trihalogenated, 4,6,7-trihalogenated or 5,6,7-trihalogenated by three halogen atoms, or
d) alstonine that is 4,5,6,7-tetrahalogenated by at least four halogen atoms, or
e) 6-fluoroalstonine, 5-fluoroalstonine, 4-fluoroaltsonine, 7-fluoroalstonine, 5,6-difluoroalstonine, 6,7-difluoroalstonine, 7-chloroalstonine or 7-bromoalstonine,
wherein each of said halogen atoms of i.-iv. is independently selected from fluorine, bromine and chlorine.

71. Halogenated strictosidine aglycone or a derivative thereof.

72. The halogenated strictosidine aglycone or derivative thereof according to claim 71, wherein said derivative is one or more of the following halogenated alstonines:
a) alstonine that is 6-halogenated, 4-halogenated, 5-halogenated, or 7-halogenated, by one halogen atom, or
b) alstonine that is 4,5-dihalogenated, 4,6-dihalogenated, 4,7-dihalogenated, 5,6-dihalogenated, 5,7-dihalogenated or 6,7-dihalogenated, by two halogen atoms, or
c) alstonine that is 4,5,6-trihalogenated, 4,5,7-trihalogenated, 4,6,7-trihalogenated or 5,6,7-trihalogenated by three halogen atoms, or
d) alstonine that is 4,5,6,7-tetrahalogenated by at least four halogen atoms, or
e) 6-fluoroalstonine, 5-fluoroalstonine, 4-fluoroaltsonine, 7-fluoroalstonine, 5,6-difluoroalstonine, 6,7-difluoroalstonine, 7-chloroalstonine or 7-bromoalstonine,
wherein each of said halogen atoms of i.-iv. is independently selected from fluorine, bromine and chlorine.

73. A method of treating a cancer, arrhythmia, malaria, fibrosis, pain, psychotic diseases or disorders, hypertension, anxiety, depression, Alzheimer's disease, Parkinson's disease, schizophrenia, bipolar disorder, addiction, neuronal diseases, or withdrawal symptoms, comprising administration of a therapeutically effective amount of a halogenated strictosidine aglycone or a derivative thereof according to claim 71, to a subject in a need thereof.

74. The method according to claim 73, wherein said derivative is one or more of the following halogenated alstonines:
a) alstonine that is 6-halogenated, 4-halogenated, 5-halogenated, or 7-halogenated, by one halogen atom, or
b) alstonine that is 4,5-dihalogenated, 4,6-dihalogenated, 4,7-dihalogenated, 5,6-dihalogenated, 5,7-dihalogenated or 6,7-dihalogenated, by two halogen atoms, or
c) alstonine that is 4,5,6-trihalogenated, 4,5,7-trihalogenated, 4,6,7-trihalogenated or 5,6,7-trihalogenated by three halogen atoms, or
d) alstonine that is 4,5,6,7-tetrahalogenated by at least four halogen atoms, or
e) 6-fluoroalstonine, 5-fluoroalstonine, 4-fluoroaltsonine, 7-fluoroalstonine, 5,6-difluoroalstonine, 6,7-difluoroalstonine, 7-chloroalstonine or 7-bromoalstonine,
wherein each of said halogen atoms of i.-iv. is independently selected from fluorine, bromine and chlorine.


SEQUENCE LISTING (154 sequences)

SEQ ID NO: 1 - DNA, 1146 bp, Abies grandis
  source 1..1146: mol_type = other DNA
atggcttactccgcgatggcgactatgggatacaatggtatggcggcgtcatgtcatactttacacccgacttcaccattgaagccatttcatggagcgtcaacgtctttggaggctttcaatggtgaacatatgggattattgagagggtacagcaaaagaaaattgtcttcatacaagaatccagcatcaagatcatctaatgctaccgtcgcccaacttttgaatccgccccagaaaggcaagaaggccgtggaattcgactttaataaatatatggattccaaagcaatgactgtgaatgaggctttaaataaggcaattccgttaagatatccacagaaaatatacgaaagtatgaggtattccttattggccggtgggaaaagagtaagacctgtattgtgcatagctgcatgtgaattagttggtggtaccgaagaacttgcaatccctacagcgtgtgctatagaaatgatacatactatgagcttgatgcacgatgatttgccatgcatcgataacgatgatttgaggaggggtaaacccacaaatcacaaaatattcggtgaagatactgccgttacagcaggtaacgcattgcactcctatgcgttcgaacacattgctgtatctacaagcaagactgttggtgcagatagaatattaagaatggtttctgaattaggtcgtgcaacaggttctgaaggagtaatgggcggtcaaatggtcgacattgcaagtgaaggggatccttcaatcgatcttcaaactttggagtggatacatattcacaagaccgctatgctgttagagtgttctgtggtgtgtggtgccataataggtggtgcttctgagatagtcattgaacgtgctaggagatatgccagatgtgttggtctattatttcaagtcgtagatgatatcttagacgttaccaaatcttcagatgaacttggtaagactgctggcaaggatttgatttccgataaggcaacctaccctaaattgatggggctggagaaagcaaaggaatttagtgacgaattacttaacagagcaaagggtgaactaagctgctttgaccctgtgaaggcagcacctttacttggtttagccgactacgttgcattcagacaaaattaa

SEQ ID NO: 2 - DNA, 1104 bp, Gallus gallus
  source 1..1104: mol_type = other DNA
atgcataagtttactggtgtcaacgccaaattccagcagccagcattaagaaacctgtcaccagtagtagtcgaacgtgaaagggaggaatttgtgggtttttttccacagatcgtcagagatcttacggaggatggtattggtcatccagaagtgggagatgctgtcgcacgtctaaaggaagttttacaatataacgctccaggtggtaagtgcaatagaggtttaaccgtcgtcgcagcttatagggagctatctggtccaggtcaaaaagatgccgaatctctgaggtgtgctctagccgtcggatggtgtatagagctgtttcaggcgttctttttagtcgccgacgatattatggaccagtcattgacccgtagaggtcaattgtgttggtataaaaaagaaggggtcggtctagacgcaatttgggactcctttttgttggaatcctcagtttatagagtgttaaaaaaatattgtaggcagaggccctattatgtacatttattagagttatttctacaaaccgcgtaccagactgaactagggcaaatgctagacctgattaccgcaccagtttctaaagttgatttgtctcattttagtgaggaaaggtacaaagctatcgtgaagtacaaaactgccttttactcattttatttaccagttgcagccgcaatgtatatggtcggcatcgattctaaggaagaacatgaaaacgctaaagcaattctgttagaaatgggcgagtatttccagattcaagatgactacttggactgtttcggcgatcccgctcttacaggtaaggtcggaaccgatattcaggacaataaatgcagttggctagttgttcaatgtctacaaagagttacaccagaacagaggcaattgcttgaagataattatgggagaaaggaaccagaaaaagtggcaaaggtgaaagaactgtatgaagctgttggaatgagagcagcttttcaacaatacgaagaatcctcatacagaagattgcaagaattaatcgaaaagcactcaaaccgtttgccaaaagaaatcttccttggtttggcacaaaaaatctacaaaagacagaagtaa

SEQ ID NO: 3 - DNA, 1623 bp, Catharanthus roseus
  source 1..1623: mol_type = other DNA
atgtcatccatgtcactaccactggccactcctttgattaaagacaatgagtcattaatcaagtttcttcgtcaaccattagtactaccgcacgaagtggatgattccacaaagaggagagaattacttgaaaggacaaggaaggaattggaattaaacgctgaaaagccattagaagcacttaaaatgatcgacattattcaaagactgggattatcctaccattttgaagacgacataaatagtattttgactggtttcagcaatatctcttctcaaactcacgaagatctattaaccgcatccttgtgttttcgtttgcttagacacaacggtcataaaataaaccctgatatcttccaaaaatttatggataacaatggcaaatttaaggatagtctaaaggatgatactctagggatgctgtctttgtatgaagcttcctatctgggcgcaaacggtgaagaaatccttatggaagcacaagaatttactaagacgcaccttaaaaattctttgcctgccatggcacctagtctatcaaagaaagtttctcaggccttggagcaacctagacacaggaggatgctaagactagaagcaagaagatttattgaagagtatggtgctgaaaatgatcataatcccgaccttttagagcttgcaaagctggactacaataaagtacagtcattgcatcaaatggagctatcagagattacccgttggtggaaacaacttggattggttgataaactaacattcgctagagatagaccattggaatgtttcctatggacagtaggacttctgcctgagccaaaatatagcggttgtagaatcgaactagcaaaaacaatcgctattttactagtgattgatgatatttttgatactcatggcactttagatgaactgttattgttcaccaatgcgattaaacgttgggacttagaagccatggaagatttaccagaatatatgagaatctgttacatggccttatataatactactaatgaaatctgttataaagttttgaaagagaatgggtggagtgtattgccttatttaaaagctacatggattgatatgattgaagggtttatggttgaagccgaatggtttaactctgactacgtgccgaacatggaagaatatgtcgagaacggtgtaagaacggctggtagctatatggcgttggtccatttgttctttttgataggacagggtgttacagaggacaatgtgaagctattaatcaaaccatacccgaaattatttagttcaagcgggagaatcttaagattatgggacgacttgggtaccgctaaagaagaacaagagaggggggacttggccagctccattcaactgttcatgagagaaaaagaaatcaagtccgaggaggaaggtagaaaggggattttagaaattatagaaaatttgtggaaagaattgaacggtgagttagtgtacagagaagaaatgccattagcgattattaaaactgcgtttaatatggcaagagccagccaagttgtctatcagcacgaagaagacacttatttctccagtgttgataattacgtcaaggctttgttcttcaccccttgtttttaa

SEQ ID NO: 4 - DNA, 2691 bp, synthetic construct
  misc_feature 1..2691: note = Artificial Sequence
  misc_feature 1..2691: note = Artificial Sequence
  source 1..2691: mol_type = other DNA
atggcttcagaaaaagaaattaggagagagagattcttgaacgttttccctaaattagtagaagagttaaatgcatccttattagcttacggtatgcctaaggaagcatgtgactggtatgcccactcattgaactacaacactccaggcggtaagttaaatagaggtttgtccgttgtggacacgtatgctattctctccaacaagaccgttgaacaattggggcaagaagaatacgaaaaggttgctattctaggttggtgcattgagttgttgcaggcttactggttggtcgccgatgatatgatggacaagtccattaccagaagaggccaaccatgttggtacaaggttcctgaagttggggaaattgccatctgggacgcattcatgttagaggctgctatctacaagcttttgaaatctcacttcagaaacgaaaaatactacatagatatcaccgaattgttccatgaagtcaccttccaaaccgaattgggccaattgatggacttaatcactgcacctgaagacaaagtcgacttgagtaagttctcattgaaaaaacattcatttatcgttactttcaagactgcttactattctttctacttgcctgtcgcattggctatgtacgttgccggtatcacagatgaaaaggatttgaaacaagccagagatgtcttgattccattgggtgaatatttccaaattcaagatgactacttagactgcttcggtaccccagaacagatcggtaagatcggtacagatatccaagataacaaatgttcttgggtaatcaacaaggcattagaacttgcttccgcagaacaaagaaagactttagacgaaaattacggtaagaaggactcagtcgcagaagccaaatgcaaaaagattttcaatgacttgaaaatcgaccagttataccacgaatatgaagagtctgttgccaaggatttgaaggccaagatctcccaagtcgacgagtctcgtggcttcaaagccgacgtcttaactgcgtttttgaacaaggtttacaagagaagtaaaggtggaggctctatgtcatccatgtcactaccactggccactcctttgattaaagacaatgagtcattaatcaagtttcttcgtcaaccattagtactaccgcacgaagtggatgattccacaaagaggagagaattacttgaaaggacaaggaaggaattggaattaaacgctgaaaagccattagaagcacttaaaatgatcgacattattcaaagactgggattatcctaccattttgaagacgacataaatagtattttgactggtttcagcaatatctcttctcaaactcacgaagatctattaaccgcatccttgtgttttcgtttgcttagacacaacggtcataaaataaaccctgatatcttccaaaaatttatggataacaatggcaaatttaaggatagtctaaaggatgatactctagggatgctgtctttgtatgaagcttcctatctgggcgcaaacggtgaagaaatccttatggaagcacaagaatttactaagacgcaccttaaaaattctttgcctgccatggcacctagtctatcaaagaaagtttctcaggccttggagcaacctagacacaggaggatgctaagactagaagcaagaagatttattgaagagtatggtgctgaaaatgatcataatcccgaccttttagagcttgcaaagctggactacaataaagtacagtcattgcatcaaatggagctatcagagattacccgttggtggaaacaacttggattggttgataaactaacattcgctagagatagaccattggaatgtttcctatggacagtaggacttctgcctgagccaaaatatagcggttgtagaatcgaactagcaaaaacaatcgctattttactagtgattgatgatatttttgatactcatggcactttagatgaactgttattgttcaccaatgcgattaaacgttgggacttagaagccatggaagatttaccagaatatatgagaatctgttacatggccttatataatactactaatgaaatctgttataaagttttgaaagagaatgggtggagtgtattgccttatttaaaagctacatggattgatatgattgaagggtttatggttgaagccgaatggtttaactctgactacgtgccgaacatggaagaatatgtcgagaacggtgtaagaacggctggtagctatatggcgttggtccatttgttctttttgataggacagggtgttacagaggacaatgtgaagctattaatcaaaccatacccgaaattatttagttcaagcgggagaatcttaagattatgggacgacttgggtaccgctaaagaagaacaagagaggggggacttggccagctccattcaactgttcatgagagaaaaagaaatcaagtccgaggaggaaggtagaaaggggattttagaaattatagaaaatttgtggaaagaattgaacggtgagttagtgtacagagaagaaatgccattagcgattattaaaactgcgtttaatatggcaagagccagccaagttgtctatcagcacgaagaagacacttatttctccagtgttgataattacgtcaaggctttgttcttcaccccttgtttttaa

SEQ ID NO: 5 - DNA, 1482 bp, Catharanthus roseus
  source 1..1482: mol_type = other DNA
atggattaccttacaattatcctaacgttgttgttcgctttaacattgtatgaagctttcagctacttatcacgtagaactaaaaatcttcctcctggaccttccccgttgccatttatcgggtctttgcatctgttaggcgaccaaccccataaatcattggctaagttatctaagaaacatggacctatcatgtctctgaaactaggccaaattacaactatcgttatttcttcttctacaatggctaaagaggtactgcaaaagcaagacttagcattcagttctagatcagtcccaaatgctttgcatgctcataaccagtttaagttttccgtcgtctggttgccggttgcatcaagatggagatctttgagaaaggtgctaaactctaacattttctccggtaacaggttggatgcaaatcaacatttgaggacgcgtaaggtccaagaactaatagcgtactgtagaaagaatagccaaagcggtgaagccgttgacgttggtagggctgcattcagaacctcattgaatttgctgtcaaacttgatctttagtaaggatttaacagatccatattcagactccgcgaaagaatttaaagatttggtatggaatataatggttgaggcgggcaagcccaatttggttgatttttttcctctattagaaaaggtagatccacaaggaataagacatagaatgacaatacattttggagaggtccttaaactttttggaggtttggtaaacgagcgtcttgaacagaggagaagtaagggtgagaaaaatgacgtactggatgttttattaactacttctcaagaatctccggaagagattgataggacccatattgaaagaatgtgtttggatttatttgttgctggcacggatacaacatcttctaccttagaatgggctatgtccgaaatgttaaagaacccagacaaaatgaagaaaacccaagatgaacttgcccaagtcataggtagaggtaagaccattgaagaatcagatataaacaggttaccatacttaagatgtgtgatgaaagagacgttgaggatccacccaccagtaccctttttgataccaagaaaagttgaacagtccgtcgaggtttgcggatacaacgttcctaaaggatcacaagtattagttaatgcttgggcaattggtagagatgaaactgtgtgggatgatgctctggctttcaagccggaaagatttatggaatccgaactagatattagaggcagagatttcgaactgatcccgttcggagcagggagaagaatctgccccgggttgcccttggctctaagaacggttccgttgatgttaggttctttgctaaacagttttaactggaagttagaaggtggtatggctcccaaggatcttgacatggaagaaaaattcggtataactttgcaaaaagcgcatcctttgagggctgtgccctcaactttgtaa

SEQ ID NO: 6 - DNA, 2145 bp, Catharanthus roseus
  source 1..2145: mol_type = other DNA
atggactcatcctccgagaagttgtcaccattcgaacttatgtcagcaattcttaagggagccaagctggacggtagtaacagttctgattccggtgtcgctgtatcacctgctgttatggcaatgttactagaaaataaagagttagtaatgatattgacgacatctgtcgctgtcttgattggttgcgtcgttgtgctaatttggcgtagatcttcagggtccggtaagaaggttgtggagccacccaagttgatagtcccaaaaagtgtagtggagccagaagaaatagatgaaggaaaaaaaaaattcactatcttctttggtacacaaactgggacagctgaaggttttgctaaggctttagccgaagaagcaaaggctagatacgaaaaggcagttataaaagtaatcgatattgacgattatgcagcagacgatgaggagtatgaggaaaaattcagaaaagagactttggccttctttatattggcaacatatggcgatggtgagcctactgataacgctgcaaggttttacaaatggtttgtagagggtaatgatagaggtgactggcttaagaacttacagtatggcgtcttcggtttgggcaatagacagtatgaacatttcaataagattgcaaaagttgtagatgaaaaggttgccgagcaaggagggaagaggatagtgcctttagttttaggagacgatgatcaatgtattgaagatgactttgctgcatggagagaaaacgtctggcctgaactggataatctgctaagagacgaggatgatactacagtgtctactacctatacagccgctataccagaatacagagttgttttccctgataaaagtgattctttgatttctgaggccaacggccacgctaacggctatgcgaacggcaatactgtatacgatgctcaacacccttgccgtagtaacgtcgctgtcaggaaagagttacataccccagcttctgataggtcttgtacacatttggattttgatatagcgggtactggattatcatatggtacaggagatcacgtcggtgtctattgtgacaatttatctgagaccgtagaagaagcagaaaggttgttgaacttgccccctgaaacgtatttcagtttgcacgctgataaagaagatggtactccattagcaggatcatcattgccccctccttttcctccgtgcacattgaggactgcccttactagatatgctgatctgctgaataccccaaaaaagtccgccttgctggctctagctgcttatgcaagcgatccaaatgaggctgatcgtttgaagtacttggcaagcccagctggcaaagacgagtatgctcaatctttggtagctaatcagagaagcctgctagaagtaatggctgaatttccttccgccaagccaccgttaggtgtattcttcgcagcaatagctcccagattacaacccagattctactctatatcttctagcccaaggatggccccttccagaattcatgtcacttgcgctctagtttatgagaaaactccaggcgggaggattcataaaggcgtatgttcaacttggatgaagaacgctattcctctggaggaatctcgtgattgtagctgggcaccgatctttgtgagacagtctaactttaagctgcctgccgatccaaaagttcctgtaatcatgataggtccaggcaccgggctagcaccttttagaggtttccttcaagaaagacttgctctgaaagaggaaggagctgaattaggaactgctgtatttttttttgggtgtaggaacagaaaaatggattatatatatgaagatgaattgaatcatttcttggaaatcggcgcgttatcagaattgctggttgcattcagtagggaaggtcctactaagcaatatgttcaacacaaaatggccgaaaaagccagtgatatttggcgtatgatctctgatggtgcttatgtttatgtctgcggagatgccaagggcatggccagagacgttcataggacattacacactatagctcaagagcaaggatcaatggactctactcaggccgaaggatttgtgaaaaacttacaaatgaccggtagatatttaagagacgtatggtaa

SEQ ID NO: 7 - DNA, 405 bp, Catharanthus roseus
  source 1..405: mol_type = other DNA
atggcctctgatcaaaagttgcataagttcgatgaagtctcaaaacataataaaacgaaagattgttggctgattattaatggtaaggtctacgacgtcactccgtttatggacgatcatccaggtggtgacgaagtcttattatccgccacaggcaaggacgcaacaaatgactttgaagatgttggtcactctgacagcgctagagaaatgatggataaatattacattggtgagatggatatggctactgttccacttaaaagaacatacattcctccacagcaagctcaatataatcctgacaagacaccagagttcgtgattaagatccttcaatttttagtacccttgctgatattgggtttagcgttcgctgttagacattacaccaaggaaaaataa

SEQ ID NO: 8 - DNA, 1128 bp, Vinca minor
  source 1..1128: mol_type = other DNA
atgactaaaatttctccatctgttattacttgtaaagctgctgttatttggaaatctggtgaaccagctaaagttgaagaaattcaagttgatccaccaaaagcttctgaagttagaattaaaatgttgtgtgcttctttgtgtcatactgattttttgtgttgtaatggtttgccagttccattgtttccaagaattccaggtcatgaaggtgttggtatgattgaatctgttggtgaaaatgttactgaattgaaaggtggtgatattgttatgccattgtatttgggtgaatgtggtgaatgtttgaattgtaaatctggtagaactaatttgtgtcataaatatccattgggtttttctggtttgttgttggatggtacttctagaatgtctattggtggtcaaaaaatttatcatcatttttcttgttctacttggtctgaatatattgttattgaagaagcttatgctgttaaagttgatccaagagtttctttgtctcatgcttcttttttgtgttgtggttttactactggttttggtgctacttggagagatgttaatgttgaaaaaggtgctactgttgctgttttgggtttgggtgctgttggtttgggtgctgttcaaggtgctagatctcaaggtgcttcttctattattggtgttgatattaatgataaaaaaagagaaaaaggtgaagcttttggtattactgaatttattaatccaaaaggttctaataaatctgtttctgatttgattaaagaagctactggtggtttgggtgttgattatgtttatgaatgtactggtgttccatctttgttgaatgaagctattgaagcttctaaagttggtttgggtactgctgttttgattggtgctggtttggaaacttctggtgaaattaaatttattccattgttgtgtggtagaactgttaaaggttctatttatggtggtgttagaccaaaatctgatttgccaactattattgaaaaatgtattaataaagaaattccaatggatgctttgttgactcatgaagtttctttgtctgaaattaataaaggtttggaatatttgaaacatccagattgtgttaaagttgttattaaattttaa

SEQ ID NO: 9 - DNA, 1194 bp, Nepeta cataria
  source 1..1194: mol_type = other DNA
atgtctatgaattggtggagagatggtgctgctaaaaaaagaatggatgaatcttctgctgttaaattgcaacaacaacaatgtgttgctttgattgttggtgttactggtttggttggtaattctttggctgaaatgttgccattgtctgatactccaggtggtccttggaaagtttatggtgttgctagaagaccaagaccatcttggaatgaagatcatccaattaattatatttcttgtgatgtttctaatactgctgaagttgaagctaaattgccaccattgtctgatgttactcatattttttatgctacttggacttctagatcttctgaagaagaaaattgtgaagctaatggtaaaatgttgaaaaatgttttggatactatgattccaaattgtccaaatttgaaacatatttgtttgcaaactggtagatttcattatgttgcttctgttgttgattggaaaattaatggttctcatgatactccattgactgaagatttgccaagattgaatactaataatttttattatactcaagaagatattttgttggaagaagttaaaagaaaagaaggtttgacttggtctgttcatagaccaggtactatttttggtttttctccatattctatgatgaatttggttggtactttgtgtgtttatgctgctatttgtaaacaagaaggtgctgttttgagatttccaggttgtaaaggtgcttgggatggttattctgattgtgctgatgctgatttgattgctgaacattatatttgggctgctttagatccacatgctaaaaatcaatcttttaatgtttctaatggtgatgtttttaaatggaaacatttgtggaaagttttggctgaacaatttggtgttgaatgtggtggttatgaatatgaagaaggtcaacaagttagattgcaagatgttatgaaagataaaggtccagtttgggataaaattgttagagaaaatggtttgtctaatactaaattggaagatgttggtaaatggtggttttctgatactattttgtggaatgaatgtagattggattctatgaataaatctaaagaacatggttttttgggttttagaaattctaaaaattgttttttgtattggattcataaattgaaagcttataaaattgttccatcttctactatttcttaa

SEQ ID NO: 10 - DNA, 483 bp, Nepeta cataria
  source 1..483: mol_type = other DNA
atggcttctaaattggaaattgaaattgaattgaaatctgatgttgaaaaaatgtggaaacattttaaagaatttactaaattgtttccaaaagcttgtccacatttgtatgaaaaaattgatgttattcaaggtgatggtatttctgttggtactatttttgtttctactttgaaaccaactgaattgaatccagttgttatggttactaaagaaaaaattgattttttggatgatgataataaaatgttgagatattcttatatggaaggtgaaattttgaaaaattataaaaatttgagaggtattgttcatatgtcttcttctaaaaatggtggtactatttttaaatattctggtgaatttgaaaaagctaatgaacaagttccagatccagttttttttaaagattttatggttattgtttttcaaagattggatgattatgttttgaatggtatgaatcatacttgtcaaaattaa

SEQ ID NO: 11 - DNA, 1548 bp, Catharanthus roseus
  source 1..1548: mol_type = other DNA
atggccacaattacttttgattctcttaaccctgttaccgtagctattagtgcgggatttttattgcttttgatcatattcgtgaaaagtagaacgggtagttctaaaaggaagccgccaggccccccgggttggcctatattcggtaatatgtttgaccttggcgatctaccccaccaaacgttgtataaacttaagtctaaatatggtccgattgtgtggttgcaacttggtagtattaatacgatggttgttcaaaacgccgtgtcagcagcagaattattcaaaaaacatgacgttcctttctgcgataggaaggtaccagatacactaacggcgtttaactttaaccagggttccttgggtatgaatacttatggaggacattggagggttttgagaaggttatgttcaatggagttcctagtaaataagagaatgaatgagacgaccgatttaagaagaagaatcgaagataatatggtaagatggattgaagaagattctctggcatcaaaagcacaaggtgggacaggggcagttcagttatctcgtttccttttcttaatggcgttcaatcttgtcggcaatttgatgctatcccgtgatttgatggacaataaggacccagaaggacgtgaattcttcgattgcatgaatgaaatccttgaactagctggaaccccaaatatcgcagacttcttgccgctactaaagaaattagatccactgggaatgaagaaaaggatggttgataatatgtcaagaacaatgaagatttcttcaaaatttgtgcaagagagattagataataggaaggccggaaaaatcaatgaaaagaaagattttctggatgtgatgttggaatatcagggggacgggaaggacggtccagacaaattcacagagcaacatgtgaatatcgttattatggaaatgttttttgcaggttctgaaacaacctctattagcatagaatggggtttcactgaactgttaagaaacccccacgcattcaagaaagtgagagaggaaattgatagagttgtcggtgtgaacagaatggttgaggagacagatatggaaaaccttccatacttacaagctgtggtaaaagagactttgagattacatcccgccctaccaatgttattaccaagaaacacgatggaagacactgaatatatggggtatcttatacccaagggtacccaagttttcgttaatgcttgggccatcgggagagatcccgaatattggcaagacccgctgagtttcaagccagaaaggtttataaatagttctgttgagtataaaggtcagcactttgagctaataccattcggatccggcagaagaatctgtgttgggtttcctttggctcatagagtggtccatttaaccttagcgacactggtccaagcatttgattgggacttaggtgcgggggtcaaacctcaggacatcgacctggaggaaaggcttggattgacgttgagaaagaaaaacccattaaatgtgattccaaaaaagagagtccatatctaa

SEQ ID NO: 12 - DNA, 1131 bp, Catharanthus roseus
  source 1..1131: mol_type = other DNA
atgcaaattataacttgtaaagccgttgtctgctgggctgctggagaaccgcccgttgttgaagaaatactagttgaaccccctagatccggtgaagtgaggatcaaaattctgtttgcgagtctatgtcatactgatgttctagcatgtaagggtttcccaactccaatgtttccgagagtactaggtcatgaaggtgtaggtgtcgtagagtgcgttggcgagggtgtatctgagttgagagagggagacgtggttattccaacttatttgggtgagtgtggagaatgcgaaaattgtgaaagtgggaggaccaacctatgtagaacatatccactgcaagcttttacaggcttgatgccagatggttcaagcaggatgtcatcagctaaagggggtgaaatgttatatcagttcttatcatgtagtacctggtcagagtatactgtcatagatgccaattatgcagtaaaaatcgattcaaggattccattgcctcatgcttcattcttaagctgtggctttacaaccggctttggagcaacatggaaggaggctaagttgcaagagggcagttcaactgttgcagtgctgggattaggagccgtcggtttaggtgccgttgaaggcgcaagagtacaaggcgtcactcaaataatcggaattgatattaacgataacaagagggaaaagggcgaggccttcggtatgactcattttatcaatcctaagaaagacaataacaagtctatttccgagttggtgaaagaattaactaagggccaaggggttgacgtttgtttcgaatgcacaggggtaccagaccttgtcaatgaagccttagagtccactaaaattggtacaggcaacatgatcatgttaggcgccggtactcaaaagtctatgacaattaactttgtgagtctattggggtgtagaacttttaagtatagcgtcttcggcggagttaaggtgcagagcgacttgccacttataattcaaaagtgcttgaacaaggaaatccaaaaaattgaacagttgctgacacatcaagtccaactggaagacattaatcgtgcctttgagttgcttaaagaaccagattgcgtaaaggttttaattacactataa

SEQ ID NO: 13 - DNA, 1449 bp, Catharanthus roseus
  source 1..1449: mol_type = other DNA
atgggttcacaagagacaaatctgccaccacatgtattaatcttcccactaccaattcaaggacacgtgaatagcatgcttagattggcagagcttttgtgtctagctgaattagatattaccttcattgtttccgaatttagccattctagattgattaagcatactaatgtagctagtaggttcgcgagatacccaggttttcaatttcaaccgatttcagatggcctacccgatgaccatcctagagccggagagagagtaatggatattctaccttccactaaaaatgtcactggtcctttgtttaaacaaatgatggtggaaaataaatgtttctcctccgccacccgtcgtccgattacatgcatcattgctgatggtgtgctgtcctttgcaggtgacttcgcacaagaaaagggtatcccactgatttattttagaactgtatctgcatgttctttttgggcatgtttttgtatgcccgaactaattgaatccggtgatataccaatcaaaggtaatgggatggatttaatcgtaaaatcagttccaggtatggaaacttttttgagaaggagagatttacctggcttctgtcgtgtgaatgatatcaatgagcccaagctacaaatattaaaaaccgaaaccagacagaccacgcgtgctcaggctgccattttgaacaccttcgaagatttggaaggccctattttatcacaaataagaaagcatatgcctagactgttcacgataggaccaagccattcccatctaacaagcagactggaaactaagaatattaaaacattgatttcatccggttccttttgggaagaggataggtcatgcgtggattggttagatgcacaacctcccagatcagtcctatacgtgtctttcggatcaataacggttgtaacaagggatcagttgttggaattttggtatgggctagttaactctggtcaaaggtttttatgggttatgagaccagattctatcatggggaaggacggacaaagccagatccccgctgatttggaagagggtactaaagcaaggggttacatggttggttgggctccacaagaagaagttcttaaccacccagctatcggtggatttcttacccattctggttggaacagcaccttagaatctatcgtggcgggcgtaccaatgatttgttggccttatttcgcagatcaaatgataaatagcagattcgtatcagaaatttggaaaatcggattggatatgaaagacacgtgcgaccgtgaaacaattgttaaaatggtcagggaactaatggaaataagaaaagatgagtttttgcaaagagccgatcatatggccaaattggcaaaagaagctgtgtctgaaggcggctcatcatatagcaatcttgacgggctagtagattatattaagtccctgatcatttaa

SEQ ID NO: 14 - DNA, 1566 bp, Catharanthus roseus
  source 1..1566: mol_type = other DNA
atggaattgaactttaagtcaattatattcttagtgtttgtgtctctaaccttgtactgggtctatagaattctggactgggtttggtttaaacctaaaaaattggaaaaatgcctaagggagcaaggatttaaaggtaatccttatcgtttatttcttggcgatcaatatgattctgggaagttaatcagacaagcgttgactaaaccaataggcgtagaggaagatgtgaaaaagaggatagttcctcatattctgaagacagtaggaactcacggcaaaaaatcctttatgtgggttggtagaatcccaagagttaatattacagatccagaattaattaaggaagttttgactaaatactataagttccaaaaaaaccatcatgaccttgatcccatcacaaagttacttttaacaggtattggttcattagaaggagacccatgggcaaagagaaggaagatcattaatgcagcttttcattttgaaaagttgaaactaatgttgccagcattttatttaagttgcagagacatggtaactaagtgggataataaggttcccgagggtggaagcgcagaagtggatgtttggcatgacatagaaaccttaactggtgatgttataagtagaaccctgttcggcagtaattttgaagaggggagaagaatattcgaattaatgaaggaattaactgcattaacaattgacgttataaggtctgtttatatcccaggtcaaagatttttacctaccaaaaggaacaaccgtatgagagccatagataaggaggttagggtgaggataactgaaattattaacaaaaagatgaaagtaatgaagagtggtgaggctgctagcgccgctgacgatttcctaggtattttattagaatgtaatctgaatgaaattaaagagcaaggtaacaataaatctgctggtatgactatcggagaaattattggtgaatgtaaactgttctactttgccgggcaagatacaacttctacacttttggtttggactatggtgttattatctagattccctgaatggcagacaagagcaagggaagaggtttttcaagtctttggaaacaagacaccagattacgatggtatcagtcacttgaaagtcattacaatgattttgtatgaagttttgagattatatacaccagttgccgaactaactaaagtggctcacgaggcaactcaactaggtaagtattttatccctgctggtgtacaattgatgatgcctcaaatcctgttgcatcatgatccagaaatttggggcgaagatgtaatggaatttaagcctgaaaggttcgcagaaggcgtattaaaggctacaaagtcccaaggaagctttttcccattttccctgggaccgagaatgtgcatcggtcaaaatttcgctttactagaggccaaaatggctatgtcattgatactacgtcgtttttcttttgaactgagtccttcttacgtacacgccccttttactctaattacaatgcaaccccaatatggtgcacatctgatattacataagttgtaa

SEQ ID NO: 15 - DNA, 1116 bp, Catharanthus roseus
  source 1..1116: mol_type = other DNA
atggtagcaacgattgattcaattgaaatgcctgccttaccaactgcggtcgaagctcaccccatgaaaggtggtgatgattctcattcctatagccagaatagttgttaccagaaaggtgtcattgatgcggccaaggctgtcattgttgaggccgtcaatgaaaaactggatctagaaaataatccaatttttgatcctattaaacctttcaggattgctgatttcggttgctccacgggtcctaatacttttcatgccatgcaaaatattgttgaatccgtagaaacaaagtataaatcccttcaaaagacccctgagtttcatgtcttttttaatgatcacgtcaacaatgattttaacgtcttatttcgttctcttccgcccaatagggagtttttcgcggctggtgtaccaggttcattttatacaagggtattcccaaagaatagtattcattttgcccactgttcttacgcattgcattggctatctaaggttcctaaggaaattcaagacaaaaactctttggcgtacaataagggtaggattcattacactggcactgagaaacacgtagtcaaagcttactttggtcaattccagagagatttcgaaggctttctaaaagctagagcccaggagatagtcgtgggtggtttaatggttatacaaataccgggcttaccttccggagaagtgctattcagtagaactggtgcaggtctattgcatttcttactaggtacgagcttaatggagttggtaaataagggtataatcaacgaagaaagcgttgacagcttcaacttaccgcaatatcatccctcagttgaagacttggaaatggttatagaaatgaacgattgcttcacaattgagagagtcggcacattgccccatccaatgaagaacctacctttcgatgtccaaagaacatctttacaagtgagagcaatcatggaatgtattcttactgaacattttggcgagaacatcttggatccactattcgaaatctacactaagaacctacaagagaacttccatgttttcgataaggaaatcagaaaggatgcagatttgtaccttgttttgaagagaaaagggaactaa

SEQ ID NO: 16 - DNA, 1575 bp, Catharanthus roseus
  source 1..1575: mol_type = other DNA
atggaaatggatatggatacaatcagaaaagctatcgccgctactatttttgcattagtgatggcgtgggcatggagagttcttgattgggcgtggtttaccccgaagagaatagagaaaagacttaggcaacagggtttcaggggcaatccgtatcgtttcttggtcggggatgttaaagaatccgggaagatgcaccaggaagccttgtctaagccaatggagttcaacaacgatatcgtccctaggcttatgccacatatcaatcatacgattaacacatatggtaggaactcttttacttggatgggacgtatacctagaatacatgtaatggaacctgagttgatcaaggaagtcttaactcatagttccaaatatcaaaagaattttgacgtccataatccacttgtgaagttccttttgacgggcgttgggtcatttgagggcgcgaagtggtcaaaacataggaggataatttcaccagcattcacacttgagaagctgaaatctatgctacctgctttcgcaatatgttaccacgatatgcttacaaagtgggagaaaattgctgagaagcaaggatcccatgaggtagacatatttcctacttttgatgtgttaacttctgatgtaatttctaaggtggcgtttggctctacttatgaggaaggtggtaagatatttagattgttgaaggagcttatggatcttactattgactgcatgagggatgtatatattcctggatggtcttacttgccgaccaagagaaataaaagaatgaaggagataaacaaagaaatcactgatatgttaagattcattattaacaagagaatgaaggctctaaaggccggcgaaccgggagaagatgatttattaggtgtactgttggaaagcaatattcaagagattcaaaaacaggggaataagaaagacggcggtatgagtattaacgatgtaatcgaagagtgcaagctattttactttgctggccaagaaacaactggggtgctgctaacttggacaactatcttattatctaagcatccagaatggcaagagagggctagagaagaagtgttacaagcatttggaaaaaataaacctgaattcgaaagattgaaccaccttaagtatgtttcaatgattttatatgaagtgttaagactgtacccgcctgtaatagatttgactaaaatagtgcataaagatacgaaattaggatcatatactattccagcaggaacgcaggttatgttaccgactgtgatgttacaccgtgagaagtctatttggggggaagatgcgatggaatttaatccaatgagatttgtggatggtgtcgccaatgctacgaaaaacaatgttacatatttaccgttttcctggggtccgagagtctgtctgggtcaaaactttgctttgctacaagctaagcttggcttggcaatgatattgcagcgttttaaattcgacgttgctcctagttatgtccatgcccctttcaccattttaacggttcaacctcaatttgggagccacgtcatatacaagaaacttgaaagctaa

SEQ ID NO: 17 - DNA, 1503 bp, Catharanthus roseus
  source 1..1503: mol_type = other DNA
atgggtagcatcgactctacgaatgtagcgatgagtaattcaccagtgggtgaattcaagcctctagaggctgaagaatttcgtaagcaagcacatcgtatggttgacttcatcgctgattactataagaatgttgaaacttacccagtgttgtctgaagttgagccagggtaccttaggaagcgtatacctgagactgcgccttatctgccagaaccactggatgatataatgaaagatatacaaaaagacattatccctggtatgactaactggatgagtccaaacttctacgctttcttcccggctacagtatcttctgcagctttcctaggtgagatgttatcaactgccttaaattccgtaggattcacttgggtatcaagtccagccgctactgagctggaaatgatcgtcatggattggctggctcaaatccttaagttacctaaatctttcatgttcagtggaaccggggggggcgttattcaaaatacgacatctgaaagcattttgtgtacaattattgctgctagggaacgtgcgctagaaaaattggggccagattccatcgggaaactggtttgttacggatcagatcagactcacactatgtttccaaaaacttgtaaacttgcgggaatttatcctaacaatattcgtctaatacccactactgtcgaaacagacttcggaataagccctcaagtcttgagaaagatggtggaggatgatgttgctgcaggatatgtaccattgtttttatgtgctacattaggtactacatcaacaaccgctacagatccagtggattctttgtctgaaatcgcaaacgaatttggtatatggattcacgttgatgcagcttacgcaggttcagcttgcatttgtcccgagtttaggcattacttggacggtatcgaaagagtggactccttgtcactatctccccacaaatggttgcttgcttacttagattgtacttgtttgtgggttaaacaacctcatctattgttgagagctttaacgactaacccagaatacttaaaaaacaaacaatccgatttggataaagttgtggattttaaaaattggcaaatcgcaacgggtaggaagtttaggagtcttaagttatggctgatattgcgtagctacggggttgttaacctacaatcacatattaggtctgatgttgctatgggtaaaatgtttgaagagtgggttagaagtgatagtcgttttgagattgtagttcctagaaacttcagtctagtgtgcttccgtcttaaacctgatgtcagttcactgcacgttgaagaggtgaataagaaattacttgacatgttaaattcaactggtagagtgtacatgactcacaccattgtgggaggtatatatatgttacgtttagcagtgggttccagcttgacagaggaacaccacgttagaagggtatgggacttgatacaaaaattgactgatgacttgttaaaagaggcgtaa

SEQ ID NO: 18 - DNA, 1473 bp, Ruminococcus gnavus
  source 1..1473: mol_type = other DNA
atgtcacaagtgatcaagaagaagaggaacaccttcatgataggcacggagtacatccttaactcgacccagttagaagaggcaatcaagtccttcgtccacgacttctgcgcggagaagcacgagatccacgaccagcccgtggtagtcgaggccaaggagcaccaagaggacaagataaagcagataaagatccccgagaagggacgtcccgtcaacgaggtcgtctcggagatgatgaacgaggtctaccgttaccgtggagacgccaaccaccctcgtttcttctcattcgtgcctggccctgcatcgtcggtatcgtggctaggggacatcatgacctctgcatacaacatccacgccggcggatcaaagctggcaccgatggtgaactgcatagagcaggaagtgttaaagtggcttgcgaagcaggtcgggttcacagagaaccccggtggggtattcgtatccggcggaagtatggccaacataacggccctgacagccgcgcgtgacaacaagttaacagacatcaacctacacctaggcacggcatacatatccgaccagacacacagttccgtagcgaagggcctaaggatcataggaataaccgactcacgtataaggcgtatccctacaaactcccacttccagatggacacaaccaagttagaggaagcaatcgagaccgacaagaagtcaggatacatccctttcgtggtgatagggaccgcgggcacaacaaacacggggtccatagaccctctgacagagatctcagccttatgcaagaagcacgacatgtggttccacatagacggggcctacggcgcctcagtactattatcacctaagtacaagagtttactgacaggaaccggactagcagactcaataagttgggacgcacacaagtggctattccagacgtacggctgcgcaatggtcctggtcaaggacatccgtaacttattccactcattccacgtcaacccggagtacctaaaggacctggagaacgacatagacaacgtaaacacatgggacatcggcatggagctgacccgtcctgcgcgtggactgaagctatggcttacattacaggtactaggctcagacctaataggatcagcaatcgagcacggattccagttagccgtatgggcagaagaggcattaaaccctaagaaggactgggagatcgtgagtcctgcccagatggcaatgataaacttccgttacgcccccaaggacctgacaaaagaggagcaggacatactgaacgagaagatctcacaccgtatacttgagtccggctacgcggcaatcttcaccacagtgttaaacggaaagacagtcctacgtatctgcgccatacaccccgaggcaacccaggaagacatgcagcacaccatcgacctacttgaccagtacggacgtgagatctacacagagatgaagaaggcgtga

SEQ ID NO: 19 - DNA, 1059 bp, Catharanthus roseus
  source 1..1059: mol_type = other DNA
atggcaaatttctcagaatccaaatcaatgatggctgtcttttttatgttctttctgttgctgttatcatcctcatcttcatcatcctcctcaagtcctattttgaaaaagatattcattgaatctccaagctatgctccaaacgcctttacttttgatagtactgacaaaggcttttacacttcagtgcaagatggtagagttattaaatatgagggtcctaattctggctttacagattttgcttacgcatccccattttggaacaaagctttttgcgaaaatagtacagatccggaaaaaagaccactatgtggtagaacatatgatatctcatatgattacaagaacagtcaaatgtacattgttgatggtcactaccatttgtgtgtcgtcggtaaagaaggtggatatgctacgcaattagctacgtcagtgcaaggagtccctttcaaatggctatatgcggtgaccgtcgatcaaaggactggtatcgtatatttcactgatgtcagctctatacacgacgatagcccagaaggggttgaagaaattatgaatacttcagataggactgggagactgatgaagtacgacccatctaccaaggaaaccacattattgttaaaggaactacacgtaccaggaggtgccgaaatctctgctgatggctccttcgtcgttgtagctgaattcctatcaaacagaatcgtaaaatattggttagaaggtccaaagaaaggttctgctgaattcttagtaacgattcccaaccctggaaacattaagagaaatagtgatgggcatttctgggtaagttcttccgaagaacttgacggaggtcaacatggtagagttgtttccagaggtataaagttcgatggatttggcaacatattgcaagtcatccctcttccaccgccttacgaaggcgaacattttgaacaaatacaagaacatgatggtttattgtacattggaagcctgttccattcaagtgttggaattttagtttacgatgatcacgacaataaaggtaactcatacgtcagttcataa

SEQ ID NO: 20 - DNA, 1599 bp, Rauvolfia serpentina
  source 1..1599: mol_type = other DNA
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 21 - DNA, 1071 bp, Catharanthus roseus
  source 1..1071: mol_type = other DNA
atggctatggctagtaagagcccttctgaggaggtctatccagtaaaagcattcgggctggcagcgaaagactcctccggactattttcaccattcaacttctctaggagggccacgggtgagcacgatgtacagctaaaggttttatattgcggtacctgtcaatacgatcgtgagatgtcaaaaaataagttcggcttcacaagctatccgtacgtacttggacacgagatagtgggtgaggttacagaggtggggtccaaagtacagaagttcaaagtgggggataaagtcggggtcgcctcaattattgaaacgtgcggcaagtgtgaaatgtgcacaaatgaagtggagaattactgtcctgaggcaggatcaatcgacagcaactatggtgcatgctccaacatcgctgtcatcaatgagaattttgtcattcgttggcctgagaacctgcctttagattcaggcgtgcccttgttgtgcgcgggtattacggcttattctcccatgaagagatatggactagacaaaccgggaaagagaattggtatagccggcttgggaggtttgggtcatgtcgcgctacgttttgcaaaggcgtttggcgcgaaagtaaccgtcattagttcatcacttaaaaagaagagggaggcgtttgaaaagttcggggcagattcattcttggtgtccagtaaccctgaagaaatgcaaggggccgcgggaactcttgacgggataattgacactatccctggaaaccatagcctagagcccttactagcgttgttgaaacccttaggaaagctaattattctgggtgccccggagatgccatttgaggttccagcgccgtcattattgatgggaggcaaggtgatggctgcgtcaacggccggtagtatgaaagaaatccaggaaatgattgagtttgcagcagaacacaatatcgttgctgacgtcgaagttattagtattgattatgtgaacaccgcgatggaacgtcttgacaactctgatgtgcgttacaggtttgtcatagacatcgggaacactctgaagagtaattaa

SEQ ID NO: 22 - DNA, 1080 bp, Catharanthus roseus
  source 1..1080: mol_type = other DNA
atggcagctaagtcaccagagaatgtctatcccgtgaaaaccttcggtttcgctgcgaaggattccagtggcttcttctctcccttcaatttttctcgtagggccactggcgagaacgatgtgcagtttaaagtgttgtattgcgggacctgtaattacgaccttgaaatgtcaacgaacaagtttggaatgaccaaatatccctttgtaatagggcatgagatcgtgggtgtagtaacggagataggctccaaggtccaaaagttcaaagtcggtgataaggtcggcgttggtggctttgtgggcgcctgtgaaaaatgcgaaatgtgcgttaatggcgttgaaaataactgttcaaaagttgaaagtaccgatggacacttcggtaacaactttggtggatgctgtaacataatggtagtgaatgagaagtatgcagtagtgtggccagaaaatctgcccttacacagcggtgttccccttctgtgcgctggaatcacgacatattctcccttgcgtcgttatgggttggacaaaccgggcctgaatattgggatagctggactggggggactgggacacctggctattcgtttcgcaaaagcattcggcgccaaggtcactctaataagttctagcgttaaaaagaagcgtgaagctcttgaaaaatttggggtagacagcttcctgctgaattctaaccctgaagaaatgcagggggcatatgggaccttagatgggattatcgatacaatgcccgttgcccactctattgtgccgtttttagcacttctaaaaccgttaggcaagctaattattttaggagtacctgaggagcccttcgaggtccccgcacccgccttgctgatgggtggtaagctgatcgcgggctcagctgctggaagtatgaaggagactcaagaaatgattgattttgctgctaaacataatatcgttgcggacgtggaagttatacctatagattacttaaacactgcaatggaaagaattaaaaactcagatgtcaaatacagattcgtgatagacgttgggaacactttaaaatccccttcattctaa

SEQ ID NO: 23 - DNA, 1095 bp, Catharanthus roseus
  source 1..1095: mol_type = other DNA
atggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaataa

SEQ ID NO: 24 - DNA, 1089 bp, Catharanthus roseus
  source 1..1089: mol_type = other DNA
atggctggtgaaactactaaattggatttgtctgttaaagctattggttggggtgctgctgatgcttctggtgttttgcaaccaattagattttatagaagagctccaggtgaaagagatgttaaaattagagttttgtattgtggtgtttgtaattttgatatggaaatggttagaaataaatggggttttactagatatccatatgtttttggtcatgaaactgctggtgaagttgttgaagttggtaaaaaagttgaaaaatttaaagttggtgataaagttggtgttggttgtatggttggttcttgtggtaaatgttttcattgtcaaaatgaaatggaaaattattgtccagaaccaaatttggctgatggttctacttatagagaagaaggtgaaagatcttatggtggttgttctaatgttatggttgttgatgaaaaatttgttttgagatggccagaaaatttgccacaagataaaggtgttccattgttgtgtgctggtgttgttgtttattctccaatgaaatatatgggttttgataaaccaggtaaacatattggtgtttttggtttgggtggtttgggttctattgctgttaaatttattaaagcttttggtggtaaagctactgttatttctacttctagaagaaaagaaaaagaagctattgaagaacatggtgctgatgcttttgttgttaatactgattctgaacaattgaaagctttggaaggtactatggatggtgttgttgatactactccaggtggtagaactccaatgccattgatgttgaatttggttaaatttgatggtgctgttattttggttggtgctccagaaactttgtttgaattgccattggaaccaatgggtagaaaaaaaattattggttcttctactggtggtttgaaagaatatcaagaagttttggatattgctgctaaacataatattgtttgtgatactgaagttattggtattgattatttgtctactgctatggaaagaattaaaaatttggatgttaaatatagatttgctattgatattggtaatactttgaaatttgaagaataa

SEQ ID NO: 25 - protein, 14 aa, Simian virus 40
MPKKKRKVGGGGGG

SEQ ID NO: 26 - DNA, 1506 bp, Catharanthus roseus
  source 1..1506: mol_type = other DNA
atggagttttccttttcttcccccgctttgtatatagtgtattttctgttgttcttcgttgttaggcagttgctgaaacccaaatcaaagaagaaactaccaccaggcccaagaacgctgcctctgatagggaatttacatcagttgagcggaccattgccgcaccgtacattaaagaacctatcagataaacacggtccgctgatgcacgtgaagatgggcgagagatctgccatcatagttagcgacgcaaggatggcgaagatagtcttgcacaataacggattggccgttgcagataggtcagtcaatactgtcgcgtccattatgacctacaactcactgggcgtcacgtttgctcaatatggcgactacctgaccaaattgcgtcagatctataccttggagctactttcccagaagaaagtcagaagtttttattcttgtttcgaggacgaactagacactttcgtaaagtctatcaagtccaatgtgggccagccgatggttttgtacgaaaaagcatctgcgtatttgtatgccacaatttgtagaaccatcttcgggagcgtttgcaaagaaaaagagaagatgataaaaatagtcaagaaaaccagcctattgagcgggactcctctaagactagaagacttgtttccaagcatgtctattttctgtcgtttttctaagactctgaatcagctgagaggcctgcttcaagaaatggacgatatccttgaagagatcatagttgagcgtgaaaaagcatctgaggtttcaaaagaagcgaaagacgatgaagacatgttaagtgtactactgcgtcacaaatggtataatccaagtggagccaaatttagaatcaccaatgctgatatcaaagctataatctttgaacttatacttgcggcaacgctatcagtggcagatgttacggaatgggcaatggttgaaatcttacgtgatccgaagtctcttaagaaagtatatgaggaggtacgtggcatttgtaaagagaaaaagagggtcacaggatatgacgtggagaagatggagttcatgcgtttgtgcgttaaagaatccactagaattcatccagctgcaccattgttagttccccgtgaatgtcgtgaggattttgaggttgatgggtacacagtccccaagggcgcatgggtgataaccaactgttgggcggttcagatggaccccacagtctggcccgagcctgaaaaattcgatcctgaacgttatattcgtaaccccatggacttctatggatctaattttgagctaatcccatttggtaccggcaggagaggctgccccggcatattgtatggcgttactaacgcagaatttatgttagctgctatgttttatcactttgattgggagatagccgatggtaagaaaccggaagaaattgacctgacggaagatttcggtgctggctgcataatgaagtacccactaaagttagttccgcatttagttaatgactaa

SEQ ID NO: 27 - DNA, 1506 bp, Tabernaemontana elegans
  source 1..1506: mol_type = other DNA
atggaattgtctttttcttctccagctttgtatattttgtattttttgttgtttttgttggttaaacaattgttgaaaccaaaatctcaaaaaaaattgccaccaggtccaagaactttgccaattattggtaatttgcatcaattgatgggtccattgccacatagaactttgaaagatttgtctgataaacatggtccattgatgcatttgaaaatgggtgaaagatctgctattattgtttctgatgctagaatggctaaaattgttttgcataatcatggtttggctgttgctgatagatctgttaatactgttgcttctattatgacttataattctttgggtgttacttttgctcaatatggtgattatttgactaaattgagacaaatttatactttggaattgttgtctccaaaaaaagttagatctttttattcttgttttgaagatgaattggatggttttgttaaatctattaaatctcaagttggtcaaccaatggttttgtatgaaaaagcttctacttatttgtatgctactatttgtagaactatttttggttctatttgtaaagaaagagaaaaaatgattaaaattgttaaaagaacttctttgttgtctggtactccattgagattggaagatttgtttccatctatgagagttttttgtagattttctaaaactttgaatcaattgagaggtttgttgcaagaaatggatgatattttggaagatattattattgaaagagaaaaaactactgaaatttctactgaagctaaagatgatgaagatatgttgtctgttttgttgagacataaatggcataatccatctggtgctaaatttagaattactaatgctgatattaaagctattatttttgaattgattttggctgctactttgtctgttgctgatgttactgaatgggctatggttgaaattttgagagatccaaaatctttgaaaaaagtttatgatgaagttagagaagtttgtaaagaaaaaaaaagagttactggttatgatgttgaaaaattggaatatatgcatttgtgtgttaaagaatctactagaattcatccagctgctccattgttggttccaagagaatgtagagaagattttgaagttgatggttatactgttccaaaaggtgcttgggttttgactaattgttgggctgttcaaatggatccaaaaatttggccagaaccagaaaaatttgatccagaaagatatattagaaatccaatggatttttatggttctaattttgaattgattccatttggtactggtagaagaggttgtccaggtattttgtttggtgttactaatgctgaattgttgttggctgctatgttttatcattttgattgggaaattgctgatggtaaaaaaccagaagaaattgatttgactgaagattttggtgctggttgtattatgaaatatccattgaaattggttccacatttggctaatgaataa

SEQ ID NO: 28 - DNA, 1506 bp, Vinca minor
  source 1..1506: mol_type = other DNA
atggagttcagtttctcctcactgcctctatacatcgcaagtttcctattattcttccttgtcaagcagatcttaaagcctaagagcggcaagaagttaccaccgggccctcgtaccctacctataatagggaaccttcaccagcttatgggacctctacctcaccgtacactgaagaacctgtcagacaagcacggaccgcttatgcacttaaagcttggcgagcgttccgccataatagtctcagacgcacgtcttgcgaagatcgtgttacacaacaacggactggccgtagcggaccgtagcgtaaacaccgtcgcgtcaatcatgacgtacaactccctgggcgtcacattcgcccagtacggagactacttaacaaagttaaggcagatctacacgcttgagttactaagtcagaagaaggtcaggagtttctacaactgcttcgaggacgagcttgacacattcgtcaagtccataaagagctccaacggacagcctatggtactttacgaggtcgcctcgacgtacctatacgcaaccatatgcaggacaatcttcggctcggtgtgcaaggagcgtgagaagatgatcaagatagtaaagcgtacttccttactgagcggaacacccttaaggttagaggacctattccccagtatgagcgtgttctgccgtttctccaagacacttaaccagcttcgtggcctacttgacgagatggaccacatcttagaggacatcatcgtcgacagggagaagaacacggagatattaaaagagggaaaggacgacgaggacatgctttcggtactattaaggcacaagtggcacaaccccagtggcgcaaagttcaggatagcaaacgccgacataaaggccatcatattcgagcttatcttagccgcaacgttatcagtagcagacgtaaccgagtgggcaatggtcgagataataagggaccctaagtccctgaagaaggtccacgaagaggtcaggcaggtatgcaaggacaagggcaaggtgacaggatacgacgtagagaagttagactacctaaggttatgcctgaaggagtcaaccaggatacaccctgccgcccctctactggtcccgagggagtgccgtgaggacttccaggtagacggcttcacagtcccgaagggagcatgggtccttacaaactgctgggcagtgcagatggaccccgaggtctggcccgagcctgagaagttcgaccctgagaggtacataaggaagccgatggacttctacggaaactccttcgagcttatacctttcggaacgggaaggcgtggatgccctggcatactttacggagtcacaaacgccgagatgatgttagcggccatgctgtaccacttcgactgggagatcgccgacggaaagaaggccgaggagatagacatgaccgaggacttcggagcgggctgcatcatgaagtaccccttaaagttaatccctcacatagtgaacgagtga

SEQ ID NO: 29 - DNA, 1506 bp, Amsonia hubrichtii
  source 1..1506: mol_type = other DNA
atggagttctcattctcgagtccgttactttacatagtatacttcttactattcttcttagtcaggtacttacttaagcccaagtcaaacaagaagttaccacccggaccccgtacattaccgataataggaaacctacaccagctaatggggcctctgccccacaggaccttaaaggagctttcggacaggcacggccctcttatgcacgtaaagatgggcgagaggagtgcgatcatagtatcaaacgcaaggatggccaagatagtcttacacaaccacggattagccgtcgccgacaggtcggtaaacacagtagcatcgatcatgacatacaacagcttaggcgtcacgttcgcccagtacggagactaccttacgaagctacgtcagatctacaccctagagctgttatcacctaagaaggtcaggagtttctacgggtgcttcgaagaggagctagacgtattcgtagagtccataaagtcatcagtcggccagccgatggtcatgtacgagaagtgcagtaagtacttatacgcaaccatatgccgtaccatattcgggagcgtctgcaaggagagggagaagatgataaagatcgtgaagaagacatcacttctgagtggaacccctttacgtttagaggaccttttcccttcaatgtcggtattcgcaaggttcagtaagaccctgaagcagttacgtggactactacaggagatggacgacatattagaggacatcatcacagagagggagaagacaacagagatcagcacggagtccaaggacgacgaggacatgctaagtgtcttattaaggcacaagtggcacaacccttccggcgccaagttccgtataacaaacgcggacataaaggccataatattcgagctaatactggcagccaccctaagtgtggcagacgtagccgagtgggcaatggtcgagatattaagggaccctaagtcactgaagaaggtgtacgaagaggtaagggaagtctgcaaggagaagaagaaggtcacaggacacgacgtagagaagctggagttcatgcgtttatgcttaaaggagagcacacgtatacaccctgcagcccctcttctagtccctcgtgagtgcagggaagacttcgaggtcgacggatacaccgtacctaagggcgcatgggtcctaacgaactgctgggccgtacagatggacccggaagtatggcctgagccggagaagttcgaccccgagaggtacatacgtaaccctatggacttctacggcagctccttcgagctgatccctttcggaacgggaagaaggggatgccccgggatcttatacggcgtaaccaacgcagagttaatgttagcagcgatgttctaccacttcgactgggagatcgcagacgggaagaagcctgaggagatagacatgacagaggacttcggggcgggctgcataatgaagtaccccctaaagctggtacctcacctagcaaagaactga

SEQ ID NO: 30 - DNA, 1065 bp, Catharanthus roseus
  source 1..1065: mol_type = other DNA
atggccgacagggtgaagactgttggatgggctgcacacgactcctctggattcttatctccatttcaattcacgagaagggctaccggggaggaagacgttaggttgaaagtgctatattgcggggtatgccattcagacctacataacatcaaaaatgaaatgggttttacgtcctacccctgcgtccctggacacgaggtagtgggagaggtaacggaagttggaaataaagtaaagaaattcataattggtgacaaagtcggggtagggttgtttgtggatagctgtggagagtgtgaacaatgcgttaacgatgttgagacttactgcccgaaacttaaaatggcatatttaagtatcgacgacgatggcacggttattcagggtgggtatagcaaagaaatggttataaaggagaggtatgtttttcgttggccggagaaccttcccttgccagcgggaacccccttactaggggctggttctactgtgtacagcccaatgaaatactacgggctagataagagtggccaacatttgggagtcgttggcctgggggggctgggccacctggctgtaaagtttgctaaggcatttggtcttaaagtcactgtaatttccacatccccatctaaaaaggacgaggccatcaaccatcttggggctgacgccttccttgttagcactgaccaggaacagactcaaaaagctatgagcaccatggacggaatcatagacactgttagtgccccacatgctcttatgccccttttctcactgttgaagcctaacggaaagttgatcgtcgtaggcgctcccaataaacctgtagagttagatatattgtttctagtaatgggtagaaaaatgttaggaacctctgcagtaggtggagtcaaggagacacaggaaatgattgacttcgcagcgaagcacggaattgttgctgatgtggaagtggtggagatggaaaatgttaataacgcgatggaaagactagccaaaggtgatgttaggtatcgttttgtattagatataggtaatgcgacagtcgcagtttaa

SEQ ID NO: 31 - DNA, 972 bp, Catharanthus roseus
  source 1..972: mol_type = other DNA
atggaaaagcaagttgagatacctgaggtcgagttaaactccggccacaagatgcctatcgttggatatgggacctgtgtcccggaaccaatgccaccgttagaggaacttaccgctattttcctggacgctattaaggttgggtaccgtcacttcgacactgcgtcttcttatggaaccgaagaagctcttggaaaggcaatagccgaagcgattaactcagggttggtcaaatcccgtgaagaattctttatttcctgtaagttatggatcgaagatgccgaccatgacttaatacttcctgccttaaaccagagtcttcaaattcttggggtggactacttagacctatacatgatccatatgccagtgagggtccgtaaaggcgcacctatgttcaactatagtaaagaagacttcctgccatttgacattcaggggacatggaaagcgatggaggagtgcagcaaacaaggtttagccaaaagcatcggtgtatccaactactccgtggaaaaacttacgaaattactagagacatccaccatcccccctgccgttaaccaagtcgaaatgaatgtcgcttggcaacaaaggaaactattaccgttctgtaaggagaaaaacatacacatcaccagttggagccctttactatcctacggcgtcgcttggggtagcaacgccgtcatggagaatcctgtgttacagcaaattgccgctagtaaagggaagacagtggcacaggttgcactgcgttggatatacgagcagggcgctagcctgatcacaaggacgagtaataaggatagaatgtttgagaacgtgcagatatttgactgggaattgtccaaagaagagctagaccaaatacacgaaattccccaacgtcgtggaacgcttggggaggaattcatgcacccggaaggcccaattaaaagtccggaggagttatgggatggtgatttataa

SEQ ID NO: 32 - DNA, 1266 bp, Catharanthus roseus
  source 1..1266: mol_type = other DNA
atggctcctcagatgcagattctgtccgaggaattgatccagcctagctccccgacaccccaaacgttaaagacacataaactaagtcatctggaccaggtgctactgacttgccatatccccattattttattttacccgaatcaattagactcaaacttagacagggcgcagagatcagaaaacttgaaacgttcactatctactgtactgacgcagttctacccactggcgggaaggataaacataaatagttccgtggattgtaatgattcaggagttccttttctggaggcccgtgtccactcacagctaagtgaggcaataaagaacgtggcaatcgacgaattaaaccagtatctaccattccagccttatcctggaggagaggaatctggactaaaaaaggacatcccactggccgtaaagataagttgtttcgagtgtggggggacagctataggagtctgcatatctcacaaaatagcggatgcattaagtttggccactttcctaaacagttggacggctacatgtcaagaggagacagatattgtgcaaccgaacttcgacttgggctctcaccatttccccccaatggaaagcattccagcgcctgagtttcttcccgatgaaaatatcgtcatgaaaaggtttgtctttgacaaagagaaacttgaggccttgaaagcacagctagcgtctagtgccactgaagtgaaaaactcatccagggtccagatcgtaattgctgttatatggaaacagttcatagacgttacaagagctaaatttgacacgaaaaacaagcttgtggctgcacaagcagtcaacctgcgtagcagaatgaacccaccatttccgcagtccgcgatgggcaatatagcaaccatggcttacgcagtcgctgaagaggataaggattttagtgatttagtaggcccattgaaaacttcattggcaaaaatcgatgacgaacatgtgaaggagcttcagaagggtgtaacctaccttgattacgaagctgaaccgcaagagcttttctctttttcatcctggtgtaggttaggcttttatgatctggattttggctggggaaagcctgttagtgtttgtacgacaacggtcccgatgaagaatcttgtatacttaatggatacaaggaacgaagacgggatggaagcgtggatcagtatggcggaggatgagatgtcaatgcttagctcagatttcttgtcactactagatactgatttttctaattaa

SEQ ID NO: 33 - DNA, 1590 bp, Catharanthus roseus
  source 1..1590: mol_type = other DNA
atgataaaaaaggtccctatcgttttatccatcttctgttttttgttattactatcttcttcccacggatccattccggaggcgttcctaaattgtatttctaataaattctcattagacgtaagcatattgaacatactgcacgtcccctcaaatagtagttacgactctgtacttaaatccacgatacagaatccgaggttccttaaaagtccgaaaccactagccattattacccctgttctgcacagccatgtacaatccgctgtaatctgtaccaagcaagcgggactacagattagaattagatcagggggagctgactatgaaggcctgagctataggtccgaagtacccttcatactgcttgatttacagaatttacgtagtatttccgtcgacattgaggacaattctgcgtgggtggaaagtggtgcgactataggcgagttctaccacgaaatcgcacaaaacagcccagtgcacgcgttccctgctggagtcagctcatccgttggcatcggtggacacctgtcttccggcgggttcgggactctacttagaaagtacggcttggcagcggacaacattatagatgcgaaaatagtagatgcaaggggtcgtatcttagacagggagtccatgggtgaagacctattctgggctataagagggggaggcggcgcgagttttggggtcattgtgagctggaaagtcaagttagtaaaagtaccaccgatggtgactgtatttattttgagtaaaacatacgaggaaggggggctagatttactgcacaaatggcaatacatcgagcataagctacccgaggatctgttcttagcggtctcaattatggacgacagtagtagcggcaataaaacgctgatggctggctttatgtccctattccttggcaagactgaagacctactgaaggtcatggcggagaactttccccaattaggtctgaagaaagaggattgtctagagatgaattggattgacgcagcgatgtactttagtggccacccaattggtgagagccgttctgtgttgaaaaatagggaaagtcacctaccaaagacttgcgtgagcataaagtccgacttcattcaagaaccacaaagcatggacgccttggagaaattatggaaattctgtagggaggaagagaactctcctatcatattgatgttacccctaggaggtatgatgagtaagatcagcgagtcagagataccttttccctaccgtaaggatgttatttactcaatgatttatgagatagtatggaattgcgaggacgacgaatctagtgaagaatatatcgacggtctgggcaggttggaagagttgatgactccttatgtcaagcaaccgaggggctcctggttctctacaaggaacctttataccggaaaaaacaagggaccgggtactacctacagcaaagcgaaggagtggggatttagatatttcaacaacaacttcaagaaattggcattgatcaaagggcaagtagacccagagaactttttctattatgaacagtccattccacctctgcatcttcaagttgagctataa

SEQ ID NO: 34 - DNA, 1593 bp, Amsonia hubrichtii
  source 1..1593: mol_type = other DNA
atgtatactgatgttcataaagtttttactttttcttgttttttgttgttggtttcttcttgtcattgtttgactttggaatcttttatttcttgtttttcttctgaatttccatctgatgaaccaattttttctgttttgcatgatccaagaaatgcttcttatcaaccattgttggaatctactattcataatttgagatttttgaaatctccaaaaccattggctattattactccatttagatattctcatgttcaagctgctgttgtttgttgtaaacaagttagattgcaaattagaattagatctggtggtcatgattatgaaggtttgtcttatagatctgaagttccatttattattttggatttgagaaatttgagatctattactgttgatattgaagattcttctgcttgggttgaatctggtgctacttctggtgaattgtattatcaaattgctatgaaatctccaattcatgcttttccagctggtatttattctactgttggtgttggtggtcatttttctggtggtggttttggtactatgttgagaaaatatggtttggctgctgataatattattgatgcttatattgttgatgctaaaggtagattgttgaatagagaatctatgggtaaagatttgttttgggctattagaggtggtggtggtgcttcttttggtgttattgttgcttggaaaattaaattggttcatgttccaccagttgttactgtttttgatttgccaaaaacttttgaagaaggtgctattgatttgattcataaatggcaaaatgttggtccagaagtttctgaagatttgtttttggctgtttctgttatggctgatgcttctggtggtaataaaactattatggctggttttacttctttgtttttgggtactgctgatttgttgttgaaagaaatggaagaaagatttccagaattgggtttgagaactgaacattgttctgaaatgtcttggattaatgctgttatgcatttttctggttatccatctggtgaaactatttctgctttgaaaaatggtgaagctccattgccaaaaaattgtgttgctactaattctgattttgttcaagaaccattgtctgtttctgctttggaaaaattgtggaatttttgtttggaagaaagaaatactccaattattttgatgttgccacatggtggtatgatgtctaaaatttctgaaactgaaactccatttccatatagacaaggtgttatttattctttgatttatgaagttgtttgggattgtcaagatgattatttttctgaagaatatatttcttctttgagaagatttcatgatcatttgactccacatgttttgaaacatccaagaggtacttttttgaatactagaaatttggatactggtaaacatgataattatgatattacttattctaaagctaaagaatggggtttgaaatattttaaaaaaaattttagaagattggctattattaaaggtgaagttgatgctgaaaatttttttcattttgaacaatctattccaccattggtttctattgatgaattgtaa

SEQ ID NO: 35 - DNA, 1590 bp, Tabernanthe iboga
  source 1..1590: mol_type = other DNA
atggttgaagtttctaaagttttgtcttgtttttgttttttgttgttgttgtctttgtctcatggtttgattccagaagcttttattacttgtatttctcaagaatttgaatcttatgaatctattttgagagttttgcatgatccaggtaattcttcttatcattctgttttgcaatctagaattcaaaatttgagatttttgaaatctccaaaaccattggctattattactccattgttgtattctcatgttcaagctgctattgtttgttctaaacaagttggtttgcaaattagaattagatctggtggtaatgattatgaaggtttgtcttatagatctgaagttccatttattttgttggatttgcataatttgagatctattgctgttgatattgaagataattctgcttgggttgaatctggtgctactattggtgaattgtattatgaaattgctcaaaaatctccaattcatgcttttccagctggtgtttatgctactgttggtgttggtggtcatttttctggtggtggttttggtactatgttgagaaaatatggtttggctgctgataatgttattgatgcttatattgttgatgctagaggtagattgttggatagagaatctatgggtgaagatttgttttgggctattagaggtggtggtggtgcttcttttggtgttattgtttcttggaaaattaaattggttcatgttccaccagttgttactgtttttgatatgccaaaaactttggaacaaggtgctttggatttgttgaataaatggcaatatattggtcataaagtttctgaagatttgtttttggctgtttctattatggctgatacttctggtggtaataaaactttgatggctggttttaattctttgtttttgggtactgctgatcaattgttgaaagaaatggctgaatcttttccagaattgggtttgagaaaagaacattgttctgaaatgggttggattaaagctgctatgcatttttctggttatccatctgttgaaactacttctccattgaaatctagagaaccaccattgccaaaaacttgtattgctactaaatctgattttattcaagaaccattgtctttgccagctttggaaaaattgtggaaattgttgtgggaagaagaaaatactccaattattttgatgttgccacatggtggtatgatgtctaaaatttctgaatgggaattgccatttccatatagacaagatgttatttattctatgattgatgaagttgtttgggattgtgaagatgattattcttctgaagaacatatttctggtttgagaaaattgtatgatttgatgactccatatgtttctaaacatccaagaggtacttttttgaatatgagaaatttggatactggtagaaatggtgattattatggtactacttattctaaagctaaagaatggggtttgaaatattttaaaaataattttagaaaattggctattactaaaggtgctgttgatccagataattttttttattttgaacaatctattccaccattgacttctcaagatgaataa

SEQ ID NO: 36 - DNA, 1596 bp, Tabernaemontana elegans
  source 1..1596: mol_type = other DNA
atgtatactactgaagttcataaagtttttatttcttattgttttttgttgttgttgtctcaatgtcattgtttgattccagaatcttttatttcttgtttgtctaaaaaatttccatctgatgaaccaattttttctgttttgcatgatccaagaaatgcttcttatcaagctgttttggaatctaatttgcaaaatttgagatttttgaaatctgctaaaccattggctattattactccattgcattatactcatgttcaagctgctgttgtttgttgtaaaagagctggtttgcaaattagaattagatctggtggttctgattatgaaggtttgtcttatagatctgaagttccatatattattttggatttgcaaaatttgagatctattactgttgatattgaagataattctgcttgggttgaatctggtgctactattggtgaattgtattatgaaattgctgatcaatctccagttcatgcttttccagctggtgtttattctactgttggtgttggtggtcatttgtctggtggtggttttggtactatgttgagaaaatatggtttggctgctgataatattttggatgctcatattgttgatgctgaaggtagattgttgaatagagaatctatgggtactgatttgttttgggctattagaggtggtggtggtgcttcttttggtgttattgttgcttggaaagttaaattggttcatgttccaccagttgttactgtttttgatttggctaaaactttggaagaaggtgctattgatttgattcataaatggcaaactgttggtccaaatttgaatgaagatgcttttttggctgcttctattatggctgatccatcttctgaatctaaaactttggttgctggttttttttctttgtttttgggtattgctgatcaattgttgaaagaaatgaaagaatcttttccagaattgggtttgagaaaagaagattgtttggaaatgtcttggattaaagctgctttgcatttttctggttatgaaccaggtgaaactgtttatgctttgaaaaatagaaaaccaccacaaccaaaacaatgtattactgttagatctgattttattcaagaaccattgtctttgccagctttggataaattgtggaaatttttgtctgaagaagaaaatactccaattattgttatgttgccacatggtggtatgatgtctaaaatttctgaaactgaaattccatatccatatagagaaggtgttatttattcttttttgtatgaattgaattgggattgtgaagatgattctttttctgaaagatatgtttctgctttgactagattgtatgatcatatgactccatatgttttgaaacatccaagaggtggttttttgaatatgagatgtttggaaattggtaaaaatgatgattatggtactacttattctaaagctaaagaatggggtttgaaatattttaaaaataattttaaaagattggctattactaaaggtgctgttgatccagataattttttttattttgaacaatctattccaccattggcttctaaagatgaattgtaa

SEQ ID NO: 37 - DNA, 1098 bp, Catharanthus roseus
  source 1..1098: mol_type = other DNA
atggcaggcaagagcgcggaggaggaacatcccatcaaggcttatggttgggcagtcaaagacaggacgacaggtatcctgtcccccttcaagttctccaggagagcgaccggggacgacgatgttaggataaaaatactatactgtgggatatgtcacacagatctagcatctatcaagaacgaatatgaattcctatcctatccgctagtacccggaatggaaatagttggaatagcaacagaggttggcaaagatgttactaaagtaaaggtcggtgaaaaggttgctttgagcgcctatttagggtgctgtgggaagtgttatagctgtgtgaacgaactagaaaattactgccctgaggtcattatagggtatggaacaccgtaccatgacggcacgatatgttacggtggattatccaacgagacagttgccaaccagtccttcgttctaagattcccagagagactatctccagccggcggcgcccctctattatctgcgggaattacgtcatttagcgcgatgcgtaattcagggatcgacaaacccggtcttcatgtaggcgttgtcggtttaggggggttgggtcacctagcagtcaagtttgcaaaagctttcggcttaaaggtcactgtaattagcaccacaccgtccaagaaagatgatgcaatcaacggtcttggggccgatgggttcctgttaagccgtgacgatgagcagatgaaagccgccattggaacgctggatgccattatagacactttggcagtagtccacccgattgcgcccctactagatcttctgcgtagccagggcaaatttctgctgctaggcgccccttctcagagtttggaactacctccgattcccttgttaagtggtggcaagagcattattggtagtgctgctggaaacgtaaagcaaacacaagagatgcttgatttcgccgctgaacatgatatcacggcgaatgtggaaattatacccatagagtatataaacacggctatggaaagactagacaaaggcgacgtaagatacaggtttgtggtcgacatcgaaaataccttaacccccccttccgaactgtaa

SEQ ID NO: 38 - DNA, 1098 bp, Tabernanthe iboga
  source 1..1098: mol_type = other DNA
atggctggtaaatctccagaagaagaacatccagttaaagcttatggttgggctgttaaagatagaactactggtattttgtctccatttaaattttctagaagagctactggtgataatgatattagaattaaaattttgtattgtggtatttgtcatactgatttgacttctgttaaaaatgaatatgaatttttgtcttatccattggttccaggtatggaaattgttggtattgctactgaagttggttctaaagttactaaaattaaagttggtgaaaaagttgctgttgctgcttatttgggtacttgtggtaaatgttataattgtgttaatgatttggaaaattattgtccagaagttattattggttatggtactccatatcatgatggtactattaattatggtggtttgtctaatgaaactgttgttaatgaaagatttgttttgagatttccagaaaaattgtctccagctggtggtgctccattgttgtctgctggtattactgcttattctgctatgagaaatcatggtttggataaaccaggtattcaattgggtgttgttggtttgggtggtttgggtcatttggctgttaaatttgctaaagcttttggtgttagagttactgttatttctactactccatctaaaaaagatgaagctattaataatttgggtgctgatgcttttttgttttctagagatgataaacaaatgagagctgctattggtacttttgatgctattattgatactttggctgttgttcatccaattgctccattgttggatttgttgagatctcatggtaaattggttttggttggtgctccatctaaaccattggaattgccaactattccattgttgtctggtggtaaatctttgattggttctgctgctggtaatgttaaacaaactcaagaaatgttggattttgctgctgaacatgatattactgctaatattgaagttattccaattgattatattaatactgctatggaaagattggataaaggtgatattagatttagatttgttgttgatattgaaaatactttgactccaccaccagaaccataa

SEQ ID NO: 39 - DNA, 963 bp, Catharanthus roseus
  source 1..963: mol_type = other DNA
atgggctcaagtgacgagactatcttcgacttaccgccgtacataaaagtcttcaaagacggacgtgtagagaggctacatagtagcccctacgtgcctcctagcttgaacgatccagagaccgggggtgtgtcatggaaggatgttccgatatccagcgtggtcagtgctcgtatttacctacctaagattaataatcacgacgagaaattacctatcatagtttatttccacggagcagggttctgtctggaatcagcgtttaagtcattttttcacacttatgtcaaacacttcgtggccgaagccaaggccattgccgtcagtgttgagtttaggctggctccggagaatcacttgcccgctgcctatgaagattgttgggaagcgttacagtgggtagccagtcacgtgggactggacataagtagtttaaagacgtgtatcgacaaagatccgtggattataaattatgcagatttcgacaggctgtacttgtggggggattccacgggtgcgaatatagttcacaacactcttataagaagcggaaaagaaaagttaaatggtggtaaggtcaagattctaggtgcgatcttatattatccgtatttcttgattcgtacttctagcaagcaaagtgattacatggagaatgagtatagatcctattggaaacttgcgtatccggatgcgccgggcggaaatgataatccgatgattaatccaactgcagagaatgcgccggatctagctggatatggatgttcccgtttgttaatatcaatggtcgctgatgaggccagagacataaccttgttgtatatcgacgctcttgagaaaagcggttggaaaggggaactagatgttgcggattttgataagcagtatttcgaattgtttgagatggaaacggaggttgctaagaatatgttaagaaggttagcatcttttatcaaataa

SEQ ID NO: 40 - DNA, 993 bp, Catharanthus roseus
  source 1..993: mol_type = other DNA
atgaatagcagcacggacccgaccagtgatgaaacaatctgggatctgtccccgtatattaagatcttcaaggacggaagagtagaacgtctacacaactccccatacgtgcccccgtcactaaatgatcctgagacgggggtgagttggaaggacgttcccatttccagtcaagtttcagcgagagtttacatccctaagatttccgaccatgagaagctgccgattttcgtctacgtgcacggtgcgggtttttgcctagaatcagccttcaggtccttcttccatacttttgtaaaacatttcgtcgctgaaacgaaggttatcggtgtatctatagaataccgtttggcgcccgaacaccttctgccggccgcctatgaagattgctgggaggcgttacagtgggtagcgtctcatgtaggattggataatagcggtttgaagacggctattgacaaagacccttggataataaactatggagactttgatagattatatcttgcgggggatagcccaggagccaacatcgtacacaatacacttataagggccgggaaagagaaattaaaaggaggagttaaaatacttggagctatactttactacccgtactttatcatcccaacgagcactaagttgtctgacgattttgaatataactacacatgctactggaaattggcttaccccaatgcccctggcgggatgaacaacccaatgataaaccctatagctgagaatgctcctgatcttgcggggtacggttgttctagacttttggtaaccttggtttccatgatttccactacgcccgatgaaactaaagatatcaatgcggtctatattgaggccctggagaagagtggctggaagggagagttagaagtggccgattttgacgcagactacttcgagttattcaccctagaaacagagatgggtaagaacatgtttagacgtctggccagtttcattaaacatgagtaa

SEQ ID NO: 41 - DNA, 1539 bp, Catharanthus roseus
  source 1..1539: mol_type = other DNA
atggaactgtactattttagcacatttgcctttttgcttttctgcttcatacttgccaaaacgttaaagaagtcaggacaatcaaatcttaaactacccttgggaccacccccgattcctatacttggtaatgcacaccagttaatcggaggacacacacaccacattttgcgtgacttggcgaagaaatacggacccttaatgcatctaaaaaccggagaagtgtcaacaatagtagccagtagccctgaaattgccgaagagatgtttaagacccatgatgtactgttcgcagatcgtccatctaacatagttgcgtttaaaattttaagctacgattactcagacgtcgttatatccccttacgggaattactggaggcaactacgtaagatttccatgatggagttgttcagccaaaggagtgtgcaatcatttaggtcaatccgtgaggaagaggtcttaaactttataaagagtataggtagcagagaggggactaaaataaacctgagtaaagagattagccttcttatttatggtattacaaccagagctgcttttggagagaagaacaagaatactgaagagttcataaggttgctggaccaactgacggttgcggtcgcagagcctaatatagccgatatgtttccgtccatcaatttccttaaattaatctctcgtagcaaatataaaatcgaaaaaatacataagaattttgatgccattgtgcaaactattctaaatcaccataaggataggttggcgaatcataagtcttctagccacgaagagaatggcgagcaaaacaaggatctagtcgatgtcctgcttaacattcagcagcgtggagacttcgacactccgttgggagacagatcagtgaaagctgtaattttcaatatattttcagcgggtactgagacaagttctacaacagtcgattgggctatgtgcgaaatgataaagaatcctaccattatgaagaaggctcaagaggaggtacgtaaagtatataacgaagaagggaatgttaacgagacgaagttacatcagctgaagtatttgaaagcggtgataaaggagactttaaggcttcaccctcccgtaccacttctgttgccgagagagtgtagagaacaatgtgagattaagggatatacgatacccagcaagtcccgtgtaatcgtgaacgcctgggctattggtagagatcccaactactggattgagccggagaattttaatccagaaaggtttttagagagcgaagtagattttaaaggaaactcattcgagtacctaccgtttggagggggtaggaggatctgtccagggatcactttcgcacttgcgaatatagaattgccattggcccagctactgtttcatttcgactggaaattggcaagcgacgaaacaaacatcgataagctggacatgacggaatctagaggagtgactgtaagacgtgaagacgatctttgtttgatcccttttccctactctgcttccagcctgaagggcaaatattaa

SEQ ID NO: 42 - DNA, 1068 bp, Catharanthus roseus
  source 1..1068: mol_type = other DNA
atggatgtccaatctgaagaatttagaggggcgcaggcccaaatctggtctcagtcatgttcctttataacttccgcaagtttaaaatgcgccgtcaagctgggtattcctgacaccatcgataatcatggaaaaccgatcacattatctgagcttaccaatgccctggtgccaccggtacatccaagtaaagcaccttttatctataggctgatgagagtattggcaaaaaatgggttctgctcagaggaacagctggacggagaaactgaaccactgtactccttgaccccgtctagtcgtattttgcttaaaaaggagccattaaatcttcgtggaatagtgttgactatggcagatcccgtgcagttgaaagcgtgggaatctctgagcgattggtatcagaacgaagatgattcatctaccgcgttcgagaccgcccatgggaaaaacttttggggctatagctccgagcacatggaacacgcggaattttttaacgaagccatggcgagcgattctcaactgatcagtaagcttttgatcggagagtataagttcttatttgagggtttagccagtttggtagacataggcggagggacagggaccatagcgaaggcaatagctaaaaactttccgcaattgaaatgtacagtatttgatttaccgcatgtagtcgccaacctagaatcaaaggaaaacgtggaattcgtcgctggggatatgttcgagaaaatcccgtccgcgaatgcgatttttctgaaatggatcttacacgactggaacgacgaggactgcgttaagattttgaagagttgtaagaaagccataccagcgaagggcggtaaggtcataattatcgatatggttatgtattccgacaagaaggacgatcacctagtaaagactcagacctctatggacatggccatgctggtcaatttcgctgccaaggagaggtgtgagaaagaatgggcttttttattcaaagaggcaggttttagcgactataaaatatatccgaaacttgacttcacacgttctcttatcgaagtttacccttaa

SEQ ID NO: 43 - DNA, 1521 bp, Catharanthus roseus
  source 1..1521: mol_type = other DNA
atggagttccacgaaagtagtccgtttgtcttcattaccaggggtttcatttttatagcgatcagtatcgcggtgttgaggcgtatcatctcaaagaaaacaaagactttacccccaggtccttggaagctaccgcttattggcaatttacaccaattcttgggttctgtaccgtaccaaatccttagagatctggcccagaaaaacggtcctctaatgcatttacaactgggagaagttagtgcgatagtagcagcgtctccacaaatggcaaaagagatcactaagacccttgatttacaatttgctgatcgtccagtcatacaggcgttgagaatcgtaacctacgattaccttgacatctccttcaacgcctacgggaaatattggaggcaactgcgtaaaatcttcgtccaggaactactaacatcaaagagagtaagatcattttgttctattagggaggatgaattttccaacttagttaagacgataaattctgctaatgggaaaagcatcaatctatcaaaacttatgacctcctgcacgaactccataattaataaagtggccttcgggaaggttaggtacgaacgtgaggtttttatcgatttgataaatcagattttagcattagctgggggattcaaactggtagatctgttcccgagttataaaattttgcatgtgctagaggggaccgagagaaagctatgggaaattaggggaaagatagataagatcctagataaagtgatcgacgagcatagggagaatagttcacgtactggtaagggcaatggatgtaatggccaggaagatatcgtcgatatcttgcttcgtattgaggagggaggagacctagatttagatatcccattcggcaataataatattaaggcgttactatttgacatcatcgcaggcggtacggagacgtccagcacagctgtcgactgggcgatgtcagaaatgatgaggaatccgcatgttatgagtaaagcgcaaaaggagattagggaggctttcaacggaaaggaaaagatcgaagaaaatgatatccaaaacttgaaatatttgaagttagtaattcaagagactctaaggctacaccctccagcaccccttttgatgcgtcaatgccgtgagaagtgtgaaatcggtggttatcatatacctgttgggacaaaggcgttcataaacgtatgggcaatcgggcgtgacccggcatactggcctaaccctgagagctttatcccggagcgttttgacgataatacgtacgaatttactaaatccgaacaccacgcgtttgagtatttgccatttggcgcagggagaaggatgtgtcctggcatcagcttcggcttggcaaatgtagaattgcctctagcattattgctgtatcatttcaactggcagctgccggacggctcaactactctagatatgacggaggccaccgggctggcggcccgtagaaaatatgacctacaactgatagctacttcatatgcctaa

SEQ ID NO: 44 - DNA, 1071 bp, Catharanthus roseus
  source 1..1071: mol_type = other DNA
atgagttctgagatggccgcgaaatccgtaaaggcattcgggcttgccttgaaggactcatctggtcttttctcaccttttaacttcagcagacgtgccacaggagagcacgacgtgcaactaaaggtactatattgtggggtctgtaattttgacaacttgatgcgtagaaataaatacggaaggaccaagtttccatacgtctttggtcacgaaatagtcggggtagtaactgaggttggcagtaatgtaaagaagtttaagatcggaaataaagtcggagtgtcttttatcgtcgatacctgcagagagtgtgagagatgtaaaattggacaacaaatagcgtgtaaaaaggcggtaagtagcgacggtttcttcgagactcccggctatggcgggtgctcaaacatctttgtagcggacgaaaattacgttattctgtggcctgaaaatttgccaatggatagtggtgcgccactattatgcataggcataacctgttacaatccacttaggagatttgggctagacaagccgggcgtaagggtaggtatagtcggcttgggagccgtggggcatttagccatcaagttcgctaaagcctttggggcacgtgtcacgttgatcagctcatctccgggaaagaaggatgaagcgtttcaaaaatttggagttgactcatttctagttagtagcaacgccgaagagatgcaagcagctgcagaaacactagatggcatcctggacacggtccctgtggtgcatcctcttgaacctctgtttgcgcttctaaaacctctaggaaaattgatcattattggcgagccgcacaagccatttgaagtctccgcaatgtcattgatggaaggcgggaaaatcatatctgcaagtacgggtgggtcaataaaagatactcaagaaattgtggatttcgcggcagagcacaacgtcgttgccgatgtcgaggttatccctgtagattacgtaaatactgcgatggaaagacttgacaaagcagacgtgaaatatagattcgtgatcgacatcggaaacacctttaaaagtccgtaa

SEQ ID NO: 45 - DNA, 870 bp, Catharanthus roseus
  source 1..870: mol_type = other DNA
atggaagagaaacaagagaaagttgcagagttttatgataaagtgacaggggcgtgggaccttttttatggtgtccacctacatgatgggtactacgaaccgggcactacggcgacaatggcaatttcacaggacgctgtcattcgtatgattgacgaattacttagatttgcgggggtctcagaagaccctgcaaagaaacccaggagtatgttggacgtaggatccgggttgggaggaacctgtgtttatgtggccaaaaagtatgacatccaatgcacggggattaccatcagtccaaaccaggtaaaatacgcgcaagactacgcagcaactgagggcgtggaaaacaaggtctcctttgactgtggggacgccttagacatgccatacagcgacggtaagttcgacgttgtcttcacaatcaactgcattgagcacgtccatgacaaggagaagtttatcagagaaatggtcagagtagccgcgcccggagcagcaattatcattgcaagtcaagcacatcctaacctaagtcccggtgagtcccttaagccaagggataaaaaaattctgcagaaaatttgcgacggtgcaggggccgtttcactgtgtagcagtgatgactatgtacgttggcttactcccttgcctgttaaagagatcaaggccgcggattggacgcaaaacataacaccactgtatccgctacttatgaaggaagcattcacttggaaagggttcacatccatagtattgaaaggtggctggcgtgcgatcaacctgatcaatgcggttcgtttggtcgctaaagcagctaatgatggcattcttaagttcgccgttgtaaccggtcgtaaaagcatctaa

SEQ ID NO: 46 - DNA, 1206 bp, Catharanthus roseus
  source 1..1206: mol_type = other DNA
atgccaaaatcttggcccatcgtgataagttcccattctttctgctttctaccgaactcagagcaagaaagaaagatgaaagacctgaacttccacgccgccactctgtccgaggaagaatcattgagagaactgaaagcattcgacgagacgaaggcaggagtaaaggggatcgtcgacacagggattaccaagataccgaggatctttatcgaccaaccaaagaacttggacaggataagcgtatgccgtgggaaatccgatattaaaatccccgtcatcaacctaaatggcctatcatcaaatagtgagatcagaagggaaatagtagagaaaattggggaggcctctgaaaagtacggtttctttcagattgttaatcacggaataccgcaggatgttatggacaagatggttgacggggttcgtaagtttcatgaacaggacgatcaaatcaagcgtcaatactactcaagggaccgtttcaacaaaaactttttatatagttctaactatgtgttaatcccggggatcgcttgcaactggagggatactatggagtgtatcatgaacagtaatcagccggatcctcaggagtttcctgatgtgtgcagggatattctaatgaaatactcaaactacgtgcgtaatcttgggcttatactgtttgaattactgagcgaagctctgggcttgaagccaaaccacctggaggagatggactgtgccgaggggctaatcctacttggtcactactaccctgcatgtccacagcccgaattgacgtttgggacgtccaaacactcagattctggcttccttacgattttaatgcaagaccaaataggcggtttgcagattctacttgaaaatcaatggattgatgttccattcatccctggtgctttagtcattaatatcgcagacttattacaattaatcacaaacgataaattcaagtcagttgagcatcgtgtgttagccaacaaggttggcccgagaatatctgttgctgttgcgtttggtatcaaaacgcaaacccaagaaggagtgagcccacgtctgtatggtccgataaaagagttgataagcgaagagaatcctcctatttacaaggaagtaacggtcaaagattttatcactatcaggtttgcgaaaaggtttgatgatagctctagtctgtctccgtttagactaaataactaa

SEQ ID NO: 47 - DNA, 1320 bp, Catharanthus roseus
  source 1..1320: mol_type = other DNA
atggagagtggaaagatatccgtcgagacggaaacgctgagcaaaacgttgataaagcctagctcccccaccccacagtccttaagcagatacaacctgagctacaatgatcagaatatttaccaaacatgcgtatccgtcgggttcttctacgagaaccccgatggaatagagattagcactattagggagcaactacaaaattctcttagtaaaactctagtctcctattacccgttcgcaggcaaagtcgtcaaaaatgattacattcactgtaacgatgacggcatcgagttcgttgaagtccgtattagatgtaggatgaatgacattcttaaatatgaacttcgttcttatgctagagacttagtgcttcctaagcgtgtaacggtaggcagcgaggatactacggctattgtgcaattatcacactttgattgtggtgggttagccgtggctttcggtataagccacaaagtagcggacggaggtactatagcgtcatttatgaaggactgggccgccagtgcctgttacctatcatcaagccatcatgtgcccactcccctgttagttagcgacagcatcttcccacgtcaagacaacattatctgcgagcagtttccaacaagcaaaaattgcgtcgagaagacattcatatttccccccgaagcgatcgagaaacttaagagcaaagccgtcgagtttggtattgagaaaccgactcgtgtcgaggtgctgacggcttttcttagtaggtgtgcaacggttgcggggaagtctgccgccaaaaataacaactgcggtcaatcactgcccttccctgttctgcaagcaattaacctgagacctattttagaattaccacagaattcagttgggaatttggtctccatctatttttcacgtactatcaaggagaacgactatttgaacgagaaggaatataccaaattagttataaacgagttgcgtaaggagaagcaaaaaatcaaaaacttgtctagggaaaagctaacttatgttgctcagatggaggagtttgtgaagtccttgaaagagttcgatataagtaattttttagatatagacgcctacttgtccgactcctggtgccgttttccgttttatgacgttgattttggttggggaaagcccatttgggtctgcctgtttcaaccttatattaagaactgtgtcgtcatgatggattacccctttggtgacgattacggcatagaagcgattgtctctttcgagcaggaaaaaatgtccgcctttgaaaaaaacgagcagcttcttcagttcgtctccaactaa

SEQ ID NO: 48 - DNA, 1536 bp, Catharanthus roseus
  source 1..1536: mol_type = genomic DNA
atggagatccgagacctcttctggtctctccctgcaatagcattgctgcaagtttttcttttctttttgttcaaaaaccctaaaaagaccactcttaaattaccacctggtccgcccactcttcccataattggaaatctacaccaaatggcaagtccacttccacataagaagcttaaggacttagctgataaatatggaccattgatgcacttgaaattgggggaaatttcaacagtagtaatttcttcgtcaagattaaccaaagaatttatgcaaactcatggcctaaactttgcggataggccacagactgttattgccaagatcatgatgtacaattgttcaggcgttacactttcaatgtacggcgattattggagaaaattgcggcaaatttatgtcacggaattacttaacacaaaaagtgtacaatcattttcttccatcatggaagaagagctcattcttatggttaaatctattgagtcagaagttgggaaacctatggaattgattgagaaaattcggtcatatttatttgacacactttgtagatcagcacttggtaaaatacatggtaaagggaaggagacattaattgagataagcagagaaatggtggcactttctggagtacaaaccctagaagatatttttccttcagtgaagttattttcatatttgaatccattgaggcccaaagcaaagaagttgtttaagagacttgatagtgttcttgaagacattattaatcaacaggagaataagttgctcagtttaaaagatggcgataatcaacaggagaaggaagaagataatatgctaagtgttttgttgaggctaaggaatggcaaggactcaaaagtaaaacttaccaacaatgacatcaaggctatcatttttgaattgttcgtcggtggaatttccacttcttcaacaactatagaatgggcaatgtcggaattgatgaaaaatcctgagatgatggaaaagggaaaacatgaagtgagacaagtcctgaaaggaaagaaaagagtttgccaaattgatgtggagaacatgagttatataaagctagtgctgaaagaaactcttagatttcatcctcccggtcctctattgttccctagaaaatcaagagaacaatgtgaaatcgatgggtatactatccctgccaaagcaatgattttaataaataattgggtattaggaagagatcctgaatattgggttgatcctgaaaagtttgaaccagagagatttaaggataatttggttgattataaaggaaatcatttcgagttaataccatttggtgttggaagaaggatttgtcctggaatatcatttgccgttactaatattgagcttttgttggctgcattgctctttcactttgactggaaacttccgcatggaatggatccaaaagatttagacatgattgagttatatcgttcaggttgcacaagaaaaaaccctttggttttaatccccaaaatttatattcccactggagatgaaaattattaa

SEQ ID NO: 49 - DNA, 1593 bp, synthetic construct
  source 1..1593: mol_type = other DNA
atgtctggtaaaattgataaaattttgattgttggtggtggtactgctggttggatggctgcttcttatttgggtaaagctttgcaaggtactgctgatattactttgttgcaagctccagatattccaactttgggtgttggtgaagctactattccaaatttgcaaactgctttttttgattttttgggtattccagaagatgaatggatgagagaatgtaatgcttcttataaagttgctattaaatttattaattggagaactgctggtgaaggtacttctgaagctagagaattggatggtggtccagatcatttttatcattcttttggtttgttgaaatatcatgaacaaattccattgtctcattattggtttgatagatcttatagaggtaaaactgttgaaccatttgattatgcttgttataaagaaccagttattttggatgctaatagatctccaagaagattggatggttctaaagttactaattatgcttggcattttgatgctcatttggttgctgattttttgagaagatttgctactgaaaaattgggtgttagacatgttgaagatagagttgaacatgttcaaagagatgctaatggtaatattgaatctgttagaactgctactggtagagtttttgatgctgatttgtttgttgattgttctggttttagaggtttgttgattaataaagctatggaagaaccatttttggatatgtctgatcatttgttgaatgattctgctgttgctactcaagttccacatgatgatgatgctaatggtgttgaaccatttacttctgctattgctatgaaatctggttggacttggaaaattccaatgttgggtagatttggtactggttatgtttattcttctagatttgctactgaagatgaagctgttagagaattttgtgaaatgtggcatttggatccagaaactcaaccattgaatagaattagatttagagttggtagaaatagaagagcttgggttggtaattgtgtttctattggtacttcttcttgttttgttgaaccattggaatctactggtatttattttgtttatgctgctttgtatcaattggttaaacattttccagataaatctttgaatccagttttgactgctagatttaatagagaaattgaaactatgtttgatgatactagagattttattcaagctcatttttatttttctccaagaactgatactccattttggagagctaataaagaattgagattggctgatggtatgcaagaaaaaattgatatgtatagagctggtatggctattaatgctccagcttctgatgatgctcaattgtattatggtaattttgaagaagaatttagaaatttttggaataattctagttattattgtgttttggctggtttgggtttggttccagatgctccatctccaagattggctcatatgccacaagctactgaatctgttgatgaagtttttggtgctgttaaagatagacaaagaaatttgttggaaactttgccatctttgcatgaatttttgagacaacaacatggtagataa

SEQ ID NO: 50 - DNA, 576 bp, Escherichia coli str. K-12 substr. MG1655
  source 1..576: mol_type = other DNA
atgagagttattactttggctggttctccaagatttccatctagatcttcttctttgttggaatatgctagagaaaaattgaatggtttggatgttgaagtttatcattggaatttgcaaaattttgctccagaagatttgttgtatgctagatttgattctccagctttgaaaacttttactgaacaattgcaacaagctgatggtttgattgttgctactccagtttataaagctgcttattctggtgctttgaaaactttgttggatttgttgccagaaagagctttgcaaggtaaagttgttttgccattggctactggtggtactgttgctcatttgttggctgttgattatgctttgaaaccagttttgtctgctttgaaagctcaagaaattttgcatggtgtttttgctgatgattctcaagttattgattatcatcatagaccacaatttactccaaatttgcaaactagattggatactgctttggaaactttttggcaagctttgcatagaagagatgttcaagttccagatttgttgtctttgagaggtaatgctcatgcttaa

SEQ ID NO: 51 - DNA, 1824 bp, synthetic construct
  misc_feature 1..1824: note = Artificial Sequence
  misc_feature 1..1824: note = Artificial Sequence
  source 1..1824: mol_type = other DNA
atggctagtaccagagttttagcttccagattggcttctcaaatggctgcttcagcaaaggtagcaagaccagctgtcagagtggctcaggtgtctaaaaggactatacaaaccggcagtccattgcaaactctaaagagaacacaaatgacaagtatcgtcaatgcaacaacaagacaagccttccaaaagagagcttactcctccggtggaggcggaggcggtatggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 52 - DNA, 1320 bp, synthetic construct
  misc_feature 1..1320: note = Artificial Sequence
  misc_feature 1..1320: note = Artificial Sequence
  source 1..1320: mol_type = other DNA
atggctagtaccagagttttagcttccagattggcttctcaaatggctgcttcagcaaaggtagcaagaccagctgtcagagtggctcaggtgtctaaaaggactatacaaaccggcagtccattgcaaactctaaagagaacacaaatgacaagtatcgtcaatgcaacaacaagacaagccttccaaaagagagcttactcctccggtggaggcggaggcggtatggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaataa

SEQ ID NO: 53 - DNA, 1701 bp, synthetic construct
  misc_feature 1..1701: note = Artificial Sequence
  misc_feature 1..1701: note = Artificial Sequence
  source 1..1701: mol_type = other DNA
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagaccggtggaggcggaggcggtatattagagcaacctctgaaatttgtgcttactgcggccgtcgtgctcttgacgacgtcggttctttgttgtgtagtatttacataa

SEQ ID NO: 54 - DNA, 1197 bp, synthetic construct
  misc_feature 1..1197: note = Artificial Sequence
  misc_feature 1..1197: note = Artificial Sequence
  source 1..1197: mol_type = other DNA
atggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaaggtggaggcggaggcggtatattagagcaacctctgaaatttgtgcttactgcggccgtcgtgctcttgacgacgtcggttctttgttgtgtagtatttacataa

SEQ ID NO: 55 - DNA, 1710 bp, synthetic construct
  misc_feature 1..1710: note = Artificial Sequence
  misc_feature 1..1710: note = Artificial Sequence
  source 1..1710: mol_type = other DNA
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagaccggtggaggcggaggcggttctacctctgaaaaccaaagtaaaggtagtggtacattggttgtcatattggccattttaatgctaggtgttgcttattatttgttgaacgaataa

SEQ ID NO: 56 - DNA, 1206 bp, synthetic construct
  misc_feature 1..1206: note = Artificial Sequence
  misc_feature 1..1206: note = Artificial Sequence
  source 1..1206: mol_type = other DNA
atggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaaggtggaggcggaggcggttctacctctgaaaaccaaagtaaaggtagtggtacattggttgtcatattggccattttaatgctaggtgttgcttattatttgttgaacgaataa

SEQ ID NO: 57 - DNA, 1626 bp, synthetic construct
  misc_feature 1..1626: note = Artificial Sequence
  misc_feature 1..1626: note = Artificial Sequence
  source 1..1626: mol_type = other DNA
atggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagaccttgggaagaggtagaagatccaaattgtaa

SEQ ID NO: 58 - DNA, 1122 bp, synthetic construct
  misc_feature 1..1122: note = Artificial Sequence
  misc_feature 1..1122: note = Artificial Sequence
  source 1..1122: mol_type = other DNA
atggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaattgggaagaggtagaagatccaaattgtaa

SEQ ID NO: 59 - DNA, 1649 bp, synthetic construct
  misc_feature 1..1649: note = Artificial Sequence
  misc_feature 1..1649: note = Artificial Sequence
  source 1..1649: mol_type = other DNA
atgaaggccatctctttacagaggccattgggtttagacaaggacgtcttatcgacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 60 - DNA, 1148 bp, synthetic construct
  misc_feature 1..1148: note = Artificial Sequence
  misc_feature 1..1148: note = Artificial Sequence
  source 1..1148: mol_type = other DNA
atgaaggccatctctttacagaggccattgggtttagacaaggacgtcttatcatggcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaataa

SEQ ID NO: 61 - DNA, 1641 bp, synthetic construct
  misc_feature 1..1641: note = Artificial Sequence
  misc_feature 1..1641: note = Artificial Sequence
  source 1..1641: mol_type = other DNA
atgcccaagaagaaaaggaaagttggtggaggcggaggcggtatggacaacactcaggccgagccgctggtggtagcgatagttccaaaaccgaatgctagcaccgaacacaccaatagtcatttgatacccgtgactcgtagtaagatcgtcgtccaccgtagagatttcccccaggattttatctttggtgctggcggttctgcgtaccaatgtgaaggtgcatacaatgaagggaatagagggccttcaatttgggatactttcacacaacgtagccccgctaagatttcagatggaagcaacgggaatcaggctataaactgctatcacatgtacaaagaagatataaagattatgaaacaaactggcttagaatcatatcgtttcagtatctcttggtccagggttttacccgggggtaggttagccgcaggtgttaacaaagacggtgtaaaattctatcacgactttatcgatgagttgctggctaacggtattaaaccgtctgtcactctgtttcactgggaccttcctcaggctcttgaggatgagtatggcggctttcttagccacaggatagttgacgatttttgtgaatatgccgagttttgtttctgggaattcggtgataagatcaagtattggactacgtttaatgaaccccatacttttgcagtgaacgggtacgccctaggcgaattcgcaccaggccgtgggggcaaaggggatgagggggaccctgctattgagccctacgtagtaacccacaacattctgctggctcataaggcagccgtcgaggaatacagaaacaaattccagaaatgccaggagggtgagataggaatcgttttgaactctatgtggatggaacctctgagcgatgtgcaggcggatatagatgcacaaaaacgtgcattagacttcatgcttggttggtttctagagccgcttacaacgggagattacccgaagtcaatgcgtgagttagttaaaggaaggctaccaaagttttcagccgatgacagcgagaaattgaaaggatgttacgattttataggtatgaactactacaccgccacttacgtgactaacgccgtaaaaagcaatagcgaaaaactgtcctacgagacggacgatcaggtgacaaagacattcgagagaaatcagaaaccaatcggccatgcgctttacgggggctggcaacatgtggtgccgtggggcctatacaaactgttggtttacacaaaagaaacgtaccatgtcccagttttgtacgtcacggaaagtggtatggtggaagaaaacaaaaccaaaatattactgagtgaggcgaggcgtgacgccgaacgtaccgactatcatcaaaaacatcttgcttccgtaagagacgccattgacgatggtgtcaacgtaaaaggatactttgtatggtcattcttcgataattttgaatggaatcttggctacatatgtcgttacgggataatccacgttgactataagagctttgaaagataccctaaggaatccgccatttggtataaaaatttcatcgctgggaaatccactaccagccccgctaaaagaaggagggaagaggcacaggtcgaattagtgaaacgtcaaaagacctaa

SEQ ID NO: 62 - DNA, 1134 bp, synthetic construct
  misc_feature 1..1134: note = Artificial Sequence
  misc_feature 1..1134: note = Artificial Sequence
  source 1..1134: mol_type = other DNA
atgcccaagaagaaaaggaaagttggtggaggcggaggcggtgcaggagagactacaaagttggatttgtcagtaaaggctgtgggttggggggctgccgatgcttccggcgtgttgcagccgatcaagttttacagacgtgtaccaggcgagagggacgtgaaaataagggtactttattcaggcgtgtgcaattttgacatggagatggtacgtaataagtggggtttcacgaggtatccgtatgtgttcggtcatgaaacggcgggcgaagtggttgaggttggatctaaggttgagaaatttaaggttggggataaagttgctgttggctgcatggtcggtagctgcggacagtgctacaactgtcaaagtgggatggaaaactattgtccagagccgaacatggcagacggcagcgtgtacagggagcagggcgagcgtagctatggcggctgctcaaacgtaatggtggtcgatgaaaaattcgtgctgaggtggcctgaaaatctaccacaggacaagggtgtcgccttgttgtgcgccggagtcgtggtatattccccaatgaagcacttaggcctagacaaaccggggaaacacataggcgtattcggacttggtggtcttggttcagtggctgttaagttcattaaggcattcggtggtaaggccacggtgatttcaacaagtaggcgtaaggaaaaggaggcgatagaggagcatggagccgatgccttcgttgtaaacacggatagcgagcagcttaaagccttggcgggcacgatggacggggtagtcgatacgacaccgggggggaggacacccatgagtcttatgttaaacttacttaaattcgacggtgctgtaatgttggtgggcgcgccagaatcactattcgaacttcctgcagccccgttaataatgggacgtaaaaaaattatcgggtccagcaccggaggtttaaaagaataccaggaaatgttagattttgccgctaaacacaacatagtttgtgatacagaggtgatcggtattgattacctgtctaccgcaatggaaaggatcaagaatttagacgtaaaatatcgttttgctattgacattggtaatacactgaaatttgaagaataa

SEQ ID NO: 63 - protein, 381 aa, Abies grandis
MAYSAMATMGYNGMAASCHTLHPTSPLKPFHGASTSLEAFNGEHMGLLRGYSKRKLSSYKNPASRSSNATVAQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 64 - protein, 367 aa, Gallus gallus
MHKFTGVNAKFQQPALRNLSPVVVEREREEFVGFFPQIVRDLTEDGIGHPEVGDAVARLKEVLQYNAPGGKCNRGLTVVAAYRELSGPGQKDAESLRCALAVGWCIELFQAFFLVADDIMDQSLTRRGQLCWYKKEGVGLDAIWDSFLLESSVYRVLKKYCRQRPYYVHLLELFLQTAYQTELGQMLDLITAPVSKVDLSHFSEERYKAIVKYKTAFYSFYLPVAAAMYMVGIDSKEEHENAKAILLEMGEYFQIQDDYLDCFGDPALTGKVGTDIQDNKCSWLVVQCLQRVTPEQRQLLEDNYGRKEPEKVAKVKELYEAVGMRAAFQQYEESSYRRLQELIEKHSNRLPKEIFLGLAQKIYKRQK

SEQ ID NO: 65 - protein, 540 aa, Catharanthus roseus
MSSMSLPLATPLIKDNESLIKFLRQPLVLPHEVDDSTKRRELLERTRKELELNAEKPLEALKMIDIIQRLGLSYHFEDDINSILTGFSNISSQTHEDLLTASLCFRLLRHNGHKINPDIFQKFMDNNGKFKDSLKDDTLGMLSLYEASYLGANGEEILMEAQEFTKTHLKNSLPAMAPSLSKKVSQALEQPRHRRMLRLEARRFIEEYGAENDHNPDLLELAKLDYNKVQSLHQMELSEITRWWKQLGLVDKLTFARDRPLECFLWTVGLLPEPKYSGCRIELAKTIAILLVIDDIFDTHGTLDELLLFTNAIKRWDLEAMEDLPEYMRICYMALYNTTNEICYKVLKENGWSVLPYLKATWIDMIEGFMVEAEWFNSDYVPNMEEYVENGVRTAGSYMALVHLFFLIGQGVTEDNVKLLIKPYPKLFSSSGRILRLWDDLGTAKEEQERGDLASSIQLFMREKEIKSEEEGRKGILEIIENLWKELNGELVYREEMPLAIIKTAFNMARASQVVYQHEEDTYFSSVDNYVKALFFTPCF

SEQ ID NO: 66 - protein, 896 aa, synthetic construct
  REGION 1..896: note = Artificial Sequence
  REGION 1..896: note = MISC_FEATURE - Artificial Sequence
MASEKEIRRERFLNVFPKLVEELNASLLAYGMPKEACDWYAHSLNYNTPGGKLNRGLSVVDTYAILSNKTVEQLGQEEYEKVAILGWCIELLQAYWLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIDQLYHEYEESVAKDLKAKISQVDESRGFKADVLTAFLNKVYKRSKGGGSMSSMSLPLATPLIKDNESLIKFLRQPLVLPHEVDDSTKRRELLERTRKELELNAEKPLEALKMIDIIQRLGLSYHFEDDINSILTGFSNISSQTHEDLLTASLCFRLLRHNGHKINPDIFQKFMDNNGKFKDSLKDDTLGMLSLYEASYLGANGEEILMEAQEFTKTHLKNSLPAMAPSLSKKVSQALEQPRHRRMLRLEARRFIEEYGAENDHNPDLLELAKLDYNKVQSLHQMELSEITRWWKQLGLVDKLTFARDRPLECFLWTVGLLPEPKYSGCRIELAKTIAILLVIDDIFDTHGTLDELLLFTNAIKRWDLEAMEDLPEYMRICYMALYNTTNEICYKVLKENGWSVLPYLKATWIDMIEGFMVEAEWFNSDYVPNMEEYVENGVRTAGSYMALVHLFFLIGQGVTEDNVKLLIKPYPKLFSSSGRILRLWDDLGTAKEEQERGDLASSIQLFMREKEIKSEEEGRKGILEIIENLWKELNGELVYREEMPLAIIKTAFNMARASQVVYQHEEDTYFSSVDNYVKALFFTPCF

SEQ ID NO: 67 - protein, 493 aa, Catharanthus roseus
MDYLTIILTLLFALTLYEAFSYLSRRTKNLPPGPSPLPFIGSLHLLGDQPHKSLAKLSKKHGPIMSLKLGQITTIVISSSTMAKEVLQKQDLAFSSRSVPNALHAHNQFKFSVVWLPVASRWRSLRKVLNSNIFSGNRLDANQHLRTRKVQELIAYCRKNSQSGEAVDVGRAAFRTSLNLLSNLIFSKDLTDPYSDSAKEFKDLVWNIMVEAGKPNLVDFFPLLEKVDPQGIRHRMTIHFGEVLKLFGGLVNERLEQRRSKGEKNDVLDVLLTTSQESPEEIDRTHIERMCLDLFVAGTDTTSSTLEWAMSEMLKNPDKMKKTQDELAQVIGRGKTIEESDINRLPYLRCVMKETLRIHPPVPFLIPRKVEQSVEVCGYNVPKGSQVLVNAWAIGRDETVWDDALAFKPERFMESELDIRGRDFELIPFGAGRRICPGLPLALRTVPLMLGSLLNSFNWKLEGGMAPKDLDMEEKFGITLQKAHPLRAVPSTL

SEQ ID NO: 68 - protein, 714 aa, Catharanthus roseus
MDSSSEKLSPFELMSAILKGAKLDGSNSSDSGVAVSPAVMAMLLENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFRKETLAFFILATYGDGEPTDNAARFYKWFVEGNDRGDWLKNLQYGVFGLGNRQYEHFNKIAKVVDEKVAEQGGKRIVPLVLGDDDQCIEDDFAAWRENVWPELDNLLRDEDDTTVSTTYTAAIPEYRVVFPDKSDSLISEANGHANGYANGNTVYDAQHPCRSNVAVRKELHTPASDRSCTHLDFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEADRLKYLASPAGKDEYAQSLVANQRSLLEVMAEFPSAKPPLGVFFAAIAPRLQPRFYSISSSPRMAPSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRKMDYIYEDELNHFLEIGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 69 - protein, 134 aa, Catharanthus roseus
MASDQKLHKFDEVSKHNKTKDCWLIINGKVYDVTPFMDDHPGGDEVLLSATGKDATNDFEDVGHSDSAREMMDKYYIGEMDMATVPLKRTYIPPQQAQYNPDKTPEFVIKILQFLVPLLILGLAFAVRHYTKEK

SEQ ID NO: 70 - protein, 375 aa, Vinca minor
MTKISPSVITCKAAVIWKSGEPAKVEEIQVDPPKASEVRIKMLCASLCHTDFLCCNGLPVPLFPRIPGHEGVGMIESVGENVTELKGGDIVMPLYLGECGECLNCKSGRTNLCHKYPLGFSGLLLDGTSRMSIGGQKIYHHFSCSTWSEYIVIEEAYAVKVDPRVSLSHASFLCCGFTTGFGATWRDVNVEKGATVAVLGLGAVGLGAVQGARSQGASSIIGVDINDKKREKGEAFGITEFINPKGSNKSVSDLIKEATGGLGVDYVYECTGVPSLLNEAIEASKVGLGTAVLIGAGLETSGEIKFIPLLCGRTVKGSIYGGVRPKSDLPTIIEKCINKEIPMDALLTHEVSLSEINKGLEYLKHPDCVKVVIKF

SEQ ID NO: 71 - protein, 397 aa, Nepeta cataria
MSMNWWRDGAAKKRMDESSAVKLQQQQCVALIVGVTGLVGNSLAEMLPLSDTPGGPWKVYGVARRPRPSWNEDHPINYISCDVSNTAEVEAKLPPLSDVTHIFYATWTSRSSEEENCEANGKMLKNVLDTMIPNCPNLKHICLQTGRFHYVASVVDWKINGSHDTPLTEDLPRLNTNNFYYTQEDILLEEVKRKEGLTWSVHRPGTIFGFSPYSMMNLVGTLCVYAAICKQEGAVLRFPGCKGAWDGYSDCADADLIAEHYIWAALDPHAKNQSFNVSNGDVFKWKHLWKVLAEQFGVECGGYEYEEGQQVRLQDVMKDKGPVWDKIVRENGLSNTKLEDVGKWWFSDTILWNECRLDSMNKSKEHGFLGFRNSKNCFLYWIHKLKAYKIVPSSTIS

SEQ ID NO: 72 - protein, 160 aa, Nepeta cataria
MASKLEIEIELKSDVEKMWKHFKEFTKLFPKACPHLYEKIDVIQGDGISVGTIFVSTLKPTELNPVVMVTKEKIDFLDDDNKMLRYSYMEGEILKNYKNLRGIVHMSSSKNGGTIFKYSGEFEKANEQVPDPVFFKDFMVIVFQRLDDYVLNGMNHTCQN

SEQ ID NO: 73 - protein, 515 aa, Catharanthus roseus
MATITFDSLNPVTVAISAGFLLLLIIFVKSRTGSSKRKPPGPPGWPIFGNMFDLGDLPHQTLYKLKSKYGPIVWLQLGSINTMVVQNAVSAAELFKKHDVPFCDRKVPDTLTAFNFNQGSLGMNTYGGHWRVLRRLCSMEFLVNKRMNETTDLRRRIEDNMVRWIEEDSLASKAQGGTGAVQLSRFLFLMAFNLVGNLMLSRDLMDNKDPEGREFFDCMNEILELAGTPNIADFLPLLKKLDPLGMKKRMVDNMSRTMKISSKFVQERLDNRKAGKINEKKDFLDVMLEYQGDGKDGPDKFTEQHVNIVIMEMFFAGSETTSISIEWGFTELLRNPHAFKKVREEIDRVVGVNRMVEETDMENLPYLQAVVKETLRLHPALPMLLPRNTMEDTEYMGYLIPKGTQVFVNAWAIGRDPEYWQDPLSFKPERFINSSVEYKGQHFELIPFGSGRRICVGFPLAHRVVHLTLATLVQAFDWDLGAGVKPQDIDLEERLGLTLRKKNPLNVIPKKRVHI

SEQ ID NO: 74 - protein, 376 aa, Catharanthus roseus
MQIITCKAVVCWAAGEPPVVEEILVEPPRSGEVRIKILFASLCHTDVLACKGFPTPMFPRVLGHEGVGVVECVGEGVSELREGDVVIPTYLGECGECENCESGRTNLCRTYPLQAFTGLMPDGSSRMSSAKGGEMLYQFLSCSTWSEYTVIDANYAVKIDSRIPLPHASFLSCGFTTGFGATWKEAKLQEGSSTVAVLGLGAVGLGAVEGARVQGVTQIIGIDINDNKREKGEAFGMTHFINPKKDNNKSISELVKELTKGQGVDVCFECTGVPDLVNEALESTKIGTGNMIMLGAGTQKSMTINFVSLLGCRTFKYSVFGGVKVQSDLPLIIQKCLNKEIQKIEQLLTHQVQLEDINRAFELLKEPDCVKVLITL

SEQ ID NO: 75 - protein, 482 aa, Catharanthus roseus
MGSQETNLPPHVLIFPLPIQGHVNSMLRLAELLCLAELDITFIVSEFSHSRLIKHTNVASRFARYPGFQFQPISDGLPDDHPRAGERVMDILPSTKNVTGPLFKQMMVENKCFSSATRRPITCIIADGVLSFAGDFAQEKGIPLIYFRTVSACSFWACFCMPELIESGDIPIKGNGMDLIVKSVPGMETFLRRRDLPGFCRVNDINEPKLQILKTETRQTTRAQAAILNTFEDLEGPILSQIRKHMPRLFTIGPSHSHLTSRLETKNIKTLISSGSFWEEDRSCVDWLDAQPPRSVLYVSFGSITVVTRDQLLEFWYGLVNSGQRFLWVMRPDSIMGKDGQSQIPADLEEGTKARGYMVGWAPQEEVLNHPAIGGFLTHSGWNSTLESIVAGVPMICWPYFADQMINSRFVSEIWKIGLDMKDTCDRETIVKMVRELMEIRKDEFLQRADHMAKLAKEAVSEGGSSYSNLDGLVDYIKSLII

SEQ ID NO: 76 - protein, 521 aa, Catharanthus roseus
MELNFKSIIFLVFVSLTLYWVYRILDWVWFKPKKLEKCLREQGFKGNPYRLFLGDQYDSGKLIRQALTKPIGVEEDVKKRIVPHILKTVGTHGKKSFMWVGRIPRVNITDPELIKEVLTKYYKFQKNHHDLDPITKLLLTGIGSLEGDPWAKRRKIINAAFHFEKLKLMLPAFYLSCRDMVTKWDNKVPEGGSAEVDVWHDIETLTGDVISRTLFGSNFEEGRRIFELMKELTALTIDVIRSVYIPGQRFLPTKRNNRMRAIDKEVRVRITEIINKKMKVMKSGEAASAADDFLGILLECNLNEIKEQGNNKSAGMTIGEIIGECKLFYFAGQDTTSTLLVWTMVLLSRFPEWQTRAREEVFQVFGNKTPDYDGISHLKVITMILYEVLRLYTPVAELTKVAHEATQLGKYFIPAGVQLMMPQILLHHDPEIWGEDVMEFKPERFAEGVLKATKSQGSFFPFSLGPRMCIGQNFALLEAKMAMSLILRRFSFELSPSYVHAPFTLITMQPQYGAHLILHKL

SEQ ID NO: 77 - protein, 371 aa, Catharanthus roseus
MVATIDSIEMPALPTAVEAHPMKGGDDSHSYSQNSCYQKGVIDAAKAVIVEAVNEKLDLENNPIFDPIKPFRIADFGCSTGPNTFHAMQNIVESVETKYKSLQKTPEFHVFFNDHVNNDFNVLFRSLPPNREFFAAGVPGSFYTRVFPKNSIHFAHCSYALHWLSKVPKEIQDKNSLAYNKGRIHYTGTEKHVVKAYFGQFQRDFEGFLKARAQEIVVGGLMVIQIPGLPSGEVLFSRTGAGLLHFLLGTSLMELVNKGIINEESVDSFNLPQYHPSVEDLEMVIEMNDCFTIERVGTLPHPMKNLPFDVQRTSLQVRAIMECILTEHFGENILDPLFEIYTKNLQENFHVFDKEIRKDADLYLVLKRKGN

SEQ ID NO: 78 - protein, 524 aa, Catharanthus roseus
MEMDMDTIRKAIAATIFALVMAWAWRVLDWAWFTPKRIEKRLRQQGFRGNPYRFLVGDVKESGKMHQEALSKPMEFNNDIVPRLMPHINHTINTYGRNSFTWMGRIPRIHVMEPELIKEVLTHSSKYQKNFDVHNPLVKFLLTGVGSFEGAKWSKHRRIISPAFTLEKLKSMLPAFAICYHDMLTKWEKIAEKQGSHEVDIFPTFDVLTSDVISKVAFGSTYEEGGKIFRLLKELMDLTIDCMRDVYIPGWSYLPTKRNKRMKEINKEITDMLRFIINKRMKALKAGEPGEDDLLGVLLESNIQEIQKQGNKKDGGMSINDVIEECKLFYFAGQETTGVLLTWTTILLSKHPEWQERAREEVLQAFGKNKPEFERLNHLKYVSMILYEVLRLYPPVIDLTKIVHKDTKLGSYTIPAGTQVMLPTVMLHREKSIWGEDAMEFNPMRFVDGVANATKNNVTYLPFSWGPRVCLGQNFALLQAKLGLAMILQRFKFDVAPSYVHAPFTILTVQPQFGSHVIYKKLES

SEQ ID NO: 79 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 80 - protein, 490 aa, Ruminococcus gnavus
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 81 - protein, 352 aa, Catharanthus roseus
MANFSESKSMMAVFFMFFLLLLSSSSSSSSSSPILKKIFIESPSYAPNAFTFDSTDKGFYTSVQDGRVIKYEGPNSGFTDFAYASPFWNKAFCENSTDPEKRPLCGRTYDISYDYKNSQMYIVDGHYHLCVVGKEGGYATQLATSVQGVPFKWLYAVTVDQRTGIVYFTDVSSIHDDSPEGVEEIMNTSDRTGRLMKYDPSTKETTLLLKELHVPGGAEISADGSFVVVAEFLSNRIVKYWLEGPKKGSAEFLVTIPNPGNIKRNSDGHFWVSSSEELDGGQHGRVVSRGIKFDGFGNILQVIPLPPPYEGEHFEQIQEHDGLLYIGSLFHSSVGILVYDDHDNKGNSYVSS

SEQ ID NO: 82 - protein, 532 aa, Rauvolfia serpentina
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 83 - protein, 356 aa, Catharanthus roseus
MAMASKSPSEEVYPVKAFGLAAKDSSGLFSPFNFSRRATGEHDVQLKVLYCGTCQYDREMSKNKFGFTSYPYVLGHEIVGEVTEVGSKVQKFKVGDKVGVASIIETCGKCEMCTNEVENYCPEAGSIDSNYGACSNIAVINENFVIRWPENLPLDSGVPLLCAGITAYSPMKRYGLDKPGKRIGIAGLGGLGHVALRFAKAFGAKVTVISSSLKKKREAFEKFGADSFLVSSNPEEMQGAAGTLDGIIDTIPGNHSLEPLLALLKPLGKLIILGAPEMPFEVPAPSLLMGGKVMAASTAGSMKEIQEMIEFAAEHNIVADVEVISIDYVNTAMERLDNSDVRYRFVIDIGNTLKSN

SEQ ID NO: 84 - protein, 359 aa, Catharanthus roseus
MAAKSPENVYPVKTFGFAAKDSSGFFSPFNFSRRATGENDVQFKVLYCGTCNYDLEMSTNKFGMTKYPFVIGHEIVGVVTEIGSKVQKFKVGDKVGVGGFVGACEKCEMCVNGVENNCSKVESTDGHFGNNFGGCCNIMVVNEKYAVVWPENLPLHSGVPLLCAGITTYSPLRRYGLDKPGLNIGIAGLGGLGHLAIRFAKAFGAKVTLISSSVKKKREALEKFGVDSFLLNSNPEEMQGAYGTLDGIIDTMPVAHSIVPFLALLKPLGKLIILGVPEEPFEVPAPALLMGGKLIAGSAAGSMKETQEMIDFAAKHNIVADVEVIPIDYLNTAMERIKNSDVKYRFVIDVGNTLKSPSF

SEQ ID NO: 85 - protein, 364 aa, Catharanthus roseus
MAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEE

SEQ ID NO: 86 - protein, 362 aa, Catharanthus roseus
MAGETTKLDLSVKAIGWGAADASGVLQPIRFYRRAPGERDVKIRVLYCGVCNFDMEMVRNKWGFTRYPYVFGHETAGEVVEVGKKVEKFKVGDKVGVGCMVGSCGKCFHCQNEMENYCPEPNLADGSTYREEGERSYGGCSNVMVVDEKFVLRWPENLPQDKGVPLLCAGVVVYSPMKYMGFDKPGKHIGVFGLGGLGSIAVKFIKAFGGKATVISTSRRKEKEAIEEHGADAFVVNTDSEQLKALEGTMDGVVDTTPGGRTPMPLMLNLVKFDGAVILVGAPETLFELPLEPMGRKKIIGSSTGGLKEYQEVLDIAAKHNIVCDTEVIGIDYLSTAMERIKNLDVKYRFAIDIGNTLKFEE

SEQ ID NO: 87 - protein, 414 aa, Saccharomyces cerevisiae
MFVRVKLNKPVKWYRFYSTLDSHSLKLQSGSKFVKIKPVNNLRSSSSADFVSPPNSKLQSLIWQNPLQNVYITKKPWTPSTREAMVEFITHLHESYPEVNVIVQPDVAEEISQDFKSPLENDPNRPHILYTGPEQDIVNRTDLLVTLGGDGTILHGVSMFGNTQVPPVLAFALGTLGFLSPFDFKEHKKVFQEVISSRAKCLHRTRLECHLKKKDSNSSIVTHAMNDIFLHRGNSPHLTNLDIFIDGEFLTRTTADGVALATPTGSTAYSLSAGGSIVSPLVPAILMTPICPRSLSFRPLILPHSSHIRIKIGSKLNQKPVNSVVKLSVDGIPQQDLDVGDEIYVINEVGTIYIDGTQLPTTRKTENDFNNSKKPKRSGIYCVAKTENDWIRGINELLGFNSSFRLTKRQTDND

SEQ ID NO: 88 - protein, 501 aa, Catharanthus roseus
MEFSFSSPALYIVYFLLFFVVRQLLKPKSKKKLPPGPRTLPLIGNLHQLSGPLPHRTLKNLSDKHGPLMHVKMGERSAIIVSDARMAKIVLHNNGLAVADRSVNTVASIMTYNSLGVTFAQYGDYLTKLRQIYTLELLSQKKVRSFYSCFEDELDTFVKSIKSNVGQPMVLYEKASAYLYATICRTIFGSVCKEKEKMIKIVKKTSLLSGTPLRLEDLFPSMSIFCRFSKTLNQLRGLLQEMDDILEEIIVEREKASEVSKEAKDDEDMLSVLLRHKWYNPSGAKFRITNADIKAIIFELILAATLSVADVTEWAMVEILRDPKSLKKVYEEVRGICKEKKRVTGYDVEKMEFMRLCVKESTRIHPAAPLLVPRECREDFEVDGYTVPKGAWVITNCWAVQMDPTVWPEPEKFDPERYIRNPMDFYGSNFELIPFGTGRRGCPGILYGVTNAEFMLAAMFYHFDWEIADGKKPEEIDLTEDFGAGCIMKYPLKLVPHLVND

SEQ ID NO: 89 - protein, 501 aa, Tabernaemontana elegans
MELSFSSPALYILYFLLFLLVKQLLKPKSQKKLPPGPRTLPIIGNLHQLMGPLPHRTLKDLSDKHGPLMHLKMGERSAIIVSDARMAKIVLHNHGLAVADRSVNTVASIMTYNSLGVTFAQYGDYLTKLRQIYTLELLSPKKVRSFYSCFEDELDGFVKSIKSQVGQPMVLYEKASTYLYATICRTIFGSICKEREKMIKIVKRTSLLSGTPLRLEDLFPSMRVFCRFSKTLNQLRGLLQEMDDILEDIIIEREKTTEISTEAKDDEDMLSVLLRHKWHNPSGAKFRITNADIKAIIFELILAATLSVADVTEWAMVEILRDPKSLKKVYDEVREVCKEKKRVTGYDVEKLEYMHLCVKESTRIHPAAPLLVPRECREDFEVDGYTVPKGAWVLTNCWAVQMDPKIWPEPEKFDPERYIRNPMDFYGSNFELIPFGTGRRGCPGILFGVTNAELLLAAMFYHFDWEIADGKKPEEIDLTEDFGAGCIMKYPLKLVPHLANE

SEQ ID NO: 90 - protein, 501 aa, Vinca minor
MEFSFSSLPLYIASFLLFFLVKQILKPKSGKKLPPGPRTLPIIGNLHQLMGPLPHRTLKNLSDKHGPLMHLKLGERSAIIVSDARLAKIVLHNNGLAVADRSVNTVASIMTYNSLGVTFAQYGDYLTKLRQIYTLELLSQKKVRSFYNCFEDELDTFVKSIKSSNGQPMVLYEVASTYLYATICRTIFGSVCKEREKMIKIVKRTSLLSGTPLRLEDLFPSMSVFCRFSKTLNQLRGLLDEMDHILEDIIVDREKNTEILKEGKDDEDMLSVLLRHKWHNPSGAKFRIANADIKAIIFELILAATLSVADVTEWAMVEIIRDPKSLKKVHEEVRQVCKDKGKVTGYDVEKLDYLRLCLKESTRIHPAAPLLVPRECREDFQVDGFTVPKGAWVLTNCWAVQMDPEVWPEPEKFDPERYIRKPMDFYGNSFELIPFGTGRRGCPGILYGVTNAEMMLAAMLYHFDWEIADGKKAEEIDMTEDFGAGCIMKYPLKLIPHIVNE

SEQ ID NO: 91 - protein, 501 aa, Amsonia hubrichtii
MEFSFSSPLLYIVYFLLFFLVRYLLKPKSNKKLPPGPRTLPIIGNLHQLMGPLPHRTLKELSDRHGPLMHVKMGERSAIIVSNARMAKIVLHNHGLAVADRSVNTVASIMTYNSLGVTFAQYGDYLTKLRQIYTLELLSPKKVRSFYGCFEEELDVFVESIKSSVGQPMVMYEKCSKYLYATICRTIFGSVCKEREKMIKIVKKTSLLSGTPLRLEDLFPSMSVFARFSKTLKQLRGLLQEMDDILEDIITEREKTTEISTESKDDEDMLSVLLRHKWHNPSGAKFRITNADIKAIIFELILAATLSVADVAEWAMVEILRDPKSLKKVYEEVREVCKEKKKVTGHDVEKLEFMRLCLKESTRIHPAAPLLVPRECREDFEVDGYTVPKGAWVLTNCWAVQMDPEVWPEPEKFDPERYIRNPMDFYGSSFELIPFGTGRRGCPGILYGVTNAELMLAAMFYHFDWEIADGKKPEEIDMTEDFGAGCIMKYPLKLVPHLAKN

SEQ ID NO: 92 - protein, 354 aa, Catharanthus roseus
MADRVKTVGWAAHDSSGFLSPFQFTRRATGEEDVRLKVLYCGVCHSDLHNIKNEMGFTSYPCVPGHEVVGEVTEVGNKVKKFIIGDKVGVGLFVDSCGECEQCVNDVETYCPKLKMAYLSIDDDGTVIQGGYSKEMVIKERYVFRWPENLPLPAGTPLLGAGSTVYSPMKYYGLDKSGQHLGVVGLGGLGHLAVKFAKAFGLKVTVISTSPSKKDEAINHLGADAFLVSTDQEQTQKAMSTMDGIIDTVSAPHALMPLFSLLKPNGKLIVVGAPNKPVELDILFLVMGRKMLGTSAVGGVKETQEMIDFAAKHGIVADVEVVEMENVNNAMERLAKGDVRYRFVLDIGNATVAV

SEQ ID NO: 93 - protein, 323 aa, Catharanthus roseus
MEKQVEIPEVELNSGHKMPIVGYGTCVPEPMPPLEELTAIFLDAIKVGYRHFDTASSYGTEEALGKAIAEAINSGLVKSREEFFISCKLWIEDADHDLILPALNQSLQILGVDYLDLYMIHMPVRVRKGAPMFNYSKEDFLPFDIQGTWKAMEECSKQGLAKSIGVSNYSVEKLTKLLETSTIPPAVNQVEMNVAWQQRKLLPFCKEKNIHITSWSPLLSYGVAWGSNAVMENPVLQQIAASKGKTVAQVALRWIYEQGASLITRTSNKDRMFENVQIFDWELSKEELDQIHEIPQRRGTLGEEFMHPEGPIKSPEELWDGDL

SEQ ID NO: 94 - protein, 421 aa, Catharanthus roseus
MAPQMQILSEELIQPSSPTPQTLKTHKLSHLDQVLLTCHIPIILFYPNQLDSNLDRAQRSENLKRSLSTVLTQFYPLAGRININSSVDCNDSGVPFLEARVHSQLSEAIKNVAIDELNQYLPFQPYPGGEESGLKKDIPLAVKISCFECGGTAIGVCISHKIADALSLATFLNSWTATCQEETDIVQPNFDLGSHHFPPMESIPAPEFLPDENIVMKRFVFDKEKLEALKAQLASSATEVKNSSRVQIVIAVIWKQFIDVTRAKFDTKNKLVAAQAVNLRSRMNPPFPQSAMGNIATMAYAVAEEDKDFSDLVGPLKTSLAKIDDEHVKELQKGVTYLDYEAEPQELFSFSSWCRLGFYDLDFGWGKPVSVCTTTVPMKNLVYLMDTRNEDGMEAWISMAEDEMSMLSSDFLSLLDTDFSN

SEQ ID NO: 95 - protein, 529 aa, Catharanthus roseus
MIKKVPIVLSIFCFLLLLSSSHGSIPEAFLNCISNKFSLDVSILNILHVPSNSSYDSVLKSTIQNPRFLKSPKPLAIITPVLHSHVQSAVICTKQAGLQIRIRSGGADYEGLSYRSEVPFILLDLQNLRSISVDIEDNSAWVESGATIGEFYHEIAQNSPVHAFPAGVSSSVGIGGHLSSGGFGTLLRKYGLAADNIIDAKIVDARGRILDRESMGEDLFWAIRGGGGASFGVIVSWKVKLVKVPPMVTVFILSKTYEEGGLDLLHKWQYIEHKLPEDLFLAVSIMDDSSSGNKTLMAGFMSLFLGKTEDLLKVMAENFPQLGLKKEDCLEMNWIDAAMYFSGHPIGESRSVLKNRESHLPKTCVSIKSDFIQEPQSMDALEKLWKFCREEENSPIILMLPLGGMMSKISESEIPFPYRKDVIYSMIYEIVWNCEDDESSEEYIDGLGRLEELMTPYVKQPRGSWFSTRNLYTGKNKGPGTTYSKAKEWGFRYFNNNFKKLALIKGQVDPENFFYYEQSIPPLHLQVEL

SEQ ID NO: 96 - protein, 530 aa, Amsonia hubrichtii
MYTDVHKVFTFSCFLLLVSSCHCLTLESFISCFSSEFPSDEPIFSVLHDPRNASYQPLLESTIHNLRFLKSPKPLAIITPFRYSHVQAAVVCCKQVRLQIRIRSGGHDYEGLSYRSEVPFIILDLRNLRSITVDIEDSSAWVESGATSGELYYQIAMKSPIHAFPAGIYSTVGVGGHFSGGGFGTMLRKYGLAADNIIDAYIVDAKGRLLNRESMGKDLFWAIRGGGGASFGVIVAWKIKLVHVPPVVTVFDLPKTFEEGAIDLIHKWQNVGPEVSEDLFLAVSVMADASGGNKTIMAGFTSLFLGTADLLLKEMEERFPELGLRTEHCSEMSWINAVMHFSGYPSGETISALKNGEAPLPKNCVATNSDFVQEPLSVSALEKLWNFCLEERNTPIILMLPHGGMMSKISETETPFPYRQGVIYSLIYEVVWDCQDDYFSEEYISSLRRFHDHLTPHVLKHPRGTFLNTRNLDTGKHDNYDITYSKAKEWGLKYFKKNFRRLAIIKGEVDAENFFHFEQSIPPLVSIDEL

SEQ ID NO: 97 - protein, 529 aa, Tabernanthe iboga
MVEVSKVLSCFCFLLLLSLSHGLIPEAFITCISQEFESYESILRVLHDPGNSSYHSVLQSRIQNLRFLKSPKPLAIITPLLYSHVQAAIVCSKQVGLQIRIRSGGNDYEGLSYRSEVPFILLDLHNLRSIAVDIEDNSAWVESGATIGELYYEIAQKSPIHAFPAGVYATVGVGGHFSGGGFGTMLRKYGLAADNVIDAYIVDARGRLLDRESMGEDLFWAIRGGGGASFGVIVSWKIKLVHVPPVVTVFDMPKTLEQGALDLLNKWQYIGHKVSEDLFLAVSIMADTSGGNKTLMAGFNSLFLGTADQLLKEMAESFPELGLRKEHCSEMGWIKAAMHFSGYPSVETTSPLKSREPPLPKTCIATKSDFIQEPLSLPALEKLWKLLWEEENTPIILMLPHGGMMSKISEWELPFPYRQDVIYSMIDEVVWDCEDDYSSEEHISGLRKLYDLMTPYVSKHPRGTFLNMRNLDTGRNGDYYGTTYSKAKEWGLKYFKNNFRKLAITKGAVDPDNFFYFEQSIPPLTSQDE

SEQ ID NO: 98 - protein, 531 aa, Tabernaemontana elegans
MYTTEVHKVFISYCFLLLLSQCHCLIPESFISCLSKKFPSDEPIFSVLHDPRNASYQAVLESNLQNLRFLKSAKPLAIITPLHYTHVQAAVVCCKRAGLQIRIRSGGSDYEGLSYRSEVPYIILDLQNLRSITVDIEDNSAWVESGATIGELYYEIADQSPVHAFPAGVYSTVGVGGHLSGGGFGTMLRKYGLAADNILDAHIVDAEGRLLNRESMGTDLFWAIRGGGGASFGVIVAWKVKLVHVPPVVTVFDLAKTLEEGAIDLIHKWQTVGPNLNEDAFLAASIMADPSSESKTLVAGFFSLFLGIADQLLKEMKESFPELGLRKEDCLEMSWIKAALHFSGYEPGETVYALKNRKPPQPKQCITVRSDFIQEPLSLPALDKLWKFLSEEENTPIIVMLPHGGMMSKISETEIPYPYREGVIYSFLYELNWDCEDDSFSERYVSALTRLYDHMTPYVLKHPRGGFLNMRCLEIGKNDDYGTTYSKAKEWGLKYFKNNFKRLAITKGAVDPDNFFYFEQSIPPLASKDEL

SEQ ID NO: 99 - protein, 365 aa, Catharanthus roseus
MAGKSAEEEHPIKAYGWAVKDRTTGILSPFKFSRRATGDDDVRIKILYCGICHTDLASIKNEYEFLSYPLVPGMEIVGIATEVGKDVTKVKVGEKVALSAYLGCCGKCYSCVNELENYCPEVIIGYGTPYHDGTICYGGLSNETVANQSFVLRFPERLSPAGGAPLLSAGITSFSAMRNSGIDKPGLHVGVVGLGGLGHLAVKFAKAFGLKVTVISTTPSKKDDAINGLGADGFLLSRDDEQMKAAIGTLDAIIDTLAVVHPIAPLLDLLRSQGKFLLLGAPSQSLELPPIPLLSGGKSIIGSAAGNVKQTQEMLDFAAEHDITANVEIIPIEYINTAMERLDKGDVRYRFVVDIENTLTPPSEL

SEQ ID NO: 100 - protein, 365 aa, Tabernanthe iboga
MAGKSPEEEHPVKAYGWAVKDRTTGILSPFKFSRRATGDNDIRIKILYCGICHTDLTSVKNEYEFLSYPLVPGMEIVGIATEVGSKVTKIKVGEKVAVAAYLGTCGKCYNCVNDLENYCPEVIIGYGTPYHDGTINYGGLSNETVVNERFVLRFPEKLSPAGGAPLLSAGITAYSAMRNHGLDKPGIQLGVVGLGGLGHLAVKFAKAFGVRVTVISTTPSKKDEAINNLGADAFLFSRDDKQMRAAIGTFDAIIDTLAVVHPIAPLLDLLRSHGKLVLVGAPSKPLELPTIPLLSGGKSLIGSAAGNVKQTQEMLDFAAEHDITANIEVIPIDYINTAMERLDKGDIRFRFVVDIENTLTPPPEP

SEQ ID NO: 101 - protein, 320 aa, Catharanthus roseus
MGSSDETIFDLPPYIKVFKDGRVERLHSSPYVPPSLNDPETGGVSWKDVPISSVVSARIYLPKINNHDEKLPIIVYFHGAGFCLESAFKSFFHTYVKHFVAEAKAIAVSVEFRLAPENHLPAAYEDCWEALQWVASHVGLDISSLKTCIDKDPWIINYADFDRLYLWGDSTGANIVHNTLIRSGKEKLNGGKVKILGAILYYPYFLIRTSSKQSDYMENEYRSYWKLAYPDAPGGNDNPMINPTAENAPDLAGYGCSRLLISMVADEARDITLLYIDALEKSGWKGELDVADFDKQYFELFEMETEVAKNMLRRLASFIK

SEQ ID NO: 102 - protein, 330 aa, Catharanthus roseus
MNSSTDPTSDETIWDLSPYIKIFKDGRVERLHNSPYVPPSLNDPETGVSWKDVPISSQVSARVYIPKISDHEKLPIFVYVHGAGFCLESAFRSFFHTFVKHFVAETKVIGVSIEYRLAPEHLLPAAYEDCWEALQWVASHVGLDNSGLKTAIDKDPWIINYGDFDRLYLAGDSPGANIVHNTLIRAGKEKLKGGVKILGAILYYPYFIIPTSTKLSDDFEYNYTCYWKLAYPNAPGGMNNPMINPIAENAPDLAGYGCSRLLVTLVSMISTTPDETKDINAVYIEALEKSGWKGELEVADFDADYFELFTLETEMGKNMFRRLASFIKHE

SEQ ID NO: 103 - protein, 512 aa, Catharanthus roseus
MELYYFSTFAFLLFCFILAKTLKKSGQSNLKLPLGPPPIPILGNAHQLIGGHTHHILRDLAKKYGPLMHLKTGEVSTIVASSPEIAEEMFKTHDVLFADRPSNIVAFKILSYDYSDVVISPYGNYWRQLRKISMMELFSQRSVQSFRSIREEEVLNFIKSIGSREGTKINLSKEISLLIYGITTRAAFGEKNKNTEEFIRLLDQLTVAVAEPNIADMFPSINFLKLISRSKYKIEKIHKNFDAIVQTILNHHKDRLANHKSSSHEENGEQNKDLVDVLLNIQQRGDFDTPLGDRSVKAVIFNIFSAGTETSSTTVDWAMCEMIKNPTIMKKAQEEVRKVYNEEGNVNETKLHQLKYLKAVIKETLRLHPPVPLLLPRECREQCEIKGYTIPSKSRVIVNAWAIGRDPNYWIEPENFNPERFLESEVDFKGNSFEYLPFGGGRRICPGITFALANIELPLAQLLFHFDWKLASDETNIDKLDMTESRGVTVRREDDLCLIPFPYSASSLKGKY

SEQ ID NO: 104 - protein, 355 aa, Catharanthus roseus
MDVQSEEFRGAQAQIWSQSCSFITSASLKCAVKLGIPDTIDNHGKPITLSELTNALVPPVHPSKAPFIYRLMRVLAKNGFCSEEQLDGETEPLYSLTPSSRILLKKEPLNLRGIVLTMADPVQLKAWESLSDWYQNEDDSSTAFETAHGKNFWGYSSEHMEHAEFFNEAMASDSQLISKLLIGEYKFLFEGLASLVDIGGGTGTIAKAIAKNFPQLKCTVFDLPHVVANLESKENVEFVAGDMFEKIPSANAIFLKWILHDWNDEDCVKILKSCKKAIPAKGGKVIIIDMVMYSDKKDDHLVKTQTSMDMAMLVNFAAKERCEKEWAFLFKEAGFSDYKIYPKLDFTRSLIEVYP

SEQ ID NO: 105 - protein, 506 aa, Catharanthus roseus
MEFHESSPFVFITRGFIFIAISIAVLRRIISKKTKTLPPGPWKLPLIGNLHQFLGSVPYQILRDLAQKNGPLMHLQLGEVSAIVAASPQMAKEITKTLDLQFADRPVIQALRIVTYDYLDISFNAYGKYWRQLRKIFVQELLTSKRVRSFCSIREDEFSNLVKTINSANGKSINLSKLMTSCTNSIINKVAFGKVRYEREVFIDLINQILALAGGFKLVDLFPSYKILHVLEGTERKLWEIRGKIDKILDKVIDEHRENSSRTGKGNGCNGQEDIVDILLRIEEGGDLDLDIPFGNNNIKALLFDIIAGGTETSSTAVDWAMSEMMRNPHVMSKAQKEIREAFNGKEKIEENDIQNLKYLKLVIQETLRLHPPAPLLMRQCREKCEIGGYHIPVGTKAFINVWAIGRDPAYWPNPESFIPERFDDNTYEFTKSEHHAFEYLPFGAGRRMCPGISFGLANVELPLALLLYHFNWQLPDGSTTLDMTEATGLAARRKYDLQLIATSYA

SEQ ID NO: 106 - protein, 356 aa, Catharanthus roseus
MSSEMAAKSVKAFGLALKDSSGLFSPFNFSRRATGEHDVQLKVLYCGVCNFDNLMRRNKYGRTKFPYVFGHEIVGVVTEVGSNVKKFKIGNKVGVSFIVDTCRECERCKIGQQIACKKAVSSDGFFETPGYGGCSNIFVADENYVILWPENLPMDSGAPLLCIGITCYNPLRRFGLDKPGVRVGIVGLGAVGHLAIKFAKAFGARVTLISSSPGKKDEAFQKFGVDSFLVSSNAEEMQAAAETLDGILDTVPVVHPLEPLFALLKPLGKLIIIGEPHKPFEVSAMSLMEGGKIISASTGGSIKDTQEIVDFAAEHNVVADVEVIPVDYVNTAMERLDKADVKYRFVIDIGNTFKSP

SEQ ID NO: 107 - protein, 289 aa, Catharanthus roseus
MEEKQEKVAEFYDKVTGAWDLFYGVHLHDGYYEPGTTATMAISQDAVIRMIDELLRFAGVSEDPAKKPRSMLDVGSGLGGTCVYVAKKYDIQCTGITISPNQVKYAQDYAATEGVENKVSFDCGDALDMPYSDGKFDVVFTINCIEHVHDKEKFIREMVRVAAPGAAIIIASQAHPNLSPGESLKPRDKKILQKICDGAGAVSLCSSDDYVRWLTPLPVKEIKAADWTQNITPLYPLLMKEAFTWKGFTSIVLKGGWRAINLINAVRLVAKAANDGILKFAVVTGRKSI

SEQ ID NO: 108 - protein, 401 aa, Catharanthus roseus
MPKSWPIVISSHSFCFLPNSEQERKMKDLNFHAATLSEEESLRELKAFDETKAGVKGIVDTGITKIPRIFIDQPKNLDRISVCRGKSDIKIPVINLNGLSSNSEIRREIVEKIGEASEKYGFFQIVNHGIPQDVMDKMVDGVRKFHEQDDQIKRQYYSRDRFNKNFLYSSNYVLIPGIACNWRDTMECIMNSNQPDPQEFPDVCRDILMKYSNYVRNLGLILFELLSEALGLKPNHLEEMDCAEGLILLGHYYPACPQPELTFGTSKHSDSGFLTILMQDQIGGLQILLENQWIDVPFIPGALVINIADLLQLITNDKFKSVEHRVLANKVGPRISVAVAFGIKTQTQEGVSPRLYGPIKELISEENPPIYKEVTVKDFITIRFAKRFDDSSSLSPFRLNN

SEQ ID NO: 109 - protein, 439 aa, Catharanthus roseus
MESGKISVETETLSKTLIKPSSPTPQSLSRYNLSYNDQNIYQTCVSVGFFYENPDGIEISTIREQLQNSLSKTLVSYYPFAGKVVKNDYIHCNDDGIEFVEVRIRCRMNDILKYELRSYARDLVLPKRVTVGSEDTTAIVQLSHFDCGGLAVAFGISHKVADGGTIASFMKDWAASACYLSSSHHVPTPLLVSDSIFPRQDNIICEQFPTSKNCVEKTFIFPPEAIEKLKSKAVEFGIEKPTRVEVLTAFLSRCATVAGKSAAKNNNCGQSLPFPVLQAINLRPILELPQNSVGNLVSIYFSRTIKENDYLNEKEYTKLVINELRKEKQKIKNLSREKLTYVAQMEEFVKSLKEFDISNFLDIDAYLSDSWCRFPFYDVDFGWGKPIWVCLFQPYIKNCVVMMDYPFGDDYGIEAIVSFEQEKMSAFEKNEQLLQFVSN

SEQ ID NO: 110 - protein, 511 aa, Catharanthus roseus
MEIRDLFWSLPAIALLQVFLFFLFKNPKKTTLKLPPGPPTLPIIGNLHQMASPLPHKKLKDLADKYGPLMHLKLGEISTVVISSSRLTKEFMQTHGLNFADRPQTVIAKIMMYNCSGVTLSMYGDYWRKLRQIYVTELLNTKSVQSFSSIMEEELILMVKSIESEVGKPMELIEKIRSYLFDTLCRSALGKIHGKGKETLIEISREMVALSGVQTLEDIFPSVKLFSYLNPLRPKAKKLFKRLDSVLEDIINQQENKLLSLKDGDNQQEKEEDNMLSVLLRLRNGKDSKVKLTNNDIKAIIFELFVGGISTSSTTIEWAMSELMKNPEMMEKGKHEVRQVLKGKKRVCQIDVENMSYIKLVLKETLRFHPPGPLLFPRKSREQCEIDGYTIPAKAMILINNWVLGRDPEYWVDPEKFEPERFKDNLVDYKGNHFELIPFGVGRRICPGISFAVTNIELLLAALLFHFDWKLPHGMDPKDLDMIELYRSGCTRKNPLVLIPKIYIPTGDENY

SEQ ID NO: 111 - protein, 530 aa, synthetic construct
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSSYYCVLAGLGLVPDAPSPRLAHMPQATESVDEVFGAVKDRQRNLLETLPSLHEFLRQQHGR

SEQ ID NO: 112 - protein, 191 aa, Escherichia coli str. K-12 substr. MG1655
MRVITLAGSPRFPSRSSSLLEYAREKLNGLDVEVYHWNLQNFAPEDLLYARFDSPALKTFTEQLQQADGLIVATPVYKAAYSGALKTLLDLLPERALQGKVVLPLATGGTVAHLLAVDYALKPVLSALKAQEILHGVFADDSQVIDYHHRPQFTPNLQTRLDTALETFWQALHRRDVQVPDLLSLRGNAHA

SEQ ID NO: 113 - protein, 607 aa, synthetic construct
  REGION 1..607: note = Artificial Sequence
  REGION 1..607: note = MISC_FEATURE - Artificial Sequence
MASTRVLASRLASQMAASAKVARPAVRVAQVSKRTIQTGSPLQTLKRTQMTSIVNATTRQAFQKRAYSSGGGGGGMDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 114 - protein, 439 aa, synthetic construct
  REGION 1..439: note = Artificial Sequence
  REGION 1..439: note = MISC_FEATURE - Artificial Sequence
MASTRVLASRLASQMAASAKVARPAVRVAQVSKRTIQTGSPLQTLKRTQMTSIVNATTRQAFQKRAYSSGGGGGGMAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEE

SEQ ID NO: 115 - protein, 566 aa, synthetic construct
  REGION 1..566: note = Artificial Sequence
  REGION 1..566: note = MISC_FEATURE - Artificial Sequence
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKTGGGGGGILEQPLKFVLTAAVVLLTTSVLCCVVFT

SEQ ID NO: 116 - protein, 398 aa, synthetic construct
  REGION 1..398: note = Artificial Sequence
  REGION 1..398: note = MISC_FEATURE - Artificial Sequence
MAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEEGGGGGGILEQPLKFVLTAAVVLLTTSVLCCVVFT

SEQ ID NO: 117 - protein, 569 aa, synthetic construct
  REGION 1..569: note = Artificial Sequence
  REGION 1..569: note = MISC_FEATURE - Artificial Sequence
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKTGGGGGGSTSENQSKGSGTLVVILAILMLGVAYYLLNE

SEQ ID NO: 118 - protein, 401 aa, synthetic construct
  REGION 1..401: note = Artificial Sequence
  REGION 1..401: note = MISC_FEATURE - Artificial Sequence
MAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEEGGGGGGSTSENQSKGSGTLVVILAILMLGVAYYLLNE

SEQ ID NO: 119 - protein, 541 aa, synthetic construct
  REGION 1..541: note = Artificial Sequence
  REGION 1..541: note = MISC_FEATURE - Artificial Sequence
MDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKTLGRGRRSKL

SEQ ID NO: 120 - protein, 373 aa, synthetic construct
  REGION 1..373: note = Artificial Sequence
  REGION 1..373: note = MISC_FEATURE - Artificial Sequence
MAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEELGRGRRSKL

SEQ ID NO: 121 - protein, 549 aa, synthetic construct
  REGION 1..549: note = Artificial Sequence
  REGION 1..549: note = MISC_FEATURE - Artificial Sequence
MKAISLQRPLGLDKDVLMDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 122 - protein, 381 aa, synthetic construct
  REGION 1..381: note = Artificial Sequence
  REGION 1..381: note = MISC_FEATURE - Artificial Sequence
MKAISLQRPLGLDKDVLMAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEE

SEQ ID NO: 123 - protein, 546 aa, synthetic construct
  REGION 1..546: note = Artificial Sequence
  REGION 1..546: note = MISC_FEATURE - Artificial Sequence
MPKKKRKVGGGGGGMDNTQAEPLVVAIVPKPNASTEHTNSHLIPVTRSKIVVHRRDFPQDFIFGAGGSAYQCEGAYNEGNRGPSIWDTFTQRSPAKISDGSNGNQAINCYHMYKEDIKIMKQTGLESYRFSISWSRVLPGGRLAAGVNKDGVKFYHDFIDELLANGIKPSVTLFHWDLPQALEDEYGGFLSHRIVDDFCEYAEFCFWEFGDKIKYWTTFNEPHTFAVNGYALGEFAPGRGGKGDEGDPAIEPYVVTHNILLAHKAAVEEYRNKFQKCQEGEIGIVLNSMWMEPLSDVQADIDAQKRALDFMLGWFLEPLTTGDYPKSMRELVKGRLPKFSADDSEKLKGCYDFIGMNYYTATYVTNAVKSNSEKLSYETDDQVTKTFERNQKPIGHALYGGWQHVVPWGLYKLLVYTKETYHVPVLYVTESGMVEENKTKILLSEARRDAERTDYHQKHLASVRDAIDDGVNVKGYFVWSFFDNFEWNLGYICRYGIIHVDYKSFERYPKESAIWYKNFIAGKSTTSPAKRRREEAQVELVKRQKT

SEQ ID NO: 124 - protein, 377 aa, synthetic construct
  REGION 1..377: note = Artificial Sequence
  REGION 1..377: note = MISC_FEATURE - Artificial Sequence
MPKKKRKVGGGGGGAGETTKLDLSVKAVGWGAADASGVLQPIKFYRRVPGERDVKIRVLYSGVCNFDMEMVRNKWGFTRYPYVFGHETAGEVVEVGSKVEKFKVGDKVAVGCMVGSCGQCYNCQSGMENYCPEPNMADGSVYREQGERSYGGCSNVMVVDEKFVLRWPENLPQDKGVALLCAGVVVYSPMKHLGLDKPGKHIGVFGLGGLGSVAVKFIKAFGGKATVISTSRRKEKEAIEEHGADAFVVNTDSEQLKALAGTMDGVVDTTPGGRTPMSLMLNLLKFDGAVMLVGAPESLFELPAAPLIMGRKKIIGSSTGGLKEYQEMLDFAAKHNIVCDTEVIGIDYLSTAMERIKNLDVKYRFAIDIGNTLKFEE

SEQ ID NO: 125 - DNA, 867 bp, Saccharomyces cerevisiae
  source 1..867: mol_type = genomic DNA
atgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataa

SEQ ID NO: 126 - DNA, 1582 bp, Saccharomyces cerevisiae
  source 1..1582: mol_type = genomic DNA
atgactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaagggtgtcgaatactcattaaagcaaatggtagaagagtatggctgggaagatatggaggttgtctccgtttctggtaactactgtaccgacaaaaaaccagctgcccaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccacatgctaccgctcctggtaccaacgcacgtcaattagcaagaatagttgcctgtgccgtcttggcaggtgaattatccttatgtgatgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 127 - DNA, 2124 bp, Saccharomyces cerevisiae
  source 1..2124: mol_type = genomic DNA
atgtcagaacaactcagacaaacatttgctaacgctaaaaaagaaaacaggaacgccttggtcacatttatgaccgcaggttacccaacagtcaaagacactgtccctattctcaagggtttccaggatggtggtgtagatatcatcgaattgggtatgcccttctctgatccaattgcagatggtcctacaattcaattatctaatactgtggctttgcaaaacggtgttaccttgcctcaaactctagaaatggtctcccaagctagaaatgaaggtgttaccgtacccataatcctaatgggttactataaccctattctaaactacggtgaagaaagatttattcaggacgctgccaaggctggtgctaatggttttatcatcgtcgatttgccaccagaggaggcgttgaaggtcagaaactacatcaatgataatggtttgagcctgatcccactagtggctccttctaccaccgacgaaagattggaattactatcgcatattgccgattcgtttgtctacgttgtgtctagaatgggtactactggtgttcaaagttctgtggccagtgatttggatgaactcatctctagagtcagaaagtacaccaaggatactcctttggccgttgggtttggtgtctctaccagagaacatttccaatcagttggtagtgttgctgacggtgtagtgattggttccaaaatcgtcacattatgtggagatgctccagagggcaaaaggtacgacgttgctaaggaatatgtacagggaattctaaatggtgctaagcataaggttctgtccaaggacgaattctttgcctttcaaaaagagtccttgaagtccgcaaacgttaagaaggaaatactggacgaatttgatgaaaatcacaagcacccaattagatttggggactttggtggtcagtatgtcccagaagctcttcatgcatgtctaagagagttggaaaagggttttgatgaagctgtcgccgatcccacattctgggaagacttcaaatccttgtattcttatattggccgtccttcttcactacacaaagctgagagattaactgagcattgtcaaggtgctcaaatctggttgaagagagaagatcttaaccacacgggatctcacaagatcaacaatgctttagcacaagttcttctagctaaaagattaggcaagaagaacgttattgctgaaaccggtgctggtcaacacggtgttgccactgccactgcatgtgctaaatttggcttaacctgtactgtgttcatgggtgcagaagatgttcgtcgccaagctttaaacgtcttcagaatgagaattctcggtgctaaagtaattgctgttactaatggtacaaagactctaagagacgctacttcagaggcattcagattttgggttactaacttgaaaactacttactacgtcgtcggttctgccattggtcctcacccatatccaactttggttagaactttccaaagtgtcattggtaaagaaaccaaggaacagtttgctgccatgaacaatggtaaattacctgacgcagttgttgcatgtgttgggggtggttccaactctacaggtatgttttcaccattcgagcatgatacttccgttaagttattgggtgtggaagccggtggtgatggtgtagatacaaagttccactctgctactctaactgccggtagacctggtgtcttccatggtgtcaagacttatgtcttgcaagatagtgatggtcaagtccatgatactcattctgtttctgctgggttagactacccaggtgtcggtccagaattggcatattggaaatctactggccgtgctcaattcattgcagctactgacgctcaggctctgcttggctttaaattattatctcaattagaaggtattattcccgctttggaatcttctcatgctgtttatggcgcttgcgaattggctaagacgatgaagcctgatcaacatttggttatcaatatttctggtagaggtgataaagatgtccaaagtgtcgctgaagtcttgccgaaattaggtccaaagataggttgggatttgagattcgaagaagacccatctgcctaa

SEQ ID NO: 128 - DNA, 419 bp, Saccharomyces cerevisiae
  source 1..419: mol_type = other DNA
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagttaaatagaggtttgtccgttgtggacacgtatgctattctctccaacaagaccgttgaacaattggggcaagaagaatacgaaaaggttgctattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttaa

SEQ ID NO: 129 - DNA, 207 bp, Neurospora crassa
  source 1..207: mol_type = other DNA
atggctagtaccagagttttagcttccagattggcttctcaaatggctgcttcagcaaaggtagcaagaccagctgtcagagtggctcaggtgtctaaaaggactatacaaaccggcagtccattgcaaactctaaagagaacacaaatgacaagtatcgtcaatgcaacaacaagacaagccttccaaaagagagcttactcctcc

SEQ ID NO: 130 - DNA, 84 bp, Saccharomyces cerevisiae
  source 1..84: mol_type = genomic DNA
atattagagcaacctctgaaatttgtgcttactgcggccgtcgtgctcttgacgacgtcggttctttgttgtgtagtatttaca

SEQ ID NO: 131 - DNA, 75 bp, Saccharomyces cerevisiae
  source 1..75: mol_type = genomic DNA
tctacctctgaaaaccaaagtaaaggtagtggtacattggttgtcatattggccattttaatgctaggtgttgct

SEQ ID NO: 132 - DNA, 27 bp, Saccharomyces cerevisiae
  source 1..27: mol_type = genomic DNA
ttgggtagaggtagaagatctaagttg

SEQ ID NO: 133 - DNA, 50 bp, Saccharomyces cerevisiae
  source 1..50: mol_type = genomic DNA
aaggccatctctttacagaggccattgggtttagacaaggacgtcttatc

SEQ ID NO: 134 - DNA, 42 bp, Simian virus 40
  source 1..42: mol_type = other DNA
atgcccaagaagaaaaggaaagttggtggaggcggaggcggt

SEQ ID NO: 135 - DNA, 1245 bp, Saccharomyces cerevisiae
  source 1..1245: mol_type = genomic DNA
atgtttgtcagggttaaattgaataaaccagtaaaatggtataggttctatagtacgttggattcacattccctaaagttacagagcggctcgaagtttgtaaaaatagaggcagtaaataacttgaggagtagttcatcagcagatttcgtgtccccaccaaattccaaattacaatctttaatctggcagaaccctttacaaaatgtttatataactaaaaaaccatggactccatccacaagagaagcgatggttgaattcataactcatttacatgagtcataccccgaggtgaacgtcattgttcaacccgatgtggcagaagaaatttcccaggatttcaaatctcctttggagaatgatcccaaccgacctcatatactttatactggtcctgaacaagatatcgtaaacagaacagacttattggtgacattgggaggtgatgggactattttacacggcgtatcaatgttcggaaatacgcaagttcctccggttttagcatttgctctgggcactctgggctttctattaccgtttgattttaaggagcataaaaaggtctttcaggaagtaatcagctctagagccaaatgtttgcatagaacacggctagaatgtcatttgaaaaaaaaggatagcaactcatctattgtgacccatgctatgaatgacatattcttacataggggtaattcccctcatctcactaacctggacattttcattgatggggaatttttgacaagaacgacagcagatggtgttgcattggccactccaacgggttccacagcatattcattatcagcaggtggatctattgtttccccattagtccctgctattttaatgacaccaatttgtcctcgctctttgtcattccgaccactgattttgcctcattcatcccacattaggataaagataggttccaaattgaaccaaaaaccagtcaacagtgtggtaaaactttctgatgatggtattcctcaacaggatttagatgttggtgatgaaagttatgttataaatgaggtcggcactatatacatagatggtactcagcttccgacgacaagaaaaactgaaaatgactttaataattcaaaaaagcctaaaaggtcagggatttattgtgtcgccaagaccgagaatgactggattagaggaatcaatgaactttgtaggattcattctagctttaggctgaccaagagacagactgataatgattaa

SEQ ID NO: 136 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 137 - protein, 527 aa, Saccharomyces cerevisiae
MTADQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCDALAAGHLVQSHMTHNRKPAEPTKPNNLDATDINRLKDGSVTCIKS

SEQ ID NO: 138 - protein, 707 aa, Saccharomyces cerevisiae
MSEQLRQTFANAKKENRNALVTFMTAGYPTVKDTVPILKGFQDGGVDIIELGMPFSDPIADGPTIQLSNTVALQNGVTLPQTLEMVSQARNEGVTVPIILMGYYNPILNYGEERFIQDAAKAGANGFIIVDLPPEEALKVRNYINDNGLSLIPLVAPSTTDERLELLSHIADSFVYVVSRMGTTGVQSSVASDLDELISRVRKYTKDTPLAVGFGVSTREHFQSVGSVADGVVIGSKIVTLCGDAPEGKRYDVAKEYVQGILNGAKHKVLSKDEFFAFQKESLKSANVKKEILDEFDENHKHPIRFGDFGGQYVPEALHACLRELEKGFDEAVADPTFWEDFKSLYSYIGRPSSLHKAERLTEHCQGAQIWLKREDLNHTGSHKINNALAQVLLAKRLGKKNVIAETGAGQHGVATATACAKFGLTCTVFMGAEDVRRQALNVFRMRILGAKVIAVTNGTKTLRDATSEAFRFWVTNLKTTYYVVGSAIGPHPYPTLVRTFQSVIGKETKEQFAAMNNGKLPDAVVACVGGGSNSTGMFSPFEHDTSVKLLGVEAGGDGVDTKFHSATLTAGRPGVFHGVKTYVLQDSDGQVHDTHSVSAGLDYPGVGPELAYWKSTGRAQFIAATDAQALLGFKLLSQLEGIIPALESSHAVYGACELAKTMKPDQHLVINISGRGDKDVQSVAEVLPKLGPKIGWDLRFEEDPSA

SEQ ID NO: 139 - protein, 139 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKL

SEQ ID NO: 140 - protein, 69 aa, Neurospora crassa
MASTRVLASRLASQMAASAKVARPAVRVAQVSKRTIQTGSPLQTLKRTQMTSIVNATTRQAFQKRAYSS

SEQ ID NO: 141 - protein, 28 aa, Saccharomyces cerevisiae
ILEQPLKFVLTAAVVLLTTSVLCCVVFT

SEQ ID NO: 142 - protein, 25 aa, Saccharomyces cerevisiae
STSENQSKGSGTLVVILAILMLGVA

SEQ ID NO: 143 - protein, 9 aa, Saccharomyces cerevisiae
LGRGRRSKL

SEQ ID NO: 144 - protein, 16 aa, Saccharomyces cerevisiae
KAISLQRPLGLDKDVL

SEQ ID NO: 145 - protein, 714 aa, Amsonia hubrichtii
MDSSSEKLSPFDLMTAILRGAKFDGSNSSESGGLVSPAVVAMLMENKELMMILTTSVAVLIGCVVVLIWRRSSGSAKKVVEPPKLQVPKVDVEPEEVDDGSKKVTIFFGTQTGTAEGFAKALAEEGKARYEKATFKVIDLDDYAADDEEYEEKLKKETLAFFFLATYGDGEPTDNAARFYKWFAEGKERGDWLKNLQYGVFGLGNRQYEHFNKIAKVVDELVAEQGGKRLVPLGLGDDDQCIEDDFAAWRENVWPELDKLLRDEDDTTVSTPYTAAILEYRVVIHDRSDTLISEANGHANGYANGNTVYDAQHPCRSNVAVKKELHTPASDRSCTHLEFDIAGTGLSYETGDHVGVYCENQIETVEEAERILNLPPDTYFSIHTDKEDGTKLGGSSLQPPFPPCTLRTALTRYADLLSSPKKSALLALAAYASDPKEADRLKYLASPAGKDEYAQWLVANQRSLLEVMAEFPSAKPPLGVFFAAVAPRLQPRFYSISSSPRMAPSRIHVTCALVYEKTPTGRIHKGVCSTWMKNAIPLEESRDCNWAPIFVRQSNFKLPADPKVPIIMIGPGTGLAPFRGFLQERLALKEEGAELGPAVFYFGCRNRKLDYIYEDELNHFLETGAVSELVVAFSREGPTKQYVQHKMAEKASNIWRLISEGAYVYVCGDAKGMARDVHRTLHTIAQEQGSMDSTKAEGFVKNLQMNGRYLRDVW

SEQ ID NO: 146 - protein, 714 aa, Rauvolfia serpentina
MDSSSEKLSPFELMSAILKGAKLDGSNSSESGGAVSPAVVAMLMENKELVMILTTSMAVLIGCVVVLMWRRSSGSAKKVVEPPKSLVPKAVVEPEDVDEGKKKFTIFFGTQTGTAEGFAKALAEEAKARYEKAIIKVIDIDDFAADDEEYEEKLKKETLAFFILATYGDGEPTDNAARFYKWFVEGNERGVWLKNLQYGVFGLGNRQYEHFNKIAKVVDEQLAEQGGKRVVPLGLGDDDQCIEDDFAAWRETVWPELDQLLRDEDDTAVATPYTAAIPEYRVVFHHKSDSLISEANGHANGYANGHTVYDAQHPCRSNVAVRKELHTPASERSCTHLEFDIAGTGLSYETGDHVGVYCENLTETVEEAERLLNLPPETYFSLHADKEDGTPLGGSSLPPPFPPCTLRTSLTQYADLLSTPKKSALLALASYASDPNEADRLKYLASPAGKDEYAQSLVANQRSLLEVLAEFPSAKPPLGVFFAAIAPRLQPRFYSISSSPRMAPSRIHVTCALVYEKTPGGRIHKGVCSTWMKNAIPLEESHDCSWAPIFVRQSNFKLPTDPKVPILMIGPGTGLAPFRGFLQERLALKEEGAELGPAVFFFGCRNRKMDFIYEDELNHFLETGAVSELIVAFSREGPTKQYVQHKMAEKASDIWRMISEGAYVYVCGDAKGMARDVHRTLHTIAQEQGSMDSSKAESLVKSLQISGRYLRDVW

SEQ ID NO: 147 - protein, 714 aa, Catharanthus longifolius
MDSSSEKLSPFELMSAILKGAKLDGSNSSDSGVAVSPAVMAMLMENKELVMILTTSVAVLIGCVVVLIWRRSSGSGKKVVEPPKLIVPKSVVEPEEIDEGKKKFTIFFGTQTGTAEGFAKALAEEAKARYEKAVIKVIDIDDYAADDEEYEEKFKKETLAFFILATYGDGEPTDNAARFYKWFVEGNDRGDWLKNLQYGVFGLGNRQYEHFNKIAKVVDEKVAEQGGKRIVPLGLGDDDQCIEDDFAAWRENVWPELDNLLRDEDDTTVSTPYTAAIPEYRFVFHDKSDSLISEANGHANGYANGNTVYDAQHPCRSNVAVRKELHTPASDRSCTHLEFDIAGTGLSYGTGDHVGVYCDNLSETVEEAERLLNLPPETYFSLHADKEDGTPLAGSSLPPPFPPCTLRTALTRYADLLNTPKKSALLALAAYASDPNEANRLKYLASPAGKDEYAQSLVANQRSLLEVMAEFPSAKPPLGVFFAAIAPRLQPRFYSISSSPRMATSRIHVTCALVYEKTPGGRIHKGVCSTWMKNAIPLEESRDCSWAPIFVRQSNFKLPADPKVPVIMIGPGTGLAPFRGFLQERLALKEEGAELGTAVFFFGCRNRRMDYIYEDELNHFLETGALSELLVAFSREGPTKQYVQHKMAEKASDIWRMISDGAYVYVCGDAKGMARDVHRTLHTIAQEQGSMDSTQAEGFVKNLQMTGRYLRDVW

SEQ ID NO: 148 - protein, 4 aa, synthetic construct
  REGION 1..4: note = Artificial sequence
  REGION 1..4: note = MISC_FEATURE - Linker
GGGS

SEQ ID NO: 149 - protein, 495 aa, Saccharomyces cerevisiae
MKTDRLLINASPETCTKGDAEMDTMDTIGRMTSVKVLAEGKVLSNFEEPGLMRCAYHDAKNWVRRLSSETIVGEDTSNLYPFYVDTAYDVRRLRKDIINAKVDLQVENLIIICNINDISTVFLMREVVEWILRNFHSMTVYVQDIFEKSTQFAVGDLCKDSNCSKNRVKYWSKEFVEKHDSFFDLMITLGGDGTVLFASSIFTKDVPPIVPFALGSLGFLTNFEFQNFKETLKHILTDEVRINLRMRLQCKLYRRNKPEIDAATGRKICYIDFISEHHVLNEVTIDRGPAPCLSLLELYGNDSLMTKVQGDGLIVATPTGSTAYSLSAGGSLISPSVNAIAVTPICPHTLSFRPIILPDSMELKVRVDMNSRGTSWVNFDGKDRVELKQGDYVVITASPYSVPTIESSASEFFESISKNLNWNDREEQKPFAHILSPKNQEKYRLDSSKNGNDTISNPLESSCISSDAQDEERKSVTETETEIVVERTRQAHFAI

SEQ ID NO: 150 - DNA, 963 bp, Catharanthus roseus
  source 1..963: mol_type = genomic DNA
atgggttcctcagatgagactatttttgatcttcctccatacatcaaagtcttcaaagatggaagagtagaaagactccattcttccccatatgttcccccatctcttaatgatccagaaaccggtggagtctcttggaaagacgtcccaatttcttcagtagtttcagctagaatttaccttcctaaaatcaacaaccatgatgaaaaactccccattatagtctatttccatggagctggtttttgtcttgaatcggccttcaaatcatttttccacacttatgtgaaacactttgtagcagaagccaaagctattgcggtttctgttgagttcaggctcgcccctgaaaaccatttacccgcagcttatgaagattgctgggaagcccttcaatgggttgcttctcatgtgggtctcgacatttccagcttgaagacatgtattgataaagatccatggataatcaactatgccgatttcgatagactctatttgtggggtgatagcaccggtgccaatattgttcacaacacacttatcagatctggtaaagagaaattgaacggcggcaaagtgaagattttgggggcaattctttactacccatatttcttaatcaggacgagttcaaaacagagtgattatatggagaatgagtatagatcttactggaaattggcttacccagatgctcctggtggaaatgataacccaatgataaaccctacagctgagaatgctcctgatctggctggatatggttgctcgaggttgctgatttccatggttgccgatgaagctagagatataactcttctttatattgatgcattggaaaagagtggatggaaaggtgaattagatgtggctgattttgataaacagtattttgaactgtttgaaatggaaacagaggttgccaagaacatgctcagacgtttagcttctttcatcaagtaa

SEQ ID NO: 151 - protein, 646 aa, synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLDGGSGGGSMSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 152 - DNA, 1941 bp, synthetic construct
  source 1..1941: mol_type = other DNA
atgagtgacaagataatacacttaactgacgatagctttgacaccgatgtattaaaagcagatggtgctattcttgtcgacttttgggccgagtggtgtggcccatgtaaaatgatcgcgccaatcttagatgaaatagcggacgagtatcaaggaaaactaacggttgcaaaattgaatatcgaccagaatccagggaccgcgcccaaatatggcataaggggtattccgactctgttgctatttaaaaatggagaggttgcggccactaaggtgggtgccttgagtaaggggcaattaaaggagtttcttgatgccaaccttgacggaggaagcggtggaggtagcatgtctggtaaaattgataaaattttgattgttggtggtggtactgctggttggatggctgcttcttatttgggtaaagctttgcaaggtactgctgatattactttgttgcaagctccagatattccaactttgggtgttggtgaagctactattccaaatttgcaaactgctttttttgattttttgggtattccagaagatgaatggatgagagaatgtaatgcttcttataaagttgctattaaatttattaattggagaactgctggtgaaggtacttctgaagctagagaattggatggtggtccagatcatttttatcattcttttggtttgttgaaatatcatgaacaaattccattgtctcattattggtttgatagatcttatagaggtaaaactgttgaaccatttgattatgcttgttataaagaaccagttattttggatgctaatagatctccaagaagattggatggttctaaagttactaattatgcttggcattttgatgctcatttggttgctgattttttgagaagatttgctactgaaaaattgggtgttagacatgttgaagatagagttgaacatgttcaaagagatgctaatggtaatattgaatctgttagaactgctactggtagagtttttgatgctgatttgtttgttgattgttctggttttagaggtttgttgattaataaagctatggaagaaccatttttggatatgtctgatcatttgttgaatgattctgctgttgctactcaagttccacatgatgatgatgctaatggtgttgaaccatttacttctgctattgctatgaaatctggttggacttggaaaattccaatgttgggtagatttggtactggttatgtttattcttctagatttgctactgaagatgaagctgttagagaattttgtgaaatgtggcatttggatccagaaactcaaccattgaatagaattagatttagagttggtagaaatagaagagcttgggttggtaattgtgtttctattggtacttcttcttgttttgttgaaccattggaatctactggtatttattttgtttatgctgctttgtatcaattggttaaacattttccagataaatctttgaatccagttttgactgctagatttaatagagaaattgaaactatgtttgatgatactagagattttattcaagctcatttttatttttctccaagaactgatactccattttggagagctaataaagaattgagattggctgatggtatgcaagaaaaaattgatatgtatagagctggtatggctattaatgctccagcttctgatgatgctcaattgtattatggtaattttgaagaagaatttagaaatttttggaataattctaattattattgtgttttggctggtttgggtttggttccagatgctccatctccaagattggctcatatgccacaagctactgaatctgttgatgaagtttttggtgctgttaaagatagacaaagaaatttgttggaaactttgccatctttgcatgaatttttgagacaacaacatggtagataa

SEQ ID NO: 153 - protein, 530 aa, Lechevalieria aerocolonigenes ATCC 39243
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 154 - DNA, 1593 bp, synthetic construct
  source 1..1593: mol_type = other DNA
atgtctggtaaaattgataaaattttgattgttggtggtggtactgctggttggatggctgcttcttatttgggtaaagctttgcaaggtactgctgatattactttgttgcaagctccagatattccaactttgggtgttggtgaagctactattccaaatttgcaaactgctttttttgattttttgggtattccagaagatgaatggatgagagaatgtaatgcttcttataaagttgctattaaatttattaattggagaactgctggtgaaggtacttctgaagctagagaattggatggtggtccagatcatttttatcattcttttggtttgttgaaatatcatgaacaaattccattgtctcattattggtttgatagatcttatagaggtaaaactgttgaaccatttgattatgcttgttataaagaaccagttattttggatgctaatagatctccaagaagattggatggttctaaagttactaattatgcttggcattttgatgctcatttggttgctgattttttgagaagatttgctactgaaaaattgggtgttagacatgttgaagatagagttgaacatgttcaaagagatgctaatggtaatattgaatctgttagaactgctactggtagagtttttgatgctgatttgtttgttgattgttctggttttagaggtttgttgattaataaagctatggaagaaccatttttggatatgtctgatcatttgttgaatgattctgctgttgctactcaagttccacatgatgatgatgctaatggtgttgaaccatttacttctgctattgctatgaaatctggttggacttggaaaattccaatgttgggtagatttggtactggttatgtttattcttctagatttgctactgaagatgaagctgttagagaattttgtgaaatgtggcatttggatccagaaactcaaccattgaatagaattagatttagagttggtagaaatagaagagcttgggttggtaattgtgtttctattggtacttcttcttgttttgttgaaccattggaatctactggtatttattttgtttatgctgctttgtatcaattggttaaacattttccagataaatctttgaatccagttttgactgctagatttaatagagaaattgaaactatgtttgatgatactagagattttattcaagctcatttttatttttctccaagaactgatactccattttggagagctaataaagaattgagattggctgatggtatgcaagaaaaaattgatatgtatagagctggtatggctattaatgctccagcttctgatgatgctcaattgtattatggtaattttgaagaagaatttagaaatttttggaataattctaattattattgtgttttggctggtttgggtttggttccagatgctccatctccaagattggctcatatgccacaagctactgaatctgttgatgaagtttttggtgctgttaaagatagacaaagaaatttgttggaaactttgccatctttgcatgaatttttgagacaacaacatggtagataa